SEQUENCE LISTING (4 sequences)

SEQ ID NO: 1 - protein, 214 aa, synthetic construct
DIQMTQSPSSVSASVGDRVTITCRASQGIGSWLAWYQQKPGKAPKLLIYAADRLQSGVPSRFSGSGSGTDFTLTISSLQPEDFATYYCQQYHTYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 453 aa, synthetic construct
QVQLVESGGGVVQPGRSLRLSCAASGFNFSDYGMHWVRQAPGKGLEWVAVIWYDGSISYYADSVKGRFTISRDNSKNTLYLQMNSLRAEDTAVYYCARGGTVEHGAVYGTDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 216 aa, synthetic construct
DIVMTQSPSSLSASVGDRVTITCRASQSISRYLNWYQQKPGKAPKLLIYAASSLQSGVPSRFSGSGSGTDFTLTISSLQPEDFATYYCQQSYSTPPEYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 448 aa, synthetic construct
EVQLVESGGGLVQPGGSLRLSCAASGFTVSSNYMSWVRQAPGKGLEWVSVIYSGGSTFYADSVKGRFTISRDNSMNTLFLQMNSLRAEDTAVYYCARVLPMYGDYLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0166167 A1
(43) Pub. Date: Jun. 18, 2026

(54) Bioactive Conjugates, Preparation Method and Use Thereof

(71) Applicant: BeOne Medicines I GmbH, Basel (CH)

(72) Inventors: Charng-Sheng Tsai, Beijing (CN); Mei-Hsuan Tsai, Beijing (CN); Maomao He, Beijing (CN); Zewei Wang, Beijing (CN); Kejie Zhu, Beijing (CN); Xiaokun Yang, Beijing (CN); Yayun Chen, Beijing (CN); Wei Luo, Beijing (CN)

(73) Assignee: BeOne Medicines I GmbH, Basel (CH)

(21) Appl. No.: 19/531,014

(22) Filed: Feb. 5, 2026

Related U.S. Application Data
(63) Continuation of application No. PCT/IB24/57756, filed on Aug. 9, 2024.

(30) Foreign Application Priority Data
Aug. 10, 2023 (WO) PCTCN2023112181

(51) Int. Cl. A61K 47/68; A61P 35/00; C07K 16/28

(52) U.S. Cl. CPC A61K 47/68035; A61K 47/6849; A61K 47/6889; A61P 35/00; C07K 16/2803

(57) ABSTRACT

Provided herein are compounds comprising pyrrolobenzodiazepine (PBD)-based dimers and conjugates thereof. In some embodiments, a compound is of Formula (I):

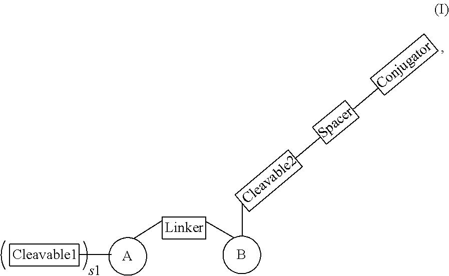

or a pharmaceutically acceptable salt, tautomer, solvate, stereoisomer, or isotopologue thereof.

### CROSS REFERENCE TO RELATED APPLICATIONS

This application is a continuation of International Application No. PCT/IB2024/057756, filed Aug. 9, 2024, which claims priority to International Application No. PCT/CN2023/112181, filed Aug. 10, 2023, the disclosures of each of which are hereby incorporated by reference in their entireties.

### FIELD

The present disclosure relates to antibody drug conjugate platform compounds comprising pyrrolobenzodiazepine (PBD)-based dimers and antibody drug conjugates (ADCs) comprising the platforms and an antibody, or antigen-binding fragment thereof, as well as uses of the ADC platforms and ADCs.

### SEQUENCE LISTING

This application contains a Sequence Listing, which has been submitted electronically in XML format. The XML file is entitled “01368-0077-00PCT-ST26.xml,” was created on Aug. 7, 2024, and is 5,839 bytes in size. The Sequence Listing is incorporated herein by reference in its entirety.

### BACKGROUND

Some pyrrolobenzodiazepines (PBDs) have the ability to recognize and bind to specific sequences of DNA; the preferred sequence is PuGPu. The first PBD antitumor antibiotic, anthramycin, was discovered in 1965 (Leimgruber, et al., J. Am. Chem. Soc., 87, 5793-5795 (1965); Leimgruber, et al., J. Am. Chem. Soc., 87, 5791-5793 (1965)). Since then, a number of naturally occurring PBDs have been reported, and more than ten synthetic routes have been developed for a variety of analogues (Thurston, et al., Chem. Rev. 1994, 433-465 (1994)). Family members include abbeymycin (Hochlowski, et al., J. Antibiotics, 40, 145-148 (1987)), chicamycin (Konishi, et al., J. Antibiotics, 37, 200-206 (1984)), DC-81 (Japanese Patent 58-180 487; Thurston, et al., Chem. Brit., 26, 767-772 (1990); Bose, et al., Tetrahedron, 48, 751-758 (1992)), mazethramycin (Kuminoto, et al., J. Antibiotics, 33, 665-667 (1980)), neothramycins A and B (Takeuchi, et al., J. Antibiotics, 29, 93-96 (1976)), porothramycin (Tsunakawa, et al., J. Antibiotics, 41, 1366-1373 (1988)), prothracarcin (Shimizu, et al, J. Antibiotics, 29, 2492-2503 (1982); Langley and Thurston, J. Org. Chem., 52, 91-97 (1987)), sibanomicin (DC-102) (Hara, et al., J. Antibiotics, 41, 702-704 (1988); Itoh, et al., J. Antibiotics, 41, 1281-1284 (1988)), sibiromycin (Leber, et al., J. Am. Chem. Soc., 110, 2992-2993 (1988)), and tomamycin (Arima, et al., J. Antibiotics, 25, 437-444 (1972)). PBDs have the following general structure:

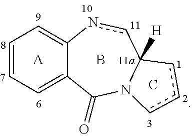

PBDs differ in the number, type, and position of substituents, in both their aromatic A rings and pyrrolo C rings, and in the degree of saturation of the C ring. In the B-ring there is an imine (N═C), a carbinolamine (NH—CH(OH)), or a carbinolamine methyl ether (NH—CH(OMe)) at the N10-C11 position, which is the electrophilic center responsible for alkylating DNA. All of the known natural products have an (S)-configuration at the chiral C11a position which provides them with a right-handed twist when viewed from the C ring towards the A ring. This gives them the appropriate three-dimensional shape for isohelicity with the minor groove of B-form DNA, leading to a snug fit at the binding site (Kohn, In Antibiotics III. Springer-Verlag, New York, pp. 3-11 (1975); Hurley and Needham-VanDevanter, Acc. Chem. Res., 19, 230-237 (1986)). The ability of PBDs to form an adduct in the minor groove enables them to interfere with DNA processing, hence their use as antitumor agents.

The biological activity of these molecules can be potentiated by joining two PBD units together through their C8/C′-hydroxyl functionalities via a flexible alkylene linker (Bose, D. S., et al., J. Am. Chem. Soc., 114, 4939-4941 (1992); Thurston, D. E., et al., J. Org. Chem., 61, 8141-8147 (1996)). The PBD dimers are thought to form sequence-selective DNA lesions such as the palindromic 5′-Pu-GATC-Py-3′ interstrand cross-link (Smellie, M., et al., Biochemistry, 42, 8232-8239 (2003); Martin, C., et al., Biochemistry, 44, 4135-4147) which is thought to be mainly responsible for their biological activity. One example of a PBD dimer is SG2000 (SJG-136):

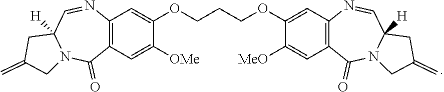

(Gregson, S., et al., J. Med. Chem., 44, 737-748 (2001); Alley, M. C., et al., Cancer Research, 64, 6700-6706 (2004); Hartley, J. A., et al., Cancer Research, 64, 6693-6699 (2004)).

Due to the manner in which these highly potent compounds act to cross-link DNA, previous PBD dimers have been made symmetrically, i.e., both monomers of the dimer are the same. This synthetic route provides for straightforward synthesis, either by constructing the PBD dimer moiety simultaneously having already formed the dimer linkage, or by reacting already constructed PBD monomer moieties with the dimer linking group. These synthetic approaches have limited the options for preparation of targeted conjugates containing PBDs.

Antibody-drug conjugates (ADCs) include antibodies operably linked to a biologically active small molecule, also known as a toxin or payload. ADCs deliver a potent payload selectively to target-expressing cells, leading to a potential reduction of off-target side effects and/or toxicity and improved therapeutic efficacy. The lipophilic nature of many payloads can adversely affect the properties of the ADC to the extent that the payloads are not efficiently delivered to the target cells. Low bioavailability of lipophilic payloads can narrow therapeutic windows for ADC treatment. Furthermore, the hydrophobic nature of payloads can present challenges to their conjugation to antibodies, a reaction performed in aqueous conditions.

There is an ongoing need for the development of hydrophilic linkers for protein conjugates, e.g., ADCs, which would allow for improved conjugation of lipophilic payloads, improved modulation of biological targets, improved bioavailability, and improved therapeutic windows. There is an ongoing need for PBD dimers, including lipophilic and/or asymmetrical PBD dimers as payloads in ADCs.

### BRIEF SUMMARY

Provided herein are antibody drug conjugate platforms and antibody drug conjugates (ADCs). Also provided are uses of the ADC platforms to prepare ADCs.

In some embodiments, provided herein are compounds of Formula (I):

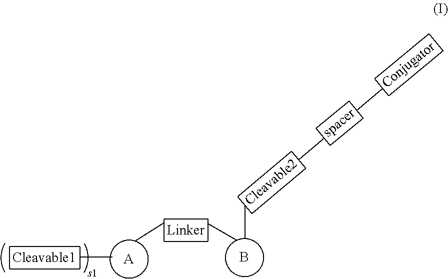

or a pharmaceutically acceptable salt, tautomer, solvate, stereoisomer, or isotopologue thereof, wherein:
each of ring A and ring B is, independently, one of formula (IIa), (IIb), (IIc), (IId), (IIe), (IIf), or (IIg):

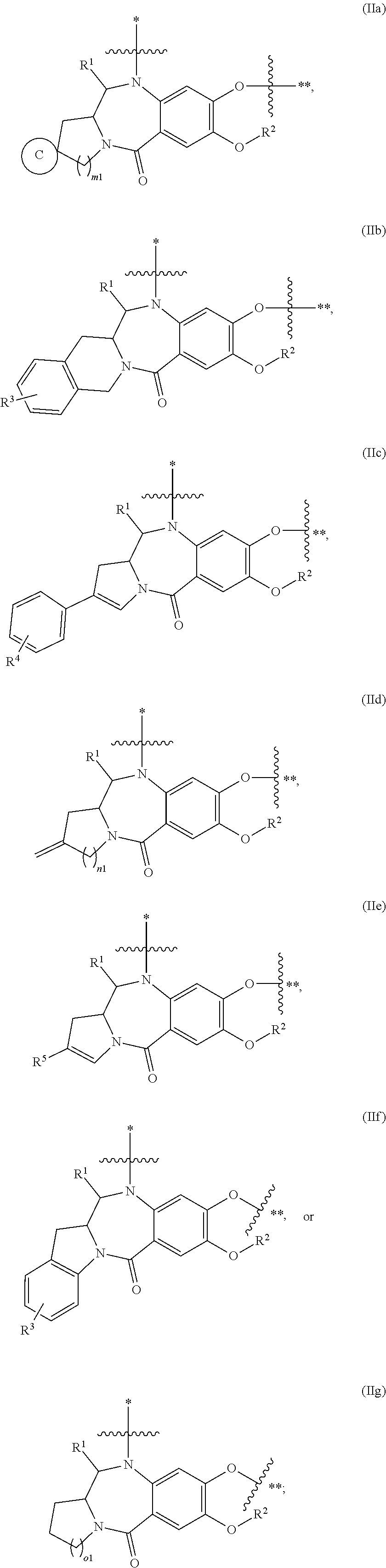

wherein:
each ring C is independently a cyclopropyl ring or a cyclobutyl ring;
each R¹ is independently H, OH, C₁₋₄ alkyl, C₁₋₄ alkoxyl, or —O—(CH₂CH₂O)ₜ₁—CH₃;
each R² is independently H or C₁₋₄ alkyl;
each R³ and R⁴ is, independently, H, NR^{a1}R^{b1}, OH, C₁₋₄ alkyl, C₁₋₄ alkoxyl, or aryl;
each R^{a1} and R^{b1} is, independently, H or C₁₋₄ alkyl;
each R⁵ is independently H, C₁₋₄ alkyl, C₁₋₄ alkoxyl, or aryl;
each m1, n1, and o1 is, independently, 1 or 2;
t1 is 1, 2, 3, 4, 5, 6, 7, or 8;
* marks the bond where ring A connects to Cleavable1 when s1 is 1 or to —H when s1 is 0, or where ring B connects to Cleavable2, and
** marks the bond where ring A or ring B connects to Linker; Linker is —(CH₂)ᵣ—, —(CH₂)ₚ—X—(CH₂)_{q}—, or —(CH₂)ₚ—CH═CH—(CH₂)_{q}—; wherein:
each r, p, and q, is, independently, 1, 2, 3, 4, 5, 6, 7, or 8;
the sum of p and q is 1, 2, 3, 4, 5, 6, 7, or 8;
X is NR⁶, NHC(═O), C(═O)NH, O, SO₂, a substituted or unsubstituted aryl ring, substituted or unsubstituted heteroaryl ring, substituted or unsubstituted heterocyclic ring, or substituted or unsubstituted cyclic ring; and
R⁶ is H or C₁₋₄ alkyl;
Cleavable1, if present, has formula (VIa) or (VIc):

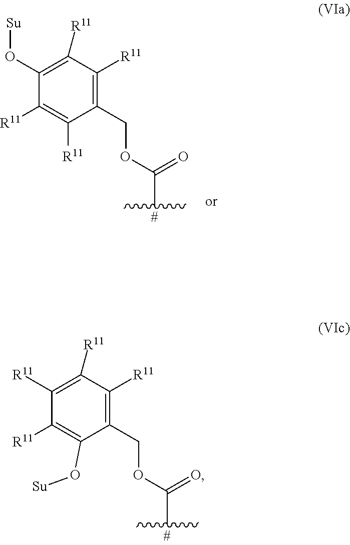

wherein:
Su is a sugar moiety;
each R¹¹ is, independently, hydrogen, halogen, substituted or unsubstituted C₁₋₄ alkyl, —CN, or —NO₂; and
# marks the bond where Cleavable1 connects to ring A;
s1 is 0 or 1;
Cleavable2 has formula (VIIa1), (VIIc1), (VIId1), or (VIIf1):

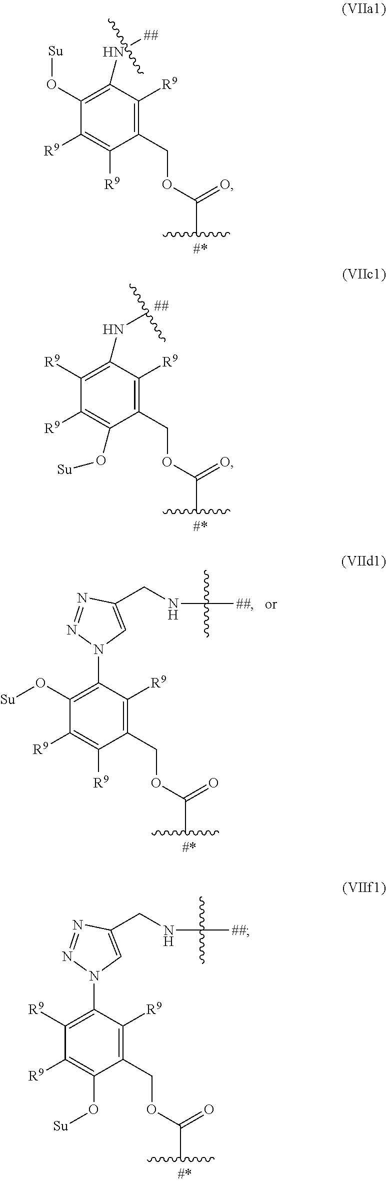

wherein:
Su is a sugar moiety;
each R⁹ is, independently, hydrogen, halogen, substituted or unsubstituted C₁₋₄ alkyl, —CN, or —NO₂;
#* marks the bond where Cleavable2 connects to ring B; and
## marks the bond where Cleavable2 connects to Spacer;
Spacer is a bond, ^{###}—NH—(CH₂CH₂O)ₘ₂—CH₂CH₂—C(═O)—, ^{###}—NH—(CH₂CH₂O)ₘ₂—CH₂—C(═O)—, ^{###}—(CH₂)ₘ₂—C(═O)—, ^{###}—CH₂—C(═O)—NH—(CH₂)ₘ₂—C(═O)—, ^{###}—(CH₂CH₂O)ₘ₂—CH₂CH₂—C(═O)—, ^{###}—CH[—(CH₂)ₘ₂—COOH]—C(═O)—, ^{###}—CH₂—C(═O)—NH—(CH₂)ₘ₂-C(═O)—NH—(CH₂)ₘ₂ —C(═O)—, ^{###}—C(═O)—(CH₂)ₘ₂—C(═O)—, or ###—NH—(CH₂)ₘ₂—C(═O)—; wherein:
each m2 is, independently, 1, 2, 3, 4, 6, 7, or 8; and
### marks the bond where Spacer connects to Conjugator; and Conjugator has formula (III), (IV), or (V):

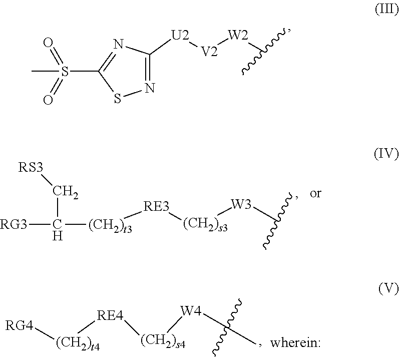

U2 is a bond, heteroarylene, or arylene;
V2 is a bond or —C≡C—(CH₂)ₙ₂—;
n2 is an integer from 0 to 10 inclusive;
W2 is —C(═O)—, —NH— or —O—;
RG3 is

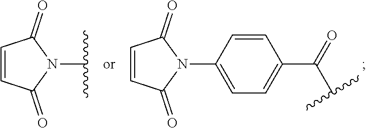 [SMILES: *N1C(=O)C=CC1=O ; *C(=O)c1ccc(N2C(=O)C=CC2=O)cc1]

RS3 is —NR^{a2}R^{b2};
each of R^{a2} and R^{b2} is, independently, H or substituted or unsubstituted C₁₋₄ alkyl;
RE3 is a bond, —O—, —OC(═O)—, —OC(═O)NR⁷—, —NHC(═O)NR⁷—, —OS(═O)₂NR⁷—, —NHS(═O)₂NR⁷—, or —OC(═O)NHS(═O)₂NR⁷—;
R⁷ is H or substituted or unsubstituted C₁₋₄ alkyl;
W3 is —C(═O)—, —NH—, or —O—;
t3 is 1 or 2;
s3 is 0, 1, or 2;

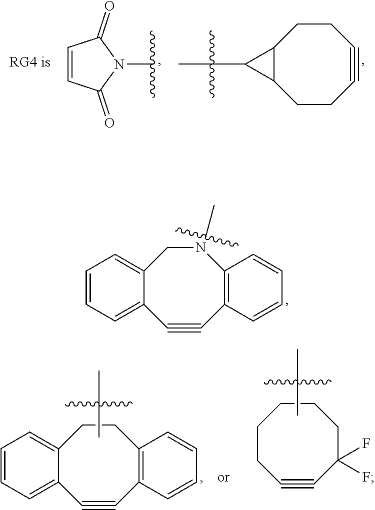

RE4 is a bond, —O—, —OC(═O)—, —OC(═O)NR⁸—, —NHC(═O)NR⁸—, —OS(═O)₂NR⁸—, —NHS(═O)₂NR⁸—, or —OC(═O)NHS(═O)₂NR⁸—;
R⁸ is H or substituted or unsubstituted C₁₋₄ alkyl;
W4 is —C(═O)—, —NH—, or —O—;
t4 is 1, 2, 3, 4, 5, 6, 7, or 8; and
s4 is 0, 1, or 2.

In some embodiments, provided herein are compounds of Formula (IA):

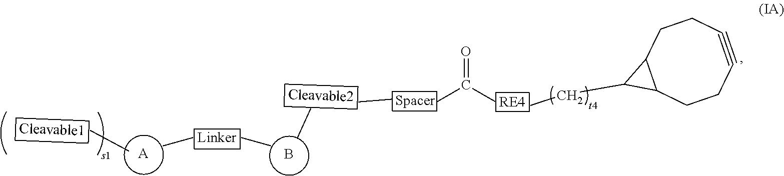

or a pharmaceutically acceptable salt, tautomer, solvate, stereoisomer, or isotopologue thereof, wherein:
each of ring A and ring B is, independently, one of formula (IIa), (IIb), (IIc), (IId), (IIe), (IIf), or (IIg):

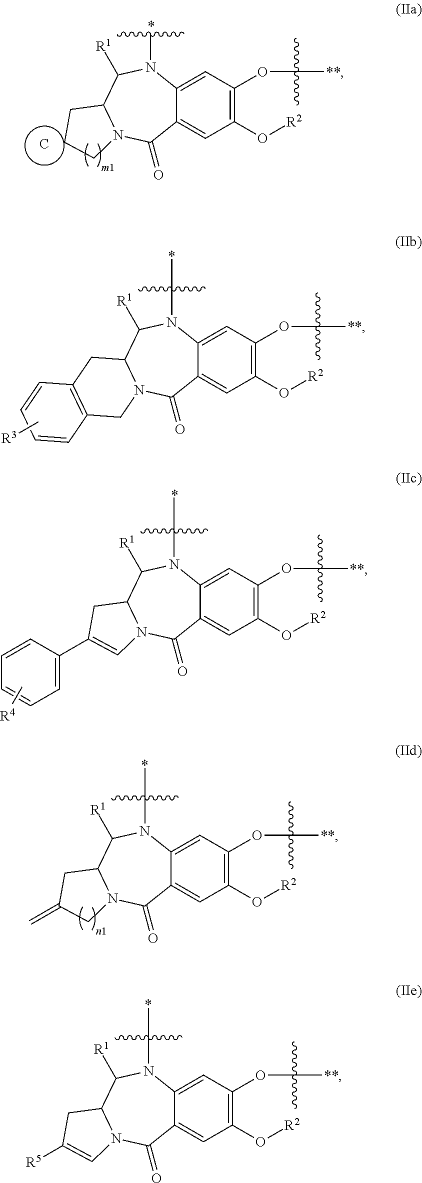

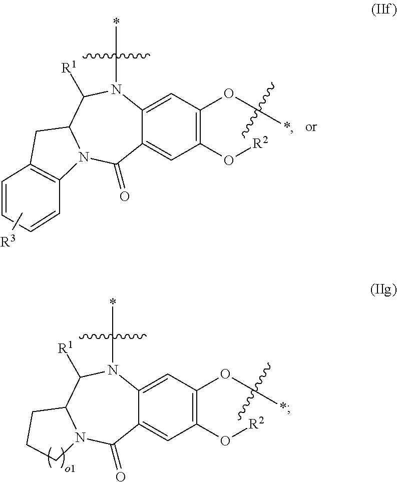

wherein:
each ring C is independently a cyclopropyl ring or a cyclobutyl ring;
each R¹ is independently H, OH, C₁₋₄ alkyl, C₁₋₄ alkoxyl, or —O—(CH₂CH₂O)ₜ₁—CH₃;
each R² is independently H or C₁₋₄ alkyl;
each R³ and R⁴ is, independently, H, NR^{a1}R^{b1}, OH, C₁₋₄ alkyl, C₁₋₄ alkoxyl, or aryl;
each R^{a1} and R^{b1} is, independently, H or C₁₋₄ alkyl;
each R⁵ is independently H, C₁₋₄ alkyl, C₁₋₄ alkoxyl, or aryl;
each m1, n1, and o1 is, independently, 1 or 2;
t1 is 1, 2, 3, 4, 5, 6, 7, or 8;
* marks the bond where ring A connects to Cleavable1 when s1 is 1 or to —H when
s1 is 0, or where ring B connects to Cleavable2; and
** marks the bond where ring A or ring B connects to Linker;
Linker is —(CH₂)ᵣ—, —(CH₂)ₚ—X—(CH₂)_{q}—, or —(CH₂)ₚ—CH═CH—(CH₂)_{q}—; wherein:
each r, p, and q, is, independently, 1, 2, 3, 4, 5, 6, 7, or;
the sum of p and q is 1, 2, 3, 4, 5, 6, 7, or;
X is NR⁶, NHC(═O), C(═O)NH, O, SO₂, a substituted or unsubstituted aryl ring, substituted or unsubstituted heteroaryl ring, substituted or unsubstituted heterocyclic ring, or substituted or unsubstituted cyclic ring; and
R⁶ is H or C₁₋₄ alkyl;
Cleavable1, if present, has the formula

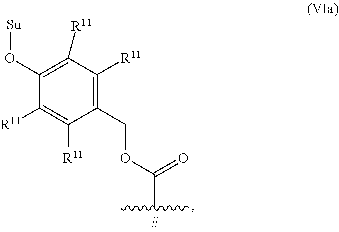

wherein:
Su is a sugar moiety;
each R¹¹ is, independently, hydrogen, halogen, substituted or unsubstituted C₁₋₄ alkyl, —CN, or —NO₂; and
# marks the bond where Cleavable1 connects to ring A;
s1 is 0 or 1;
Cleavable2 has formula (VIIa1) or (VIId1):

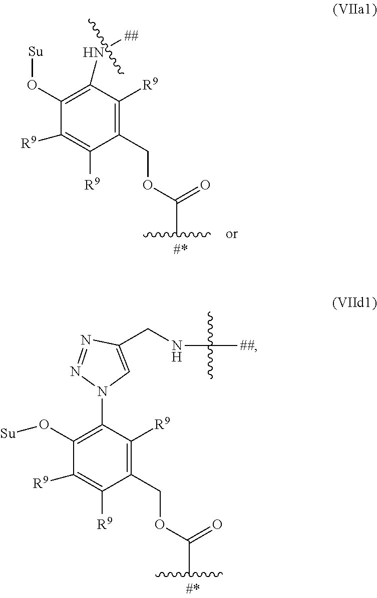

wherein:
Su is a sugar moiety;
each R⁹ is, independently, hydrogen or C₁₋₄ alkyl;
#* marks the bond where Cleavable2 connects to ring B; and
## marks the bond where Cleavable2 connects to Spacer;
Spacer is a bond, ^{###}—NH—(CH₂CH₂O)ₘ₂—CH₂CH₂—C(═O)— or ^{###}—NH—(CH₂CH₂O)ₘ₂—CH₂—C(═O)—; wherein:
each m2 is, independently, 1, 2, 3, 4, 6, 7, or 8; and
### marks the bond where Spacer connects to —C(═O)—; RE4 is a bond or —O—; and
t4 is 1, 2, 3, 4, 5, 6, 7, or 8.

### BRIEF DESCRIPTION OF FIGURES

FIGS. 1A and 1B are line graphs showing killing of NOMO-1 (1A) and K562 (1B) cells by the indicated conjugates.

FIGS. 2A and 2B are line graphs showing killing of NOMO-1 (2A) and K562 (2B) cells by the indicated conjugates.

FIGS. 3A and 3B are line graphs showing killing of NOMO-1 (3A) and K562 (3B) cells by the indicated conjugates.

FIGS. 4A and 4B are line graphs showing killing of NOMO-1 (4A) and K562 (4B) cells by the indicated conjugates.

FIGS. 5A and 5B are line graphs showing killing of NOMO-1 (5A) and K562 (5B) cells by the indicated conjugates.

FIGS. 6A and 6B are line graphs showing killing of NOMO-1 (6A) and K562 (6B) cells by the indicated conjugates.

### DETAILED DESCRIPTION

Provided herein are antibody drug conjugate platform compounds comprising pyrrolobenzodiazepine (PBD)-based dimers and antibody drug conjugates (ADCs) comprising the platforms and an antibody, or antigen-binding fragment thereof. The ADCs may be used to treat a disease or disorder, such as cancer, such as by providing a composition comprising an ADC. The conjugates include a β-glucuronide-based linker comprising a site that can be cleaved by an enzyme having β-glucuronidase activity

Within the present disclosure, it is understood that the disclosure is not limited to particular methods and/or experimental conditions described, as such methods and conditions may vary. It is also to be understood that the terminology used herein is for the purpose of describing particular embodiments only, and is not intended to be limiting,

Although any methods and materials similar or equivalent to those described herein may be used in the practice or testing of the present invention, the preferred methods and materials are now described. All patents, applications, and non-patent publications mentioned in this specification are incorporated herein by reference in their entireties.

### Definitions

In the present disclosure, the following terms have the following meanings unless indicated otherwise. Unless defined otherwise, all technical and scientific terms used herein have the same meaning as is commonly understood by one of ordinary skill in the art to which this disclosure pertains. In the event that there is a plurality of definitions for a term provided herein, these Definitions prevail unless stated otherwise.

When a trade name is used herein, reference to the trade name also refers to the product formulation, the generic drug, and the active pharmaceutical ingredient(s) of the trade name product, unless otherwise indicated by context.

The term “antibody” herein is used in the broadest sense and specifically covers intact monoclonal antibodies, polyclonal antibodies, monospecific antibodies, multispecific antibodies (e.g., bispecific antibodies), and antibody fragments that exhibit the desired biological activity. An intact antibody has primarily two regions: a variable region and a constant region. The variable region binds to and interacts with a target antigen. The variable region includes a complementary determining region (CDR) that recognizes and binds to a specific binding site on a particular antigen. The constant region may be recognized by and interact with the immune system (see, e.g., Janeway et al., 2001, Immuno. Biology, 5^{th} Ed., Garland Publishing, New York). An antibody can be of any type (e.g., IgG, IgE, IgM, IgD, and IgA), class (e.g., IgG1, IgG2, IgG3, IgG4, IgAQ1, and IgA2) or subclass. The antibody can be derived from any suitable species. In some embodiments, the antibody is of human or murine origin. An antibody can be, for example, human, humanized, or chimeric.

The term “humanized” or “humanized antibody” means forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fe), typically that of a human immunoglobulin. The prefix “hum,” “hu,” “Hu,” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions can be included to increase affinity, increase stability of the humanized antibody, remove a post-translational modification or for other reasons

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. The modifier “monoclonal” is not to be construed as requiring production of the antibody by any particular method.

An “intact antibody” is one that comprises an antigen-binding variable region as well as a light chain constant domain (CL) and heavy chain constant domains, CH1, CH2, CH3, and CH4, as appropriate for the antibody class. The constant domains may be native sequence constant domains (e.g., human native sequence constant domains) or amino acid sequence variant thereof.

An “antibody fragment” comprises a portion of an intact antibody, comprising the antigen-binding or variable region thereof. Examples of antibody fragments include Fab, Fab′, F(ab′)₂, and Fv fragments, diabodies, triabodies, tetrabodies, linear antibodies, single-chain antibody molecules, scFv, scFv-Fc, multispecific antibody fragments formed from antibody fragment(s), a fragment(s) produced by a Fab expression library, or an epitope-binding fragment of any of the above which immunospecifically binds to a target antigen (e.g., a cancer cell antigen, a viral antigen or a microbial antigen).

An “antigen” is an entity to which an antibody specifically binds.

The terms “specific binding” and “specifically binds” mean that the antibody or antibody derivative will bind, in a highly selective manner, to its corresponding target antigen and not with the multitude of other antigens. Typically, the antibody or antibody derivative binds with an affinity of at least about 1×10⁻⁷ M, 10⁻⁸ M, 10⁻⁹M, 10⁻¹⁰ M, 10⁻¹¹ M, or 10⁻¹² M and binds to the predetermined antigen with an affinity that is at least two-fold greater than its affinity for binding to a non-specific antigen (e.g., BSA, casein) other than the predetermined antigen or a closely related antigen.

The term “inhibit” or “inhibition of” means to reduce by a measurable amount, or to prevent entirely.

The term “therapeutically effective amount” refers to an amount of a drug effective to treat a disease or disorder in a mammal. In the case of cancer, the therapeutically effective amount of a drug may reduce the number of cancer cells; reduce the tumor size; inhibit (i.e., slow to some extent or stop) cancer cell infiltration into peripheral organs; inhibit (i.e., slow to some extent or stop) tumor metastasis; inhibit, to some extent, tumor growth; and/or relieve to some extent one or more of the symptoms associated with the cancer. To the extent the drug may inhibit growth and/or kill existing cancer cells, it may be cytostatic and/or cytotoxic. For cancer therapy, efficacy can, for example, be measured by assessing the time to disease progression (TTP) and/or determining the response rate (RR).

The term “substantial” or “substantially” refers to a majority, i.e. >50% of a population, of a mixture or a sample, preferably more than 50%, 55%, 60%, 65%, 70%, 75%, 80%, 85%, 90%, 91%, 92%, 93%, 94%, 95%, 96%, 97%, 98%, or 99% of a population.

The terms “intracellularly cleaved” and “intracellular cleavage” refer to a metabolic process or reaction inside a cell on a ligand drug conjugate (e.g., an antibody drug conjugate (ADC)), whereby the covalent attachment, e.g., the linker, between the drug moiety (D) and the ligand unit (e.g., an antibody (BA or Ab)) is broken, resulting in the free drug, or another metabolite of the conjugate dissociated from the antibody inside the cell. The cleaved moieties of the drug-linker-ligand conjugate are thus intracellular metabolites.

The term “cytotoxic activity” refers to a cell-killing, a cytostatic or an anti-proliferative effect of a drug-linker-ligand conjugate compound or an intracellular metabolite of a drug-linker-ligand conjugate. Cytotoxic activity may be expressed as the IC50 value, which is the concentration (molar or mass) per unit volume at which half the cells survive.

The term “cytotoxic agent” as used herein refers to a substance that inhibits the function of cells and/or causes destruction of cells. The term is intended to include radioactive isotopes (e.g., 211At, 131I, 125I, 90Y, 186Re, 188Re, 153Sm, 212Bi, 32P, 60C, and radioactive isotopes of Lu), chemotherapeutic agents, and toxins such as small molecule toxins or enzymatically active toxins of bacterial, fungal, plant, or animal origin, including synthetic analogs and derivatives thereof.

The terms “cancer” and “cancerous” refer to or describe the physiological condition or disorder in mammals that is typically characterized by unregulated cell growth. A “tumor” comprises one or more cancerous cells.

An “autoimmune disease” herein is a disease or disorder arising from and directed against an individual's own tissues or proteins.

Examples of a “patient” or “subject” include, but are not limited to, mammals such as a human, rat, mouse, guinea pig, monkey, pig, goat, cow, horse, dog, or cat, and birds or fowl. In an embodiment, the patient is a human.

The terms “treat” or “treatment,” unless otherwise indicated by context, refer to therapeutic treatment and prophylactic measures to prevent relapse, wherein the object is to inhibit or slow down (lessen) an undesired physiological change or disorder, such as the development or spread of cancer. For purposes of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder.

In the context of cancer, the term “treating” includes any or all of inhibiting growth of tumor cells, cancer cells, or of a tumor, inhibiting replication of tumor cells or cancer cells, lessening of overall tumor burden or decreasing the number of cancerous cells, and ameliorating one or more symptoms associated with the disease.

In the context of an autoimmune disease, the term “treating” includes any or all of inhibiting replication of cells associated with an autoimmune disease state including, but not limited to, cells that produce an autoimmune antibody, lessening the autoimmune-antibody burden, and ameliorating one or more symptoms of an autoimmune disease.

As used herein, and in the specification and the accompanying claims, the indefinite articles “a” and “an” and the definite article “the” include the plural as well as single referents, unless the context clearly indicates otherwise.

As used herein, and unless otherwise specified, the terms “about” and “approximately,” when used in connection with amounts, or weight percentage of ingredients of a composition, mean an amount or weight percent that is recognized by one of ordinary skill in the art to provide a pharmacological effect equivalent to that obtained from the specified amount or weight percent. In certain embodiments, the terms “about” and “approximately,” when used in this context, contemplate an amount or weight percent within 30%, within 20%, within 15%, within 10%, or within 5%, of the specified amount or weight percent.

As used herein, and unless otherwise specified, the terms “about” and “approximately,” when used in connection with a numeric value or range of values that is provided to characterize a particular solid form, e.g., a specific temperature or temperature range, such as, for example, that describes a melting, dehydration, desolvation, or glass transition temperature; a mass change, such as, for example, a mass change as a function of temperature or humidity; a solvent or water content, in terms of, for example, mass or a percentage; or a peak position, such as, for example, in analysis by, for example, IR or Raman spectroscopy or XRPD; indicate that the value or range of values may deviate to an extent deemed reasonable to one of ordinary skill in the art while still describing the solid form. Techniques for characterizing crystal forms and amorphous solids include, but are not limited to, thermal gravimetric analysis (TGA), differential scanning calorimetry (DSC), X-ray powder diffractometry (XRPD), single-crystal X-ray diffractometry, vibrational spectroscopy, e.g., infrared (IR) and Raman spectroscopy, solid-state and solution nuclear magnetic resonance (NMR) spectroscopy, optical microscopy, hot stage optical microscopy, scanning electron microscopy (SEM), electron crystallography and quantitative analysis, particle size analysis (PSA), surface area analysis, solubility studies, and dissolution studies. In certain embodiments, the terms “about” and “approximately,” when used in this context, indicate that the numeric value or range of values may vary within 30%, 20%, 15%, 10%, 9%, 8%, 7%, 6%, 5%, 4%, 3%, 2%, 1.5%, 1%, 0.5%, or 0.25% of the recited value or range of values. For example, in some embodiments, the value of an XRPD peak position may vary by up to ±0.2° 20 while still describing the particular XRPD peak.

As used herein, the term “inclusive,” when used in reference to range, includes the endpoints of the range. For example, if n is an integer from 0 to 4, n may be any of 0, 1, 2, 3, or 4.

An “alkyl” group is a saturated, partially saturated, or unsaturated straight chain or branched non-cyclic hydrocarbon having from 1 to 10 carbon atoms, typically from 1 to 8 carbons or, in some embodiments, from 1 to 6, 1 to 4, or 2 to 6 carbon atoms. Representative alkyl groups include -methyl, -ethyl, -n-propyl, -n-butyl, -n-pentyl, and n-hexyl; saturated branched alkyls include -isopropyl, -sec-butyl, -isobutyl, -tert-butyl, -isopentyl, 2-methylpentyl, 3-methylpentyl, 4-methylpentyl, 2,3-dimethylbutyl and the like. Examples of unsaturated alkyl groups include, but are not limited to, vinyl, allyl, CH═CH(CH₃), —CH═C(CH₃)₂, —C(CH₃)═CH₂, —C(CH₃)═CH(CH₃), C(CH₂CH₃)═CH₂, C≡CH, —C≡C(CH₃), —C≡C(CH₂CH₃), —CH₂C≡CH, —CH₂C≡C(CH₃), and CH₂C≡C(CH₂CH₃), among others. An alkyl group can be substituted or unsubstituted. In certain embodiments, when the alkyl groups described herein are said to be “substituted,” they may be substituted with any substituent or substituents as those found in the compounds and embodiments disclosed herein, as well as halogen (chloro, iodo, bromo, or fluoro); hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂; or O(alkyl)aminocarbonyl.

An “alkenyl” group is a straight chain or branched non-cyclic hydrocarbon having from 2 to 10 carbon atoms, typically from 2 to 8 carbon atoms, and including at least one carbon-carbon double bond. Representative straight chain and branched (C₂-C₈)alkenyls include -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, 2-hexenyl, -3-hexenyl, -1-heptenyl, -2-heptenyl, -3-heptenyl, -1-octenyl, -2-octenyl, 3-octenyl and the like. The double bond of an alkenyl group can be unconjugated or conjugated to another unsaturated group. An alkenyl group can be unsubstituted or substituted.

A “cycloalkyl” group is a saturated or a partially saturated cyclic alkyl group of from 3 to 10 carbon atoms having a single cyclic ring or multiple condensed or bridged rings which can be optionally substituted with from 1 to 3 alkyl groups. In some embodiments, the cycloalkyl group has 3 to 8 ring members, whereas in other embodiments the number of ring carbon atoms ranges from 3 to 5, 3 to 6, or 3 to 7. Such cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as adamantyl and the like. Examples of unsaturated cycloalkyl groups include cyclohexenyl, cyclopentenyl, cyclohexadienyl, butadienyl, pentadienyl, and hexadienyl, among others. A cycloalkyl group can be substituted or unsubstituted. Such substituted cycloalkyl groups include, by way of example, cyclohexanone and the like.

An “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6 to 14 carbons, and in others from 6 to 12 or even 6 to 10 carbon atoms in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

An “arylene” group is a bivalent aryl group as defined herein.

A “heteroaryl” group is an aryl ring system having one to four heteroatoms as ring atoms in a heteroaromatic ring system, wherein the remainder of the atoms are carbon atoms. In some embodiments, heteroaryl groups contain 5 to 6 ring atoms, and in others from 6 to 9 or 6 to 10 atoms in the ring portions of the groups. Suitable heteroatoms include oxygen, sulfur, and nitrogen. In certain embodiments, the heteroaryl ring system is monocyclic or bicyclic. Non-limiting examples include, but are not limited to, groups such as pyrrolyl, pyrazolyl, imidazolyl, triazolyl, tetrazolyl, oxazolyl, isoxazolyl, thiazolyl, pyrrolyl, pyridyl, pyridazinyl, pyrimidinyl, pyrazinyl, thiophenyl, benzothiophenyl, furanyl, benzofuranyl (for example, isobenzofuran-1,3-diimine), indolyl, azaindolyl (for example, pyrrolopyridyl or 1H-pyrrolo[2,3-b]pyridyl), indazolyl, benzimidazolyl (for example, 1H-benzo[d]imidazolyl), imidazopyridyl (for example, azabenzimidazolyl, 3H-imidazo[4,5-b]pyridyl or 1H-imidazo[4,5-b]pyridyl), pyrazolopyridyl, triazolopyridyl, benzotriazolyl, benzoxazolyl, benzothiazolyl, benzothiadiazolyl, isoxazolopyridyl, thianaphthalenyl, purinyl, xanthinyl, adeninyl, guaninyl, quinolinyl, isoquinolinyl, tetrahydroquinolinyl, quinoxalinyl, and quinazolinyl groups.

A “heteroarylene” group is a bivalent heteroaryl group as defined herein.

A “heterocyclyl” is an aromatic (also referred to as heteroaryl) or non-aromatic cycloalkyl in which one to four of the ring carbon atoms are independently replaced with a heteroatom from the group consisting of O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclyl group can be substituted or unsubstituted. Heterocyclyl groups encompass unsaturated, partially saturated, and saturated ring systems, such as, for example, imidazolyl, imidazolinyl, and imidazolidinyl groups. The term “heterocyclyl” includes fused ring species, including those comprising fused aromatic and non-aromatic groups, such as, for example, benzotriazolyl, 2,3-dihydrobenzo[1,4]dioxinyl, and benzo[1,3]dioxolyl. The term also includes bridged polycyclic ring systems containing a heteroatom such as, but not limited to, quinuclidyl. Representative examples of a heterocyclyl group include, but are not limited to, aziridinyl, azetidinyl, pyrrolidyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl (for example, tetrahydro-2H-pyranyl), tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl; for example, 1H-imidazo[4,5-b]pyridyl, or 1H-imidazo[4,5-b]pyridyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below A “cycloalkylalkyl” group is a radical of the formula -alkyl-cycloalkyl, wherein alkyl and cycloalkyl are defined above. Substituted cycloalkylalkyl groups may be substituted at the alkyl, the cycloalkyl, or both the alkyl and the cycloalkyl portions of the group. Representative cycloalkylalkyl groups include but are not limited to cyclopentylmethyl, cyclopentylethyl, cyclohexylmethyl, cyclohexylethyl, and cyclohexylpropyl. Representative substituted cycloalkylalkyl groups may be mono-substituted or substituted more than once.

An “aralkyl” group is a radical of the formula -alkyl-aryl, wherein alkyl and aryl are defined above. Substituted aralkyl groups may be substituted at the alkyl, the aryl, or both the alkyl and the aryl portions of the group. Representative aralkyl groups include, but are not limited to, benzyl and phenethyl groups and fused (cycloalkylaryl)alkyl groups such as 4-ethyl-indanyl.

A “heterocyclylalkyl” group is a radical of the formula -alkyl-heterocyclyl, wherein alkyl and heterocyclyl are defined above. Substituted heterocyclylalkyl groups may be substituted at the alkyl, the heterocyclyl, or both the alkyl and the heterocyclyl portions of the group. Representative heterocyclylalkyl groups include, but are not limited to, 4-ethyl-morpholinyl, 4-propylmorpholinyl, furan-2-yl methyl, furan-3-yl methyl, pyrdine-3-yl methyl, (tetrahydro-2H-pyran-4-yl)methyl, (tetrahydro-2H-pyran-4-yl)ethyl, tetrahydrofuran-2-yl methyl, tetrahydrofuran-2-yl ethyl, and indol-2-yl propyl.

A “halogen” is chloro, iodo, bromo, or fluoro.

A “hydroxyalkyl” group is an alkyl group as described above substituted with one or more hydroxy groups.

An “alkoxy” group is O(alkyl), wherein alkyl is defined above.

An “alkoxyalkyl” group is (alkyl)O(alkyl), wherein alkyl is defined above.

As used herein, “alkynyl” refers to a monovalent hydrocarbon radical moiety containing at least two carbon atoms and one or more carbon-carbon triple bonds. Alkynyl is optionally substituted and can be linear, branched, or cyclic. Alkynyl includes, but is not limited to, those radicals having 2-20 carbon atoms, i.e., C₂₋₂₀ alkynyl; 2-12 carbon atoms, i.e., C₂₋₁₂ alkynyl; 2-8 carbon atoms, i.e., C₂₋₈ alkynyl; 2-6 carbon atoms, i.e., C₂₋₆ alkynyl; and 2-4 carbon atoms, i.e., C₂₋₄ alkynyl. Examples of alkynyl moieties include, but are not limited to ethynyl, propynyl, and butynyl.

As used herein, “haloalkyl” refers to alkyl, as defined above, wherein the alkyl includes at least one substituent selected from a halogen, for example, fluorine (F), chlorine (Cl), bromine (Br), or iodine (I). Examples of haloalkyl include, but are not limited to, —CF₃, —CH₂CF₃, —CCl₂F, and —CCl₃.

As used herein, “haloalkoxy” refers to alkoxy, as defined above, wherein the alkoxy includes at least one substituent selected from a halogen, e.g., F, Cl, Br, or I.

As used herein, “arylalkyl” refers to a monovalent moiety that is a radical of an alkyl compound, wherein the alkyl compound is substituted with an aromatic substituent, i.e., the aromatic compound includes a single bond to an alkyl group and wherein the radical is localized on the alkyl group. An arylalkyl group bonds to the illustrated chemical structure via the alkyl group. An arylalkyl can be represented by the structure, e.g., B—CH₂—, B—CH₂—CH₂—, B—CH₂—CH₂—CH₂—, B—CH₂—CH₂—CH₂—CH₂—, B—CH(CH₃)—CH₂—CH₂—, B—CH₂—CH(CH₃)—CH₂—, wherein B is an aromatic moiety, e.g., phenyl. Arylalkyl is optionally substituted, i.e., the aryl group and/or the alkyl group, can be substituted as disclosed herein. Examples of arylalkyl include, but are not limited to, benzyl.

As used herein, “alkylaryl” refers to a monovalent moiety that is a radical of an aryl compound, wherein the aryl compound is substituted with an alkyl substituent, i.e., the aryl compound includes a single bond to an alkyl group and wherein the radical is localized on the aryl group. An alkylaryl group bonds to the illustrated chemical structure via the aryl group. An alkylaryl can be represented by the structure, e.g., —B—CH₃, —B—CH₂—CH₃, —B—CH₂—CH₂—CH₃, —B—CH₂—CH₂—CH₂—CH₃, —B—CH(CH₃)—CH₂—CH₃, —B—CH₂—CH(CH₃)—CH₃, wherein B is an aromatic moiety, e.g., phenyl. Alkylaryl is optionally substituted, i.e., the aryl group and/or the alkyl group, can be substituted as disclosed herein. Examples of alkylaryl include, but are not limited to, toluyl.

As used herein, “aryloxy” refers to a monovalent moiety that is a radical of an aromatic compound wherein the ring atoms are carbon atoms and wherein the ring is substituted with an oxygen radical, i.e., the aromatic compound includes a single bond to an oxygen atom and wherein the radical is localized on the oxygen atom, e.g., C₆H₅—O—, for phenoxy. Aryloxy substituents bond to the compound which they substitute through this oxygen atom. Aryloxy is optionally substituted. Aryloxy includes, but is not limited to, those radicals having 6 to 20 ring carbon atoms, i.e., C₆₋₂₀ aryloxy; 6 to 15 ring carbon atoms, i.e., C₆₋₁₅ aryloxy, and 6 to 10 ring carbon atoms, i.e., C₆₋₁₀ aryloxy. Examples of aryloxy moieties include, but are not limited to phenoxy, naphthoxy, and anthroxy.

An “amino” group is a radical of the formula NH₂.

A “hydroxyl amine” group is a radical of the formula N(R^{#})OH or NHOH, wherein R^{#} is a substituted or unsubstituted alkyl, cycloalkyl, cycloalkylalkyl, aryl, aralkyl, heterocyclyl or heterocyclylalkyl group as defined herein.

An “alkoxyamine” group is a radical of the formula —N(R^{#})O-alkyl or —NHO-alkyl, wherein R^{#} is as defined above.

An “aralkoxyamine” group is a radical of the formula N(R^{#})O-aryl or NHOaryl, wherein R^{#} is as defined above.

An “aminocarbonyl” group is a radical of the formula —C(═O)N(R^{#})₂, —C(═O)NH(R^{#}), or C(═O)NH₂, wherein each R is as defined above.

An “acylamino” group is a radical of the formula NHC(═O)(R^{#}) or N(alkyl)C(═O)(R^{#}), wherein each alkyl and R are independently as defined above.

An “O(alkyl)aminocarbonyl” group is a radical of the formula —O(alkyl)C(═O)N(R^{#})₂, —O(alkyl)C(═O)NH(R^{#}), or —O(alkyl)C(═O)NH₂, wherein each R^{#} is independently as defined above.

An “N-oxide” group is a radical of the formula —N⁺—O—.

A “carboxy” group is a radical of the formula C(═O)OH.

A “ketone” group is a radical of the formula C(═O)(R^{#}), wherein R^{#} is as defined above.

An “aldehyde” group is a radical of the formula —CH(═O).

An “ester” group is a radical of the formula C(═O)O(R^{#}) or OC(═O)(R^{#}), wherein R^{#} is as defined above.

A “urea” group is a radical of the formula —N(alkyl)C(═O)N(R^{#})₂, —N(alkyl)C(═O)NH(R^{#}), —N(alkyl)C(═O)NH₂, —NHC(═O)N(R^{#})₂, —NHC(═O)NH(R^{#}), or NHC(═O)NH₂⁴, wherein each alkyl and R^{#} are independently as defined above.

An “imine” group is a radical of the formula —N═C(R^{#})₂ or —C(R^{#})═N(R⁴), wherein each R^{#} is independently as defined above.

An “imide” group is a radical of the formula —C(═O)N(R^{#})C(═O)(R^{#}) or N((C═O)(R^{#}))₂, wherein each R^{#} is independently as defined above.

A “urethane” group is a radical of the formula —OC(═O)N(R^{#})₂, —OC(═O)NH(R^{#}), —N(R⁴)C(═O)O(R^{#}), or —NHC(═O)O(R^{#}), wherein each R^{#} is independently as defined above.

An “amidine” group is a radical of the formula —C(═N(R^{#}))N(R^{#})₂, —C(═N(R^{#}))NH(R^{#}), —C(═N(R^{#}))NH₂, —C(═NH)N(R^{#})₂, —C(═NH)NH(R^{#}), —C(═NH)NH₂, —N═C(R^{#})N(R^{#})₂, —N═C(R^{#})NH(R^{#}), —N═C(R^{#})NH₂, —N(R^{#})C(R^{#})═N(R^{#}), —NHC(R^{#})═N(R^{#}), —N(R^{#})C(R^{#})═NH, or —NHC(R^{#})═NH, wherein each R^{#} is independently as defined above.

A “guanidine” group is a radical of the formula —N(R^{#})C(═N(R^{#}))N(R^{#})₂, —NHC(═N(R^{#}))N(R^{#})₂, —N(R^{#})C(═NH)N(R^{#})₂, —N(R^{#})C(═N(R^{#}))NH(R^{#}), —N(R^{#})C(═N(R^{#}))NH₂, —NHC(═NH)N(R^{#})₂, —NHC(═N(R^{#}))NH(R^{#}), —NHC(═N(R^{#}))NH₂, —NHC(═NH)NH(R^{#}), —NHC(═NH)NH₂, —N═C(N(R^{#})₂)₂, —N═C(NH(R^{#}))₂, or —N═C(NH₂)₂, wherein each R is independently as defined above.

An “enamine” group is a radical of the formula —N(R)C(R^{#})═C(R^{#})₂, —NHC(R^{#})═C(R^{#})₂, —C(N(R^{#})₂)═C(R^{#})₂, —C(NH(R^{#}))═C(R^{#})₂, —C(NH₂)═C(R^{#})₂, —C(R^{#})═C(R^{#})(N(R^{#})₂), C(R^{#})═C(R^{#})(NH(R^{#})) or —C(R^{#})═C(R^{#})(NH₂), wherein each R^{#} is independently as defined above.

An “oxime” group is a radical of the formula —C(═NO(R^{#}))(R^{#}), —C(═NOH)(R^{#}), —CH(═NO(R^{#})), or —CH(═NOH), wherein each R^{#} is independently as defined above.

A “hydrazide” group is a radical of the formula —C(═O)N(R^{#})N(R^{#})₂, —C(═O)NHN(R^{#})₂, —C(═O)N(R^{#})NH(R^{#}), —C(═O)N(R^{#})NH₂, —C(═O)NHNH(R^{#})₂, or —C(═O)NHNH₂, wherein each R is independently as defined above.

A “hydrazine” group is a radical of the formula —N(R^{#})N(R^{#})₂, —NHN(R^{#})₂, —N(R^{#})NH(R^{#}), —N(R^{#})NH₂, —NHNH(R^{#})₂, or —NHNH₂, wherein each R is independently as defined above.

A “hydrazone” group is a radical of the formula —C(═N—N(R^{#})₂)(R^{#})₂, —C(═NNH(R^{#}))(R^{#})₂, —C(═N—NH₂)(R^{#})₂, —N(R^{#})(N═C(R^{#})₂), or —NH(N═C(R^{#})₂), wherein each R is independently as defined above.

An “azide” group is a radical of the formula —N₃.

An “isocyanate” group is a radical of the formula N═C═O.

An “isothiocyanate” group is a radical of the formula N═C═S.

A “cyanate” group is a radical of the formula OCN.

A “thiocyanate” group is a radical of the formula SCN.

A “thioether” group is a radical of the formula —S(R^{#}), wherein R^{#} is as defined above.

A “thiocarbonyl” group is a radical of the formula —C(═S)(R⁴), wherein R^{#} is as defined above.

A “sulfinyl” group is a radical of the formula —S(═O)(R^{#}), wherein R^{#} is as defined above.

A “sulfone” group is a radical of the formula —S(═O)₂(R^{#}), wherein R^{#} is as defined above.

A “sulfonylamino” group is a radical of the formula —NHSO₂(R^{#}) or —N(alkyl)SO₂(R^{#}), wherein each alkyl and R^{#} are defined above.

A “sulfonamide” group is a radical of the formula —S(═O)₂N(R^{#})₂, or —S(═O)₂NH(R^{#}), or —S(═O)₂NH₂, wherein each R is independently as defined above.

A “phosphonate” group is a radical of the formula —P(═O)(O(R^{#}))₂, —P(═O)(OH)₂, —OP(═O)(O(R^{#}))(R⁴), or —OP(═O)(OH)(R^{#}), wherein each R^{#} is independently as defined above.

A “phosphine” group is a radical of the formula —P(R^{#})₂, wherein each R^{#} is independently as defined above.

When the groups described herein, with the exception of alkyl groups, are said to be “substituted,” they may be substituted with any appropriate substituent or substituents. Illustrative examples of substituents are those found in the compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothiophenyl, or benzofuranyl) aryloxy; aralkyloxy; heterocyclyloxy; and heterocyclyl alkoxy.

As used herein, the term “pharmaceutically acceptable salt(s)” refers to a salt prepared from a pharmaceutically acceptable non-toxic acid or base including an inorganic acid or base and an organic acid or base.

As used herein and unless otherwise indicated, the term “solvate” means a compound, or a salt thereof, that further includes a stoichiometric or non-stoichiometric amount of a solvent bound by non-covalent intermolecular forces. In one embodiment, the solvate is a hydrate.

As used herein and unless otherwise indicated, the term “hydrate” means a compound, or a salt thereof, that further includes a stoichiometric or non-stoichiometric amount of water bound by non-covalent intermolecular forces.

As used herein and unless otherwise indicated, the term “prodrug” means a compound derivative that can hydrolyze, oxidize, or otherwise react under biological conditions (in vitro or in vivo) to provide an active compound. Examples of prodrugs include, but are not limited to, derivatives and metabolites of a compound that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. In certain embodiments, prodrugs of compounds with carboxyl functional groups are the lower alkyl esters of the carboxylic acid. The carboxylate esters may be formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh).

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments disclosed herein, including mixtures thereof. The use of stereomerically pure forms of such compounds, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (WileyInterscience, New York, 1981); Wilen, S. H., et al., Tetrahedron 33:2725 (1977); Eliel, E. L., Stereochemistry of Carbon Compounds (McGrawHill, NY, 1962); and Wilen, S. H., Tables of Resolving Agents and Optical Resolutions p. 268 (E. L. Eliel, Ed., Univ. of Notre Dame Press, Notre Dame, IN, 1972).

It should also be noted that the compounds can include E and Z isomers, or a mixture thereof, and cis and trans isomers, or a mixture thereof. In certain embodiments, the compounds are isolated as either the cis or trans isomer. In other embodiments, the compounds are a mixture of the cis and trans isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in an aqueous solution, pyrazoles may exhibit the following isomeric forms, which are referred to as tautomers of each other:

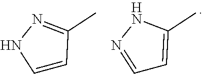

As readily understood by one skilled in the art, a wide variety of functional groups and other structures may exhibit tautomerism and all tautomers of the compounds are within the scope of the present disclosure.

It should also be noted the compounds can contain unnatural proportions of atomic isotopes at one or more of the atoms. For example, the compounds may be radiolabeled with radioactive isotopes, such as for example tritium (H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds, for example, the isotopologues are deuterium, carbon-13, or nitrogen-15 enriched compounds.

It should be noted that if there is a discrepancy between a depicted structure and a name for that structure, the depicted structure is to be accorded more weight.

As used herein, the term “residue” refers to the chemical moiety within a compound that remains after a chemical reaction. For example, the term “amino acid residue” or “N-alkyl amino acid residue” refers to the product of an amide coupling or peptide coupling of an amino acid or a N-alkyl amino acid to a suitable coupling partner; wherein, for example, a water molecule is expelled after the amide or peptide coupling of the amino acid or the N-alkylamino acid, resulting in the product having the amino acid residue or N-alkyl amino acid residue incorporated therein.

As used herein, “sugar” or “sugar group” or “sugar residue” refers to a carbohydrate moiety which may comprise 3-carbon (those) units, 4-carbon (tetrose) units, 5-carbon (pentose) units, 6-carbon (hexose) units, 7-carbon (heptose) units, or combinations thereof, and may be a monosaccharide, a disaccharide, a trisaccharide, a tetrasaccharide, a pentasaccharide, an oligosaccharide, or any other polysaccharide. In some instances, a “sugar” or “sugar group” or “sugar residue” comprises furanoses (e.g., ribofuranose, fructofuranose) or pyranoses (e.g., glucopyranose, galactopyranose), or a combination thereof. In some instances, a “sugar” or “sugar group” or “sugar residue” comprises aldoses or ketoses, or a combination thereof. Non-limiting examples of monosaccharides include ribose, deoxyribose, xylose, arabinose, glucose, mannose, galactose, and fructose. Non-limiting examples of disaccharides include sucrose, maltose, lactose, lactulose, and trehalose. Other “sugars” or “sugar groups” or “sugar residues” include polysaccharides and/or oligosaccharides, including, but not limited to, amylose, amylopectin, glycogen, inulin, and cellulose. In some instances, a “sugar” or “sugar group” or “sugar residue” is an amino-sugar. In some instances, a “sugar” or “sugar group” or “sugar residue” is a glucamine residue (1-amino-1-deoxy-D-glucitol) linked to the rest of molecule via its amino group to form an amide linkage with the rest of the molecule (i.e., a glucamide).

As used herein, “binding agent” refers to any molecule, e.g., antibody, capable of binding with specificity to a given binding partner, e.g., antigen.

As used herein, the term “amino acid” refers to an organic compound that contains amino (—NH₂) and carboxyl (—COOH) functional groups, along with a side chain (R group), which is specific to each amino acid. Amino acids may be proteinogenic or non-proteinogenic. By “proteinogenic” is meant that the amino acid is one of the twenty naturally occurring amino acids found in proteins. The proteinogenic amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. By “non-proteinogenic” is meant that either the amino acid is not found naturally in protein or is not directly produced by cellular machinery (e.g., is the product of post-translational modification). Non-limiting examples of non-proteinogenic amino acids include gamma-aminobutyric acid (GABA), taurine (2-aminoethanesulfonic acid), theanine (L-γ-glutamylethylamide), hydroxyproline, beta-alanine, ornithine, and citrulline.

As used herein “peptide,” in its various grammatical forms, is defined in its broadest sense to refer to a compound of two or more subunit amino acids, amino acid analogs, or other peptidomimetics. The subunits may be linked by peptide bonds or by other bonds, for example, ester, ether, and the like. As used herein, the term “amino acid” refers to either natural and/or unnatural, proteinogenic or non-proteinogenic, or synthetic amino acids, including glycine and both the D and L optical isomers, and amino acid analogs and peptidomimetics. If the peptide chain is short, e.g., two, three or more amino acids, it is commonly called an oligopeptide. If the peptide chain is longer, the peptide is typically called a polypeptide or a protein. Full-length proteins, analogs, mutants, and fragments thereof are encompassed by the definition. The terms also include post-expression modifications of the polypeptide, for example, glycosylation, acetylation, phosphorylation, and the like. Furthermore, as ionizable amino and carboxyl groups are present in the molecule, a particular peptide may be obtained as an acidic or basic salt, or in neutral form. A peptide may be obtained directly from the source organism or may be recombinantly or synthetically produced.

The amino acid sequence of an antibody can be numbered using any known numbering schemes, including those described by Kabat et al., (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Pluckthun, J. Mol. Biol., 2001, 309:657-70 (“AHo” numbering scheme). Unless otherwise specified, the numbering scheme used herein is the Kabat numbering scheme. However, selection of a numbering scheme is not intended to imply differences in sequences where they do not exist, and one of skill in the art can readily confirm a sequence position by examining the amino acid sequence of one or more antibodies. Unless stated otherwise, the “EU numbering scheme” is generally used when referring to a residue in an antibody heavy chain constant region (e.g., as reported in Kabat et al., supra).

As used herein, the term “cell-killing activity” refers to the activity that decreases or reduces the cell viability of the tested cell line.

In the claims that follow and in the preceding description, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments.

### Aspect 1

In some embodiments, the platform is a conjugator-linker-payload compound of Formula (I):

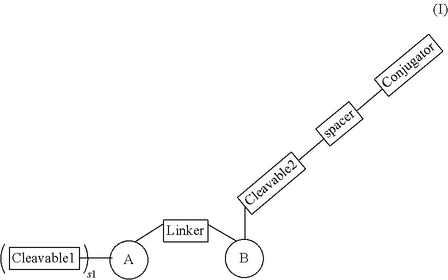

or a pharmaceutically acceptable salt, tautomer, solvate, stereoisomer, or isotopologue thereof, wherein:
each of ring A and ring B is, independently, one of formula (IIa), (IIb), (IIc), (IId), (IIe), (IIf), or (IIg):

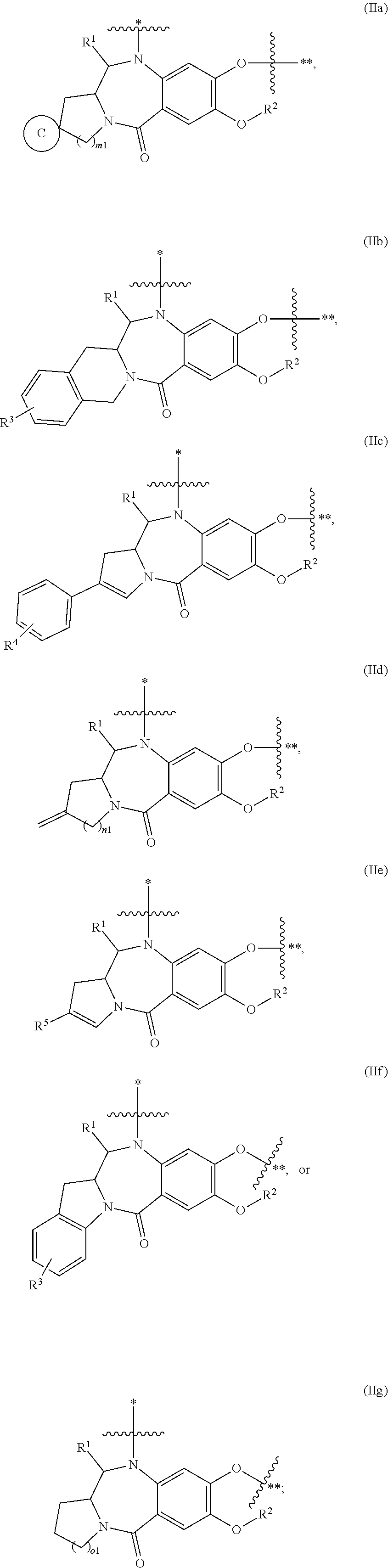

wherein:
each ring C is independently a cyclopropyl ring or a cyclobutyl ring;
each R¹ is independently H, OH, C₁₋₄ alkyl, C₁₋₄ alkoxyl, or —O—(CH₂CH₂O)ₜ₁—CH₃;
each R² is independently H or C₁₋₄ alkyl;
each R³ and R⁴ is, independently, H, NR^{a1}R^{b1}, OH, C₁₋₄ alkyl, C₁₋₄ alkoxyl, or aryl;
each R^{a1} and R^{b1} is, independently, H or C₁₋₄ alkyl;
each R⁵ is independently H, C₁₋₄ alkyl, C₁₋₄ alkoxyl, or aryl;
each m1, n1, and o1 is, independently, 1 or 2;
t1 is 1, 2, 3, 4, 5, 6, 7, or 8;
marks the bond where ring A connects to Cleavable1 when s1 is 1 or to —H when s1 is 0, or where ring B connects to Cleavable2; and
* marks the bond where ring A or ring B connects to Linker;
Linker is —(CH₂)ᵣ—, —(CH₂)ₚ—X—(CH₂)_{q}—, or —(CH₂)ₚ—CH═CH—(CH₂)_{q}—; wherein:
each r, p, and q, is, independently, 1, 2, 3, 4, 5, 6, 7, or 8;
the sum of p and q is 1, 2, 3, 4, 5, 6, 7, or 8;
X is NR⁶, NHC(═O), C(═O)NH, 0, SO₂, a substituted or unsubstituted divalent aryl ring, substituted or unsubstituted divalent heteroaryl ring, substituted or unsubstituted divalent heterocyclic ring, or substituted or unsubstituted divalent cyclic ring; and
R⁶ is H or C₁₋₄ alkyl;
Cleavable1, if present, has formula (VIa) or (VIc):

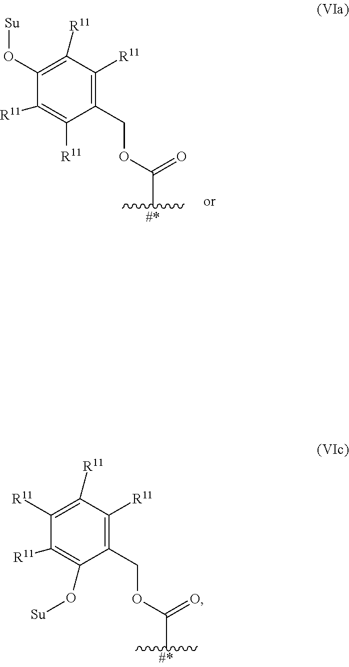

wherein:
Su is a sugar moiety;
each R¹¹ is, independently, hydrogen, halogen, substituted or unsubstituted C₁₋₄ alkyl, —CN, or —NO₂; and
# marks the bond where Cleavable1 connects to ring A;
s1 is 0 or 1;
Cleavable2 has formula (VIIa1), (VIIc1), (VIId1), or (VIIf1):

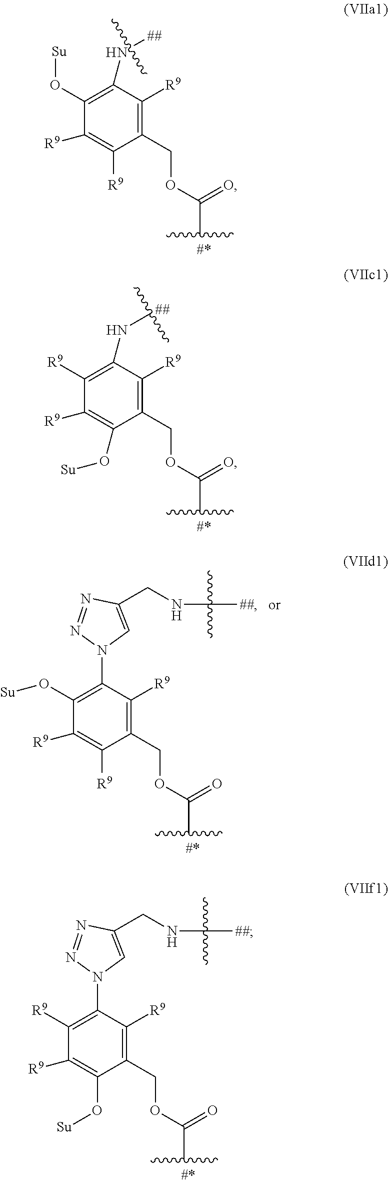

wherein:
Su is a sugar moiety;
each R⁹ is, independently, hydrogen, halogen, substituted or unsubstituted C₁₋₄ alkyl, —CN, or —NO₂;
#* marks the bond where Cleavable2 connects to ring B; and
## marks the bond where Cleavable2 connects to Spacer;
Spacer is a bond, ^{###}—NH—(CH₂CH₂O)ₘ₂—CH₂CH₂—C(═O)—, ^{###}—NH—(CH₂CH₂O)ₘ₂—CH₂—C(═O)—, ^{###}—(CH₂)ₘ₂—C(═O)—, ^{###}—CH₂—C(═O)—NH—(CH₂)ₘ₂—C(═O)—, ^{###}—(CH₂CH₂O)ₘ₂—CH₂CH₂—C(═O)—, ^{###}—CH[—(CH₂)ₘ₂—COOH]—C(═O)—, ###—CH₂—C(═O)—NH—(CH₂)ₘ₂—C(═O)—NH—(CH₂)ₘ₂—C(═O)—, ^{###}—C(═O)—(CH₂)ₘ₂—C(═O)—, or ^{###}—NH—(CH₂)ₘ₂—C(═O)—; wherein:
each m2 is, independently, 1, 2, 3, 4, 6, 7, or 8; and
### marks the bond where Spacer connects to Conjugator; and Conjugator has formula (III), (IV), or (V):

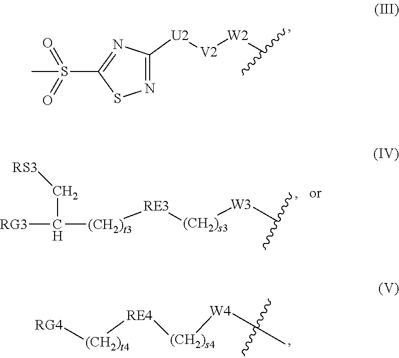

wherein:
U2 is a bond, heteroarylene, or arylene;
V2 is a bond or —C≡C—(CH₂)ₙ₂—;
n2 is an integer from 0 to 10 inclusive;
W2 is —C(═O)—, —NH—, or —O—;
RG3 is

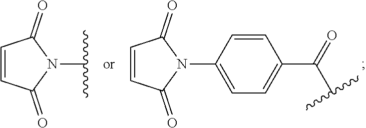 [SMILES: *N1C(=O)C=CC1=O ; *C(=O)c1ccc(N2C(=O)C=CC2=O)cc1]

RS3 is —NR^{a2}R^{b2};
each of R^{a2} and R^{b2} is, independently, H or substituted or unsubstituted C₁₋₄ alkyl;
RE3 is a bond, —O—, —OC(═O)—, —OC(═O)NR⁷—, —NHC(═O)NR⁷—, —OS(═O)₂NR⁷—, —NHS(═O)₂NR⁷—, or —OC(═O)NHS(═O)₂NR⁷—;
R⁷ is H or substituted or unsubstituted C₁₋₄ alkyl;
W3 is —C(═O)—, —NH—, or —O—;
t3 is 1 or 2;
s3 is 0, 1, or 2;
RG4 is

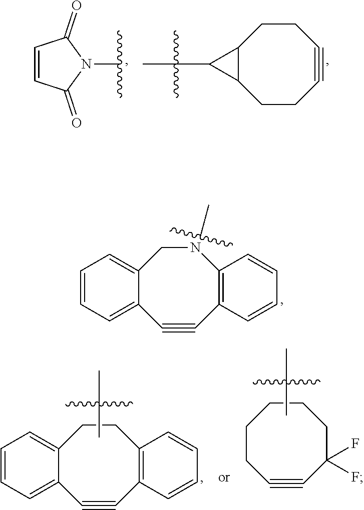

RE4 is a bond, —O—, —OC(═O)—, —OC(═O)NR⁸—, —NHC(═O)NR⁸—, —OS(═O)₂NR⁸—, —NHS(═O)₂NR⁸—, or —OC(═O)NHS(═O)₂NR⁸—;
R⁸ is H or substituted or unsubstituted C₁₋₄ alkyl;
W4 is —C(═O)—, —NH—, or —O—;
t4 is 1, 2, 3, 4, 5, 6, 7, or 8; and
s4 is 0, 1, or 2.

In some embodiments, Linker is —(CH₂)ᵣ or —(CH₂)ₚ—X—(CH₂)_{q}—; ring A has formula (IIa), (IIb), (IIc), (IId), or (IIg); and ring B has formula (IIa), (IIb), (IId), or (IIg).

In some embodiments, Linker is —(CH₂)ᵣ—, ring A has formula (IIg), and ring B has formula (IIg).

In some embodiments, Linker is —(CH₂)ᵣ—, ring A has formula (IId), and ring B has formula (IId).

In some embodiments, Linker is —(CH₂)ᵣ—, ring A has formula (IIb), and ring B has formula (IIa).

In some embodiments, Linker is —(CH₂)ᵣ—, ring A has formula (IIa), and ring B has formula (IIb).

In some embodiments, Linker is —(CH₂)ᵣ—, ring A has formula (IIa), and ring B has formula (IIa).

In some embodiments, Linker is —(CH₂)ᵣ—, ring A has formula (IIc), and ring B has formula (IIa).

In some embodiments, Linker is —(CH₂)ₚ—X—(CH₂)_{q}—, ring A has formula (IIa), and ring B has formula (IIa).

In some embodiments, Linker is —(CH₂)ᵣ—, ring A has formula (IIc), and ring B has formula (IIb).

In some embodiments, r is 3 or 5.

In some embodiments, Linker is —(CH₂)ᵣ— and r is 5.

In some embodiments, Linker is —(CH₂)ₚ—X—(CH₂)_{q}—, X is an unsubstituted aryl ring, p is 1, and q is 1. In some embodiments, X is phenylene.

In some embodiments, formula (IIa) is

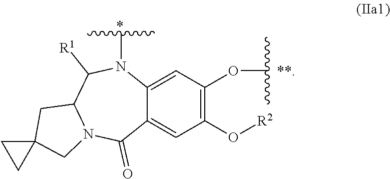

In some embodiments, formula (IId) is

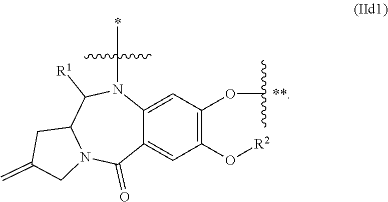

In some embodiments, formula (IIg) is

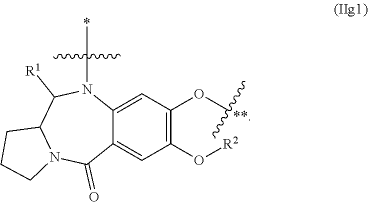

In some embodiments, formula (IIc) is

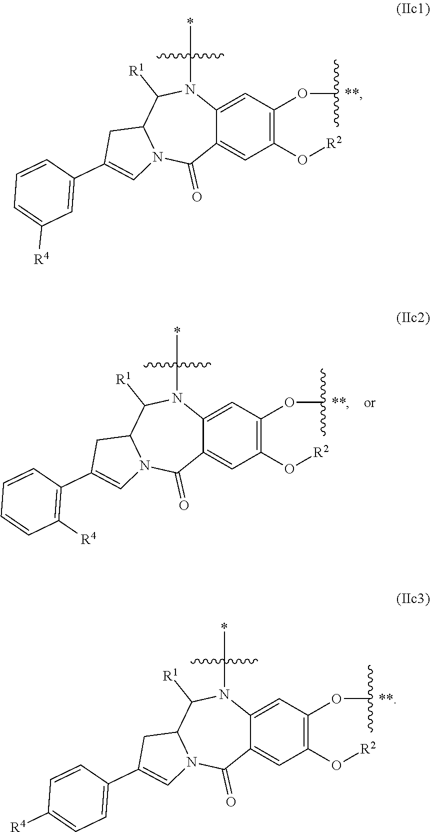

In some embodiments, formula (IIc) is

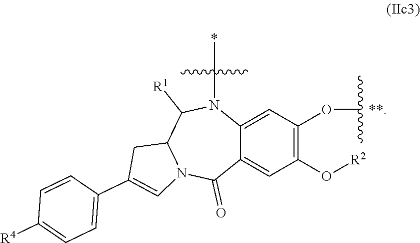

In some embodiments, ring A is

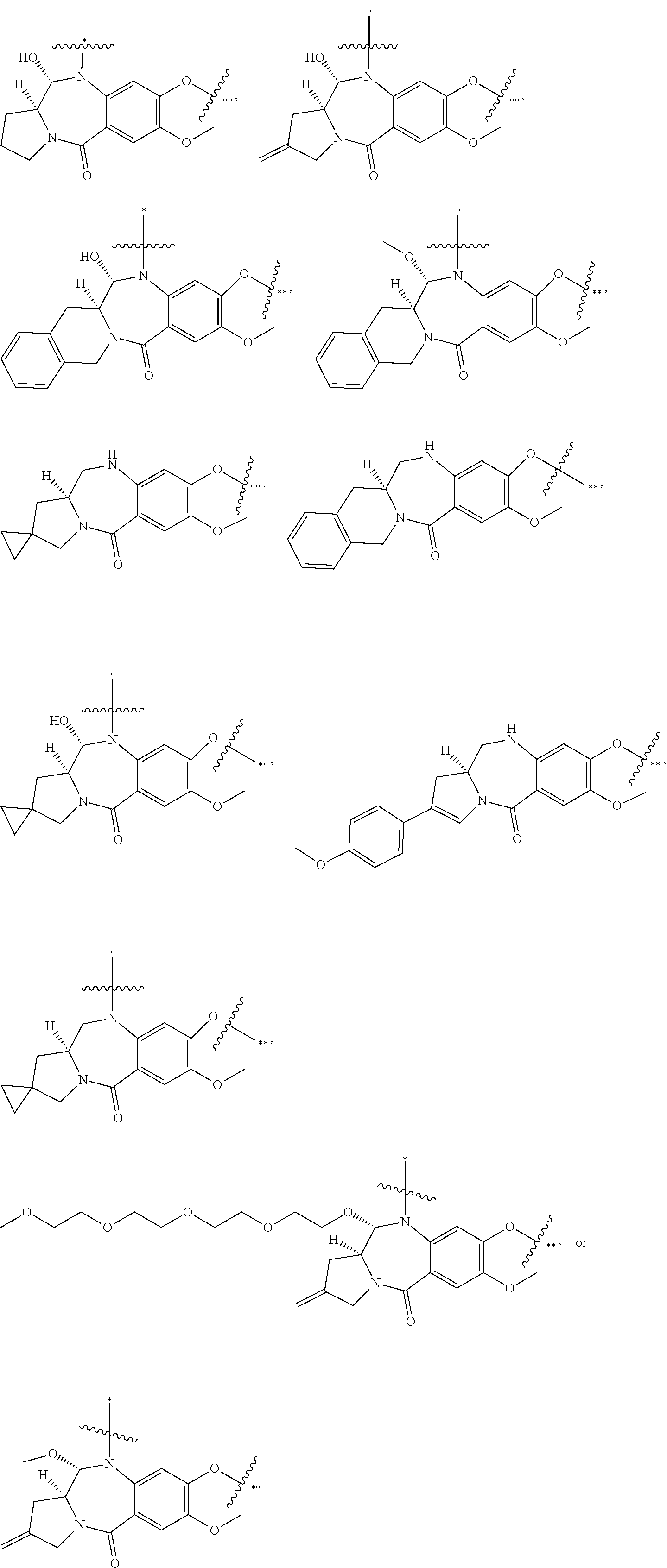

In some embodiments, ring B is

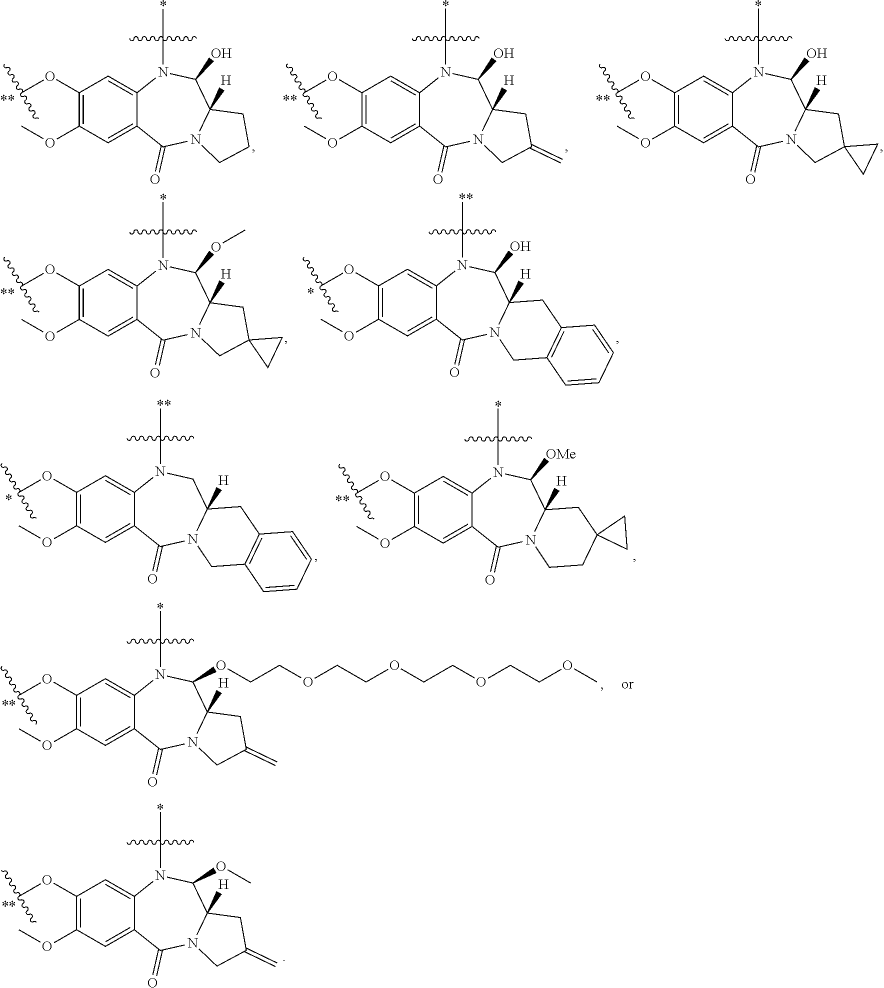

In some embodiments, when Linker is —(CH₂)ₚ—X—(CH₂)_{q}— or —(CH₂)ₚ—CH═CH—(CH₂)_{q}—, ring A has formula (IIa), m in ring A is 2, and ring B has formula (IIa), (IIb), (IIc), (IId), (IIe), (IIf), or (IIg).

In some embodiments, when Linker is —(CH₂)ₚ—X—(CH₂)_{q}— or —(CH₂)ₚ—CH═CH—(CH₂)_{q}—, ring A has formula (IIa), m in ring A is 1, and ring B has formula (IIa), (IIb), (IIc), (IId), (IIe), (IIf), or (IIg).

In some embodiments, Linker is —(CH₂)ᵣ—, —(CH₂)ₚ—X—(CH₂)_{q}— or —(CH₂)ₚ—CH═CH—(CH₂)_{q}—; and X is O, NR⁶, NHC(═O), -(m-C₆H₄)—,

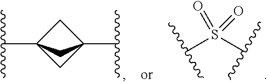 [SMILES: *[C@]12C[C@](*)(C1)C2 ; *S(*)(=O)=O]

In some embodiments, Linker is —(CH₂)ᵣ—.

In some embodiments, Linker is —(CH₂)ₚ—O—(CH₂)_{q}— or —(CH₂)ₚ—NH—(CH₂)_{q}—.

In some embodiments, the sum of p and q is 4.

In some embodiments, Linker is

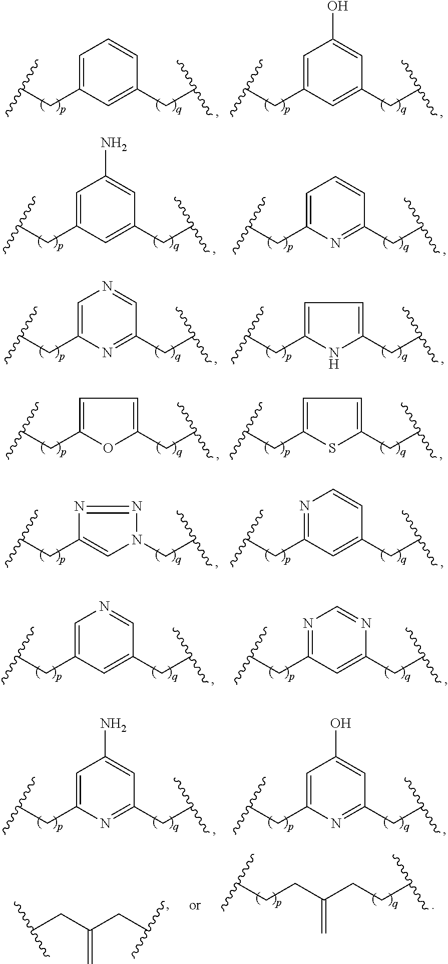

In some embodiments, the sum of p and q is 2.

In some embodiments, Linker is

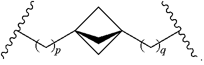

In some embodiments, Linker is —(CH₂)ₚ—CH═CH—(CH₂)_{q}—.

In some embodiments, the sum of p and q is 3.

In some embodiments, each Su is independently

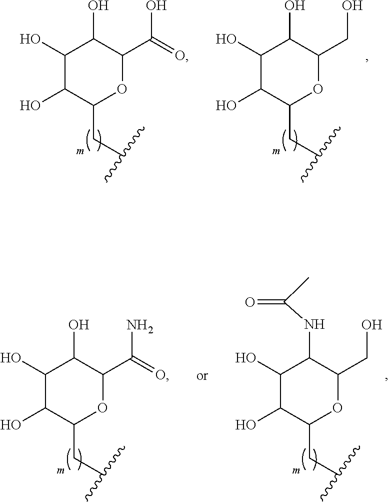

or a stereoisomer thereof; each m is independently 0 or 1; and [symbol] indicates a point of attachment to Cleavable1 or Cleavable2.

In some embodiments, each Su is independently

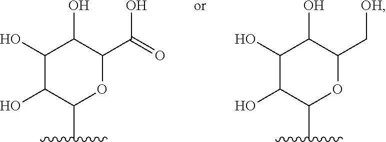 [SMILES: *C1OC(C(=O)O)C(O)C(O)C1O ; *C1OC(CO)C(O)C(O)C1O]

or a stereoisomer thereof.

In some embodiments, each Su is

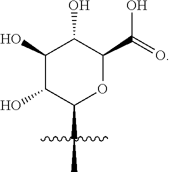 [SMILES: *[C@@H]1O[C@H](C(=O)O)[C@@H](O)[C@H](O)[C@H]1O]

In some embodiments, s1 is 1.

In alternative embodiments, Cleavable1 has formula (VIa), (VIb), (VIc), (VId), (VIe), (VIf), (VIg), (VIh), or (VIi):

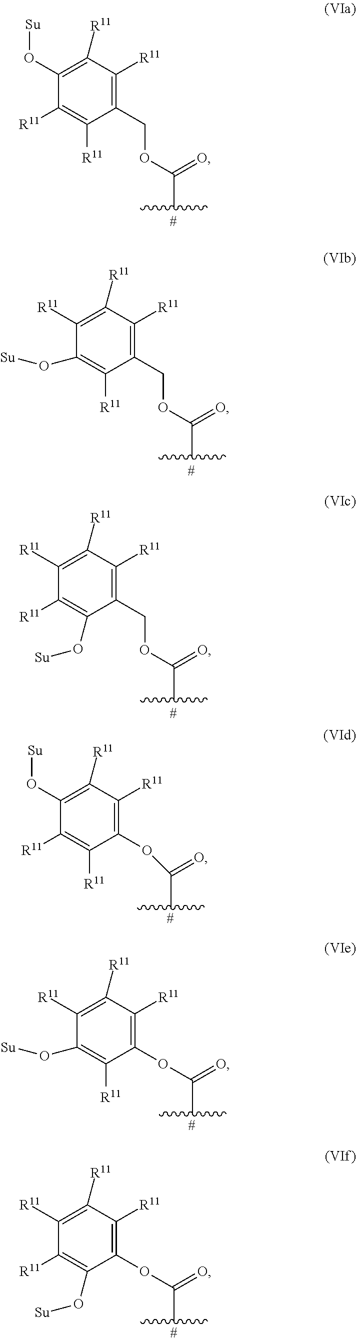

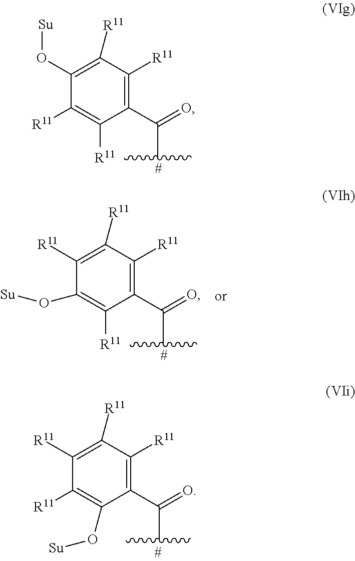

In some embodiments, Cleavable1 has formula (VIa).

In some embodiments, Cleavable1 is

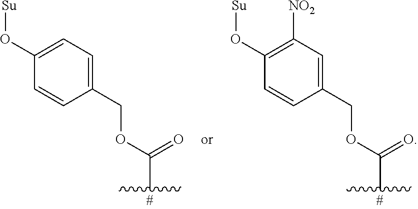

In some embodiments, s1 is 0.

In alternative embodiments, when s1 is 0, ring A has formula (IIa′), (IIb′), (IIc′), (IId′), (IIe′), (IIf′), or (IIg′):

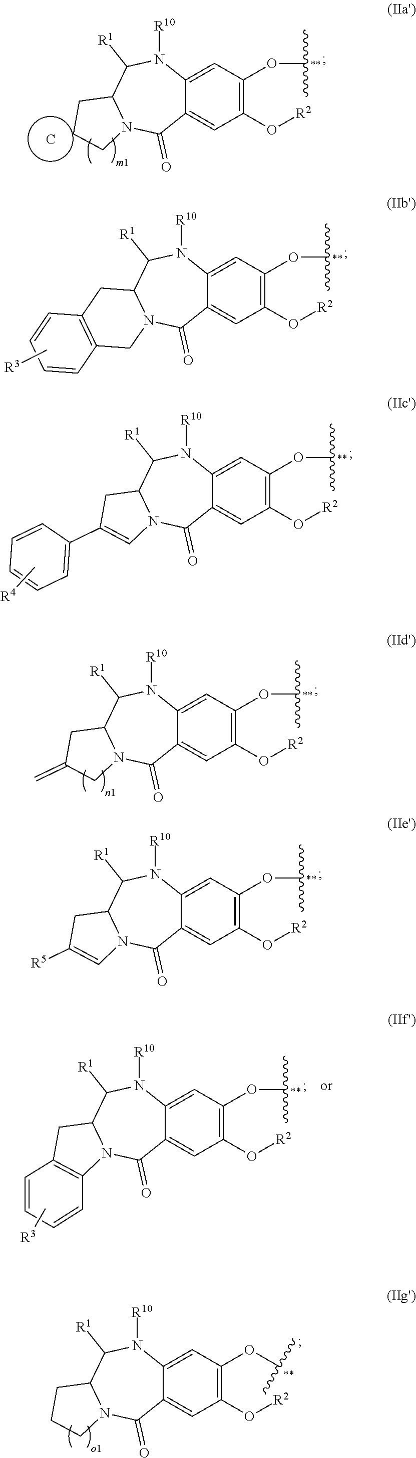

wherein R¹⁰ is, independently, H, OH, or C₁₋₄ alkyl, and the other variables (R¹, R², R³, R⁴, R⁵, m1, n1, o1) are as defined above with respect to Aspect 1.

In alternative embodiments, Cleavable2 has formula (VIIa1), (VIIb1), (VIIc1), (VIId1), (VIIe1), (VIIf1), (VIIa2), (VIIb2), (VIIc2), (VIId2), (VIIe2), (VIIf2), (VIIa3), (VIIb3), (VIIc3), (VIId3), (VIIe3), or (VIIf3):

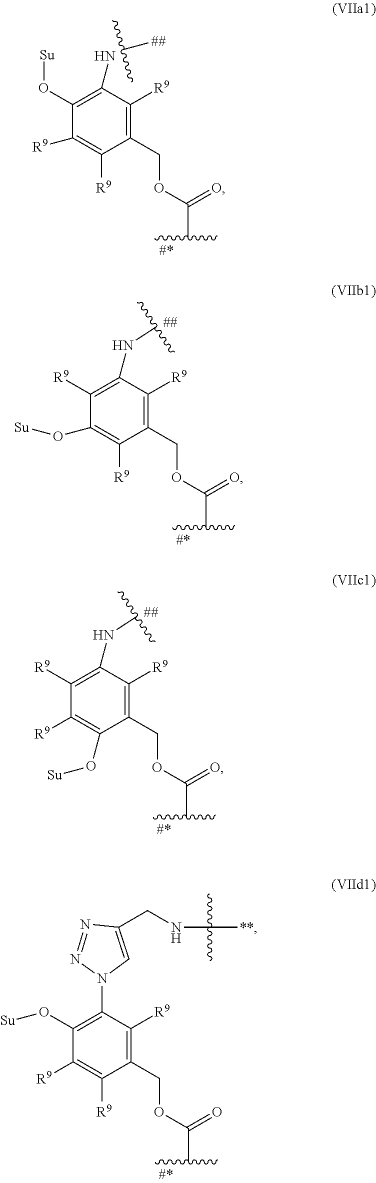

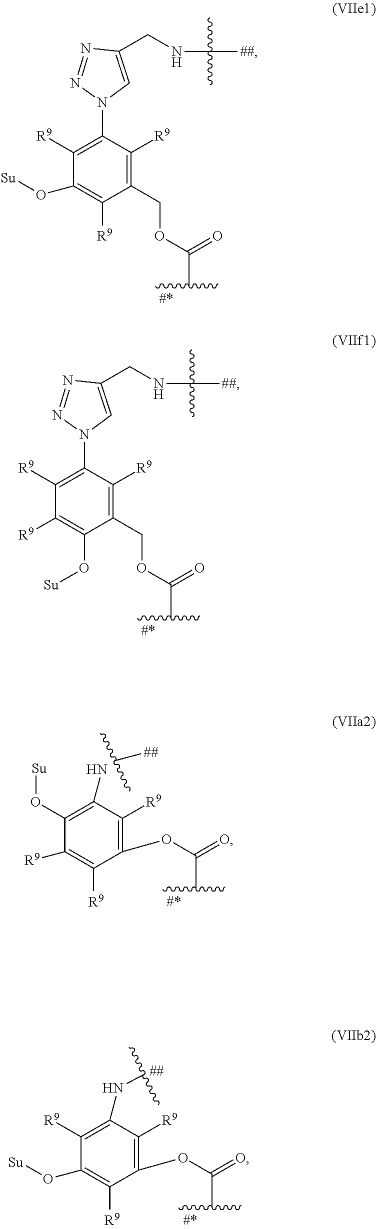

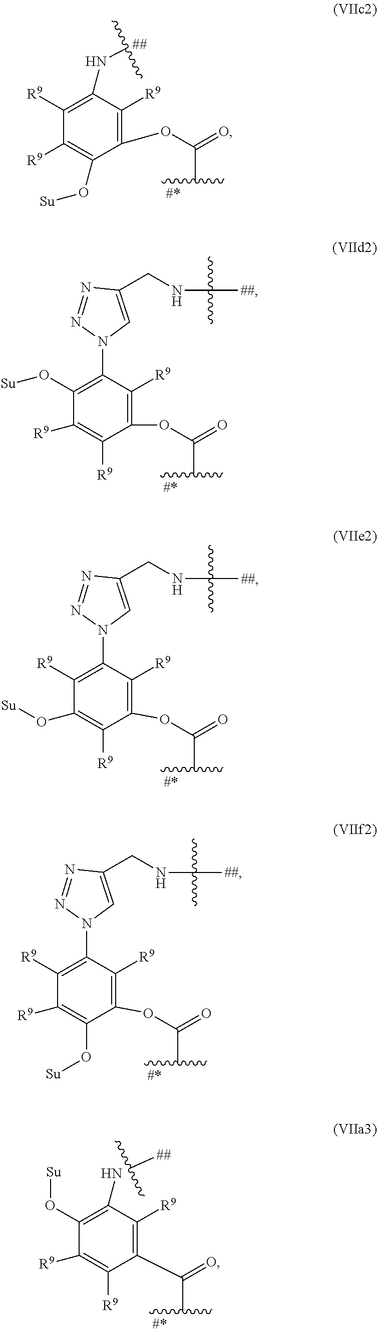

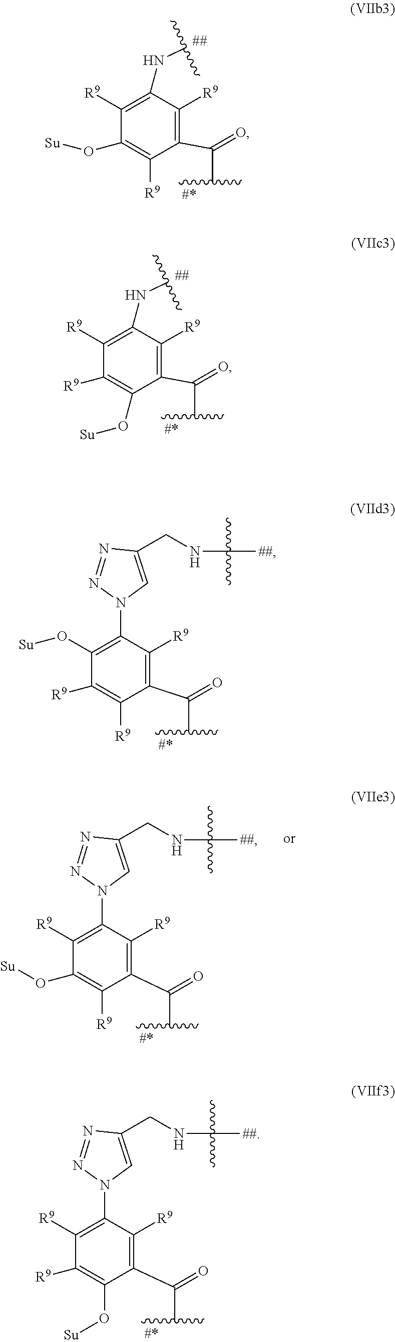

In some embodiments, s1 is 1 and Cleavable2 has one of the following formulas:

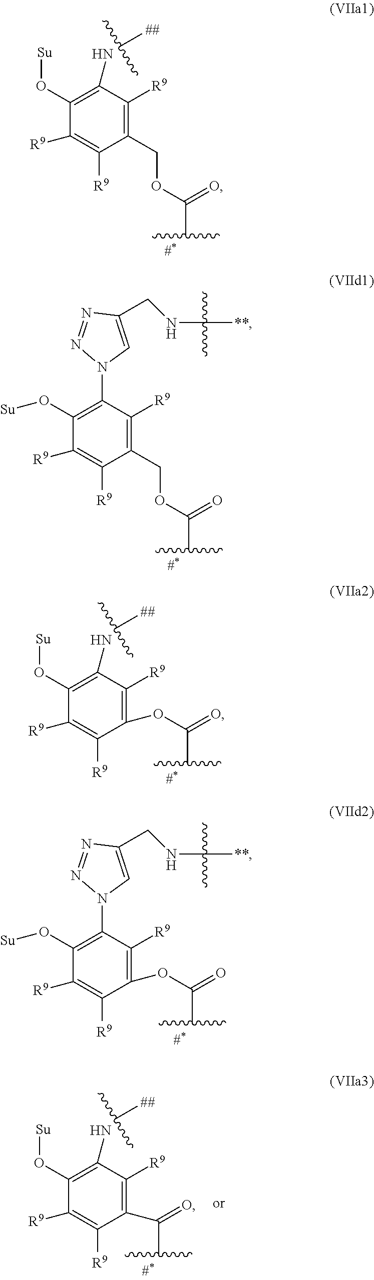

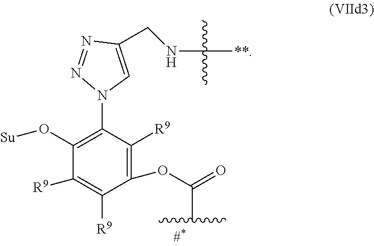

In some embodiments, s1 is 1 and Cleavable2 has formula (VIIa1) or (VIId1).

In some embodiments, Cleavable2 has formula (VIIa1) or (VIId1).

In some embodiments, each R⁹ is hydrogen.

In some embodiments, Cleavable2 is

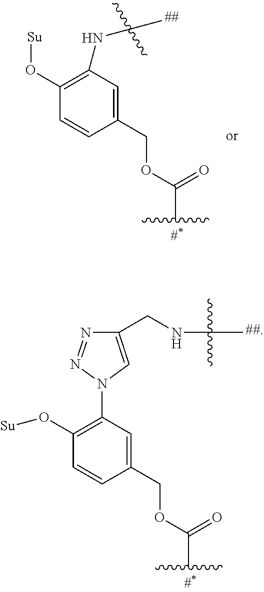

In some embodiments, Spacer is ^{###}—NH—(CH₂CH₂O)ₘ₂—CH₂CH₂—C(═O)— or ^{###}—NH—(CH₂CH₂O)ₘ₂—CH₂—C(═O)—.

In some embodiments, Spacer is ^{###}—NH—(CH₂CH₂O)₄—CH₂CH₂—C(═O)— or ^{###}—NH—(CH₂CH₂O)₄—CH₂—C(═O)—.

In some embodiments, Spacer is ^{###}—NH—(CH₂CH₂O)₄—CH₂CH₂—C(═O)—.

In some embodiments, Conjugator has formula (III).

In some embodiments, U2 is arylene.

In some embodiments, U2 is phenylene.

In some embodiments, U2 is

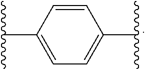 [SMILES: *c1ccc(*)cc1]

In some embodiments, V2 is a bond.

In some embodiments, V2 is —C≡C—(CH₂)ₙ—.

In some embodiments, V2 is —C≡C—(CH₂)₃—.

In some embodiments, U2 is heteroarylene.

In some embodiments, U2 is a bivalent pyrimidine ring.

In some embodiments, U2 is

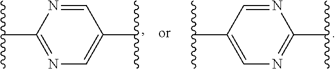 [SMILES: *c1cnc(*)nc1 ; *c1cnc(*)nc1]

In some embodiments, U2 is a bond.

In some embodiments, W2 is —C(═O)—.

In some embodiments, Conjugator is

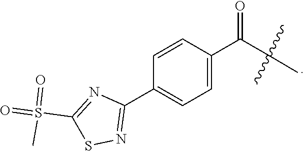 [SMILES: *C(=O)c1ccc(-c2nsc(S(C)(=O)=O)n2)cc1]

In some embodiments, the Conjugator has formula (V).

In some embodiments, RG4 is

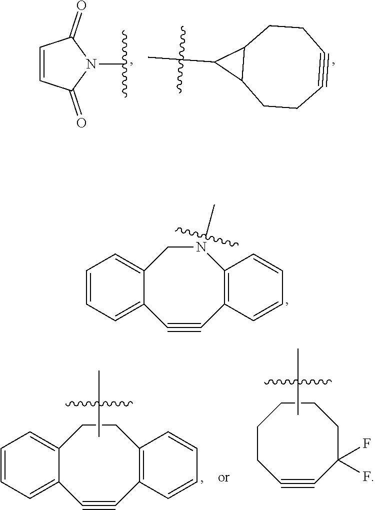

In some embodiments, RG4 is

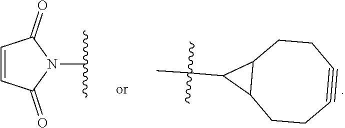 [SMILES: *N1C(=O)C=CC1=O ; *C1C2CCC#CCCC12]

In some embodiments, RG4 is

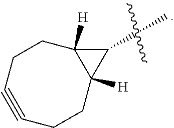 [SMILES: *[C@H]1[C@H]2CCC#CCC[C@@H]12]

In some embodiments, t4 is 1, 2, 3, 4, 5, or 6.

In some embodiments, t4 is 1, 2, 3, 4, or 5.

In some embodiments, s4 is 0.

In some embodiments, the sum of s4 and t4 is 1 or 5

In some embodiments, RE4 is a bond or —O—.

In some embodiments, W4 is —C(═O)—.

In some embodiments, Conjugator is

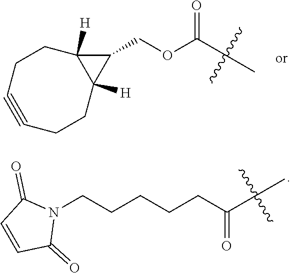

In some embodiments, Conjugator has formula (IV).

In some embodiments, RG3 is

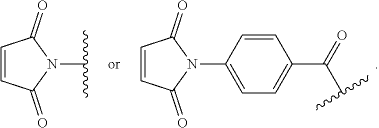 [SMILES: *N1C(=O)C=CC1=O ; *C(=O)c1ccc(N2C(=O)C=CC2=O)cc1]

In some embodiments, RG3 is

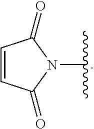 [SMILES: *N1C(=O)C=CC1=O]

In some embodiments, RS3 is NH₂ or —N(CH₃)₂.

In some embodiments, RS3 is NH₂.

In some embodiments, RE3 is a bond.

In some embodiments, t3 is 2.

In some embodiments, RE3 is —O—.

In some embodiments, s3 is 0.

In some embodiments, W3 is —C(═O)—.

In some embodiments, Conjugator is

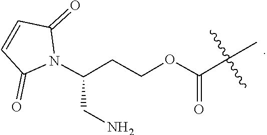 [SMILES: *C(=O)OCC[C@H](CN)N1C(=O)C=CC1=O]

In some embodiments, the compound is one of the following:

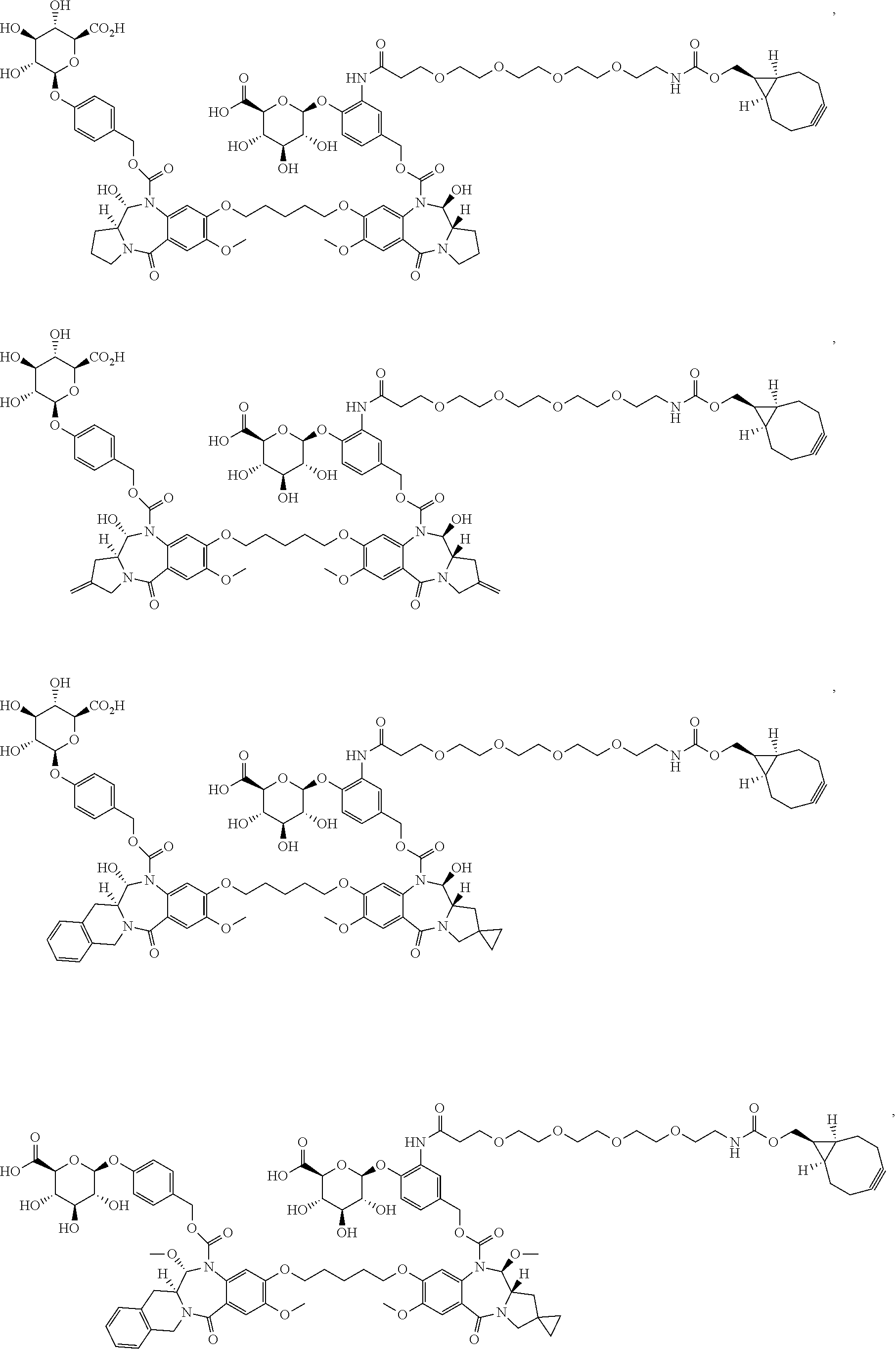

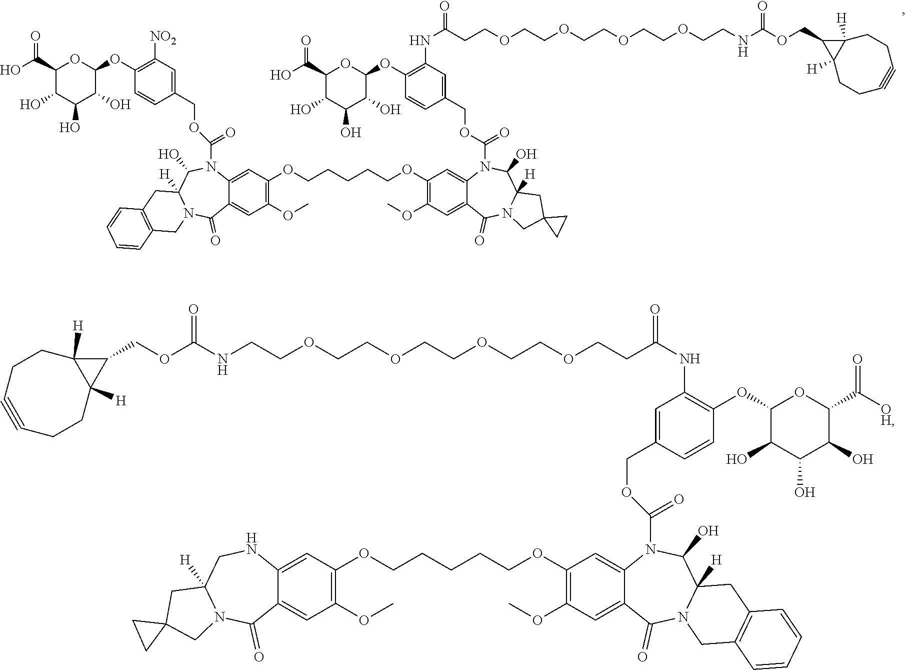

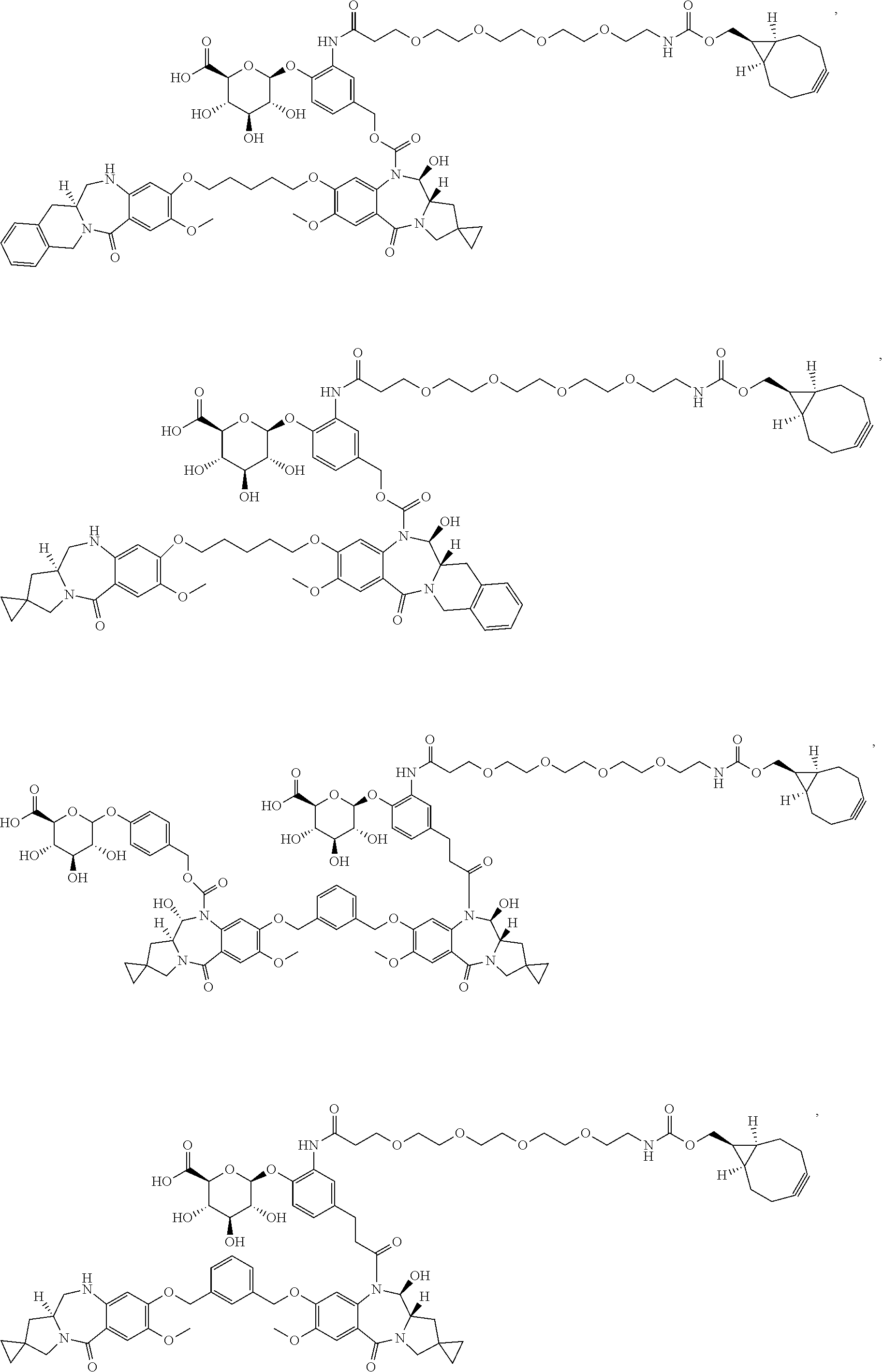

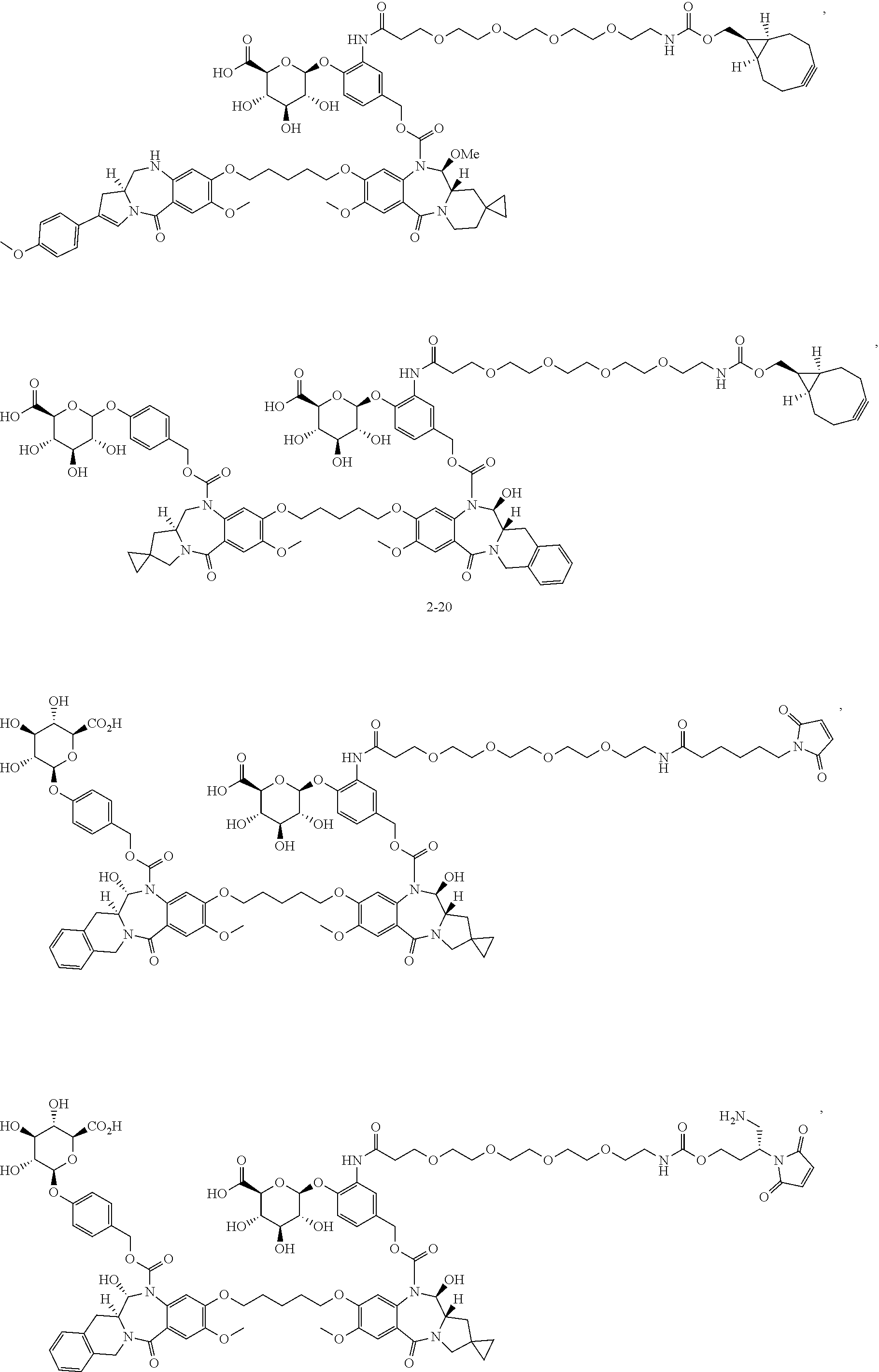

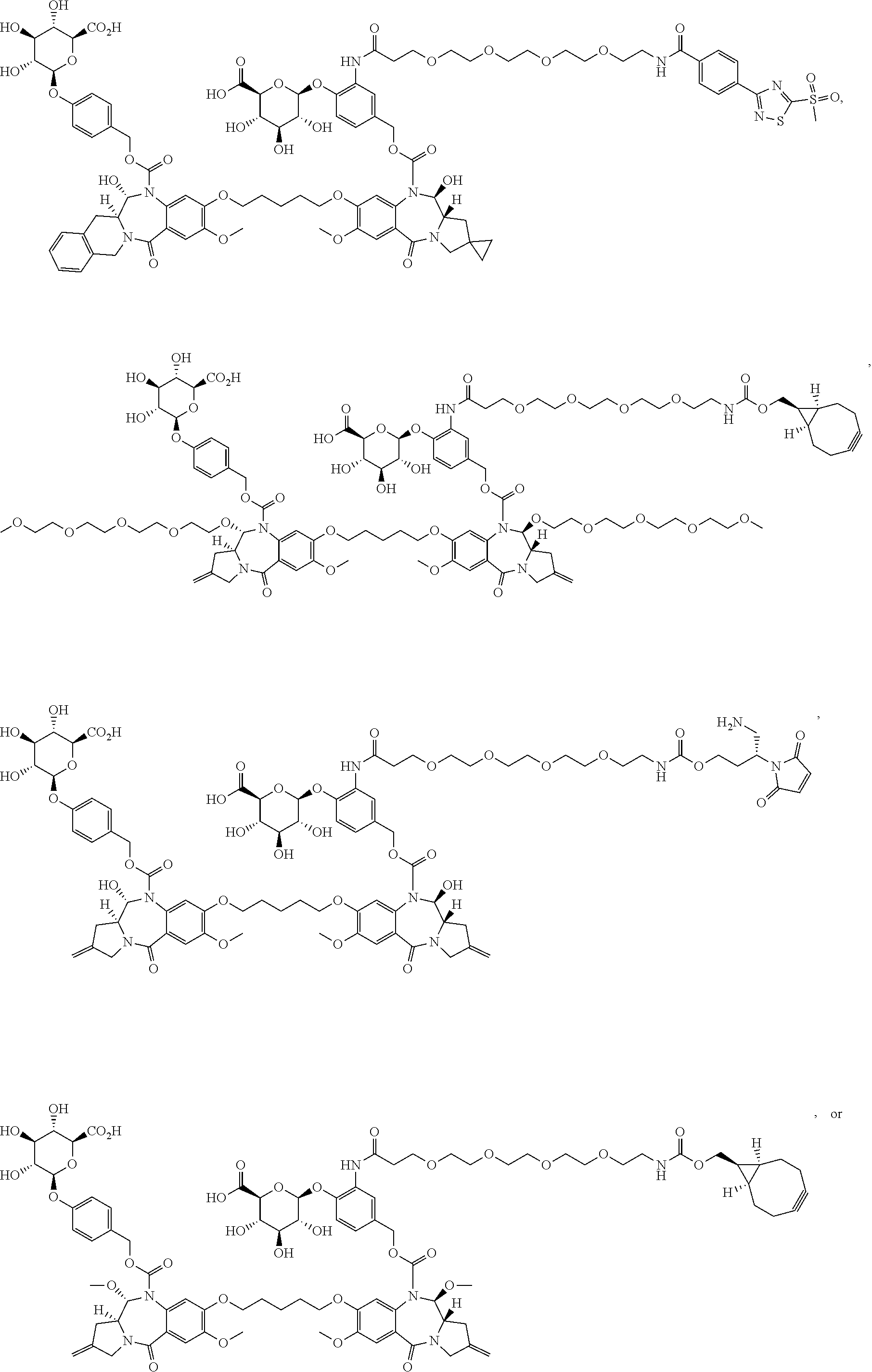

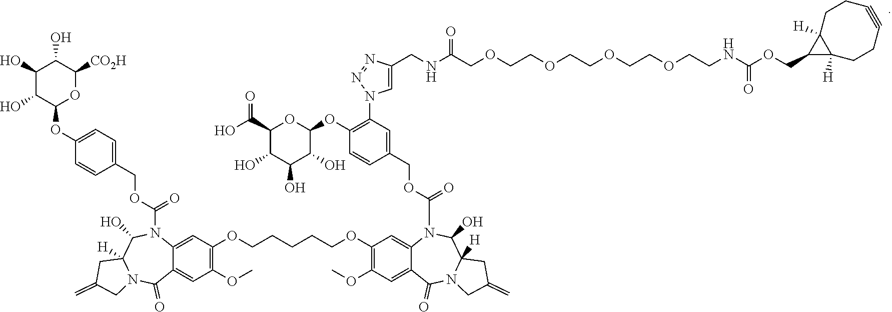 [SMILES: C=C1C[C@H]2[C@H](O)N(C(=O)OCc3ccc(O[C@@H]4O[C@H](C(=O)O)[C@@H](O)[C@H](O)[C@H]4O)cc3)c3cc(OCCCCCOc4cc5c(cc4OC)C(=O)N4CC(=C)C[C@H]4[C@H](O)N5C(=O)OCc4ccc(O[C@@H]5O[C@H](C(=O)O)[C@@H](O)[C@H](O)[C@H]5O)c(-n5cc(CNC(=O)COCCOCCOCCOCCNC(=O)OC[C@@H]6[C@@H]7CCC#CCC[C@@H]76)nn5)c4)c(OC)cc3C(=O)N2C1]

### Aspect 2

In some embodiments, the platform is a conjugator-linker-payload compound of Formula (IA):

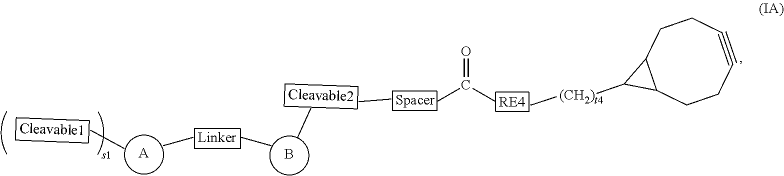

or a pharmaceutically acceptable salt, tautomer, solvate, stereoisomer, or isotopologue thereof, wherein:
each of ring A and ring B is, independently, one of formula (IIa), (IIb), (IIc), (IId), (IIe), (IIf), or (IIg):

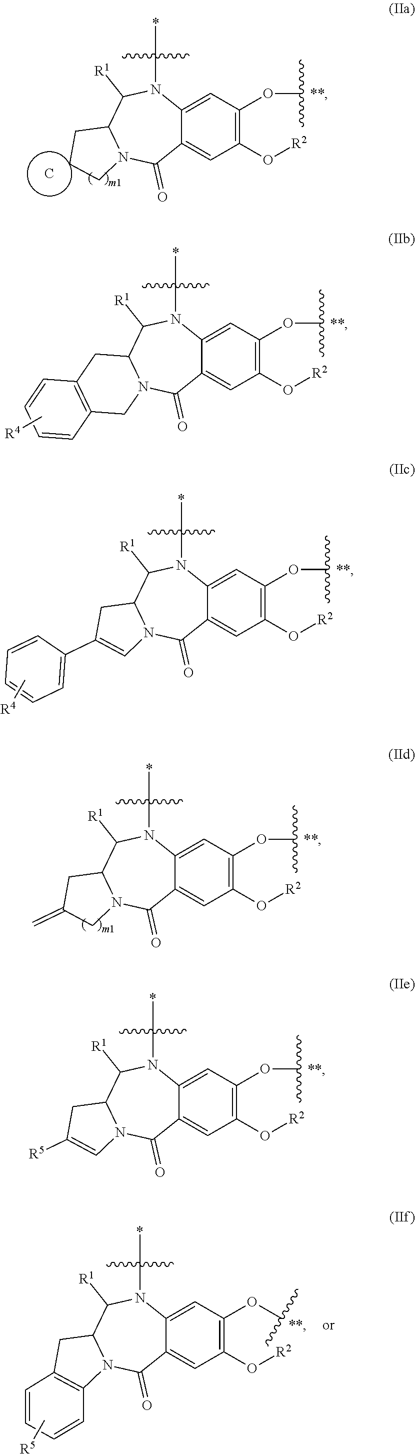

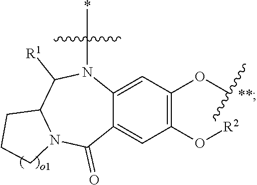

wherein:
each ring C is independently a cyclopropyl ring or a cyclobutyl ring;
each R¹ is independently H, OH, C₁₋₄ alkyl, C₁₋₄ alkoxyl, or —O—(CH₂CH₂O)ₜ₁—CH₃;
each R² is independently H or C₁₋₄ alkyl;
each R³ and R⁴ is, independently, H, NR^{a1}R^{b1}, OH, C₁₋₄ alkyl, C₁₋₄ alkoxyl, or aryl;
each of R^{a1} and R^{b1} is, independently, H or C₁₋₄ alkyl;
each R⁵ is independently H, C₁₋₄ alkyl, C₁₋₄ alkoxyl, or aryl;
each m1, n1, and o1 is, independently, 1 or 2;
t1 is 1, 2, 3, 4, 5, 6, 7, or 8;
* marks the bond where ring A connects to Cleavable1 when s1 is 1 or to —H when
s1 is 0, or where ring B connects to Cleavable2, and
** marks the bond where ring A or ring B connects to Linker;
Linker is —(CH₂)ᵣ—, —(CH₂)ₚ—X—(CH₂)_{q}—, or —(CH₂)ₚ—CH═CH—(CH₂)_{q}—; wherein:
each r, p, and q, is, independently, 1, 2, 3, 4, 5, 6, 7, or 8;
the sum of p and q is 1, 2, 3, 4, 5, 6, 7, or 8;
X is NR⁶, NHC(═O), C(═O)NH, O, SO₂, a substituted or unsubstituted divalentaryl ring, substituted or unsubstituted divalent heteroaryl ring, substituted or unsubstituted divalent heterocyclic ring, or substituted or unsubstituted divalent cyclic ring; and
R⁶ is H or C₁₋₄ alkyl;
Cleavable1, if present, has the formula

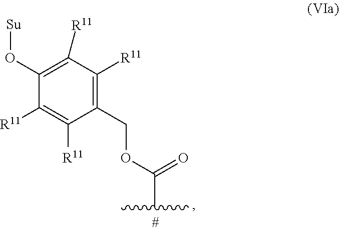

wherein:
Su is a sugar moiety;
each R¹¹ is, independently, hydrogen, halogen, substituted or unsubstituted C₁₋₄ alkyl, —CN, or —NO₂; and
# marks the bond where Cleavable1 connects to ring A;
s1 is 0 or 1;
Cleavable2 has formula (VIIa1) or (VIId1):

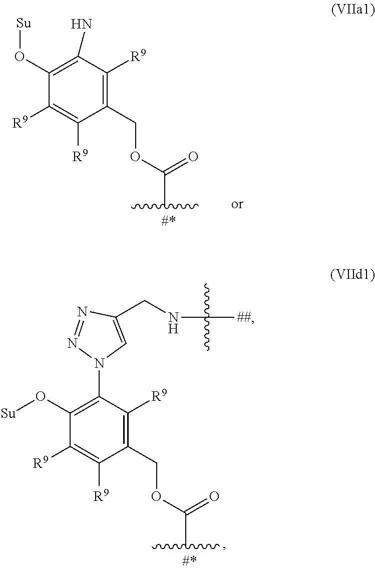

Su is a sugar moiety;
each R⁹ is, independently, hydrogen or C₁₋₄ alkyl;
#* marks the bond where Cleavable2 connects to ring B; and
## marks the bond where Cleavable2 connects to Spacer;
Spacer is a bond, ^{###}—NH—(CH₂CH₂O)ₘ₂—CH₂CH2-C(═O)— or ^{###}—NH—(CH₂CH₂O)ₘ₂—CH₂—C(═O)—; wherein:
each m2 is, independently. 1, 2, 3, 4, 6, 7, or 8; and
### marks the bond where Spacer connects to —C(═O)—; RE4 is a bond or —O—; and
t4 is 1, 2, 3, 4, 5, 6, 7, or 8.

In some embodiments, s1 is 0.

In some embodiments, Linker is —(CH₂)ᵣ or —(CH₂)ₚ—X—(CH₂)_{q}—, ring A has formula (IIa), (IIb), (IIc), (IId), or (IIg), and ring B has formula (IIa), (IIb), (IId), or (IIg).

In some embodiments, Linker is —(CH₂)ᵣ—, ring A has formula (IIg), and ring B has formula (IIg).

In some embodiments, Linker is —(CH₂)ᵣ—, ring A has formula (IId), and ring B has formula (IId).

In some embodiments, Linker is —(CH₂)ᵣ—, ring A has formula (IIb), and ring B has formula (IIa).

In some embodiments, Linker is —(CH₂)ᵣ—, ring A has formula (IIa), and ring B has formula (IIb).

In some embodiments, Linker is —(CH₂)ᵣ—, ring A has formula (IIa), and ring B has formula (IIa).

In some embodiments, Linker is —(CH₂)ᵣ—, ring A has formula (IIc), and ring B has formula (IIa)

In some embodiments, Linker is —(CH₂)ₚ—X—(CH₂)_{q}—, ring A has formula (IIa), and ring B has formula (IIa).

In some embodiments, Linker is —(CH₂)ᵣ—, ring A has formula (IIc), and ring B has formula (IIb).

In some embodiments, r is 3 or 5.

In some embodiments, Linker is —(CH₂)ᵣ— and r is 5.

In some embodiments, Linker is

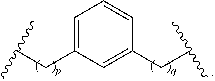

In some embodiments, the sum of p and q is 2.

In some embodiments, Linker is —(CH₂)ₚ—X—(CH₂)_{q}—, X is an unsubstituted aryl ring, p is 1, and q is 1. In some embodiments, X is phenylene.

In some embodiments, formula (IIa) is

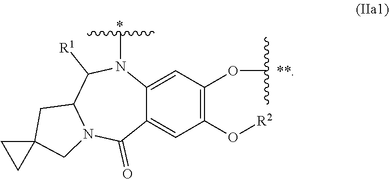

In some embodiments, formula (IId) is

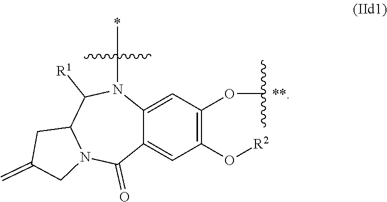

In some embodiments, formula (IIg) is

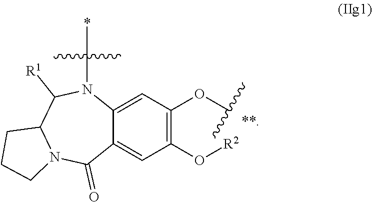

In some embodiments, formula (IIc) is

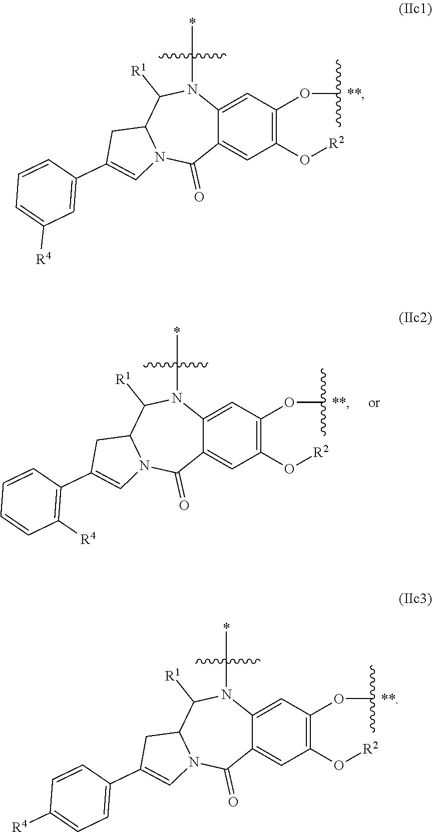

In some embodiments, formula (IIc) is

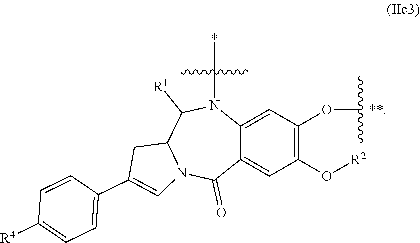

In some embodiments, ring A is

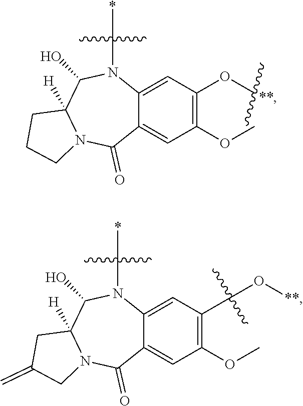

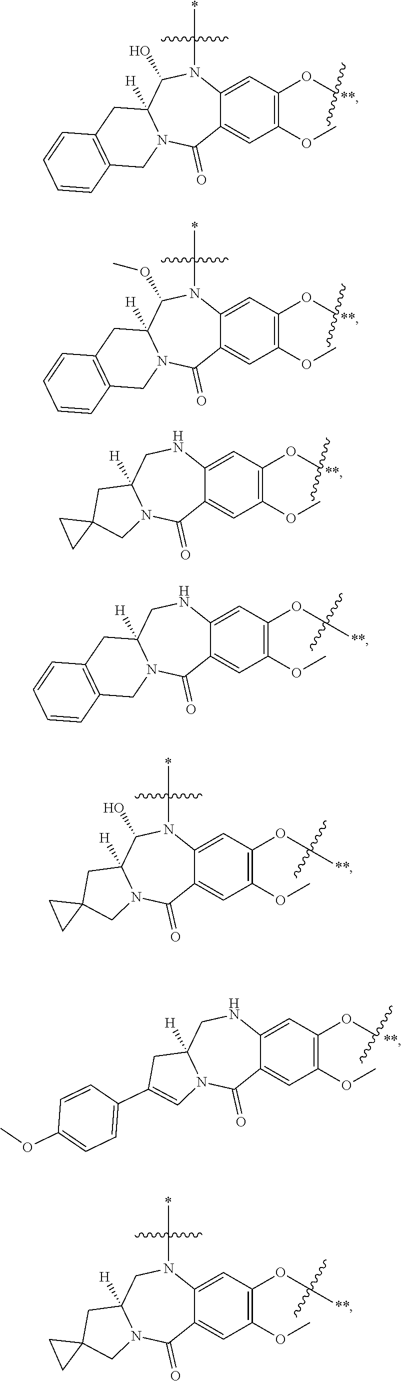

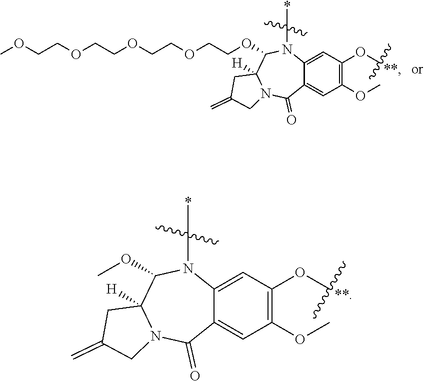

In some embodiments, ring B is

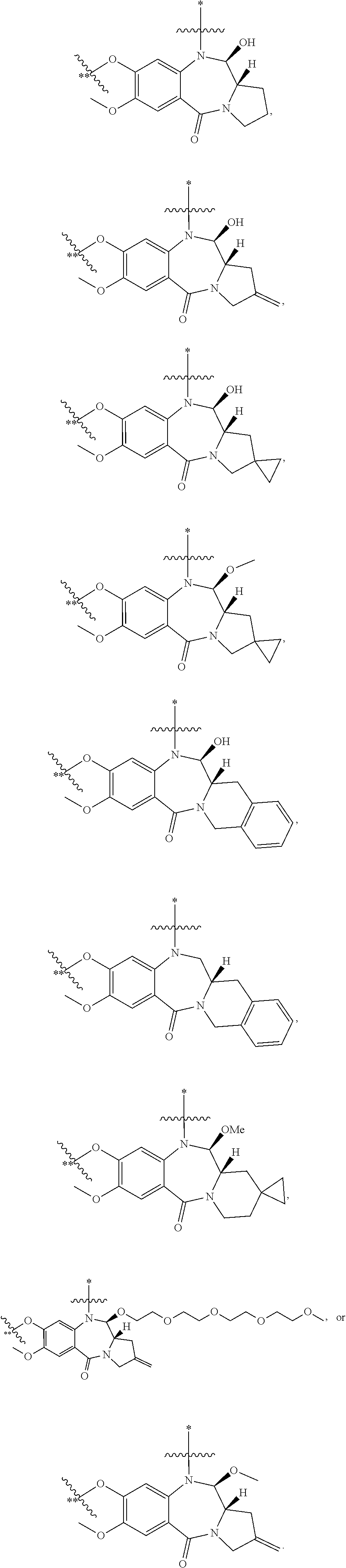

In some embodiments, when Linker is —(CH₂)ₚ—X—(CH₂)_{q}— or —(CH₂)ₚ—CH═CH—(CH₂)_{q}—, ring A has formula (IIa), m in ring A is 2, and ring B has formula (IIa), (IIb), (IIc), (IId), (IIe), (IIf), or (IIg).

In some embodiments, when Linker is —(CH₂)ₚ—X—(CH₂)_{q}— or —(CH₂)ₚ—CH═CH—(CH₂)_{q}—, ring A has formula (IIa), m in ring A is 1, and ring B has formula (IIa), (IIb), (IIc), (IId), (IIe), (IIf), or (IIg).

In some embodiments, each Su is independently

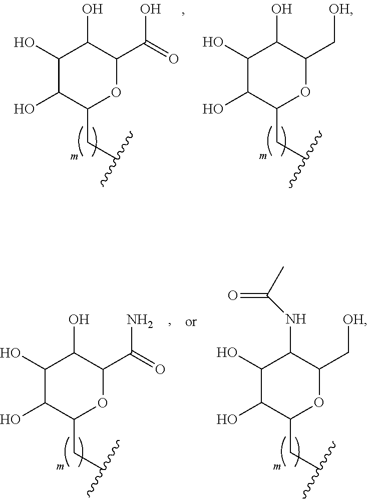

or m(or a stereoisomer thereof; each m is independently 0 or 1; and [symbol] indicates a point of attachment to Cleavable1 or Cleavable2.

In some embodiments, each Su is independently

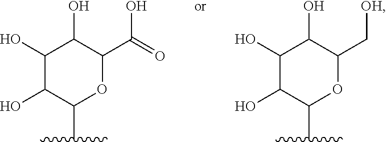 [SMILES: *C1OC(C(=O)O)C(O)C(O)C1O ; *C1OC(CO)C(O)C(O)C1O]

or a stereoisomer thereof.

In some embodiments, each Su is

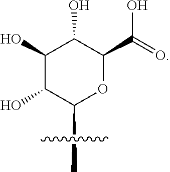

In some embodiments, Cleavable1 is

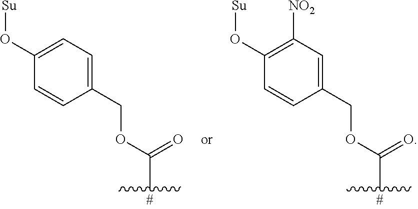

In some embodiments, s1 is 0.

In alternative embodiments, when s1 is 0, ring A has formula (IIa′), (IIb′), (IIc′), (IId′), (IIe′), (IIf′), or (IIg′):

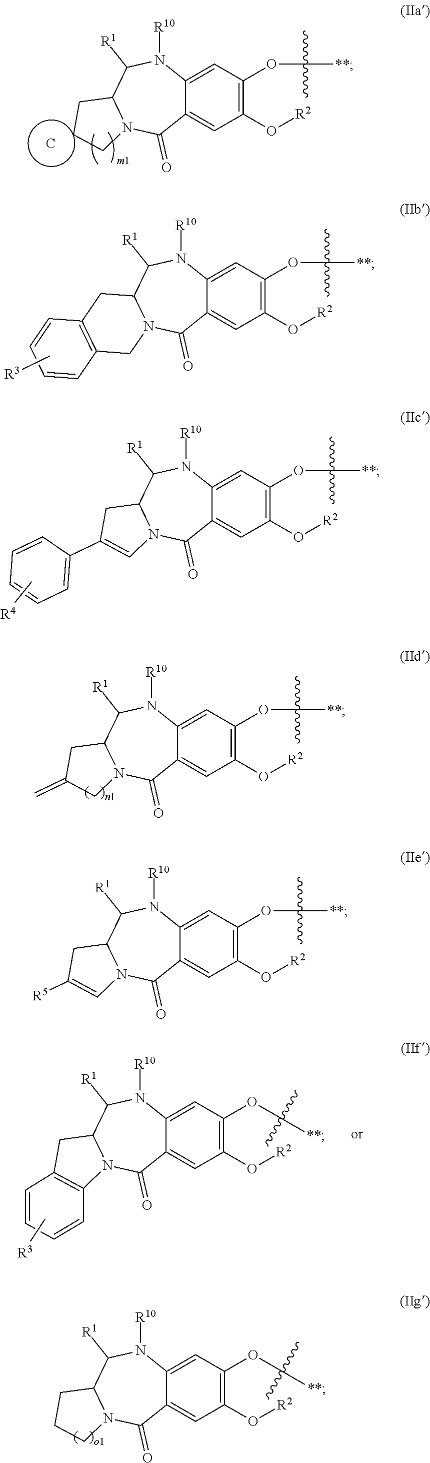

wherein R¹⁰ is, independently, H, OH, or C₁₋₄ alkyl, and the other variables (R¹, R², R³, R⁴, R⁵, m1, n1, o1) are as defined above with respect to Aspect 2.

In some embodiments, s1 is 1.

In some embodiments, Cleavable1 has formula (VIa).

In some embodiments, Cleavable2 has one of the following formulas:

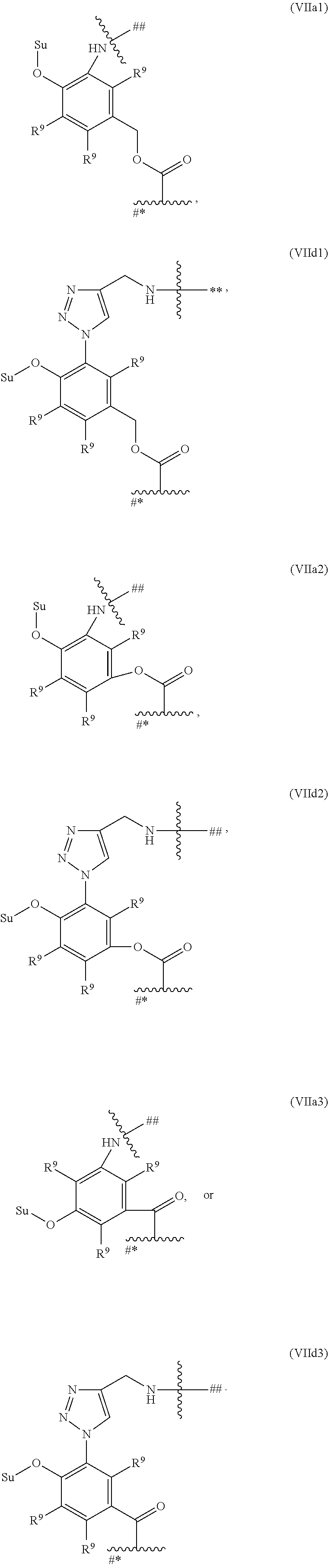

In some embodiments, Cleavable2 has formula (VIIa1) or (VIId1).

In some embodiments, each R⁹ is hydrogen.

In some embodiments, Cleavable2 is

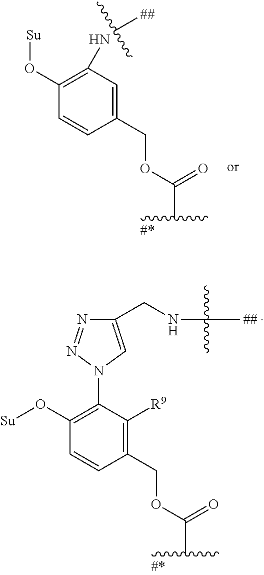

In some embodiments, Spacer is ^{###}—NH—(CH₂CH₂O)ₘ₂—CH₂CH₂—C(═O)— or ^{###}—NH—(CH₂CH₂O)ₘ₂—CH₂—C(═O)—.

In some embodiments, Spacer is ^{###}—NH—(CH₂CH₂O)₄—CH₂CH₂—C(═O)— or ^{###}—NH—(CH₂CH₂O)₄—CH₂—C(═O)—.

In some embodiments, Spacer is ^{###}—NH—(CH₂CH₂O)₄—CH₂CH₂—C(═O)—.

In some embodiments,

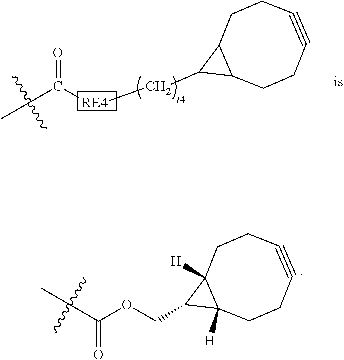

In some embodiments, the compound is one of the following:

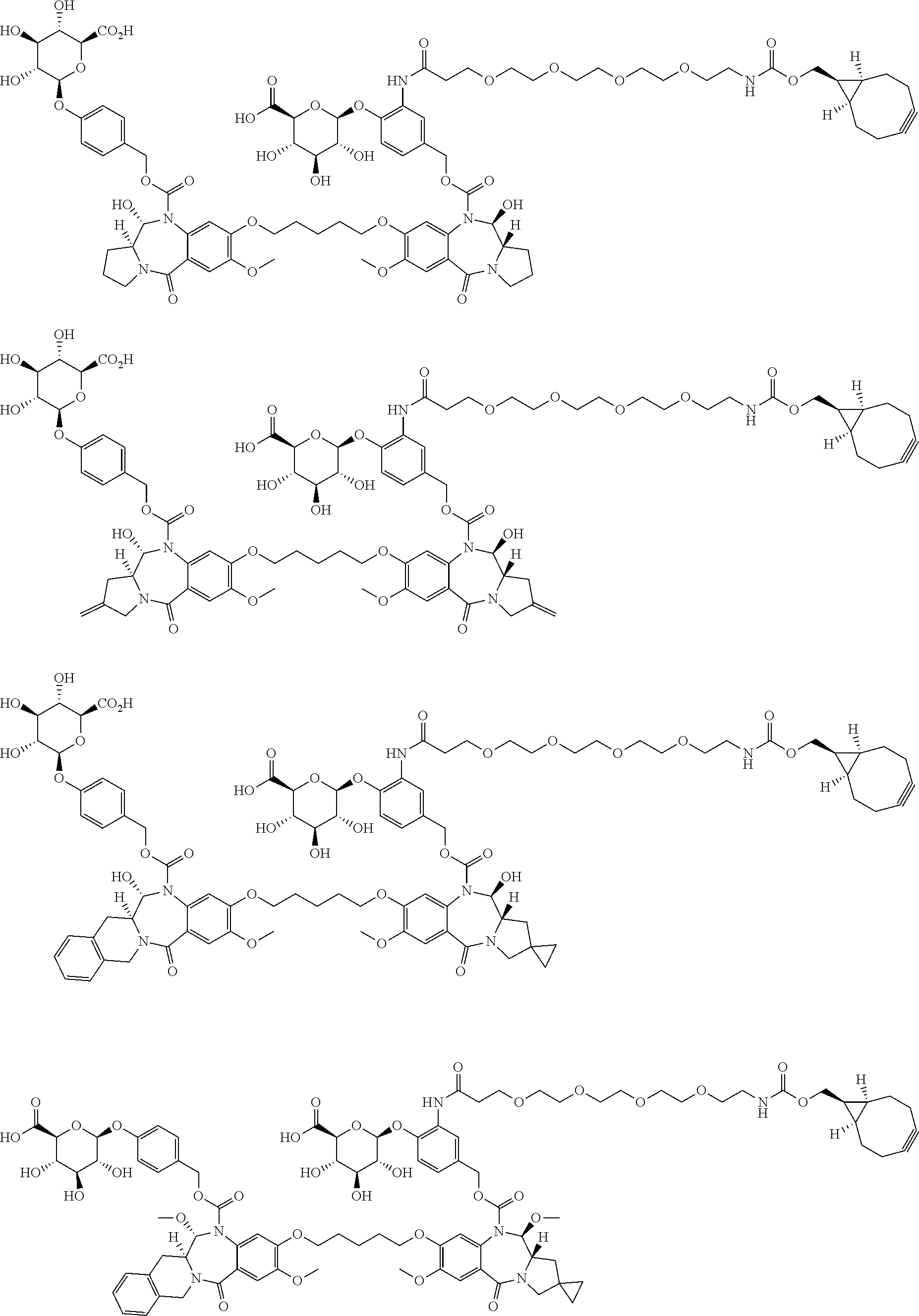

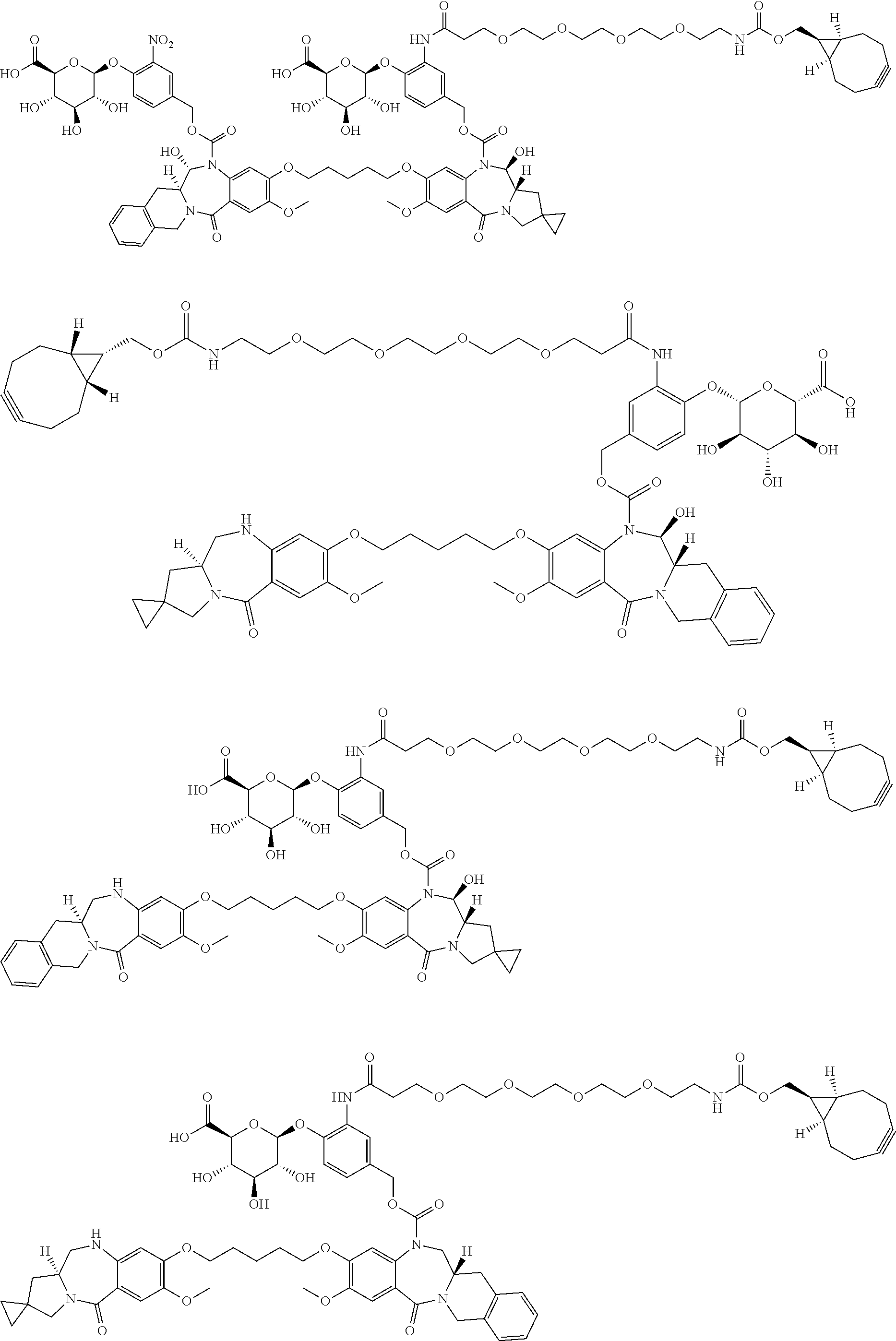

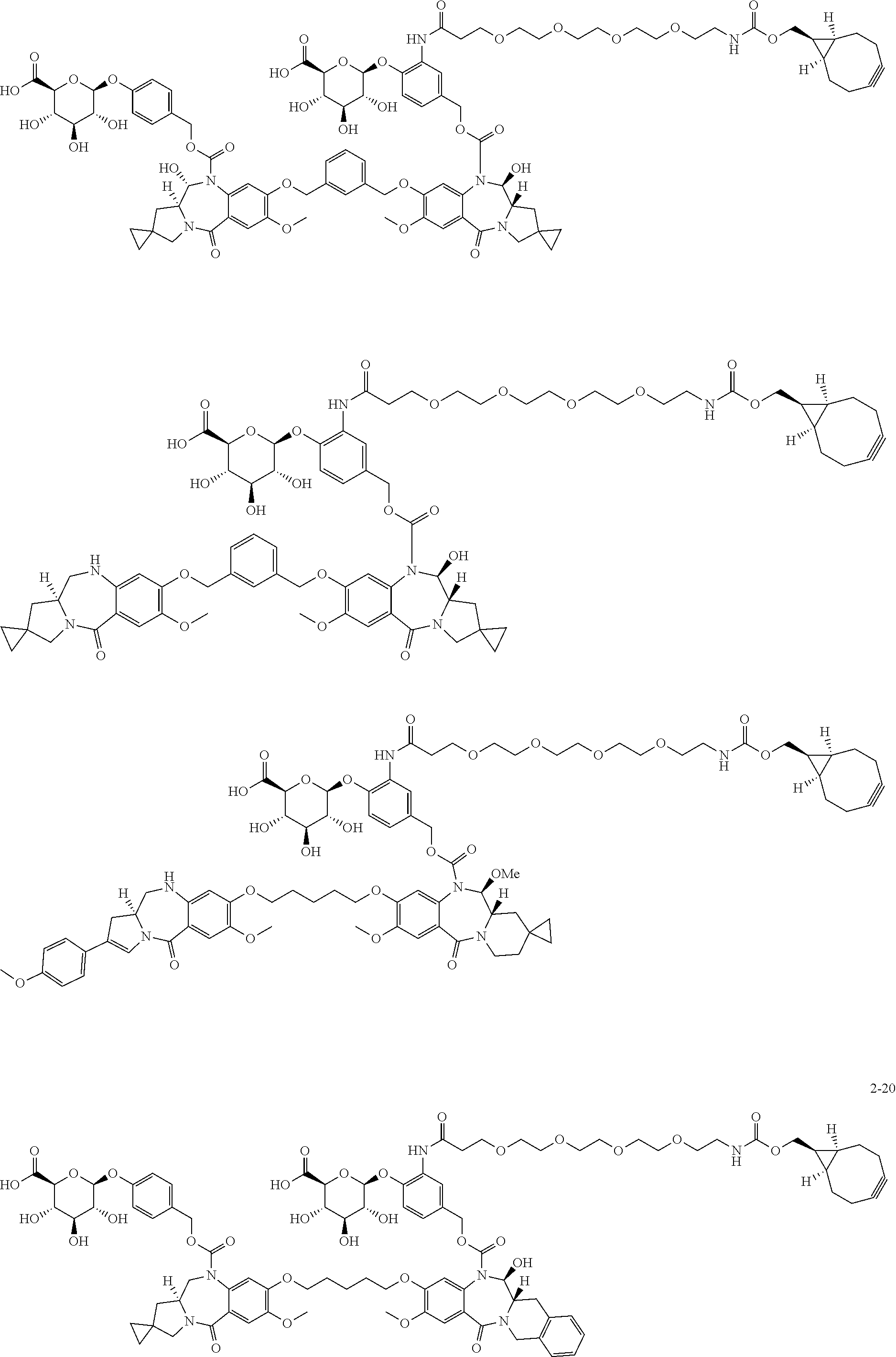

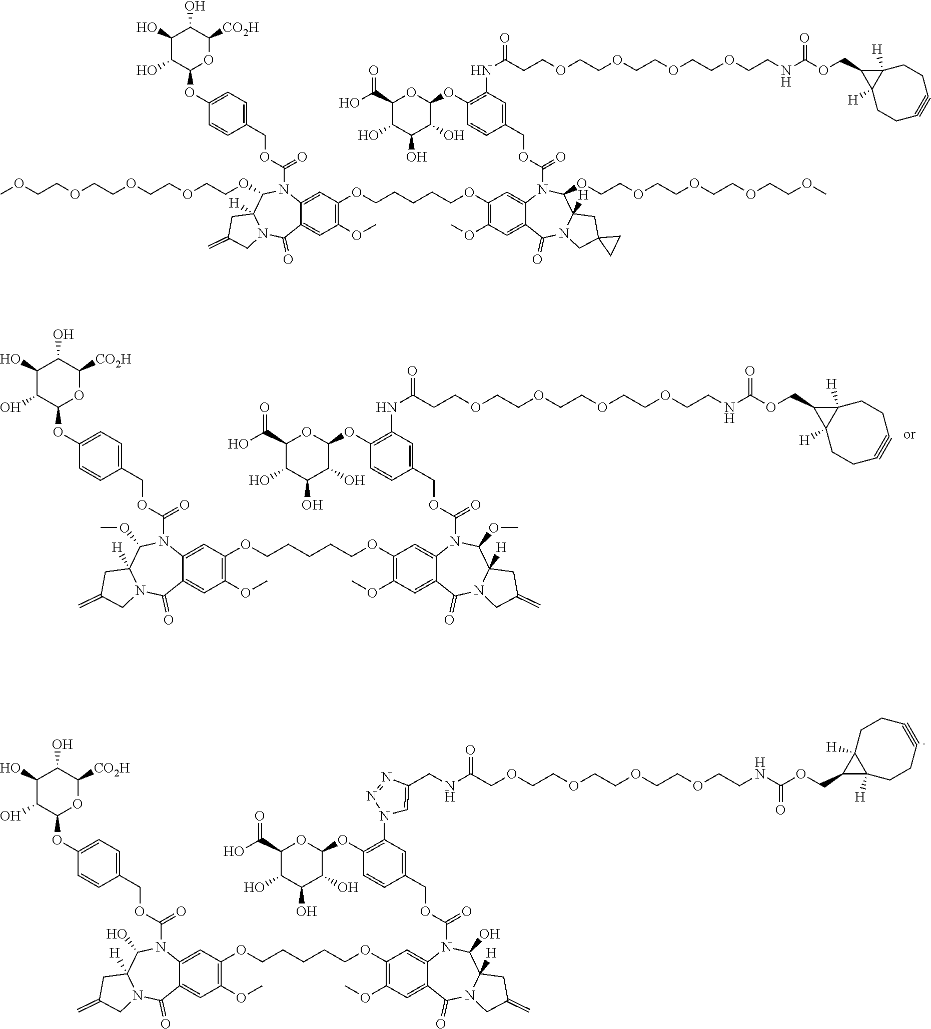

### Aspect 3

Provided herein are ligand-drug conjugates or pharmaceutically acceptable salts or solvates thereof, wherein the conjugate comprises a ligand that is conjugated to a compound provided herein via Conjugator or

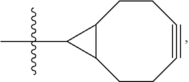 [SMILES: *C1C2CCC#CCCC12]

wherein the conjugate has one of the following formulas:

Ligand-[Formula (I)]ₓ or Ligand-[Formula (IA)]ₓ,

wherein x is from about 1 to about 12, and wherein Formula (I) is as described above with reference to Aspect 1 and Formula (IA) is as described above with reference to Aspect 2.

In one embodiment, x is from about 1 to about 10. In one embodiment, x is from about 1.5 to about 10. In one embodiment, x is from about 1.5 to about 8. In one embodiment, x is from about 1.5 to about 6. In one embodiment, x is from about 1.5 to about 4. In one embodiment, x is from about 1.8 to about 4. In one embodiment, x is from about 1.8 to about 2.5. In one embodiment, x is about 2.

In some embodiments, the ligand is a humanized, monoclonal, chimeric, or a human antibody, or an antigen binding fragment thereof.

In some embodiments, the ligand is conjugated to a compound provided herein via the

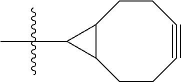 [SMILES: *C1C2CCC#CCCC12]

moiety of the compound.

In some embodiments, the conjugate has the following formula

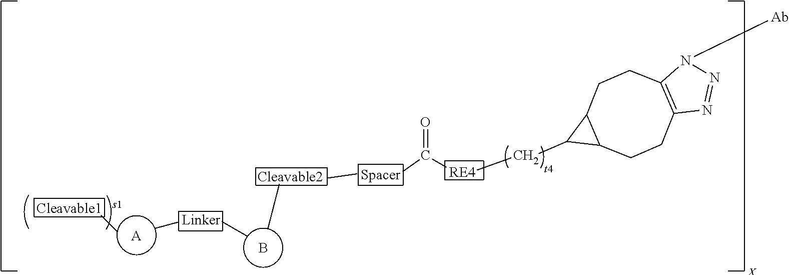

wherein Ab is a humanized, monoclonal, chimeric, or human antibody, or an antigen binding fragment thereof, and values for the remaining variables (e.g., Cleavable 1, s1, ring A, Linker, ring B, Cleavable 2, Spacer, RE4, t4) are as described herein.

In some embodiments, the ligand or Ab binds to one or more receptors chosen from B7-H3, cytokeratin 15, PTK7, HER3, HER2, CD7, CD19, CD20, CD22, CD25, CD27, CD30, CD33, CD37, CD38, CD46, CD70, CD71, CD74, CD79b, CD123, CD138, CD142, CD166, CD205, CD228, CCR2, CA6, p-Cadherin, CEA, CEACAM5, C4.4a, DLL3, EGFR, EGFRVIII, ENPP3, EphA2, EphrinA, FLOR1, FGFR2, GCC, cKIT, LIV1, LY6E, MSLN, MUC16, NaPi2b, Nectin4, gpNMB, PSMA, SLITRK6, STEAPI, TROP2, 5T4, SSEA4, GloboH, Gb5, STn, Tn, B7H3, BCMA, MUC1, cMet, ROR1 MSLN, FRa, CLDN18.2, CLDN6, PTK7, Axl, FGFR2b, CLL1, CCR7, GPC1, GPC3, ISAC, CDCP1, ITGB6, ADAM9, or CD45-iADC.

In one embodiment, Ab is an anti-CD74 antibody. In one embodiment, Ab is the anti-CD74 antibody STRO-001.

In some embodiments, the conjugate is:

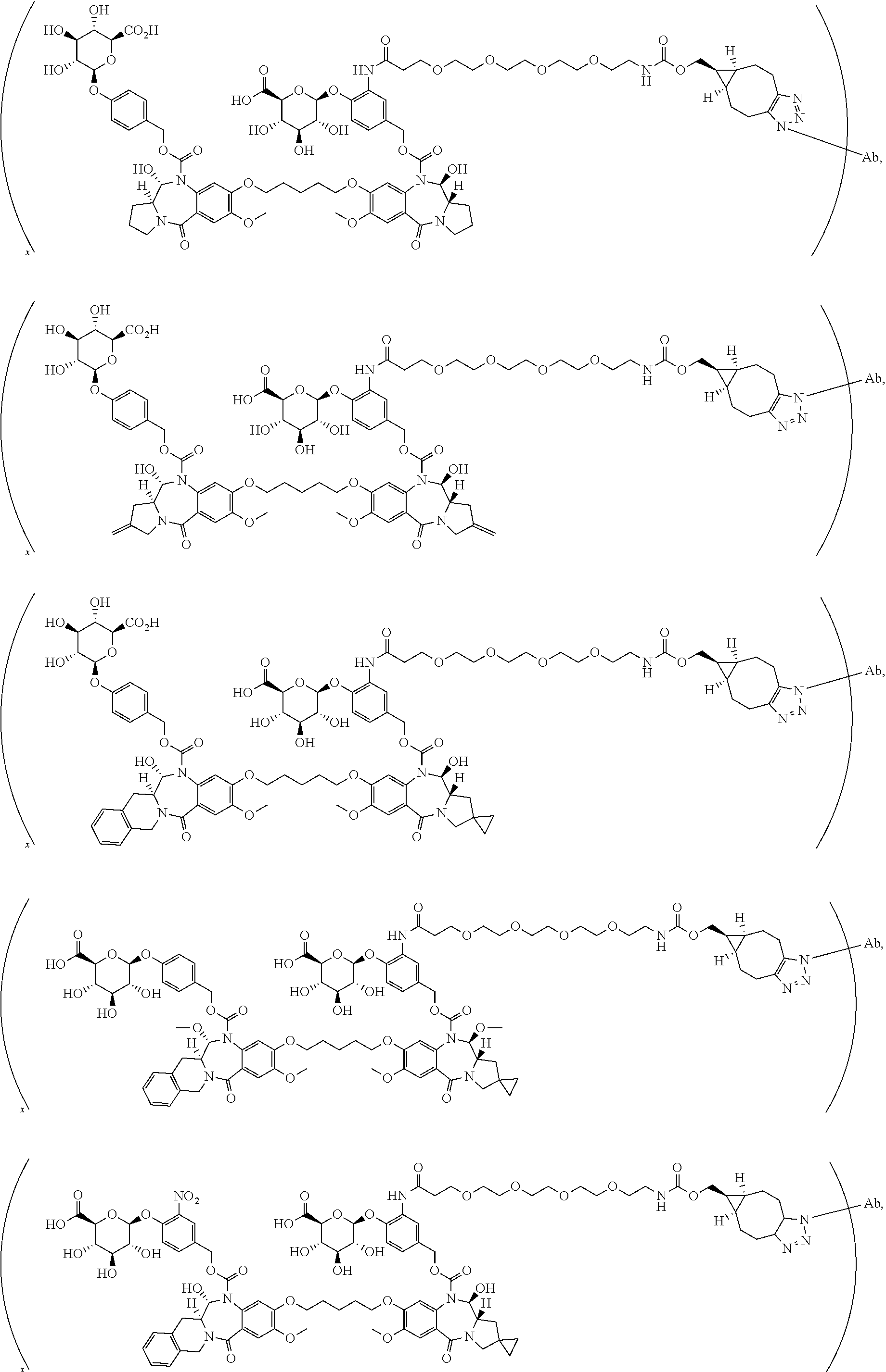

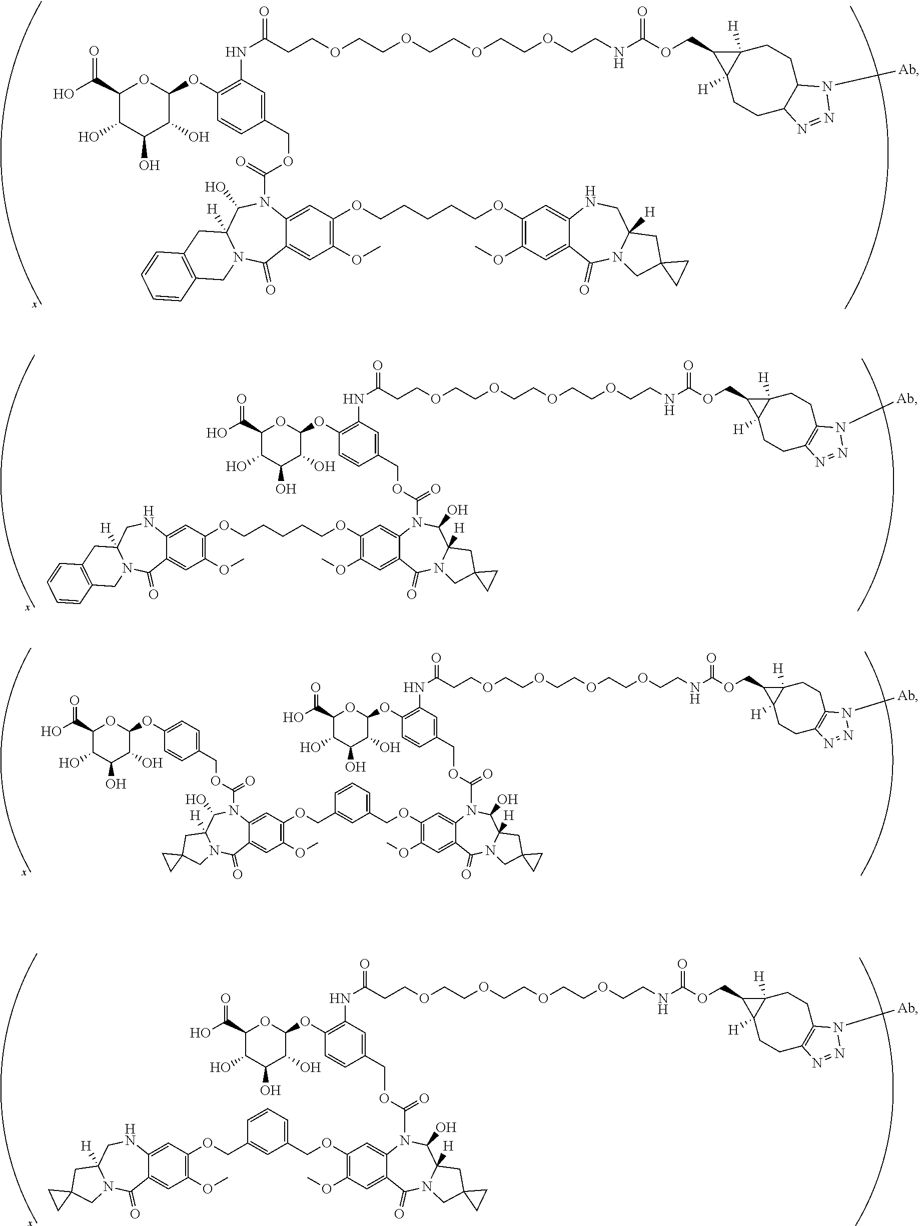

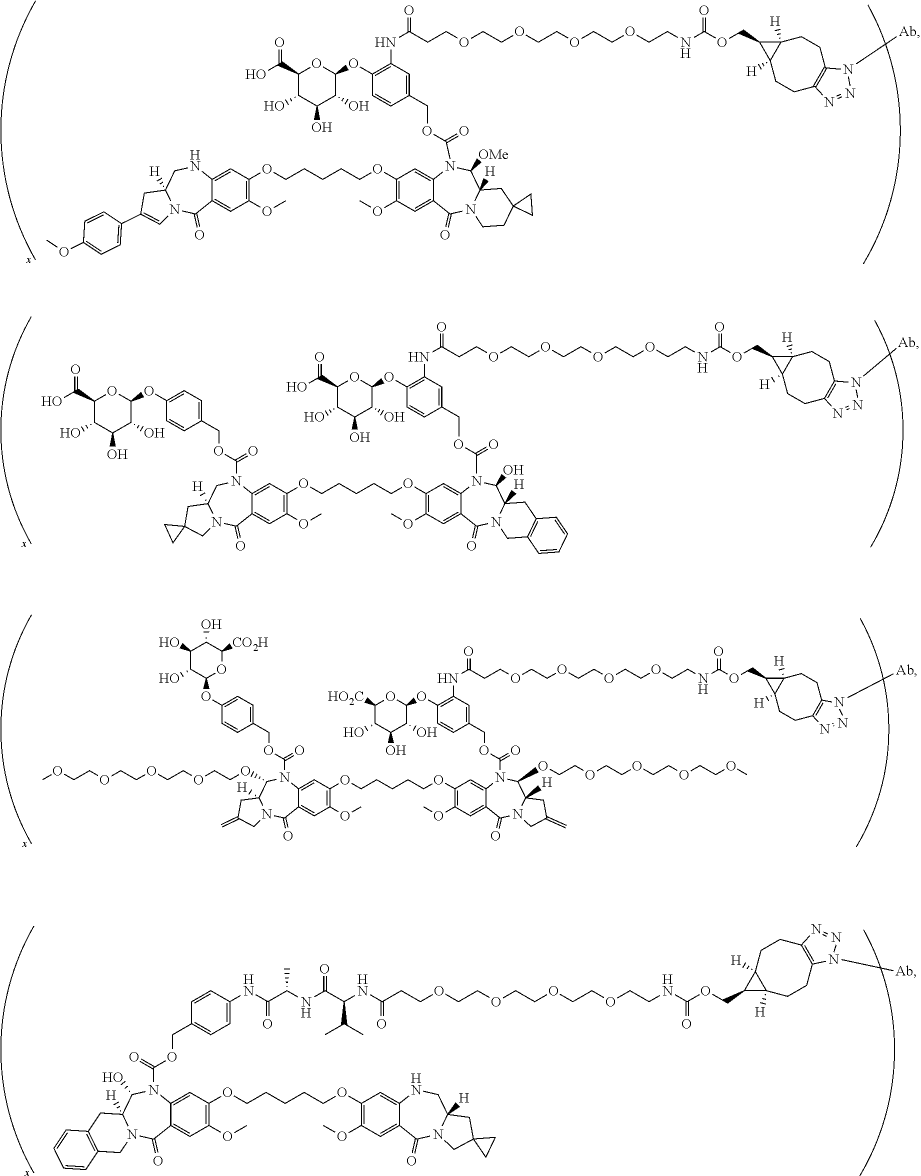

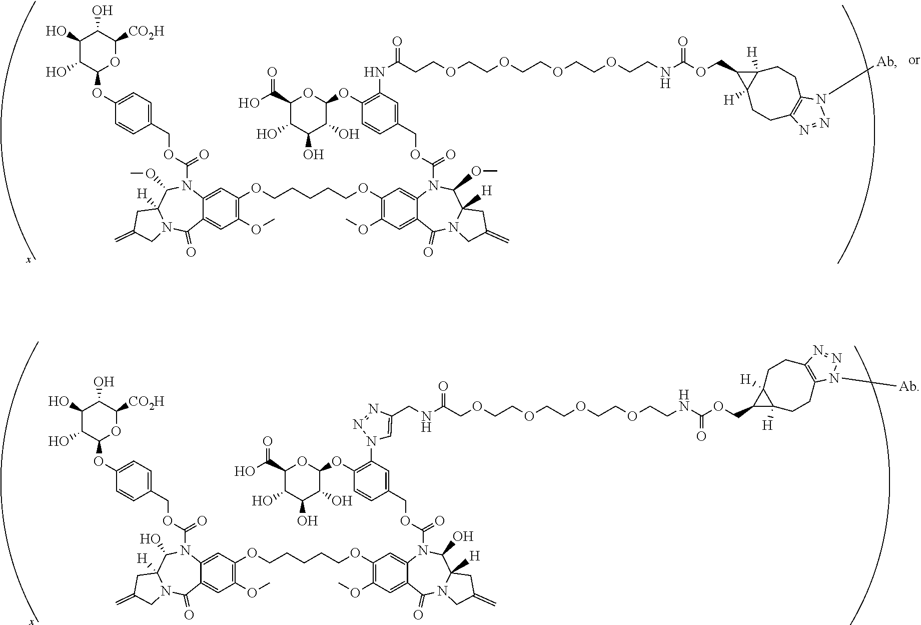

In some embodiments, the conjugate is

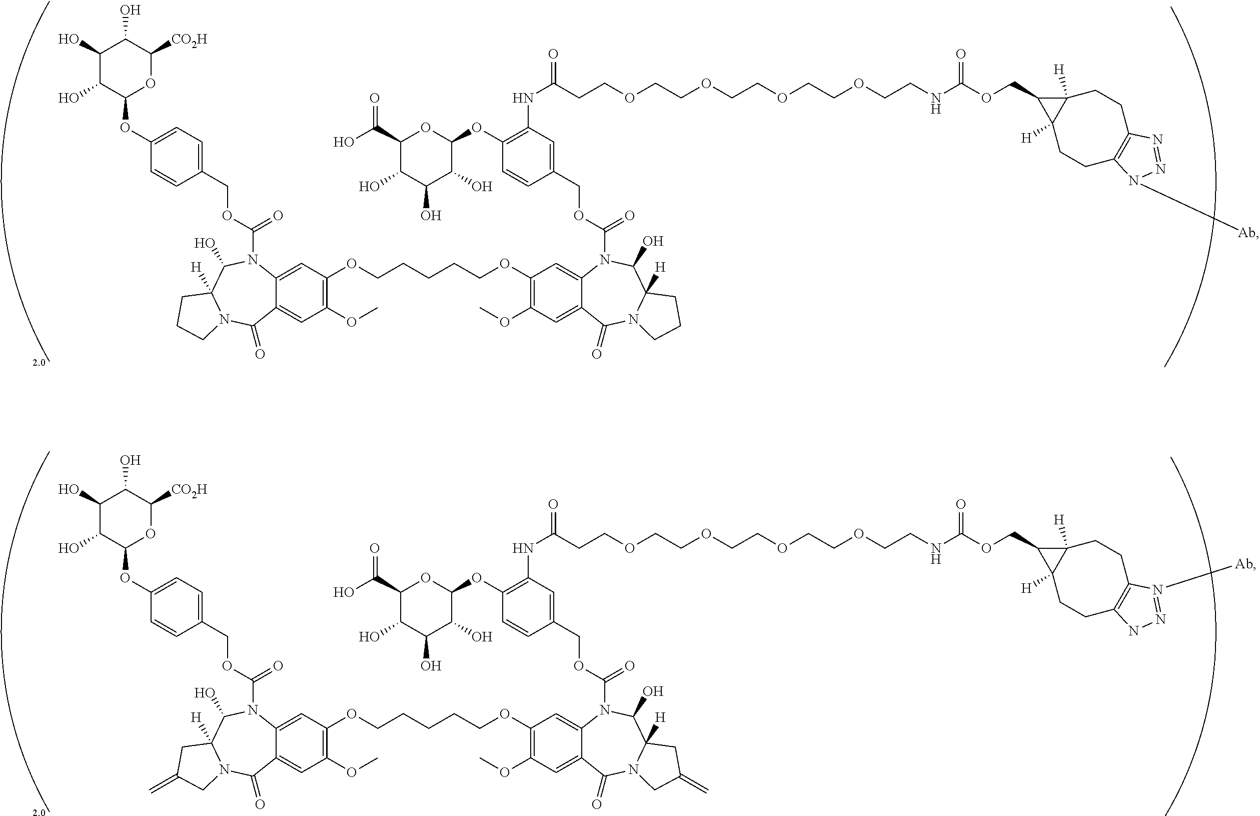

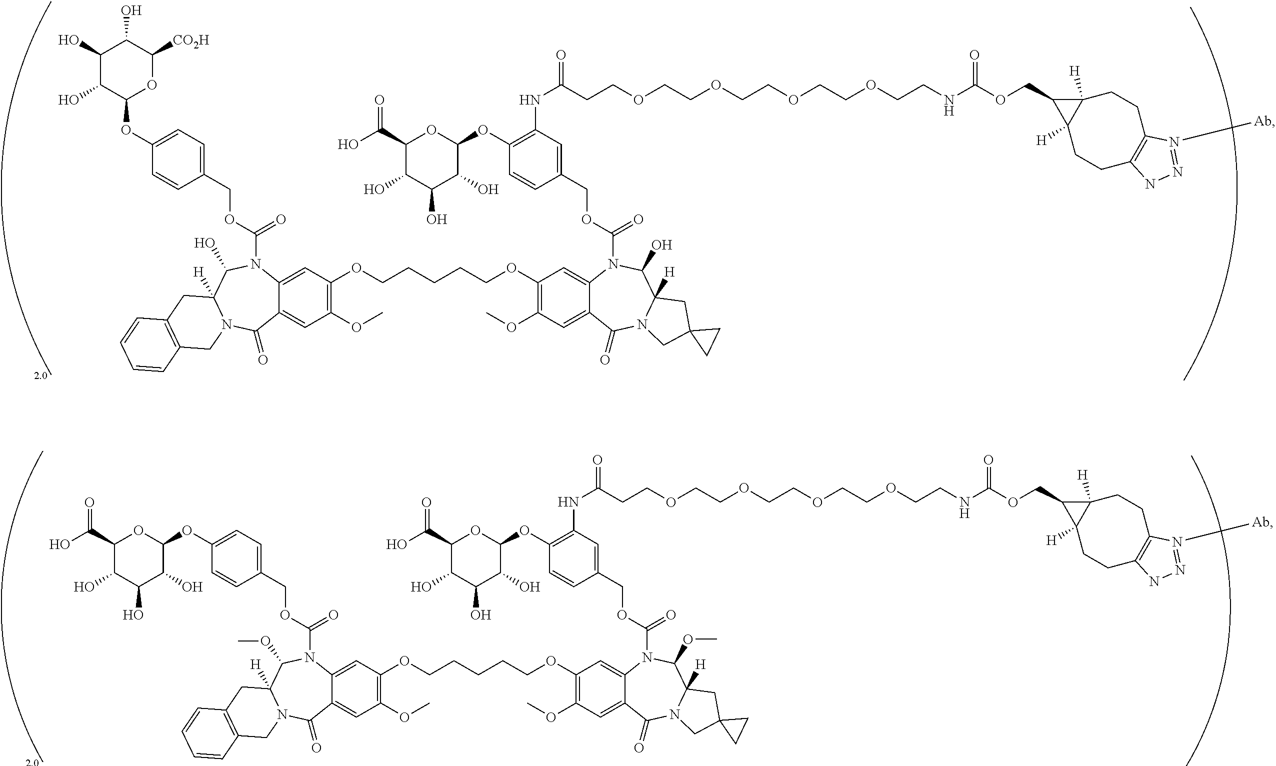

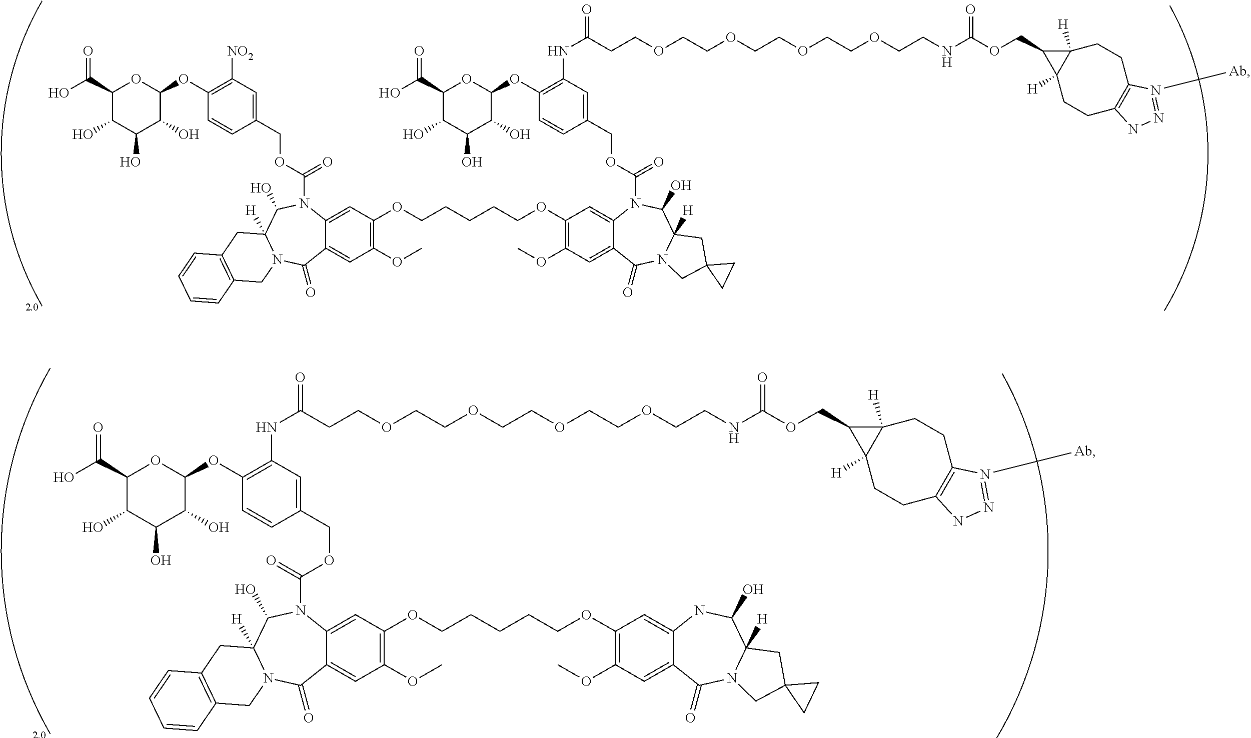

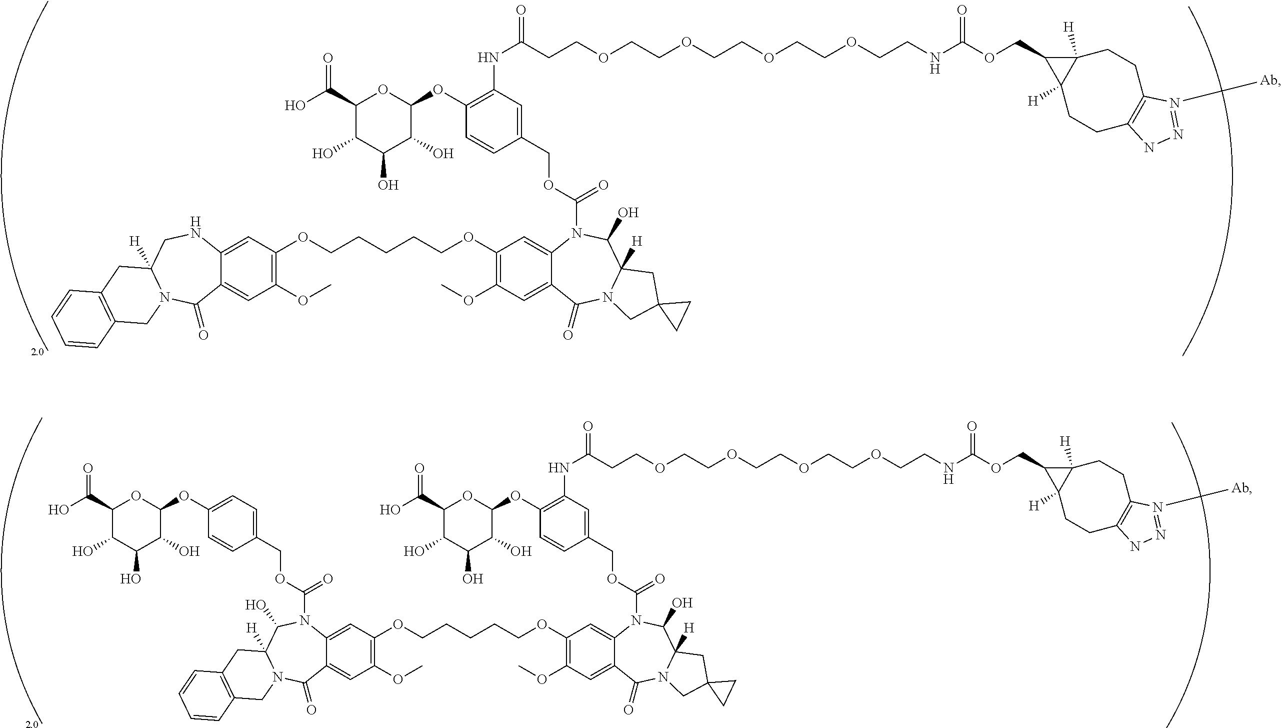

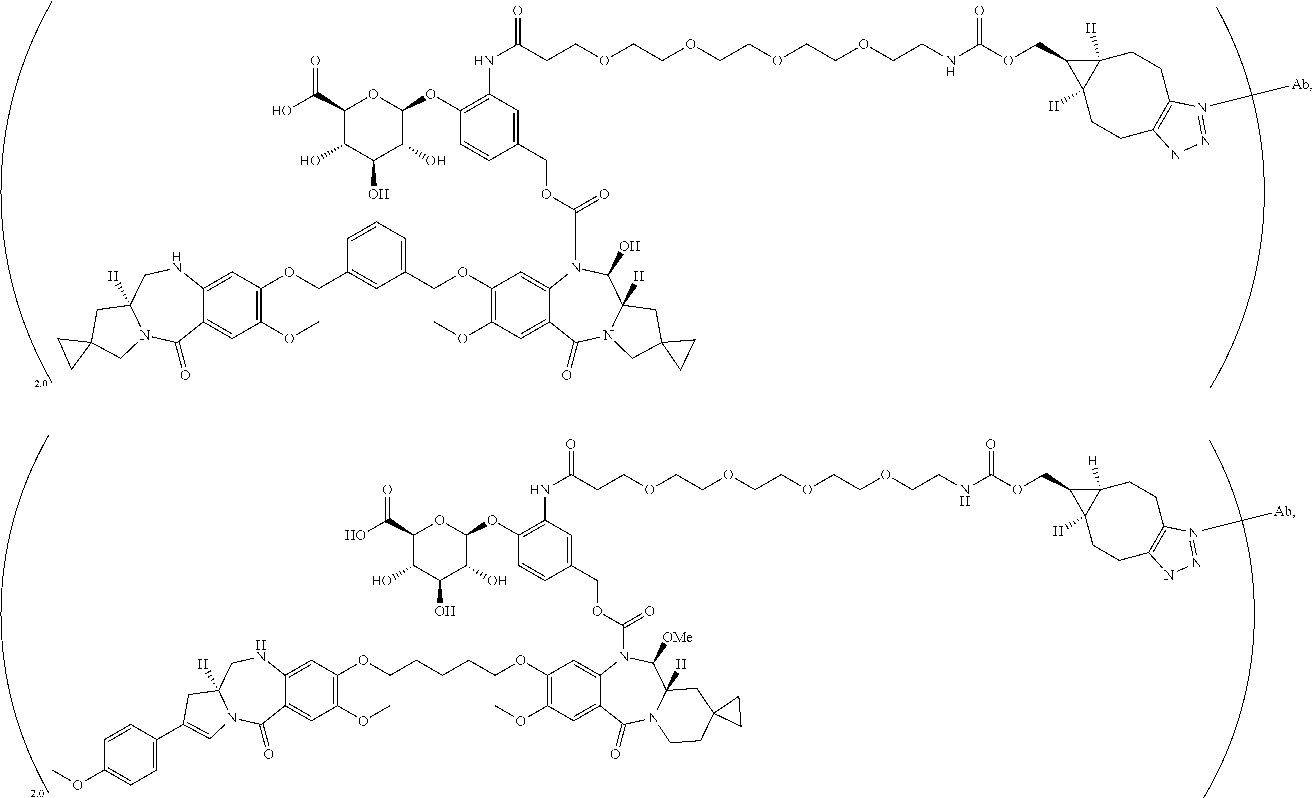

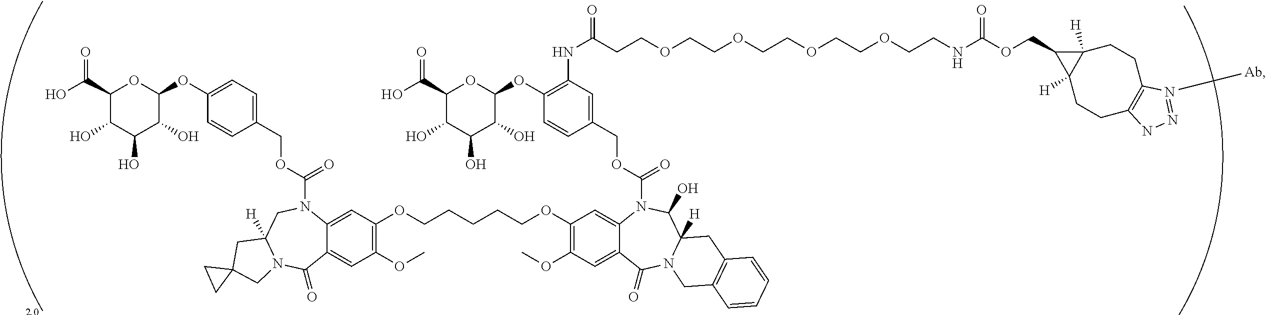

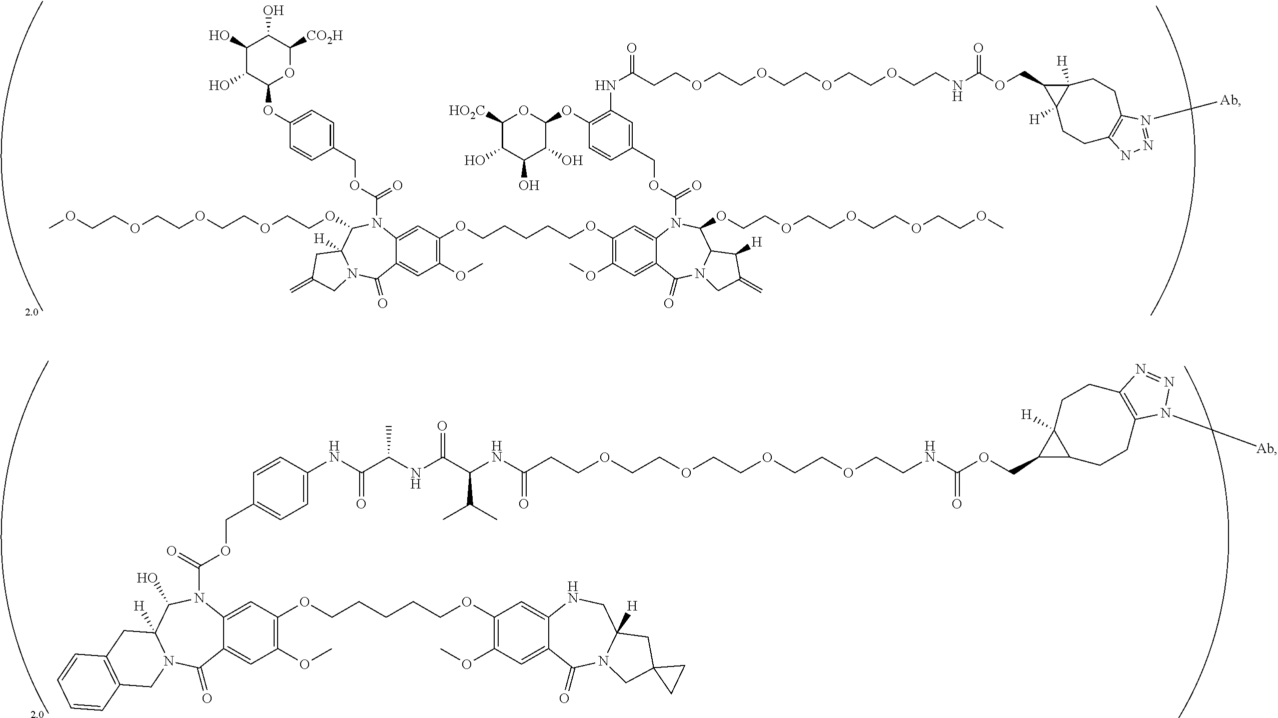

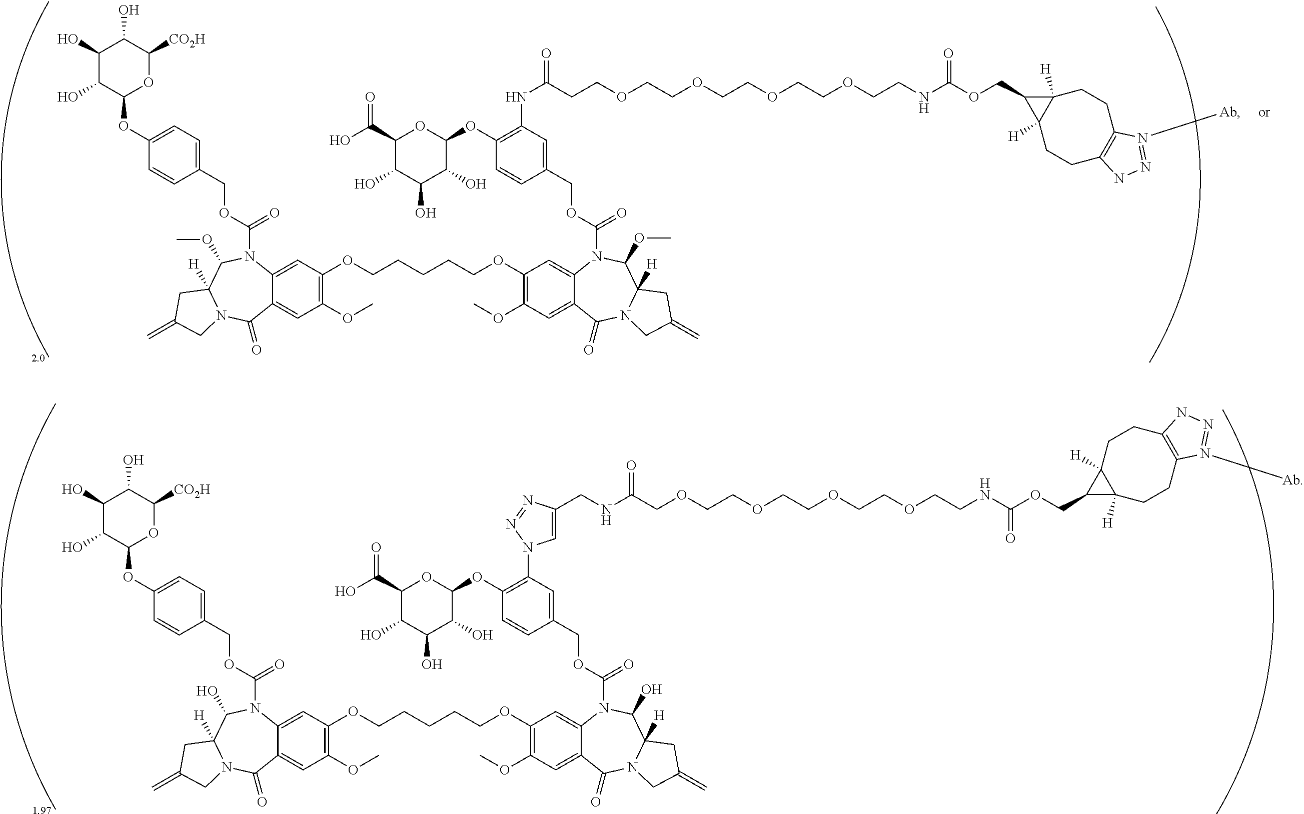

### Methods of Making the Conjugates

Provided herein are methods of preparing a conjugate by contacting a binding agent (BA) with a conjugator-linker-payload compound (platform) under conditions suitable for forming a bond between the binding agent and the conjugator-linker-payload compound. The reaction conditions may be any suitable reaction conditions known in the art. The binding agent may be an antibody and the bond may form an antibody-drug conjugate.

Examples of such reactions are provided in the Examples below.

In some embodiments, methods of making a conjugate including treating or contacting a compound with a binding agent under coupling conditions. The compound may include a reactive linker bonded to at least one payload. The compound may be any of the linker or platform compounds disclosed herein.

### Pharmaceutical Compositions

Also provided herein are compositions, including pharmaceutical compositions, comprising an ADC set forth herein. In some embodiments, the compositions (e.g., pharmaceutical compositions) further comprise a pharmaceutically acceptable excipient.

Pharmaceutical compositions in accordance with the present disclosure can be prepared by mixing an antibody drug conjugate having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to, buffers such as phosphate, citrate, and other organic acids; antioxidants including ascorbic acid and methionine; preservatives (such as octadecyl dimethyl benzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH₂O (HYLENEX®, Baxter International, Inc.). Certain exemplary sHASEGPs and methods of use, including rHuPH20, are described in U.S. Pat. No. 7,871,607 and 2006/0104968. In one aspect, a sHASEGP is combined with one or more additional glycosaminoglycanases such as chondroitinases.

### Methods of Using the Conjugates

In some embodiments, set forth herein is a method of treating a disease or disorder (e.g., a proliferative disease, a cancer, a metabolic disease, inflammation, or a neurodegenerative disease) in a subject (e.g., patient) in need thereof, comprising administering to the patient a therapeutically effective amount of a conjugate or pharmaceutical composition disclosed herein.

The conjugates disclosed herein can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injection, such as intravenous or subcutaneous injection, depending in part on whether the administration is brief or chronic. Various dosing schedules, including but not limited to, single or multiple administrations over various time-points, bolus administration, and pulse infusion are contemplated herein.

Conjugates of the disclosure can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

### EXAMPLES

The examples below are intended to be exemplary and should not be considered limiting in any way. Unless otherwise specified, the experimental methods in the Examples described below are conventional methods. Unless otherwise specified, the reagents and materials are commercially available. All solvents and chemicals employed were of analytical grade or chemical purity. Solvents were redistilled before use. Anhydrous solvents were prepared according to standard methods or reference methods. Silica gel (100-200 meshes) for column chromatography and silica gel (GF254) for thin-layer chromatography (TLC) are commercially available from Tsingdao Haiyang Chemical Co., Ltd. or Yantai Chemical Co., Ltd. of China; all were eluted with petroleum ether (60-90° C.)/ethyl acetate (v/v) and visualized by iodine or the solution of molybdphosphoric acid in ethanol unless otherwise specified. All extraction solvents, unless otherwise specified, were dried over anhydrous Na₂SO₄. ¹H NMR spectra were recorded on Bruck-400, Varian 400MR nuclear magnetic resonance spectrometer with TMS (tetramethylsilane) as the internal standard. Coupling constants were given in hertz. Peaks were reported as singlet (s), doublet (d), triplet (t), quartet (q), quintet (p), sextet (h), septet (hept), multiplet (m), or a combination thereof, br stands for broad. LC/MS data was recorded by using Agilent1100,1200 High Performance Liquid Chromatography-Ion Trap Mass Spectrometer (LC-MSD Trap) equipped with a diode array detector (DAD) detected at 214 nm and 254 nm, and an ion trap (ESI source). All compound names except the reagents were generated by ChemDraw® 18.0.

In the following examples, the following abbreviations are used:

| AcOH | Acetic acid |
|---|---|
| DIPEA/DIEA | N,N-Diisopropylethylamine |
| DMF | N,N-Dimethylformamide |
| DMP | Dess-Martin periodinane |
| EDCI | 1-(3-Dimethylaminopropyl)-3-ethylcarbodiimide hydrochloride |
| EEDQ | 2-Ethoxy-1-ethoxycarbonyl-1,2-dihydroquinoline |
| EtOAc | Ethyl acetate |
| EtOH | Ethanol |
| HATU | O-(7-Azabenzotriazol-1-y1)-N,N,N′,N′-tetraMethyluroniuM |
| | hexafluorophosphate |
| HOBt | 1-Hydroxybenzotriazole |
| MeCN/ACN | Acetonitrile |
| MeOH | Methanol |
| MMAE | Monomethyl auristatin E |
| MTBE | |
| PTSA | p-Toluenesulfonic acid |
| Prep-HPLC | Preparative high performance liquid chromatography |
| r.t. | Room temperature |
| TEA | Triethyl amine |
| TFA | Trifluoroacetic acid |
| THF | Tetrahydrofuran |
| TsOH•H₂O | p-Toluenesulfonic acid monohydrate |

### UPLC Analysis Methods

Method A: Mobile phase A: 0.1% FA in water, B: MeCN; Gradient: 10% B maintain 0.2 min, 10%-95% B, 5.8 min, 95% B maintain 0.5 min; Flow rate: 0.6 mL/min; Column: ACQUITY UPLC@BEH C18 1.7 μm.

Method B: Mobile phase A: 0.1% FA in water, B: MeCN; Gradient: 10% B maintain 0.5 min, 10%-90% B, 2.5 min, 90% B maintain 0.2 min; Flow rate: 0.6 mL/min; Column: ACQUITY UPLC@BEH C18 1.7 μm.

Method C: Mobile phase A: 0.1% FA in water, B: MeCN; Gradient: 10% B maintain 0.2 min, 10%-90% B, 1.3 min, 90% B maintain 0.3 min; Flow rate: 0.6 mL/min; Column: ACQUITY UPLC@BEH C18 1.7 μm.

### Example Int-2

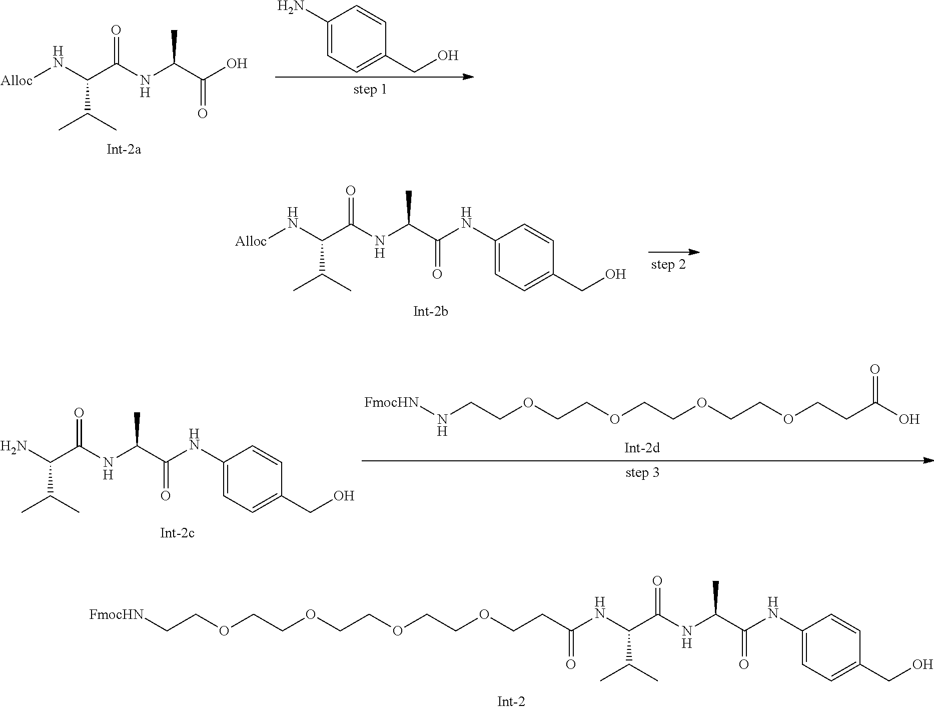

### Step 1: allyl ((S)-1-(((S)-1-((4-(hydroxymethyl)phenyl)amino)-1-oxopropan-2-yl)amino)-3-methyl-1-oxobutan-2-yl)carbamate (Int-2b)

EEDQ (4.77 g, 19.3 mmol) was added to the solution of Int-2a (5 g, 18.4 mmol) and (4-aminophenyl)methanol (2.37 g, 19.3 mmol) in 100 mL dry THF. The mixture was stirred at r.t. for 40 h. The mixture was concentrated. The residue was slurried with MTBE (30 V) and stirred for 2 h. The solid was isolated by filtration under a vacuum for 3 h to give Int-2b (5.16 g, 74% yield). MS (ESI) m/z: 378.4 [M+H]⁺.

### Step 2: (S)-2-amino-N—((S)-1-((4-(hydroxymethyl)phenyl)amino)-1-oxopropan-2-yl)-3-methylbutanamide (Int-2c)

Pd(PPh₃)₄ (76.6 mg, 0.066 mmol) was added to a solution of Int-2b (500 mg, 1.33 mmol) in CH₂Cl₂ (10 mL) and pyrrolidine (270.8 μL, 3.31 mmol) at r.t. under N₂. The reaction mixture was allowed to stir for 0.5 h at r.t. The reaction was concentrated and purified by silica gel column chromatography (CH₂Cl₂/MeOH=92/8) to give Int-2c (370 mg, 95% yield) as a white solid. MS (ESI) m/z: 294.3 [M+H]⁺.

### Step 3: (9H-fluoren-9-yl)methyl ((17S,20S)-21-((4-(hydroxymethyl)phenyl)amino)-17-isopropyl-20-methyl-15,18,21-trioxo-3,6,9,12-tetraoxa-16,19-diazahenicosyl)carbamate (Int-2)

DIEA (326 mg, 2.52 mmol) was added to the solution of Int-2c (370 mg, 1.26 mmol) and HATU (575.8 mg, 1.51 mmol) in 4 mL dry DMF. The mixture was stirred at r.t. for 10 min. Then the solution of Int-2d (645 mg, 1.33 mmol) in DMF (4 mL) was added to the mixture. The reaction was stirred for 1 h. The mixture was concentrated and purified by silica gel column chromatography (CH₂Cl₂/MeOH=95/5) to give the Int-2 (680 mg, 71% yield) as a light brown solid. MS (ESI) m/z: 763.4 [M+H]⁺.

### Example Int-7

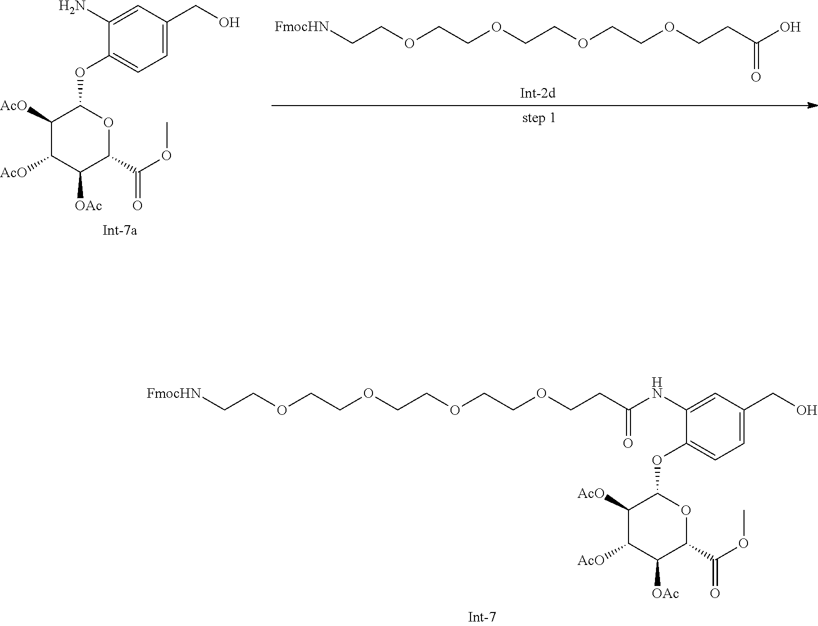

### Step 1: (2S,3R,4S,5S,6S)-2-(2-(1-(9H-fluoren-9-yl)-3-oxo-2,7,10,13,16-pentaoxa-4-azanonadecan-19-amido)-4-(hydroxymethyl)phenoxy)-6-(methoxycarbonyl)tetrahydro-2H-pyran-3,4,5-triyl triacetate (Int-7)

A mixture of Int-7a (400 mg, 0.88 mmol; purchase from Bide pharm), Int-2d (428 mg, 0.88 mmol; purchase from MCE) and EEDQ (282 mg, 1.14 mmol) in CH₂Cl₂ (5 mL) was stirred at r.t. for overnight. The mixture was concentrated under vacuum to give a residue, which was purified by silica gel column chromatography (MeOH/CH₂Cl₂=0/100-1/99) to give Int-7 (364 mg, 50.8% yield) as an off-white solid.

### Example Int-9

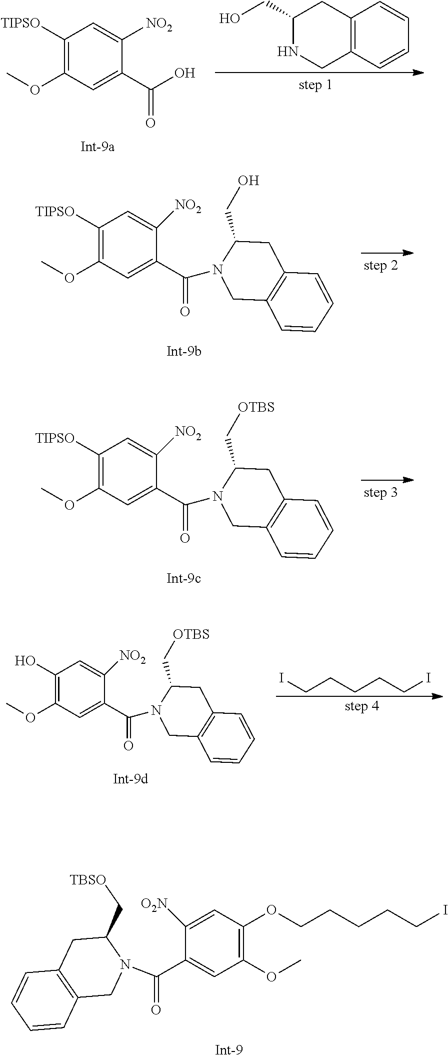

### Step 1: (S)-(3-(hydroxymethyl)-3,4-dihydroisoquinolin-2(1H)-yl)(5-methoxy-2-nitro-4-((triisopropylsilyl)oxy)phenyl)methanone (Int-9b)

To a mixture of Int-9a (1.0 g, 2.71 mmol), HOPO (452 mg, 4.07 mmol) and CH₂Cl₂ (15 mL) were added DECI (780 mg, 4.07 mmol). The mixture was stirred at r.t. for 10 min. Then a mixture of (S)-(1,2,3,4-tetrahydroisoquinolin-3-yl)methanol (442 mg, 2.71 mmol), Et₃N (943 μL, 6.78 mmol) and CH₂Cl₂ (5 mL) was added to the reaction mixture and continued to stir for 1 hour at r.t. The mixture was washed with water (20 mL*2). The organic layer was dried over Na₂SO₄ and filtrated. The filter was concentrated under vacuum to give a residue which was purified by silica gel column chromatography (Petroleum ether/EtOAc=50/50) to afford Int-9b (1.1 g, 79.1% yield) as a yellow solid. MS (ESI) m/z: 515.4 [M+H]⁺.

### Step 2: (S)-(3-(((tert-butyldimethylsilyl)oxy)methyl)-3,4-dihydroisoquinolin-2(1H)-yl)(5-methoxy-2-nitro-4-((triisopropylsilyl)oxy)phenyl)methanone (Int-9c)

To a solution of Int-9b (1.1 g, 2.14 mmol), imidazole (291 mg, 4.28 mmol) and CH₂Cl₂ (20 mL) were added dry TBSCl (484 mg, 3.21 mmol). The mixture was stirred at r.t. for 16 h. The mixture was filtrated, the filtered organic solvent was concentrated under vacuum to give a residue which was purified by silica gel column chromatography (Petroleum ether/EtOAc=70/30) to afford Int-9c (976 mg, 81.1% yield) as a yellow solid. MS (ESI) m/z: 629.4 [M+H]⁺.

### Step 3: (S)-(3-(((tert-butyldimethylsilyl)oxy)methyl)-3,4-dihydroisoquinolin-2(1H)-yl)(4-hydroxy-5-methoxy-2-nitrophenyl)methanone (Int-9d)

To a solution of Int-9c (780 mg, 1.21 mmol), DMF (10 mL) and H₂O (380 μL) were added LiOAc (80 mg, 1.21 mmol). The mixture was stirred at 40° C. for 2 h. After the reaction was completed, the mixture was diluted with water (20 mL), extracted with EtOAc (20 mL*2). The combined organic solvent was washed with brine (30 mL), dried over Na₂SO₄, filtered and the filtrate was concentrated under vacuum to give a residue which was purified by silica gel column chromatography (Petroleum ether/EtOAc=40/60) to afford Int-9d (571 mg, 99.5% yield) as a yellow oil. MS (ESI) m/z: 473.4 [M−H]⁺.

### Step 4: (S)-(3-(((tert-butyldimethylsilyl)oxy)methyl)-3,4-dihydroisoquinolin-2(1H)-yl)(4-((5-iodopentyl)oxy)-5-methoxy-2-nitrophenyl)methanone (Int-9)

To a solution of Int-9d (594 mg, 1.26 mmol), 1,5-diiodopentane (936 μL, 6.29 mmol) and DMF (5 mL) were added K₂CO₃ (523 mg, 3.78 mmol). The mixture stirred at r.t. overnight. After the reaction was completed, the mixture was diluted with water (10 mL), extracted with EtOAc (10 mL*2). The combined organics were washed with brine (20 mL), dried over Na₂SO₄, filtered and the filtrate was concentrated under vacuum to give a residue which was purified by silica gel column chromatography (Petroleum ether/EtOAc=70/30) to afford Int-9 (642 mg, 76.2% yield) as a yellow oil. MS (ESI) m/z: 669.3 [M+H]⁺.

### Example Int-10

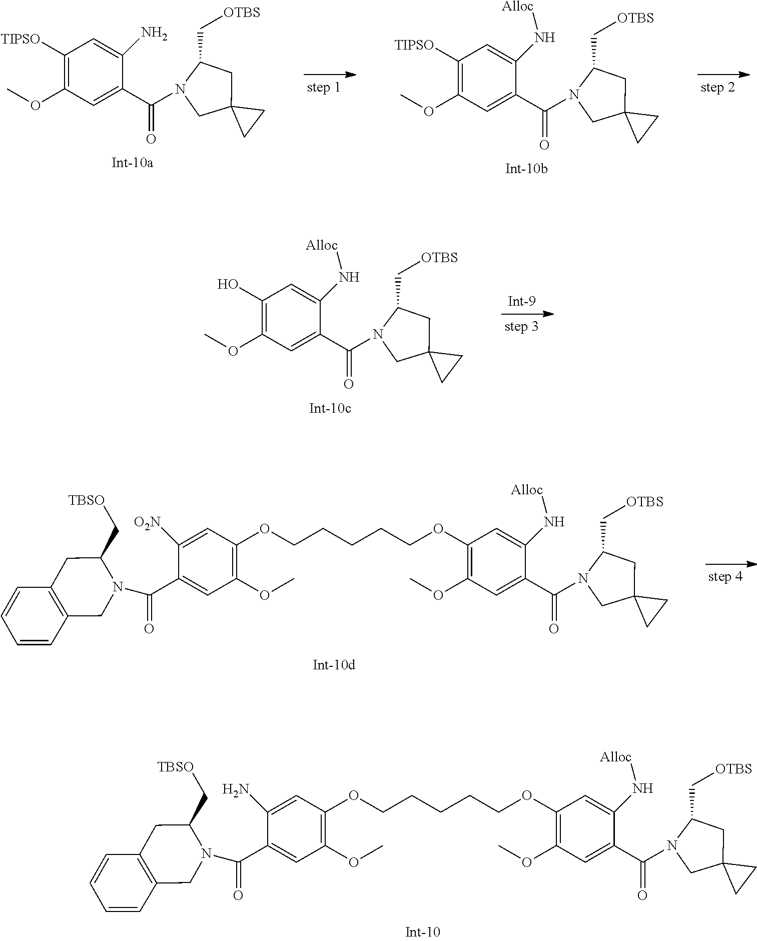

### Step 1: allyl (S)-(2-(6-(((tert-butyldimethylsilyl)oxy)methyl)-5-azaspiro[2.4]heptane-5-carbonyl)-4-methoxy-5-((triisopropylsilyl)oxy)phenyl)carbamate (Int-10b)

To a 0° C. solution of Int-10a (1.0 g, 1.78 mmol, prepared according to the procedures described in CN111164208A) and CH₂Cl₂ (15 mL) were added pyridine (158 μL, 1.96 mmol). Then AllocCl (227 μL, 2.14 mmol) was added to the mixture. The mixture was stirred at 0° C. for 30 min under N₂. The mixture was washed with water (15 mL*2). The organic layer was dried over Na₂SO₄ and filtrated. The filter was concentrated under vacuum to give a residue which was purified by silica gel column chromatography (Petroleum ether/EtOAc=70/30) to afford Int-10b (885 mg, 79.1% yield) as a yellow solid. MS (ESI) m/z: 647.3 [M+H]⁺.

### Step 2: allyl (S)-(2-(6-(((tert-butyldimethylsilyl)oxy)methyl)-5-azaspiro[2.4]heptane-5-carbonyl)-5-hydroxy-4-methoxyphenyl)carbamate (Int-10c)

Compound Int-10c (490 mg, 58.8% yield) was synthesized according to the synthetic procedure of step 3 of example Int-9. MS (ESI) m/z: 491.4 [M+H]⁺.

### Step 3: allyl (5-((5-(4-((S)-3-(((tert-butyldimethylsilyl)oxy)methyl)-1,2,3,4-tetrahydroisoquinoline-2-carbonyl)-2-methoxy-5-nitrophenoxy)pentyl)oxy)-2-((S)-6-(((tert-butyldimethylsilyl)oxy)methyl)-5-azaspiro[2.4]heptane-5-carbonyl)-4-methoxyphenyl)carbamate (Int-10d)

To a solution of Int-9 (642 mg, 0.961 mmol), Int-10c (471 mg, 0.961 mmol) and DMF (15 mL) were added K₂CO₃ (398 mg, 2.88 mmol). The mixture was stirred at r.t. for overnight. After the reaction was completed, the mixture was diluted with water (30 mL), and extracted with EtOAc (30 mL*2). The combined organics were washed with brine (40 mL), dried over Na₂SO₄, filtered and the filtrate was concentrated under vacuum to give a residue which was purified by silica gel column chromatography (Petroleum ether/EtOAc=70/30) to afford Int-10d (874 mg, 88.3% yield) as a white solid. MS (ESI) m/z: 1031.8 [M−H]⁺.

### Step 4: allyl (5-((5-(5-amino-4-((S)-3-(((tert-butyldimethylsilyl)oxy)methyl)-1,2,3,4-tetrahydroisoquinoline-2-carbonyl)-2-methoxyphenoxy)pentyl)oxy)-2-((S)-6-(((tert-butyldimethylsilyl)oxy)methyl)-5-azaspiro[2.4]heptane-5-carbonyl)-4-methoxyphenyl)carbamate (Int-10)

A mixed solution of Zn (63 mg, 0.97 mmol), EtOH (2 mL), AcOH (150 μL) and H₂O (150 μL) was stirred at r.t. 10 min. Then a solution of Int-10d in EtOH (1 mL) was added to the reaction mixture. The mixture was stirred at r.t. for 10 min. After the reaction was completed, the mixture was filtrated, the filtrate was concentrated under vacuum to give a residue which was purified by silica gel column chromatography (Petroleum ether/EtOAc=40/20) to afford Int-10 (74 mg, 76.2% yield) as a yellow oil. MS (ESI) m/z: 1002.3 [M+H]⁺.

### Example Int-12

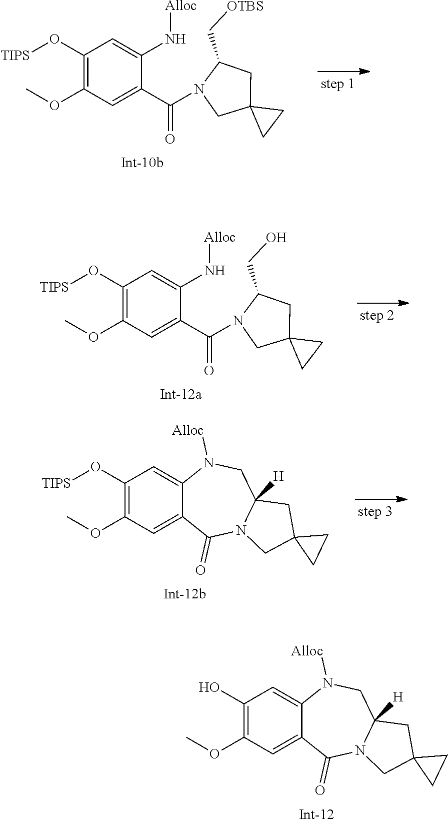

### Step 1: Allyl (S)-(2-(6-(hydroxymethyl)-5-azaspiro[2.4]heptane-5-carbonyl)-4-methoxy-5-((triisopropylsilyl)oxy)phenyl)carbamate (Int-12a)

para-Toluenesulfonic acid hydrate (417 mg, 2.19 mmol) was added to a solution of Int 10b (2.36 g, 3.65 mmol) in THF (40 mL) and water (2 mL). The reaction mixture was allowed to stir for 3 h at 22° C. The mixture was diluted with EtOAc (80 mL), and washed with water and brine. The organic phase was concentrated and purified by silica gel column chromatography (Petroleum ether/EtOAc=35/65) to give Int-12a (1.69 g, 87% yield). MS (ESI) m/z: 533.3 [M+H]⁺.

### Step 2: allyl (S)-7-methoxy-5-oxo-8-((triisopropylsilyl)oxy)-11,11a-dihydro-1H,3H-spiro[benzo[e]pyrrolo[1,2-a][1,4]diazepine-2,1′-cyclopropane]-10(5H)-carboxylate (Int-12b)

DIAD (1.14 mL, 5.75 mmol) was added to a solution of Int-12a (1.53 g, 2.87 mmol) and PPh₃ (2.26 g, 8.62 mmol) in THF (30 mL). The reaction mixture was allowed to stir for 1 h at 40° C. under N₂. The solvent was removed under vacuum and the residue was purified by silica gel column chromatography (Petroleum ether/EtOAc=35/65) to give the Int-12b (940 mg, 64% yield). MS (ESI) m/z: 515.3 [M+H]⁺.

### Step 3: allyl (S)-8-hydroxy-7-methoxy-5-oxo-11,11a-dihydro-1H,3H-spiro[benzo[e]pyrrolo[1,2-a][1,4]diazepine-2,1′-cyclopropane]-10(5H)-carboxylate (Int-12)

LiOAc (121 mg, 1.83 mmol) was added to a solution of Int-12b (940 mg, 1.83 mmol) in wet DMF (15 mL, 49/1 DMF/water). The reaction was allowed to proceed for 2 h at 25° C. The mixture was diluted with EtOAc, washed with H₂O and brine twice. The organic phase was concentrated and purified by silica gel column chromatography (Petroleum ether/EtOAc=35/65) to give Int-12 (605 mg, 92% yield). MS (ESI) m/z: 359.2 [M+H]⁺.

### Example Int-13

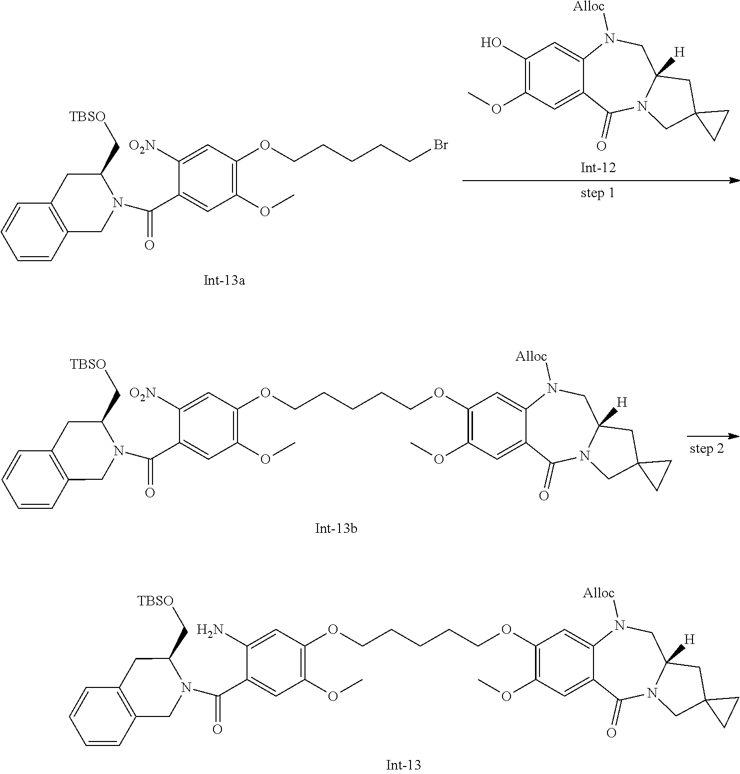

### Step 1: allyl (S)-8-((5-(4-((S)-3-(((tert-butyldimethylsilyl)oxy)methyl)-1,2,3,4-tetrahydroisoquinoline-2-carbonyl)-2-methoxy-5-nitrophenoxy)pentyl)oxy)-7-methoxy-5-oxo-11,11a-dihydro-1H,3H-spiro[benzo[e]pyrrolo[1,2-a][1,4]diazepine-2,1′-cyclopropane]-10(5H)-carboxylate (Int-13b)

Int-12 (450 mg, 1.26 mmol) and K₂CO₃ (226 mg, 1.63 mmol) were added to a solution of Int-13a (818 mg, 1.32 mmol) in 1 mL DMF. The mixture was stirred at r.t. for 24 h. The mixture was diluted with EtOAc, and washed with H₂O and brine. The organic phase was concentrated, and the crude product was purified by silica gel column chromatography (Petroleum ether/EtOAc=35/65) to give Int-13b (930 mg, 82% yield). MS (ESI) m/z: 899.5 [M+H]⁺.

### Step 2: allyl (S)-8-((5-(5-amino-4-((S)-3-(((tert-butyldimethylsilyl)oxy)methyl)-1,2,3,4-tetrahydroisoquinoline-2-carbonyl)-2-methoxyphenoxy)pentyl)oxy)-7-methoxy-5-oxo-11,11a-dihydro-1H,3H-spiro[benzo[e]pyrrolo[1,2-a][1,4]diazepine-2,1′-cyclopropane]-10(5H)-carboxylate (Int-13)

Zinc powder (2.65 g, 38.3 mmol) was added to a mixture of EtOH (10 mL), water (0.625 mL) and AcOH (0.625 mL) at 0° C. The reaction mixture was stirred at 5° C. for 30 min. A solution of Int-13b (930 mg, 1.04 mmol) in EtOH (6 mL) was added dropwise at 5° C. The reaction was allowed to proceed at 5° C. for 50 min. The reaction solution was filtrated. The filtrate was diluted with EtOAc and washed with water, saturated aq. NaHCO₃, and brine. The organic phase was dried over Na₂SO₄, filtered and the solvent removed by rotary evaporation under reduced pressure to afford the crude product which was purified by silica gel column chromatography (Petroleum ether/EtOAc=80/20) to give Int-13 (806 mg, 90% yield) as a yellow solid. MS (ESI) m/z: 869.5 [M+H]⁺.

### Example Int-14

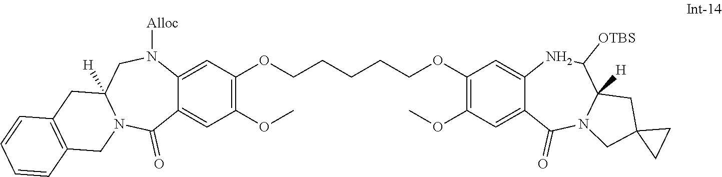

### allyl (S)-3-((5-(5-amino-4-((S)-6-(((tert-butyldimethylsilyl)oxy)methyl)-5-azaspiro[2.4]heptane-5-carbonyl)-2-methoxyphenoxy)pentyl)oxy)-2-methoxy-14-oxo-6,6a,7,12-tetrahydrobenzo[5,6][1,4]diazepino[1,2-b]isoquinoline-5(14H)-carboxylate (Int-14)

Int-14 was prepared according to a similar procedure described in the synthesis of Int-13 (440 mg). MS (ESI) m/z: 869.6 [M+H]⁺.

### Example Int-17

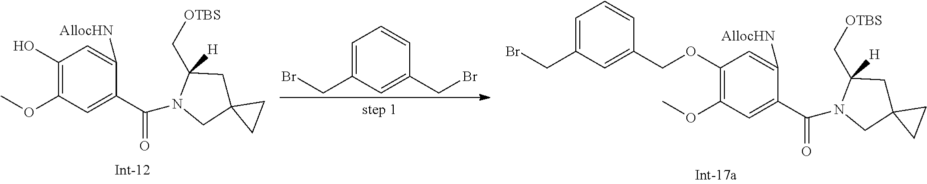

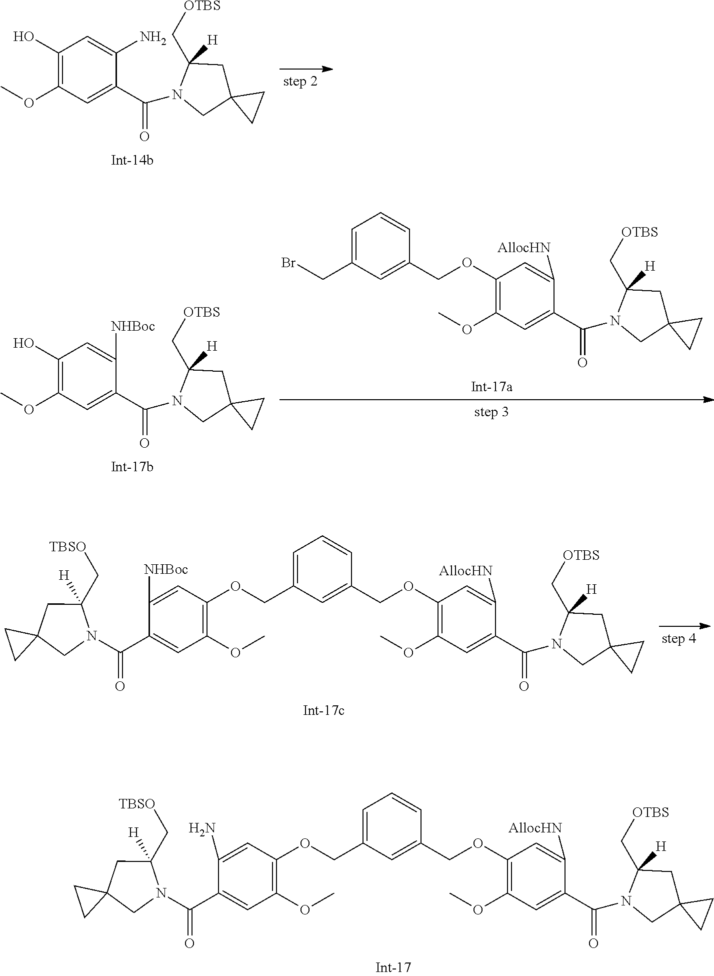

### Step 1: allyl (S)-(5-((3-(bromomethyl)benzyl)oxy)-2-(6-(((tert-butyldimethylsilyl)oxy)methyl)-5-azaspiro[2.4]heptane-5-carbonyl)-4-methoxyphenyl)carbamate (Int-17a)

To a mixture of Int-12 (1.20 g, 2.4 mmol) and 1,3-bis(bromomethyl)benzene (3.2 g, 12.2 mmol) in DMF (10 mL) was added K₂CO₃ (371.8 mg, 2.690 mmol). The mixture was stirred at r.t. for 4 hr. The mixture was diluted with EtOAc (20 mL), washed with brine (20 mL*3), dried over Na₂SO₄, filtered and the filtrate was concentrated under vacuum to give a residue. It was purified by silica gel column chromatography (EtOAc/Petroleum ether=0/100-15/85), fraction was concentrated under vacuum to give Int-17a (787.8 mg, 47.8% yield) as a colorless oil. MS (ESI) m/z: 673.4 [M+H]⁺.

### Step 2: tert-butyl (S)-(2-(6-(((tert-butyldimethylsilyl)oxy)methyl)-5-azaspiro[2.4]heptane-5-carbonyl)-5-hydroxy-4-methoxyphenyl)carbamate Int-17b

A mixture of Int-14b (447 mg, 1.10 mmol) and Boc₂O (249.1 mg, 1.43 mmol) in CH₂Cl₂ (5 mL) was stirred at 70° C. for 20 hr. The mixture was concentrated under vacuum and purified by silica gel column chromatography (EtOAc/Petroleum ether=0/100-20/80) to give Int-17b (480 mg, 86.1% yield) as a colorless oil. MS (ESI) m/z: 507.5 [M+H]⁺.

1H NMR (400 MHz, DMSO-d6) δ 9.57 (s, 1H), 8.95 (s, 1H), 7.25 (s, 1H), 6.78 (s, 1H), 4.30 (s, 1H), 3.77 (d, J=42.4 Hz, 5H), 3.49 (s, 1H), 3.17 (s, 1H), 1.99 (m, 4H), 1.44 (s, 9H), 0.88 (s, 9H), 0.64-0.42 (m, 4H), 0.05 (s, 6H).

### Step 3: allyl (5-((3-((5-((tert-butoxycarbonyl)amino)-4-((S)-6-(((tert-butyldimethylsilyl)oxy)methyl)-5-azaspiro[2.4]heptane-5-carbonyl)-2-methoxyphenoxy)methyl)benzyl)oxy)-2-((S)-6-(((tert-butyldimethylsilyl)oxy)methyl)-5-azaspiro[2.4]heptane-5-carbonyl)-4-methoxyphenyl)carbamate (Int-17c)

To a mixture of Int-17b (480 mg, 0.95 mmol) and Int-17a (637 mg, 0.95 mmol) in DMF (10 mL) was added K₂CO₃ (157 mg. 1.14 mmol) was added. The mixture was stirred at r.t. overnight. The mixture was diluted with EtOAc (30 mL), washed with brine (20 mL*3), dried over Na₂SO₄, filtered and the filtrate was concentrated under vacuum to give a residue. It was purified by silica gel column chromatography (EtOAc/Petroleum ether=0/100-30/70), the fraction was concentrated under vacuum to give Int-17c (721 mg, 69.2% yield) as a colorless oil. MS (ESI) m/z: 1121.9 [M+Na⁺]

1H NMR (400 MHz, DMSO-d6) δ 8.88 (s, 1H), 8.56 (s, 1H), 7.96 (s, 3H), 7.57 (s, 1H), 7.51 (s, 1H), 7.44 (d, J=8.2 Hz, 4H), 6.90 (d, J=5.6 Hz, 2H), 6.05-5.85 (m, 1H), 5.69 (s, 3H), 5.33 (dq, J=17.2, 1.6 Hz, 1H), 5.26-5.18 (m, 1H), 5.11 (s, 4H), 4.57 (dt, J=5.4, 1.4 Hz, 2H), 4.29 (s, 2H), 3.90-3.62 (m, 11H), 3.49-3.33 (m, 3H), 3.29 (s, 2H), 2.90 (s, 10H), 2.74 (d, J=8.4 Hz, 9H), 2.50 (dt, J=3.8, 1.9 Hz, 12H), 2.09-1.77 (m, 5H), 1.45 (s, 9H), 0.88 (s, 18H), 0.56 (ddd, J=18.4, 11.9, 5.9 Hz, 8H), 0.02 (d, J=4.1 Hz, 12H).

### Step 4: allyl (5-((3-((5-amino-4-((S)-6-(((tert-butyldimethylsilyl)oxy)methyl)-5-azaspiro[2.4]heptane-5-carbonyl)-2-methoxyphenoxy)methyl)benzyl)oxy)-2-((S)-6-(((tert-butyldimethylsilyl)oxy)methyl)-5-azaspiro[2.4]heptane-5-carbonyl)-4-methoxyphenyl)carbamate (Int-17)

To a solution of Int-17c (711 mg, 0.647 mmol) in CH₂Cl₂ (10 mL) was added TBS-OTf (312 μL, 359.0 mg, 1.36 mmol) at 0° C., then stirred at the same temperature for 1 h, followed by addition of 2,6-lutidine (395 μL, 364 mg, 3.40 mmol). The reaction was stirred at 0° C. for 10 min. The mixture was quenched with water (30 mL), extracted CH₂Cl₂ (30 mL*3). Combined organic layers were washed with brine (50 mL), dried over Na₂SO₄, filtered and the filtrate was concentrated under vacuum to give a residue. It was purified by silica gel column chromatography (EtOAc/Petroleum ether=0/100˜50/50) to give Int-17 (351.2 mg, 54.3% yield) as a white solid. MS (ESI) m/z: 999.9 [M+H]⁺.

### Example Int-18

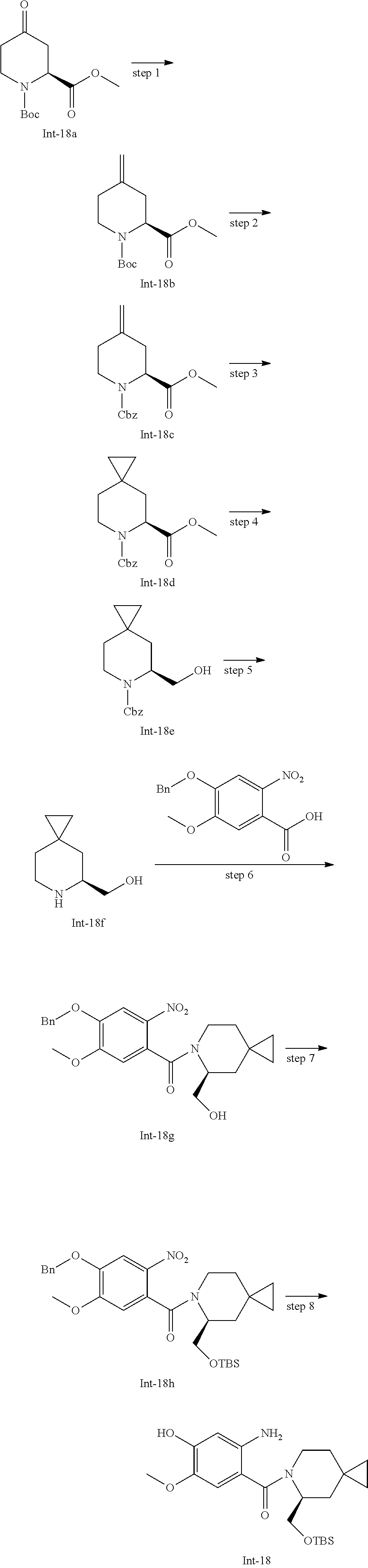

Step 1: 1-(tert-butyl) 2-methyl (S)-4-methylenepiperidine-1,2-dicarboxylate (Int-18b)

To the solution of MePh₃PBr (8.26 g, 22.7 mmol) in dry THF (70 mL) was added tBuOK in dry THF (1M, 22.7 mL, 22.7 mmol) under N₂ atmosphere at 0° C., stirred at 0° C. for 1 h. To the reaction solution was added a solution of Int-18a (5.3 g, 20.6 mmol) in dry THF (30 mL) under N₂ atmosphere at 0° C., stirred for 1 h. Water (50 mL) and sat. NH₄Cl (50 mL) were added to the solution. The organic phase was separated, and water phase was extracted with EtOAc (50 mL*3). The combined organic phase was concentrated and purified by flash column chromatography (petroleum ether/EtOAc=90/10) to afford Int-18b as a colorless oil (3.7 g, 70% yield). MS (ESI) m/z: 156.1 [M+H-Boc]⁺.

¹H NMR (400 MHz, CDCl₃) δ 5.08-4.97 (m, 0.5H), 4.87-4.78 (m, 0.5H), 4.79 (s, 2H), 4.20-3.97 (m, 1H), 3.71 (s, 3H), 3.12-2.90 (m, 1H), 2.81-2.67 (m, 1H), 2.49-2.37 (m, 1H), 2218-2.10 (m, 2H), 1.47 (s, 9H).

### Step 2: 1-benzyl 2-methyl (S)-4-methylenepiperidine-1,2-dicarboxylate (Int-18c)

To the solution of Int-18b (3.7 g, 14.5 mmol) in CH₂Cl₂ (25 mL) was added 4 M HCl in EtOAc (20 mL) under N₂ atmosphere at 0° C., stirred at r.t. for 30 min. The solution was concentrated and re-dissolved in CH₂Cl₂ (100 mL). To the solution were added CbzCl (2.5 mL, 17.4 mmol) and Et₃N (4.1 mL, 29.1 mmol) at 0° C., stirred at r.t. for another 1 h. The solution was poured into 0.5 N HCl (50 mL) and water (50 mL), extracted with CH₂Cl₂ (50 mL*2). The combined organic phase was concentrated and purified by flash column chromatography (petroleum ether/EtOAc=90/10) to give Int-18c as a colorless oil (3.58 g, 85% yield). MS (ESI) m/z: 312.2 [M+Na]⁺.

¹H NMR (400 MHz, CDCl₃) δ 7.45-7.24 (m, 5H), 5.17 (s, 2H), 5.14-4.90 (m, 1H), 4.81 (s, 2H), 4.28-4.08 (m, 1H), 3.69 (d, J=18.8 Hz, 3H), 3.23-3.01 (m, 1H), 2.77 (t, J=15.7 Hz, 1H), 2.50-2.39 (m, 1H), 2.30-2.12 (m, 2H).

### Step 3: 6-benzyl 5-methyl (S)-6-azaspiro[2.5]octane-5,6-dicarboxylate (Int-18d)

To the dry CH₂Cl₂ (70 mL) was added ZnEt₂ (2 M in hexane, 24.7 mL, 49.5 mmol) under N₂ atmosphere at 0° C., stirred at 0° C. for 10 min. Dry TFA (3.9 mL, 49.5 mmol) was added slowly, stirred at 0° C. for 1 h. Diiodomethane (4.1 mL, 49.5 mmol) was added slowly, stirred at 0° C. for 1 h. A solution of Int-18c (3.58 g, 12.4 mmol) in dry CH₂Cl₂ (30 mL) was added slowly, stirred at 0° C. for 30 min and then stirred at r.t. for 14 h. The solution was filtered through Celite. The filtrate was washed with sat. NH₄Cl (50 mL) and water (50 mL). The aq. phase was extracted with CH₂Cl₂ (50 mL*2). The organic phase was concentrated and purified by flash column chromatography (petroleum ether/EtOAc=90/10) to give Int-18d as a colorless oil (2.97 g, 79% yield). MS (ESI) m/z: 326.3 [M+Na]⁺.

¹H NMR (400 MHz, CDCl₃) δ 7.45-7.25 (m, 5H), 5.24-5.08 (m, 2H), 5.04-4.84 (m, 1H), 4.21-4.02 (m, 1H), 3.72 (d, J=6 Hz, 3H), 3.36-3.14 (m, 1H), 2.24-2.12 (m, 1H), 2.00-1.85 (m, 1H), 1.62-1.54 (m, 1H), 0.91-0.72 (m, 1H), 0.42-0.22 (m, 4H).

### Step 4: benzyl (S)-5-(hydroxymethyl)-6-azaspiro[2.5]octane-6-carboxylate (Int-18e)

To the solution of Int-18d (2.97 g, 9.79 mmol) in dry THF (50 mL) was added LiBHEt₃ (1 M in THF, 24.5 mL, 24.5 mmol) under N₂ atmosphere at 0° C., stirred at 0° C. for 2 h. To the solution was added water (2 mL) dropwise, washed with brine (15 mL), extracted with EtOAc (20 mL*3). The organic phase was concentrated and purified by flash column chromatography (petroleum ether/EtOAc=50/50) to give Int-18e as a colorless oil (2.46 g, 91% yield). MS (ESI) m/z: 276.3 [M+H]⁺.

¹H NMR (400 MHz, CDCl₃) δ 7.40-7.29 (m, 5H), 5.22-5.09 (m, 2H), 4.53-4.39 (m, 1H), 4.20-4.06 (m, 1H), 4.02 (dd, J=11.0, 9.5 Hz, 1H), 3.67 (dd, J=11.1, 5.6 Hz, 1H), 3.19-3.05 (m, 1H), 2.08-1.97 (m, 1H), 1.95-1.81 (m, 1H), 1.08-0.97 (m, 1H), 0.91-0.79 (m, 2H), 0.47-0.38 (m, 1H), 0.35-0.23 (m, 3H).

### Step 5: (S)-(6-azaspiro[2.5]octan-5-yl)methanol (Int-18f)

To the solution of Int-18e (2.46 g, 8.95 mmol) in MeOH (40 mL) were added 7 M NH₃ in MeOH (8 mL) and 10% wet Pd/C (246 mg) under N₂ atmosphere, stirred under H₂ atmosphere for 3 h. The solution was filtered and concentrated to give Int-18f as a yellow oil (1.3 g, quant.), which was used directly for the next step without further purification. MS (ESI) m/z: 142.1 [M+H]⁺.

¹H NMR (400 MHz, CDCl₃) δ 3.61 (dd, J=10.9, 3.7 Hz, 1H), 3.48 (dd, J=10.9, 7.7 Hz, 1H), 3.14 (ddd, J=12.0, 4.1, 2.6 Hz, 1H), 2.89 (ddd, J=11.1, 6.9, 3.3 Hz, 1H), 2.81 (td, J=12.1, 3.0 Hz, 1H), 1.89 (td, J=12.7, 4.2 Hz, 1H), 1.65 (t, J=12.1 Hz, 1H), 0.90-0.77 (m, 2H), 0.39-0.32 (m, 2H), 0.32-0.23 (m, 2H).

### Step 6: (S)-(4-(benzyloxy)-5-methoxy-2-nitrophenyl)(5-(hydroxymethyl)-6-azaspiro[2.5]octan-6-yl)methanone (Int-18g)

To the solution of 4-(benzyloxy)-5-methoxy-2-nitrobenzoic acid (2.7 g, 8.9 mmol) in CH₂Cl₂ (50 mL) was added oxalyl chloride (2.3 mL, 26.7 mmol) under N₂ atmosphere at 0° C., followed by addition of 3 drops of DMF, stirred for 1 h. The turbid solution became clear and no gas eluted. The solution was concentrated to remove the excess amount of oxalyl chloride. The residue was dissolved in dry CH₂Cl₂ (20 mL) and added to the solution of Int-18f (1.28 g, 8.9 mmol) and DIEA (6.3 mL, 35.6 mmol) in dry CH₂Cl₂ (30 mL) at 0° C., stirred for 20 min. The solution was washed with 0.5 N HCl (30 mL), extracted with CH₂Cl₂ (20 mL*2). The organic phase was concentrated and purified by flash column chromatography (petroleum ether/EtOAc=20/80) to give the title compound Int-18g as an off-white solid (3 g, 79% yield). MS (ESI) m/z: 427.4 [M+H]⁺.

### Step 7: (S)-(2-amino-4-(benzyloxy)-5-methoxyphenyl)(5-(hydroxymethyl)-6-azaspiro[2.5]octan-6-yl)methanone (Int-18h)

To the solution of Int-18g (500 mg, 1.17 mmol) and imidazole (163 mg, 6.16 mmol) in dry DMF (10 mL) was added TBSCl (216 mg, 1.4 mmol) under N₂ atmosphere at 0° C., stirred at r.t. for 1 h. The solution was added into water (30 mL), extracted with EtOAc (15 mL*3), concentrated, and purified by flash column chromatography (petroleum ether/EtOAc=75/25) to give Int-18 h as an off-white solid (620 mg, 97.8% yield). MS (ESI) m/z: 541.7 [M+H]⁺.

### Step 8: (S)-(2-amino-4-hydroxy-5-methoxyphenyl)(5-(((tert-butyldimethylsilyl)oxy)methyl)-6-azaspiro[2.5]octan-6-yl)methanone (Int-18)

To the solution of Int-18h (440 mg, 0.81 mmol) in MeOH (8 mL) was added 10% Pd/C (44 mg), stirred at H₂ atmosphere for 7 h. The solution was filtered and concentrated to give the title compound Int-18 as a pink solid (342 mg, quant.). MS (ESI) m/z: 421.4 [M+H]⁺.

¹H NMR (400 MHz, DMSO-d6) δ 9.04 (s, 1H), 6.60 (s, 1H), 6.19 (s, 1H), 4.77 (s, 2H), 3.92-3.82 (m, 1H), 3.81-3.71 (m, 1H), 3.63 (s, 3H), 3.05 (s, 1H), 1.97 (dd, J=13.6, 5.4 Hz, 1H), 1.84 (t, J=10.8 Hz, 1H), 1.07 (d, J=13.6 Hz, 1H), 0.84 (s, 9H), 0.45 (d, J=5.5 Hz, 1H), 0.32 (d, J=6.0 Hz, 1H), 0.27-0.18 (m, 2H), 0.03 (s, 3H), 0.01 (s, 3H).

### Example Int-21

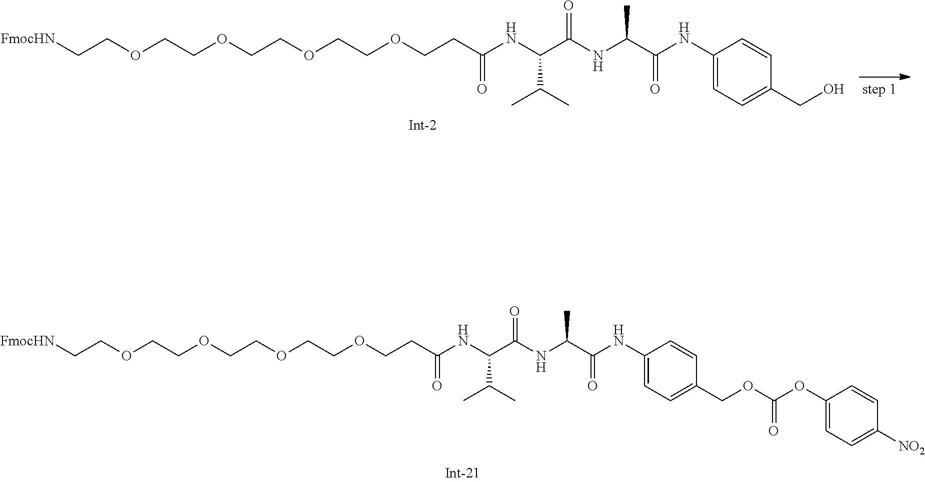

### Step 1: (9H-fluoren-9-yl)methyl ((17S,20S)-17-isopropyl-20-methyl-21-((4-((((4-nitrophenoxy)carbonyl)oxy)methyl)phenyl)amino)-15,18,21-trioxo-3,6,9,12-tetraoxa-16,19-diazahenicosyl)carbamate (Int-21)

To a mixture of Int-2 (500 mg, 0.656 mmol), bis(4-nitrophenyl) carbonate (300 mg, 0.981 mmol) and DMF (10 mL) were added DIEA (233 μL, 1.31 mmol). The mixture was stirred at r.t. overnight. The mixture was concentrated under vacuum to give a residue which was purified by silica gel column chromatography (Petroleum ether/EtOAc=0/100) to afford Int-21 (386 mg, 63.5% yield) as a white solid. MS (ESI) m/z: 928.7 [M+H]⁺.

### Example Int-22

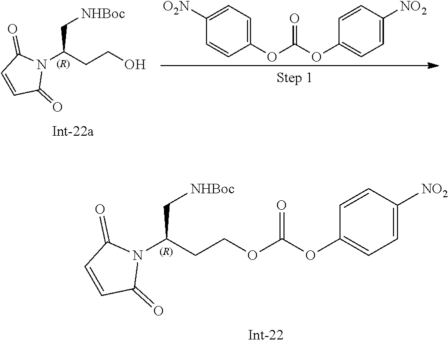

Int-22 was synthesized according to a modified synthetic procedure of reference (Tetrahedron Letters 54 (2013) 349-3495).

### Example Int-23

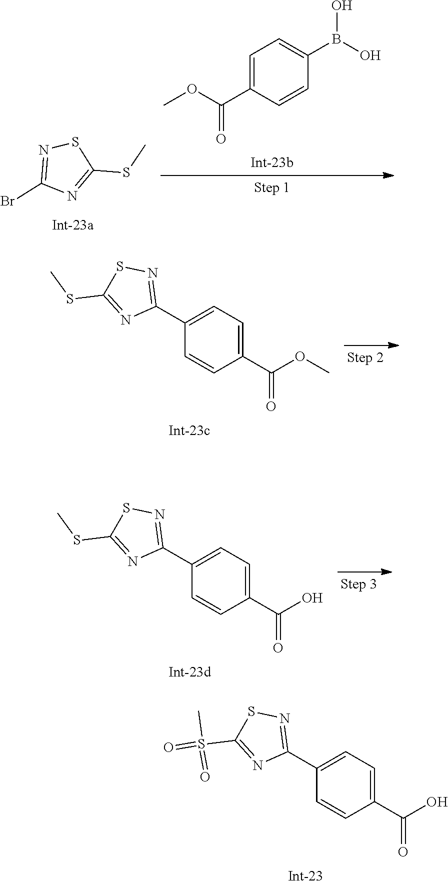

### Step 1: methyl 4-(5-(methylthio)-1,2,4-thiadiazol-3-yl)benzoate (Int-23c)

To a solution of compound Int-23a (100 mg, 0.47 mmol) in toluene (4 mL) and H₂O (1 mL) were added compound Int-23b (109.72 mg, 0.568 mmol), K₂CO₃ (168 mg, 0.947 mmol) and Pd(dppf)Cl₂·CH₂Cl₂ (34.6 mg, 0.047 mmol). The mixture was stirred at 110° C. for 3 h under N₂ atmosphere. The mixture was filtered through a pad of celite, diluted with EtOAc (100 mL), washed by brine (50 mL*4). The organic layer was dried over anhydrous Na₂SO₄, filtered and concentrated. The crude was purified by flash column chromatography (eluted with Petroleum ether/EtOAc=0-40%). Compound Int-23c (56 mg, 44.4% yield) was obtained as an off-white solid. MS (ESI) m/z: 267.1 [M+H]⁺.

### Step 2: 4-(5-(methylthio)-1,2,4-thiadiazol-3-yl)benzoic acid (Int-23d)

To a solution of compound Int-23c (54 mg, 0.20 mmol) in MeOH (3 mL) and H₂O (1 mL) was added LiOH (17 mg, 0.41 mmol). The mixture was stirred at r.t. for 2 h. The mixture was adjusted to pH 7 and purified by prep-HPLC (FA condition) to give compound Int-23d (36 mg, 70.3% yield) as a white solid. MS (ESI) m/z: 253.1 [M+H]⁺.

### Step 3: 4-(5-(methylsulfonyl)-1,2,4-thiadiazol-3-yl)benzoic acid (Int-23)

To a solution of compound Int-23d (35 mg, 0.14 mmol) in CH₂Cl₂ (3 mL) and THF (3 mL) was added m-CPBA (96 mg, 0.55 mmol). The mixture was stirred at room temperature for 16 h. The mixture was concentrated and purified by prep-HPLC (Method: column:XBridge Prep C18 OBD 5 μm 19*150 mm; Mobile phase: A-water (0.1% TFA): B-acetonitrile; Flow rate: 20 mL/min). Compound Int-23 (12 mg, 99% purity) was obtained as a white solid. MS (ESI) m/z: 284.8 [M+H]⁺.

### Example Int-25

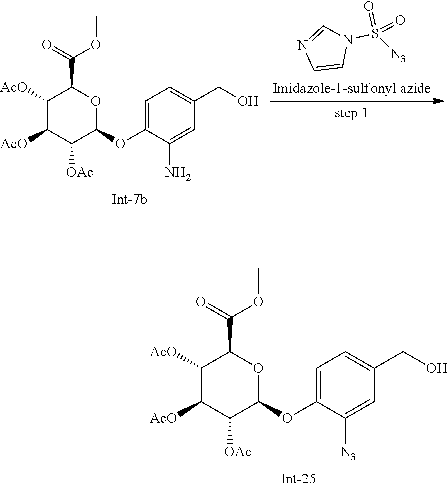 [SMILES: COC(=O)[C@H]1O[C@@H](Oc2ccc(CO)cc2N)[C@H](OC(C)=O)[C@@H](OC(C)=O)[C@@H]1OC(C)=O ; [N-]=[N+]=NS(=O)(=O)n1ccnc1 ; COC(=O)[C@H]1O[C@@H](Oc2ccc(CO)cc2N=[N+]=[N-])[C@H](OC(C)=O)[C@@H](OC(C)=O)[C@@H]1OC(C)=O]

### Step 1 (2S,3R,4S,5S,6S)-2-(2-azido-4-(hydroxymethyl)phenoxy)-6-(methoxycarbonyl)tetrahydro-2H-pyran-3,4,5-triyl triacetate (Int-25)

Int-7b (450 mg, 0.99 mmol) was dissolved in dry THF (2 mL). The mixture was cooled to 0° C., then Imidazole-1-sulfonyl azide (414.2 mg, 1.98 mmol) in 1 mL of MeOH was added, followed by addition of K₂CO₃ in 1 mL of H₂O and cat. amount of CuSO₄. The mixture was stirred at r.t. for 2 h under a nitrogen atmosphere. EtOAc (5 mL) was added, the organic phase was collected, dried over Na₂SO₄, concentrated, and then purified by column chromatography (Petroleum ether/EtOAc=50/50) to obtain Int-25 as a white solid (350 mg, 87.2%). MS (ESI) m/z: 504.2 [M+Na]⁺.

¹H NMR (400 MHz, CDCl₃) δ 7.17-7.03 (m, 3H), 5.44-5.26 (m, 3H), 5.07 (d, J=7.1 Hz, 1H), 4.64 (d, J=2.2 Hz, 2H), 4.13 (dd, J=6.8, 2.8 Hz, 1H), 3.83-3.71 (m, 3H), 2.17-2.00 (m, 9H).

### Example Int-26

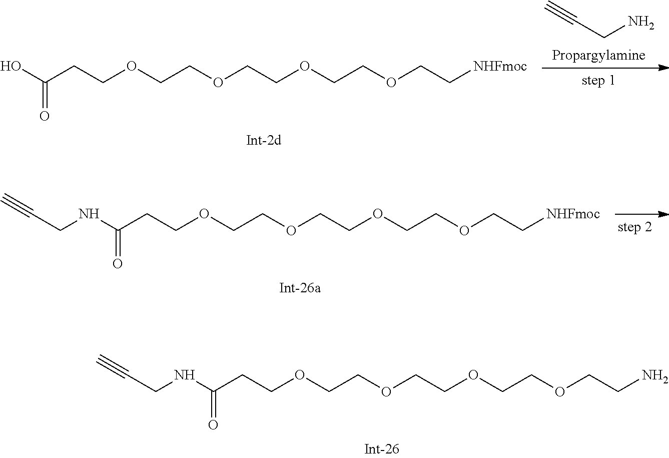

### Step 1: (9H-fluoren-9-yl)methyl (15-oxo-3,6,9,12-tetraoxa-16-azanonadec-18-yn-1-yl)carbamate (Int-26a)

To a solution of Int-2d (200 mg, 0.41 mmol) in 2 mL DMF was added HATU (312 mg, 0.82 mmol) and DIPEA (224 μL, 1.23 mmol) at 0° C. The mixture was stirred at 0° C. for 10 min. Propargylamine (25 μL, 0.45 mmol) was added. The mixture was stirred at r.t. for 1 h under a nitrogen atmosphere. The solvent was then removed by evaporation, and the crude product was purified by column chromatography (MeOH/CH₂Cl₂=5/95) to obtain Int-26a as a brown oil (200 mg, 93% yield). MS (ESI) m/z: 525.8 [M+H]⁺.

### Step 2: 1-amino-N-(prop-2-yn-1-yl)-3,6,9,12-tetraoxapentadecan-15-amide (Int-26)

To a solution of Int-26a (500 mg, 0.95 mmol) in 2 mL DMF was added diethylamine (0.98 mL, 9.5 mmol) at 0° C. The mixture was stirred at 0° C. for 30 min. The solvent was then removed by evaporation, and the crude product was purified by column chromatography (MeOH/CH₂Cl₂=5/95) to obtain Int-26 as a light-yellow solid (250 mg, 87% yield). MS (ESI) m/z: 303.3 [M+H]⁺.

### Example 1: Synthesis of Conjugator-Linker-Payloads

### Example Ref-2-1

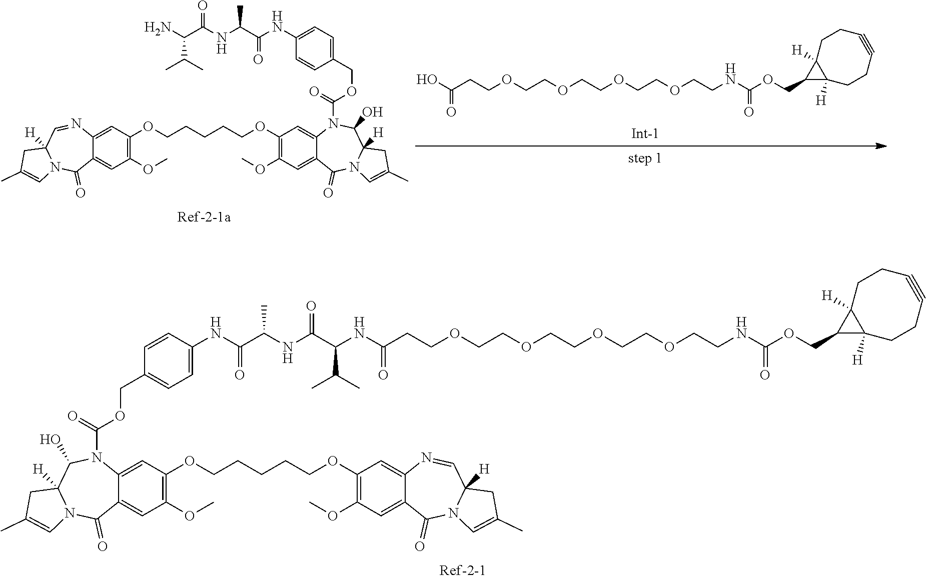

Ref-2-1a was synthesized according to the procedures described in US2015283262A1 and Org. Process Res. Dev. 2018, 22, 1241-1256.

### Step 1: 4-((21S,24S)-1-((1R,8S,9s)-bicyclo[6.1.0]non-4-yn-9-yl)-21-isopropyl-24-methyl-3,19,22-trioxo-2,7,10,13,16-pentaoxa-4,20,23-triazapentacosan-25-amido)benzyl (11S,11aS)-11-hydroxy-7-methoxy-8-((5-(((S)-7-methoxy-2-methyl-5-oxo-5,11a-dihydro-1H-benzo[e]pyrrolo[1,2-a][1,4]diazepin-8-yl)oxy)pentyl)oxy)-2-methyl-5-oxo-11,11a-dihydro-1H-benzo[e]pyrrolo[1,2-a][1,4]diazepine-10(5H)-carboxylate (Ref-2-1)

To a solution of Int-1 (5.3 mg, 0.01 mmol, prepared according to procedures described in WO2022120132 A1 and WO2022079211 A1) in 0.5 mL DMF was added HATU (5.0 mg, 0.01 mmol) and DIEA ((4 μL, 0.02 mmol) at 0° C. The mixture was stirred at r.t. for 15 min. Ref-2-1a (10 mg, 0.01 mmol) in 0.5 mL DMF was added to the mixture at 0° C. and the reaction was stirred at r.t. for 2 h. The mixture was purified by prep-HPLC (Method: column:XBridge Prep C18 OBD 5 μm 19*150 mm; Mobile phase: A-water (no formic acid): B-acetonitrile; Flow rate: 20 mL/min, the fraction was lyophilized to give Ref-2-1 (2.5 mg, 17.1% yield) as a white solid. MS (ESI) m/z: 1345.9 [M+H]⁺.

### Example Ref-2-2

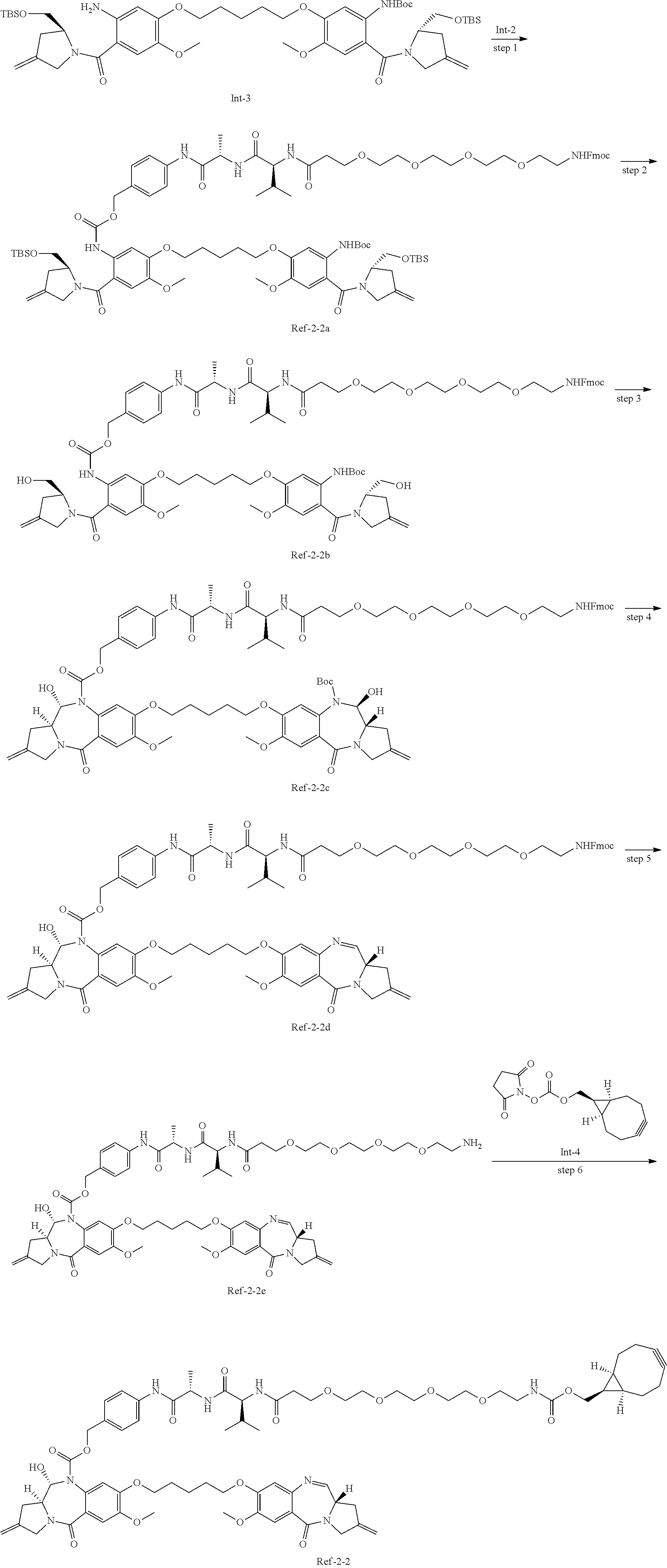

Int-3 was synthesized according to the procedures described in WO 2017059289 A1 and Org. Process Res. Dev. 2022, 26, 2155-2175.

### Step 1: (9H-fluoren-9-yl)methyl tert-butyl (5-((5-(5-((((4-((21S,24S)-1-(9H-fluoren-9-yl)-21-isopropyl-24-methyl-3,19,22-trioxo-2,7,10,13,16-pentaoxa-4,20,23-triazapentacosan-25-amido)benzyl)oxy)carbonyl)amino)-4-((S)-2-(((tert-butyldimethylsilyl)oxy)methyl)-4-methylenepyrrolidine-1-carbonyl)-2-methoxyphenoxy)pentyl)oxy)-2-((S)-2-(((tert-butyldimethylsilyl)oxy)methyl)-4-methylenepyrrolidine-1-carbonyl)-4-methoxyphenyl)carbamate (Ref-2-2a)

To a solution of Int-3 (200 mg, 0.1210 mmol) and 200 mg 4 Å MS in dry THF (2.5 mL) was added triphosgene (24.9 mg, 0.084 mmol), then Et₃N (64 μL, 0.462 mmol) was added at 0° C. under N₂. The mixture was stirred at 0° C. for 10 min under N₂. The formation of the isocyanate was monitored by LCMS analysis by quenching with MeOH. A solution of Int-2 (176 mg, 0.231 mmol), dibutyltin dilaurate (13.3 mg, 0.021 mmol), Et₃N (43.7 μL, 0.315 mmol) in dry THF (2.5 mL) was added to the mixture. The mixture was stirred for 3 h at r.t. The mixture was filtered, and the filtrate was concentrated. The residue was purified by silica gel column chromatography (CH₂Cl₂/MeOH=4/96) to give Ref-2-2a (304 mg, 83% yield) as a white solid. MS (ESI) m/z: 1742.1 [M+H]⁺.

### Step 2: tert-butyl (5-((5-(5-((((4-((21S,24S)-1-(9H-fluoren-9-yl)-21-isopropyl-24-methyl-3,19,22-trioxo-2,7,10,13,16-pentaoxa-4,20,23-triazapentacosan-25-amido)benzyl)oxy)carbonyl)amino)-4-((S)-2-(hydroxymethyl)-4-methylenepyrrolidine-1-carbonyl)-2-methoxyphenoxy)pentyl)oxy)-2-((S)-2-(hydroxymethyl)-4-methylenepyrrolidine-1-carbonyl)-4-methoxyphenyl)carbamate (Ref-2-2b)

para-Toluenesulfonic acid hydrate (40 mg, 0.21 mmol) was added to a solution of Ref-2-2a (304 mg, 0.17 mmol) in THF (3 mL) and water (0.15 mL). The reaction mixture was allowed to stir for 4 h at 22° C. The mixture was diluted with EtOAc (20 mL), washed with water, sat. NaHCO₃ and brine. The organic phase was concentrated and purified by silica gel column chromatography (CH₂Cl₂/MeOH=5/95) to give Ref-2-2b (213 mg, 81% yield) as a white solid. MS (ESI) m/z: 1513.9 [M+H]⁺.

### Step 3: 4-((21S,24S)-1-(9H-fluoren-9-yl)-21-isopropyl-24-methyl-3,19,22-trioxo-2,7,10,13,16-pentaoxa-4,20,23-triazapentacosan-25-amido)benzyl (11S,11aS)-8-((5-(((11S,11aS)-10-(tert-butoxycarbonyl)-11-hydroxy-7-methoxy-2-methylene-5-oxo-2,3,5,10,11,11a-hexahydro-1H-benzo[e]pyrrolo[1,2-a][1,4]diazepin-8-yl)oxy)pentyl)oxy)-11-hydroxy-7-methoxy-2-methylene-5-oxo-2,3,11,11a-tetrahydro-1H-benzo[e]pyrrolo[1,2-a][1,4]diazepine-10(5H)-carboxylate (Ref-2-2c)

DMP (58.9 mg, 0.139 mmol) was added slowly, portion-wise to the solution of Ref-2-2b (100 mg) in dry CH₂Cl₂ (2 mL) at 0° C. The reaction was then warmed to r.t. and stirred overnight. The reaction was quenched with sat. Na₂S₂O₃, followed by the addition of sat. NaHCO₃ and water. The layers were separated and the organic layer was washed with sat. Na₂S203, sat. NaHCO₃ and brine, dried over Na₂SO₄. The crude product was purified by silica gel column chromatography (CH₂Cl₂/MeOH=5/95) to give Ref-2-2c (80 mg, 80% yield) white solid. MS (ESI) m/z: 1509.8 [M+H]⁺.

### Step 4: 4-((21S,24S)-1-(9H-fluoren-9-yl)-21-isopropyl-24-methyl-3,19,22-trioxo-2,7,10,13,16-pentaoxa-4,20,23-triazapentacosan-25-amido)benzyl (11S,11aS)-11-hydroxy-7-methoxy-8-((5-(((S)-7-methoxy-2-methylene-5-oxo-2,3,5,11a-tetrahydro-1H-benzo[e]pyrrolo[1,2-a][1,4]diazepin-8-yl)oxy)pentyl)oxy)-2-methylene-5-oxo-2,3,11,11a-tetrahydro-1H-benzo[e]pyrrolo[1,2-a][1,4]diazepine-10(5H)-carboxylate (Ref-2-2d)

Ref-2-2c (20 mg, 0.013 mmol) was cooled to −3° C. Separately, a solution of 95% TFA in H₂O (0.5 mL) was cooled to −3° C. before adding to Ref-2-2c. The reaction mixture was stirred at −3° C. for 30 min before pouring onto a 1:1 solution of CHCl₃: sat. NaHCO₃ (16 mL) at 0° C. The organic layer was separated, dried on Na₂SO₄ before being filtered and then removed in vacuo. The crude material was used directly in the next step without further purification. MS (ESI) m/z: 1391.7 [M+H]⁺.

### Step 5: 4-((17S,20S)-1-amino-17-isopropyl-20-methyl-15,18-dioxo-3,6,9,12-tetraoxa-16,19-diazahenicosan-21-amido)benzyl (11S,11aS)-11-hydroxy-7-methoxy-8-((5-(((S)-7-methoxy-2-methylene-5-oxo-2,3,5,11a-tetrahydro-1H-benzo[e]pyrrolo[1,2-a][1,4]diazepin-8-yl)oxy)pentyl)oxy)-2-methylene-5-oxo-2,3,11,11a-tetrahydro-1H-benzo[e]pyrrolo[1,2-a][1,4]diazepine-10(5H)-carboxylate (Ref-2-2e)

To a solution of crude Ref-2-2d (18 mg, 0.013 mmol) in 0.5 mL DMF was added Et₂NH (14 μL, 0.129 mmol). The mixture was stirred at r.t. for 0.5 h. After the reaction was completed, the mixture was combined was purified by prep-HPLC (Method: column:XBridge Prep C18 OBD 5 μm 19*150 mm; Mobile phase: A—water (0.1% formic acid): B-acetonitrile; Flow rate: 20 mL/min) to give Ref-2-2e (5 mg, 33% yield). MS (ESI) m/z: 1169.7 [M+H]⁺.

### Step 6: 4-((21S,24S)-1-((1R,8S,9s)-bicyclo[6.1.0]non-4-yn-9-yl)-21-isopropyl-24-methyl-3,19,22-trioxo-2,7,10,13,16-pentaoxa-4,20,23-triazapentacosan-25-amido)benzyl (11S,11aS)-11-hydroxy-7-methoxy-8-((5-(((S)-7-methoxy-2-methylene-5-oxo-2,3,5,11a-tetrahydro-1H-benzol[e]pyrrolo[1,2-a][1,4]diazepin-8-yl)oxy)pentyl)oxy)-2-methylene-5-oxo-2,3,11,11a-tetrahydro-1H-benzo[e]pyrrolo[1,2-a][1,4]diazepine-10(5H)-carboxylate (Ref-2-2)

DIEA (8.6 μL, 0.05 mmol) was added to the solution of Ref-2-2e (14.5 mg, 0.012 mmol) and Int 4 (10.8 mg, 0.037 mmol, prepared according to procedures described in J Am. Chem. Soc. 2020, 142, 20, 9285-9301) in 1 mL DMF. The mixture was stirred at r.t. for 20 min. The mixture was purified by prep-HPLC (Method: column:XBridge Prep C18 OBD 5 μm 19*150 mm; Mobile phase: A—water (no formic acid): B-acetonitrile; Flow rate: 20 mL/min) to give Ref-2-2 (7.9 mg, 46% yield) as a light gray solid. MS (ESI) m/z: 1345.7 [M+H]⁺.

### Example 2-1

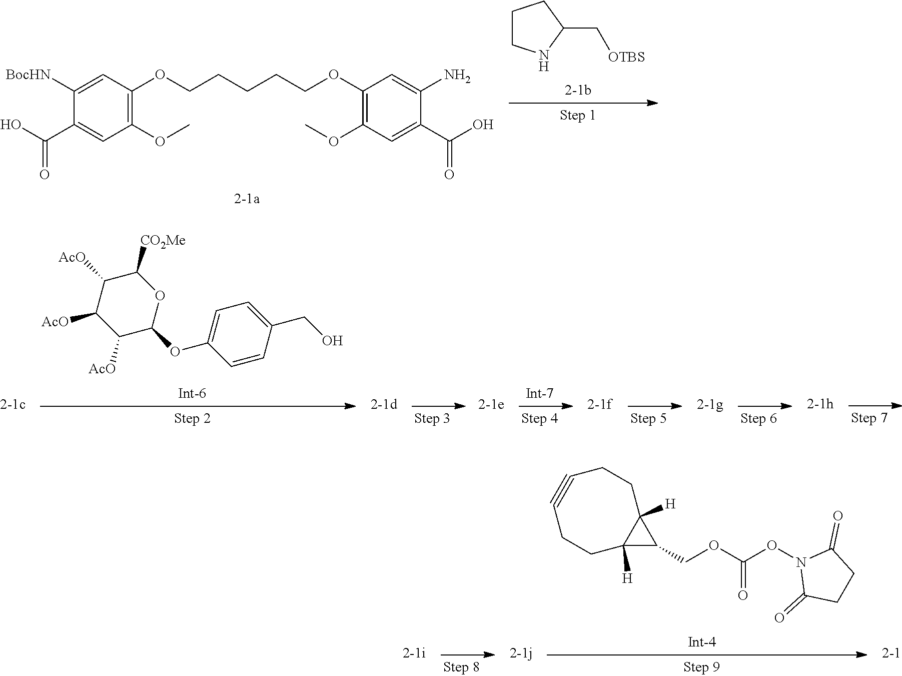 [SMILES: CC(C)(C)[Si](C)(C)OCC1CCCN1 ; COc1cc(C(=O)O)c(N)cc1OCCCCCOc1cc(NC(=O)OC(C)(C)C)c(C(=O)O)cc1OC ; COC(=O)[C@H]1O[C@@H](Oc2ccc(CO)cc2)[C@H](OC(C)=O)[C@@H](OC(C)=O)[C@@H]1OC(C)=O ; O=C(OC[C@@H]1[C@@H]2CCC#CCC[C@@H]21)ON1C(=O)CCC1=O]

### Step 1: tert-Butyl (5-((5-(5-amino-4-((S)-2-(((tert-butyldimethylsilyl)oxy)methyl)pyrrolidine-1-carbonyl)-2-methoxyphenoxy)pentyl)oxy)-2-((S)-2-(((tert-butyldimethylsilyl)oxy)methyl)pyrrolidine-1-carbonyl)-4-hydroxyphenyl)carbamate (2-1c)

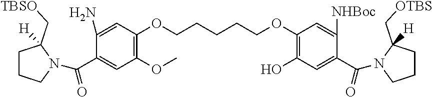 [SMILES: COc1cc(C(=O)N2CCC[C@H]2CO[Si](C)(C)C(C)(C)C)c(N)cc1OCCCCCOc1cc(NC(=O)OC(C)(C)C)c(C(=O)N2CCC[C@H]2CO[Si](C)(C)C(C)(C)C)cc1O]

Compound 2-1c (2.66 g, 66.3%) was synthesized according to similar procedures described in WO 2017059289 A1 and Org. Process Res. Dev. 2022, 26, 2155-2175. MS (ESI) m/z: 930.1 [M+H]⁺.

### Step 2: (2S,3S,4S,5R,6S)-4,5-Diacetoxy-6-(4-((((5-((5-(5-((tert-butoxycarbonyl)amino)-4-((S)-2-(((tert-butyldimethylsilyl)oxy)methyl)pyrrolidine-1-carbonyl)-2-hydroxyphenoxy)pentyl)oxy)-2-((S)-2-(((tert-butyldimethylsilyl)oxy)methyl)pyrrolidine-1-carbonyl)-4-methoxyphenyl)carbamoyl)oxy)methyl)phenoxy)-2-(methoxycarbonyl)tetrahydro-2H-pyran-3-carboxylic acid (2-1d)

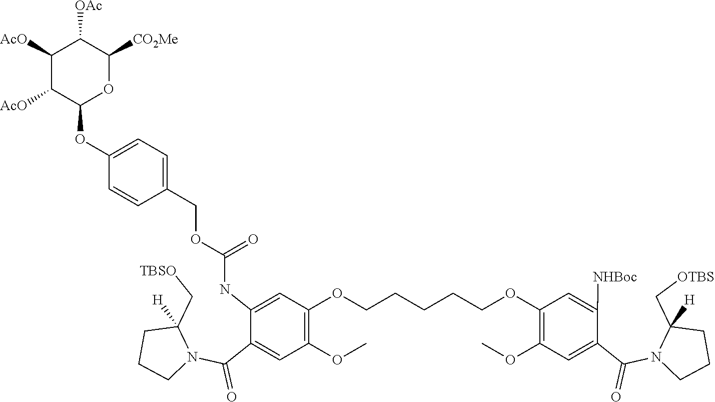 [SMILES: COC(=O)[C@H]1O[C@@H](Oc2ccc(COC(=O)Nc3cc(OCCCCCOc4cc(NC(=O)OC(C)(C)C)c(C(=O)N5CCC[C@H]5CO[Si](C)(C)C(C)(C)C)cc4OC)c(OC)cc3C(=O)N3CCC[C@H]3CO[Si](C)(C)C(C)(C)C)cc2)[C@H](OC(C)=O)[C@@H](OC(C)=O)[C@@H]1OC(C)=O]

Compound 2-1d (2.17 g, 67.4%) was synthesized according to the synthetic procedure of step 1 of example Ref-2-2. MS (ESI) m/z: 1395.8 [M+H].

### Step 3: (2S,3R,4S,5S,6S)-2-(4-((((5-((5-(5-Amino-4-((S)-2-(((tert-butyldimethylsilyl)oxy)methyl)pyrrolidine-1-carbonyl)-2-methoxyphenoxy)pentyl)oxy)-2-((S)-2-(((tert-butyldimethylsilyl)oxy)methyl)pyrrolidine-1-carbonyl)-4-methoxyphenyl)carbamoyl)oxy)methyl)phenoxy)-6-(methoxycarbonyl)tetrahydro-2H-pyran-3,4,5-triyl triacetate (2-1e)

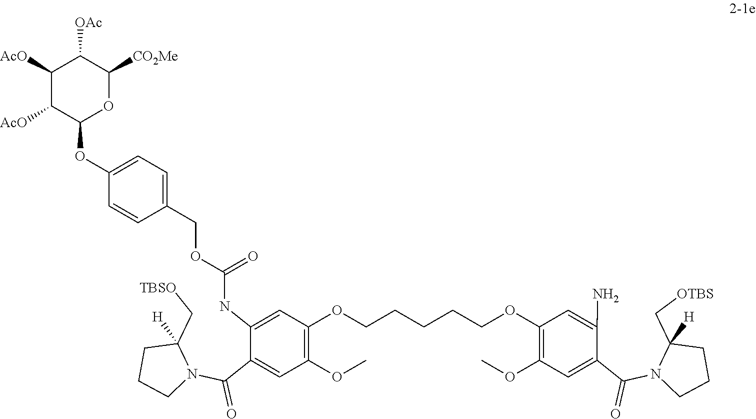 [SMILES: COC(=O)[C@H]1O[C@@H](Oc2ccc(COC(=O)Nc3cc(OCCCCCOc4cc(N)c(C(=O)N5CCC[C@H]5CO[Si](C)(C)C(C)(C)C)cc4OC)c(OC)cc3C(=O)N3CCC[C@H]3CO[Si](C)(C)C(C)(C)C)cc2)[C@H](OC(C)=O)[C@@H](OC(C)=O)[C@@H]1OC(C)=O]

To a solution of 2-1d (0.57 g, 0.41 mmol) in CH₂Cl₂ (6 mL) were added TBSOTf (162 mg, 0.61 mmol) on ice-bath. After addition, the reaction mixture was stirred at r.t. overnight, then 2,6-lutidine was added and stirred overnight. After complete reaction, the mixture was quenched with sat. NH₄Cl aq. (10 mL), extracted with CH₂Cl₂ (10 mL*3). The combined organic layers were washed with H₂O (15 mL), brine (15 mL), dried over anhydrous Na₂SO₄, filtered and concentrated under vacuum to give a residue which was purified by silica gel column chromatography (A-CH₂Cl₂; B-MeOH) to provide 2-1e (439 mg, 83.1%) as a pale-yellow solid. MS (ESI) m/z: 1295.2 [M+H]⁺.

### Step 4: (2S,3S,4S,5R,6S)-6-(2-(1-(9H-Fluoren-9-yl)-3-oxo-2,7,10,13,16-pentaoxa-4-azanonadecan-19-amido)-4-((((2-((S)-2-(((tert-butyldimethylsilyl)oxy)methyl)pyrrolidine-1-carbonyl)-5-((5-(4-((S)-2-(((tert-butyldimethylsilyl)oxy)methyl)pyrrolidine-1-carbonyl)-2-methoxy-5-((((4-(((2S,3R,4S,5S,6S)-3,4,5-triacetoxy-6-(methoxycarbonyl)tetrahydro-2H-pyran-2-yl)oxy)benzyl)oxy)carbonyl)amino)phenoxy)pentyl)oxy)-4-methoxyphenyl)carbamoyl)oxy)methyl)phenoxy)-3,4,5-trihydroxytetrahydro-2H-pyran-2-carboxylic acid (2-1f)

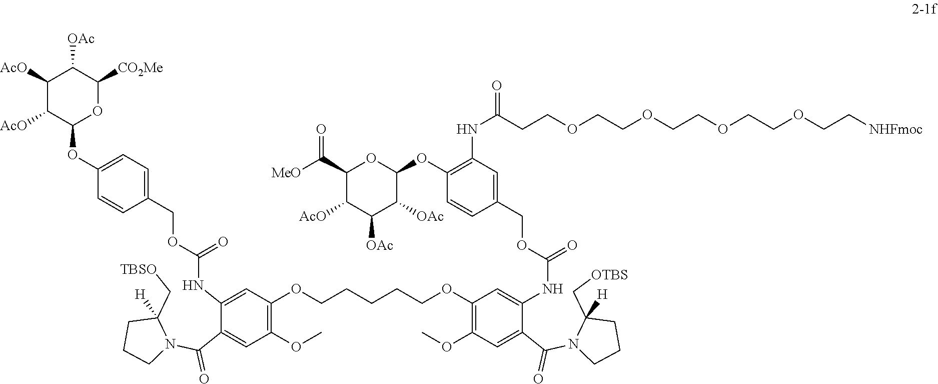

Compound 2-1f (205 mg, 91.1%) was synthesized according to the synthetic procedure of step 1 of example Ref-2-2. MS (ESI) m/z: 2246.0 [M+H]⁺.

### Step 5: (2S,3S,4S,5R,6S)-6-(2-(1-(9H-Fluoren-9-yl)-3-oxo-2,7,10,13,16-pentaoxa-4-azanonadecan-19-amido)-4-((((2-((S)-2-(hydroxymethyl)pyrrolidine-1-carbonyl)-5-((5-(4-((S)-2-(hydroxymethyl)pyrrolidine-1-carbonyl)-2-methoxy-5-((((4-(((2S,3R,4S,5S,6S)-3,4,5-triacetoxy-6-(methoxy carbonyl)tetrahydro-2H-pyran-2-yl)oxy)benzyl)oxy)carbonyl)amino)phenoxy)pentyl)oxy)-4-methoxyphenyl)carbamoyl)oxy)methyl)phenoxy)-3,4,5-trihydroxytetrahydro-2H-pyran-2-carboxylic acid (2-1g)

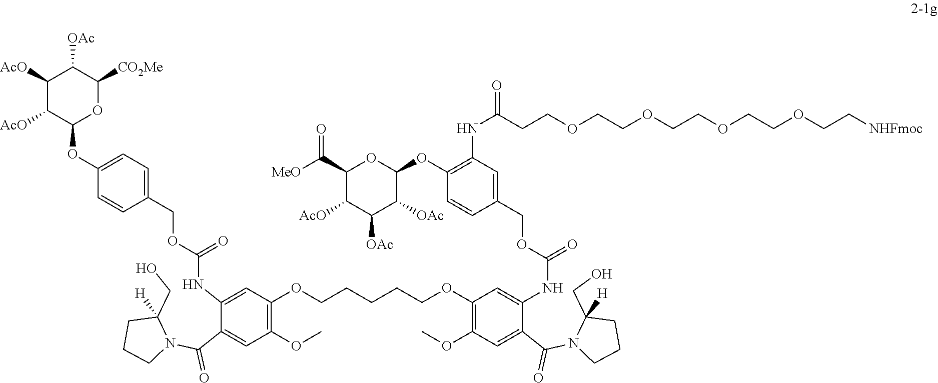

Compound 2-1g (162 mg, 88.1%) was synthesized according to the synthetic procedure of step 2 of example Ref-2-2. MS (ESI) m/z: 2018.5 [M+H]⁺.

### Step 6: (2S,3S,4S,5R,6S)-6-(2-(1-(9H-Fluoren-9-yl)-3-oxo-2,7,10,13,16-pentaoxa-4-azanonadecan-19-amido)-4-((((11S,11aS)-11-hydroxy-8-((5-(((11S,11aS)-11-hydroxy-7-methoxy-5-oxo-10-(((4-(((2S,3R,4S,5S,6S)-3,4,5-triacetoxy-6-(methoxycarbonyl)tetrahydro-2H-pyran-2-yl)oxy)benzyl)oxy)carbonyl)-2,3,5,10,11,11a-hexahydro-1H-benzo[e]pyrrolo[1,2-a][1,4]diazepin-8-yl)oxy)pentyl)oxy)-7-methoxy-5-oxo-2,3,5,10,11,11a-hexahydro-1H-benzo[e]pyrrolo[1,2-a][1,4]diazepine-10-carbonyl)oxy)methyl)phenoxy)-3,4,5-trihydroxytetrahydro-2H-pyran-2-carboxylic acid (2-1h)

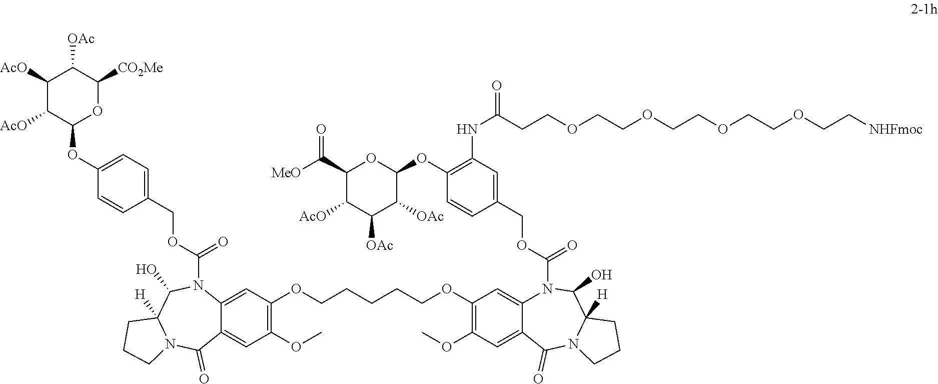

To a solution of 2-1g (152 mg, 0.075 mmol) in CH₂Cl₂ (10 mL) were added Dess-Martin periodinane (130 mg, 0.30 mmol) on ice-bath. After addition, the reaction mixture was reacted at r.t. for 3 h. After complete reaction, the mixture was diluted with CH₂Cl₂ (15 mL) and quenched with a mixed solution of sat. Na₂S203 aq. and sat. NaHCO₃ aq. (1:1, 15 mL). The aqueous phase was extracted with CH₂Cl₂ (15 mL*3). The combined organic layers were washed with H₂O (30 mL), brine (30 mL), dried over anhydrous Na₂SO₄, filtered and concentrated under vacuum to give a residue which was purified by silica gel column chromatography (A-CH₂Cl₂; B-MeOH) to provide 2-1h (136 mg, 89.3%) as a pale-yellow solid. MS (ESI) m/z: 2014.4 [M+H]⁺.

### Step 7: (2S,3S,4S,5R,6S)-6-(2-(1-(9H-Fluoren-9-yl)-3-oxo-2,7,10,13,16-pentaoxa-4-azanonadecan-19-amido)-4-((((11S,11aS)-8-((5-(((11S,11aS)-10-(((4-(((2S,3R,4S,5S,6S)-6-carboxy-3,4,5-trihydroxytetrahydro-2H-pyran-2-yl)oxy)benzyl)oxy)carbonyl)-11-hydroxy-7-methoxy-5-oxo-2,3,5,10,11,11a-hexahydro-1H-benzo[e]pyrrolo[1,2-a][1,4]diazepin-8-yl)oxy)pentyl)oxy)-11-hydroxy-7-methoxy-5-oxo-2,3,5,10,11,11a-hexahydro-1H-benzo[e]pyrrolo[1,2-a][1,4]diazepine-10-carbonyl)oxy)methyl)phenoxy)-3,4,5-trihydroxytetrahydro-2H-pyran-2-carboxylic acid (2-1i)

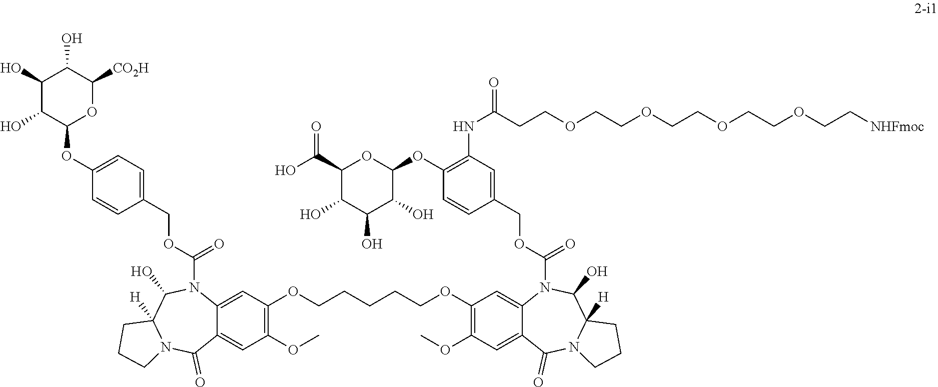

To a pre-dissolved solution of LiBr (217 mg, 2.50 mmol) in MeCN/H₂O (9/1, v/v, 0.5 mL) was added 2-1h (50 mg, 0.025 mmol) and Et₃N (25 mg, 0.25 mmol) at r.t. The resulting suspension was stirred at r.t. for 1.5 h. The mixture was acidified with AcOH (15 mg, 0.25 mmol) at 0° C. and then concentrated under vacuum. The residue was co-evaporated with toluene (10 mL*3) give the crude product as a yellow oil, which was used for next step without further purification. MS (ESI) m/z: 1733.4 [M+H]⁺.

### Step 8: (2S,3S,4S,5R,6S)-6-(2-(1-Amino-3,6,9,12-tetraoxapentadecan-15-amido)-4-((((11S,11aS)-8-((5-(((11S,11aS)-10-(((4-(((2S,3R,4S,5S,6S)-6-carboxy-3,4,5-trihydroxytetrahydro-2H-pyran-2-yl)oxy)benzyl)oxy)carbonyl)-11-hydroxy-7-methoxy-5-oxo-2,3,5,10,11,11a-hexahydro-1H-benzo[e]pyrrolo[1,2-a][1,4]diazepin-8-yl)oxy)pentyl)oxy)-11-hydroxy-7-methoxy-5-oxo-2,3,5,10,11,11a-hexahydro-1H-benzo[e]pyrrolo[1,2-a][1,4]diazepine-10-carbonyl)oxy)methyl)phenoxy)-3,4,5-trihydroxytetrahydro-2H-pyran-2-carboxylic acid (2-1j)

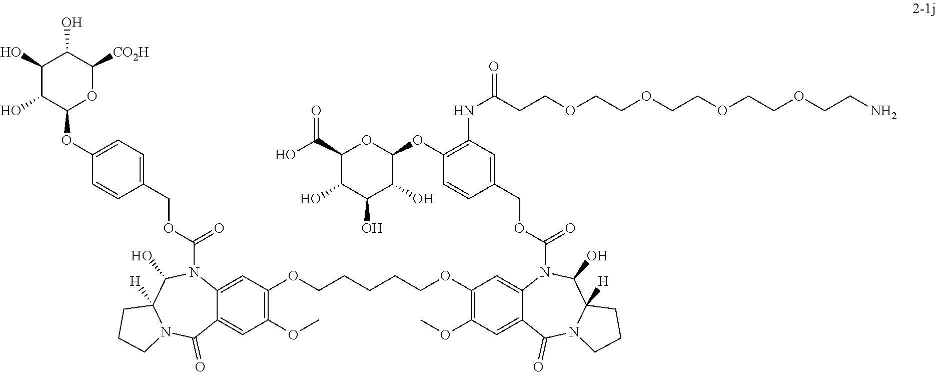 [SMILES: COc1cc2c(cc1OCCCCCOc1cc3c(cc1OC)C(=O)N1CCC[C@H]1[C@H](O)N3C(=O)OCc1ccc(O[C@@H]3O[C@H](C(=O)O)[C@@H](O)[C@H](O)[C@H]3O)c(NC(=O)CCOCCOCCOCCOCCN)c1)N(C(=O)OCc1ccc(O[C@@H]3O[C@H](C(=O)O)[C@@H](O)[C@H](O)[C@H]3O)cc1)[C@@H](O)[C@@H]1CCCN1C2=O]

To a solution of 2-1i (crude) in DMF (1 mL) was added Et₂NH (37 mg, 0.25 mmol) at r.t. The resulting solution was reacted at r.t. for 2 h, and reaction completion was observed by LCMS analysis. The mixture was diluted with DMF (1 mL) and acidified with AcOH (15 mg, 0.25 mmol) at 0° C. The mixture was purified by prep-HPLC (Method: column:XBridge Prep C18 OBD 5 μm 19*150 mm; Mobile phase: A—water (0.1% formic acid): B-acetonitrile; Flow rate: 20 mL/min) to provide 2-1j (20 mg, 53.3% yield) as a white solid. MS (ESI) m/z: 1512.0 [M+H]⁺.

### Step 9: (2S,3S,4S,5R,6S)-6-(2-(1-((1R,8S,9s)-bicyclo[6.1.0]non-4-yn-9-yl)-3-oxo-2,7,10,13,16-pentaoxa-4-azanonadecan-19-amido)-4-((((11S,11aS)-8-((5-(((11S,11aS)-10-(((4-(((2S,3R,4S,5S,6S)-6-carboxy-3,4,5-trihydroxytetrahydro-2H-pyran-2-yl)oxy)benzyl)oxy)carbonyl)-11-hydroxy-7-methoxy-5-oxo-2,3,5,10,11,11a-hexahydro-1H-benzol[e]pyrrolo[1,2-a][1,4]diazepin-8-yl)oxy)pentyl)oxy)-11-hydroxy-7-methoxy-5-oxo-2,3,5,10,11,11a-hexahydro-1H-benzo[e]pyrrolo[1,2-a][1,4]diazepine-10-carbonyl)oxy)methyl)phenoxy)-3,4,5-trihydroxytetrahydro-2H-pyran-2-carboxylic acid (2-1)

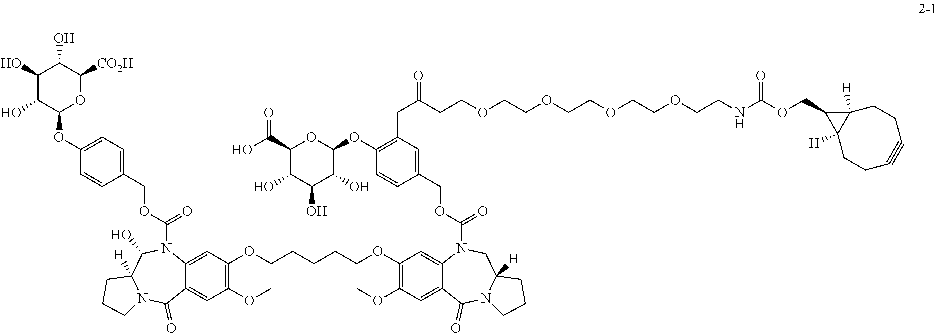 [SMILES: COc1cc2c(cc1OCCCCCOc1cc3c(cc1OC)C(=O)N1CCC[C@H]1[C@H](O)N3C(=O)OCc1ccc(O[C@@H]3O[C@H](C(=O)O)[C@@H](O)[C@H](O)[C@H]3O)cc1)N(C(=O)OCc1ccc(O[C@@H]3O[C@H](C(=O)O)[C@@H](O)[C@H](O)[C@H]3O)c(CC(=O)CCOCCOCCOCCOCCNC(=O)OC[C@@H]3[C@@H]4CCC#CCC[C@@H]43)c1)C[C@@H]1CCCN1C2=O]

To a mixed solution of 2-1j (20 mg, 0.013 mmol) and Int-4 (20 mg, 0.066 mmol) in DMF (0.5 mL) was added DIPEA (8.6 mg, 0.066 mmol) at r.t. The resulting solution was reacted at r.t. for 0.5 h and reaction completion was observed by LCMS analysis. The mixture was diluted with DMF (1 mL) and acidified with AcOH (4.0 mg, 0.066 mmol) at 0° C. The mixture was purified using prep-HPLC (Method: column:XBridge Prep C18 OBD 5 μm 19*150 mm; Mobile phase: A-water (0.1% formic acid): B-acetonitrile; Flow rate: 20 mL/min) to provide 2-1 (15.6 mg, 69.9% yield) as a white solid. MS (ESI) m/z: 1687.4 [M+H]⁺.

### Example 2-2

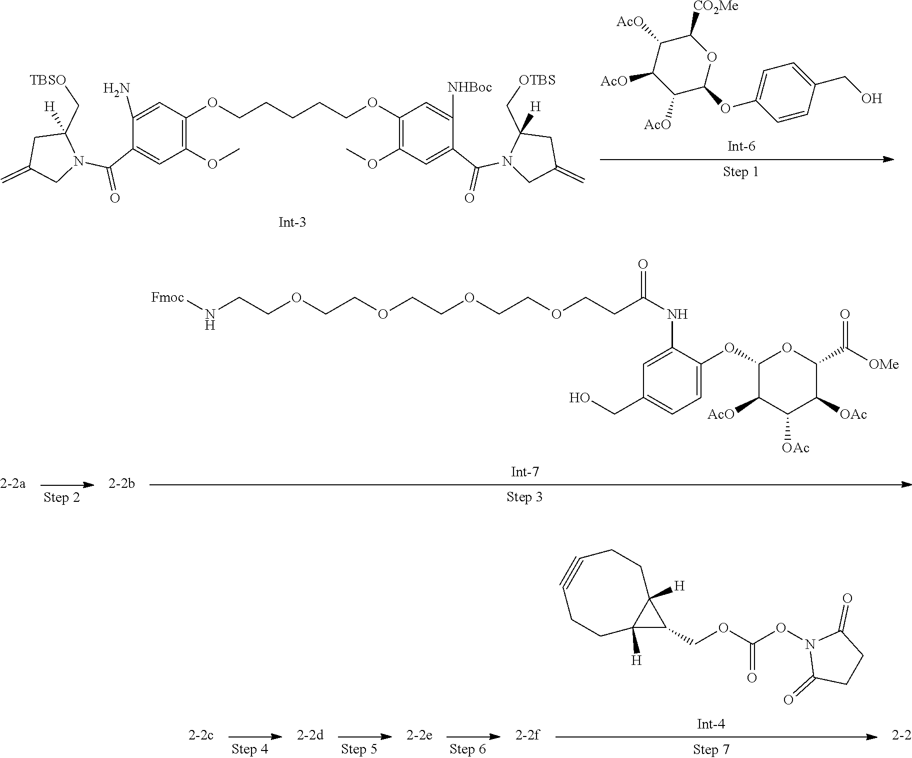

### Step 1: (2S,3R,4S,5S,6S)-2-(4-((((5-((5-(5-((tert-butoxycarbonyl)amino)-4-((S)-2-(((tert-butyldimethylsilyl)oxy)methyl)-4-methylenepyrrolidine-1-carbonyl)-2-methoxyphenoxy)pentyl)oxy)-2-((S)-2-(((tert-butyldimethylsilyl)oxy)methyl)-4-methylenepyrrolidine-1-carbonyl)-4-methoxyphenyl)carbamoyl)oxy)methyl)phenoxy)-6-(methoxycarbonyl)tetrahydro-2H-pyran-3,4,5-triyl triacetate (2-2a)

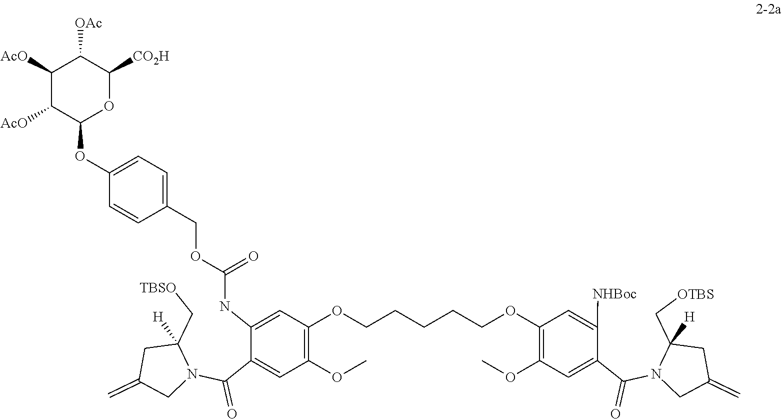 [SMILES: C=C1C[C@@H](CO[Si](C)(C)C(C)(C)C)N(C(=O)c2cc(OC)c(OCCCCCOc3cc(NC(=O)OC(C)(C)C)c(C(=O)N4CC(=C)C[C@H]4CO[Si](C)(C)C(C)(C)C)cc3OC)cc2NC(=O)OCc2ccc(O[C@@H]3O[C@H](C(=O)O)[C@@H](OC(C)=O)[C@H](OC(C)=O)[C@H]3OC(C)=O)cc2)C1]

To a mixture of Int-3 (700 mg, 0.73 mmol) (Int-3 was synthesized according to procedures described in Org. Process Res. Dev. 2022, 26, 2155-2175) and 4 Å molecular sieves (200 mg) in anhydrous THF (8 mL) were added triphosgene (80 mg, 0.27 mmol) and Et₃N (224 μL, 163 mg, 1.62 mmol) at 0° C., then stirred on ice-bath for 10 min. Then a mixture of Int-6 (355 mg, 0.81 mmol), Dibutyltin dilaurate (46 mg, 0.073 mmol) and Et₃N (153 μL, 111 mg, 1.10 mmol) in THF (4 mL) was added. The mixture was stirred at r.t. for 1.5 h. The solvent was removed by evaporation and the crude was purified by silica gel column chromatography (10 g, EtOAc/Petroleum=0/100-30/70-100/0), fraction was concentrated under vacuum to give crude product, which was further purified by reserved phased column (60 g C18, MeCN/H₂O=30/70-100/0) to give 2-2a (807 mg, 77% yield) as a white solid. MS (ESI) m/z: 1420.4 [M+H]⁺.

### Step 2: (2S,3R,4S,5S,6S)-2-(4-((((5-((5-(5-amino-4-((S)-2-(((tert-butyldimethylsilyl)oxy)methyl)-4-methylenepyrrolidine-1-carbonyl)-2-methoxyphenoxy)pentyl)oxy)-2-((S)-2-(((tert-butyldimethylsilyl)oxy)methyl)-4-methylenepyrrolidine-1-carbonyl)-4-methoxyphenyl)carbamoyl)oxy)methyl)phenoxy)-6-(methoxycarbonyl)tetrahydro-2H-pyran-3,4,5-triyl triacetate (2-2b)

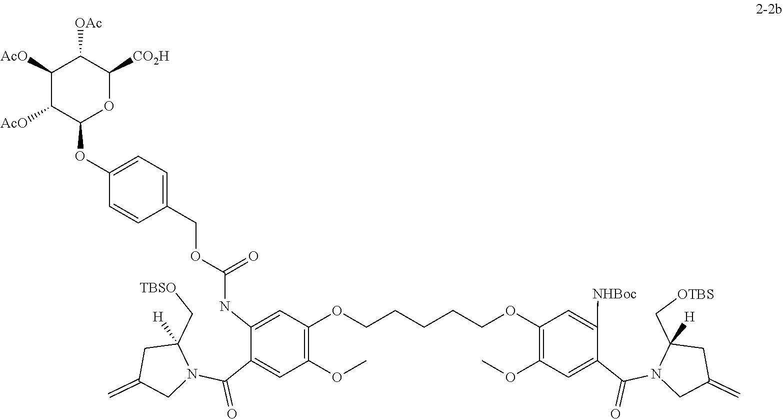 [SMILES: C=C1C[C@@H](CO[Si](C)(C)C(C)(C)C)N(C(=O)c2cc(OC)c(OCCCCCOc3cc(NC(=O)OC(C)(C)C)c(C(=O)N4CC(=C)C[C@H]4CO[Si](C)(C)C(C)(C)C)cc3OC)cc2NC(=O)OCc2ccc(O[C@@H]3O[C@H](C(=O)O)[C@@H](OC(C)=O)[C@H](OC(C)=O)[C@H]3OC(C)=O)cc2)C1]

A solution of 2-2a (784 mg, 0.55 mmol) in hexafluoro isopropanol (8 mL) in the sealed tube was heated to 80° C. for 4 days. The mixture was concentrated under vacuum to give crude product then purified by silica gel column chromatography (10 g, MeOH/CH₂Cl₂=0/100-3/97) to give 2-2b (516 mg, 71% yield) as a white solid. MS (ESI) m/z: 1319.9 [M+H]⁺.

### Step 3: (2S,3R,4S,5S,6S)-2-(2-(1-(9H-fluoren-9-yl)-3-oxo-2,7,10,13,16-pentaoxa-4-azanonadecan-19-amido)-4-((((2-((S)-2-(((tert-butyldimethylsilyl)oxy)methyl)-4-methylenepyrrolidine-1-carbonyl)-5-((5-(4-((S)-2-(((tert-butyldimethylsilyl)oxy)methyl)-4-methylenepyrrolidine-1-carbonyl)-2-methoxy-5-((((4-(((2S,3R,4S,5S,6S)-3,4,5-triacetoxy-6-(methoxy carbonyl)tetrahydro-2H-pyran-2-yl)oxy)benzyl)oxy)carbonyl)amino)phenoxy)pentyl)oxy)-4-methoxyphenyl)carbamoyl)oxy)methyl)phenoxy)-6-(methoxycarbonyl)tetrahydro-2H-pyran-3,4,5-triyl triacetate (2-2c)

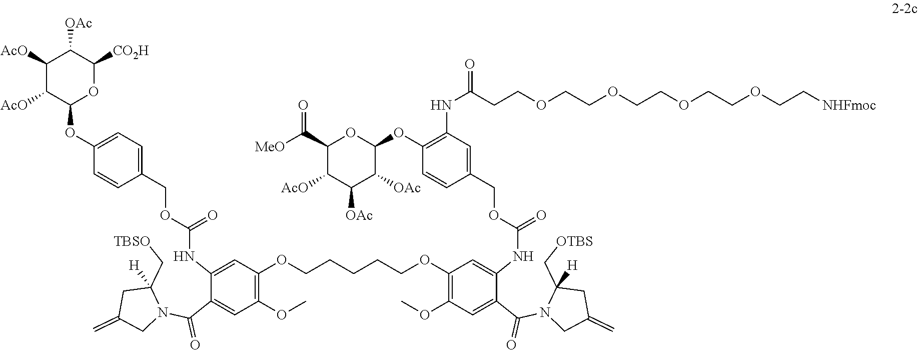

To a mixture of 2-2b (500 mg, 0.38 mmol) and 4 Å molecular sieves (100 mg) in anhydrous THF (5 mL) were added triphosgene (42 mg, 0.14 mmol) and Et₃N (116 μL, 84 mg, 0.42 mmol) at 0° C., then stirred on ice-bath for 10 min. LCMS (quenched with MeOH) showed full consumption of 2-2b. Then a mixture of Int-7 (357 mg, 0.39 mmol), Dibutyltin dilaurate (12 mg, 0.019 mmol) and Et₃N (80 μL, 57 mg, 0.28 mmol) in THF (5 mL) was added. The mixture was stirred at r.t. for 1.5 h. After reaction was complete then purified by silica gel column chromatography (10 g, MeOH/CH₂Cl₂=0/100˜1/99), the fraction was concentrated under vacuum to give 2-2c (857 mg, 99% yield) as a yellow foam solid. MS (ESI) m/z: 2293.4 [M+Na⁺]

### Step 4: (2S,3R,4S,5S,6S)-2-(2-(1-(9H-fluoren-9-yl)-3-oxo-2,7,10,13,16-pentaoxa-4-azanonadecan-19-amido)-4-((((2-((S)-2-(hydroxymethyl)-4-methylenepyrrolidine-1-carbonyl)-5-((5-(4-((S)-2-(hydroxymethyl)-4-methylenepyrrolidine-1-carbonyl)-2-methoxy-5-((((4-(((2S,3R,4S,5S,6S)-3,4,5-triacetoxy-6-(methoxycarbonyl)tetrahydro-2H-pyran-2-yl)oxy)benzyl)oxy)carbonyl)amino)phenoxy)pentyl)oxy)-4-methoxyphenyl)carbamoyl)oxy)methyl)phenoxy)-6-(methoxycarbonyl)tetrahydro-2H-pyran-3,4,5-triyl triacetate (2-2d)

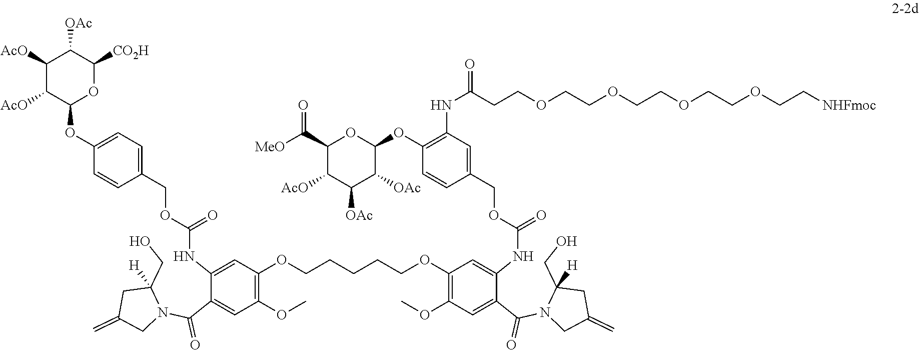

To a solution of 2-2c (200 mg, 0.088 mmol) in THF/H₂O (2 mL/0.1 mL) was added PTSA (36 mg, 0.21 mmol). The mixture was stirred at r.t. for 1.5 h. The mixture was quenched with sat. NaHCO₃ (10 mL), extracted with EtOAc (20 mL*3). Combined organic layers were washed with brine (10 mL), dried over Na₂SO₄, filtered, and concentrated under vacuum to give a residue then purified by silica gel column chromatography (10 g, MeOH/CH₂Cl₂=0/100-10/90) to give 2-2d (165 mg, 92% yield) as a yellow foam solid. MS (ESI) m/z: 2064.4 [M+Na⁺]

### Step 5: (2S,3R,4S,5S,6S)-2-(2-(1-(9H-fluoren-9-yl)-3-oxo-2,7,10,13,16-pentaoxa-4-azanonadecan-19-amido)-4-((((11S,11aS)-11-hydroxy-8-((5-(((11S,11aS)-11-hydroxy-7-methoxy-2-methylene-5-oxo-10-(((4-(((2S,3R,4S,5S,6S)-3,4,5-triacetoxy-6-(methoxycarbonyl)tetrahydro-2H-pyran-2-yl)oxy)benzyl)oxy)carbonyl)-2,3,5,10,11,11a-hexahydro-1H-benzo[e]pyrrolo[1,2-a][1,4]diazepin-8-yl)oxy)pentyl)oxy)-7-methoxy-2-methylene-5-oxo-2,3,5,10,11,11a-hexahydro-1H-benzo[e]pyrrolo[1,2-a][1,4]diazepine-10-carbonyl)oxy)methyl)phenoxy)-6-(methoxycarbonyl)tetrahydro-2H-pyran-3,4,5-triyl triacetate (2-2e)

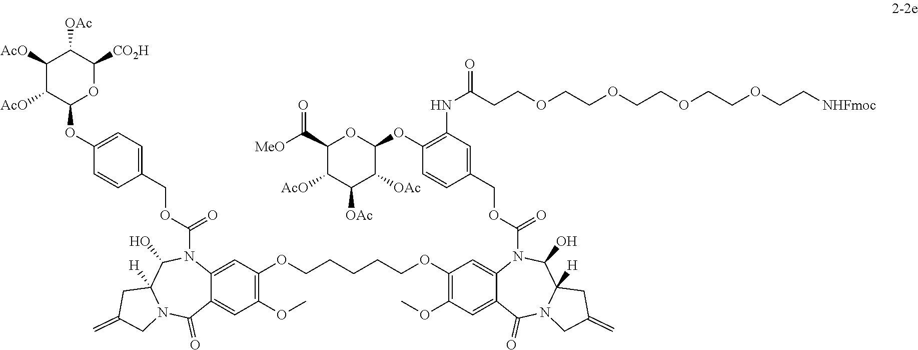

A mixture of 2-2d (165 mg, 0.080 mmol), BAIB (55 mg, 0.17 mmol) and TEMPO (1.3 mg, 0.008 mmol) in CH₂Cl₂ (3 mL) was stirred at r.t. for 30 h. The mixture was quenched by addition of 10% Na₂S203 solution (10 mL), extracted with CH₂Cl₂ (10 mL*3). Combined organic layers were washed with brine (10 mL), dried over Na₂SO₄, filtered and the filtrate was concentrated under vacuum to give a residue, which was purified by silica gel column chromatography (5 g, EtOAc/Petroleum ether=30/80-100/0 and MeOH/CH₂Cl_{2=0/100}-1/99) to give 2-2e (123 mg, 45% yield) as a brown foam solid. MS (ESI) m/z: 2060.5 [M+Na⁺]

### Step 6: (2S,3S,4S,5R,6S)-6-(2-(1-amino-3,6,9,12-tetraoxapentadecan-15-amido)-4-((((11S,11aS)-8-((5-(((11S,11aS)-10-(((4-(((2S,3R,4S,5S,6S)-6-carboxy-3,4,5-trihydroxytetrahydro-2H-pyran-2-yl)oxy)benzyl)oxy)carbonyl)-11-hydroxy-7-methoxy-2-methylene-5-oxo-2,3,5,10,11,11a-hexahydro-1H-benzo[e]pyrrolo[1,2-a][1,4]diazepin-8-yl)oxy)pentyl)oxy)-11-hydroxy-7-methoxy-2-methylene-5-oxo-2,3,5,10,11,11a-hexahydro-1H-benzo[e]pyrrolo[1,2-a][1,4]diazepine-10-carbonyl)oxy)methyl)phenoxy)-3,4,5-trihydroxytetrahydro-2H-pyran-2-carboxylic acid (2-2f)

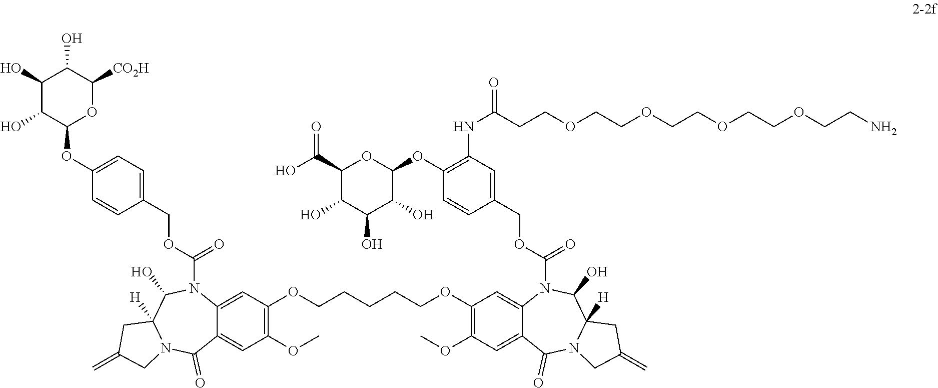 [SMILES: C=C1C[C@H]2[C@H](O)N(C(=O)OCc3ccc(O[C@@H]4O[C@H](C(=O)O)[C@@H](O)[C@H](O)[C@H]4O)cc3)c3cc(OCCCCCOc4cc5c(cc4OC)C(=O)N4CC(=C)C[C@H]4[C@H](O)N5C(=O)OCc4ccc(O[C@@H]5O[C@H](C(=O)O)[C@@H](O)[C@H](O)[C@H]5O)c(NC(=O)CCOCCOCCOCCOCCN)c4)c(OC)cc3C(=O)N2C1]

To a solution of 2-2e (123 mg, 0.06 mmol) in THF/H₂O (3 mL/1 mL) was added LiOH (10.1 mg, 0.42 mmol) on ice-bath then stirred at the same temperature for 1 h and warmed to r.t. for 1 h. The mixture was diluted with H₂O (3 mL), washed with EtOAc (10 mL). The aqueous phase was acidified with 1N KHSO₄ to pH 6, then purified by prep-HPLC (0.1% FA) (Method: column:XBridge Prep C18 OBD 5 μm 19*150 mm; Mobile phase: A—water (0.1% formic acid): B-acetonitrile; Flow rate: 20 mL/min) to give 2-2f (21.7 mg, 23.3% yield) as a white solid. MS (ESI) m/z: 1535.8 [M+H]⁺.

### Step 7: (2S,3S,4S,5R,6S)-6-(2-(1-((1R,8S,9s)-bicyclo[6.1.0]non-4-yn-9-yl)-3-oxo-2,7,10,13,16-pentaoxa-4-azanonadecan-19-amido)-4-((((11S,11aS)-8-((5-(((11S,11aS)-10-(((4-(((2S,3R,4S,5S,6S)-6-carboxy-3,4,5-trihydroxytetrahydro-2H-pyran-2-yl)oxy)benzyl)oxy)carbonyl)-11-hydroxy-7-methoxy-2-methylene-5-oxo-2,3,5,10,11,11a-hexahydro-1H-benzo[e]pyrrolo[1,2-a][1,4]diazepin-8-yl)oxy)pentyl)oxy)-11-hydroxy-7-methoxy-2-methylene-5-oxo-2,3,5,10,11,11a-hexahydro-1H-benzo[e]pyrrolo[1,2-a][1,4]diazepine-10-carbonyl)oxy)methyl)phenoxy)-3,4,5-trihydroxytetrahydro-2H-pyran-2-carboxylic acid (2-2)

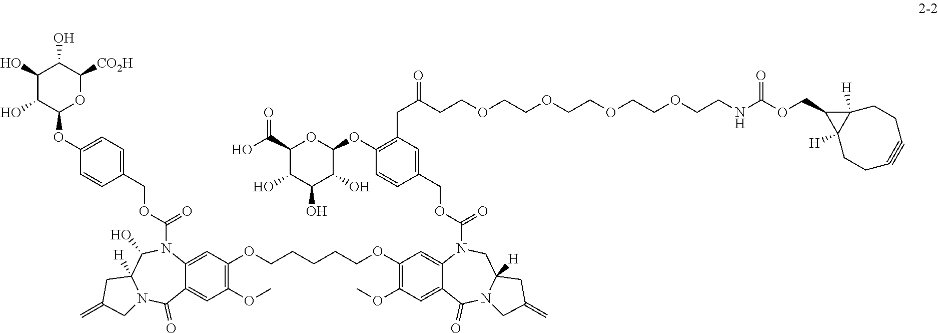 [SMILES: C=C1C[C@H]2CN(C(=O)OCc3ccc(O[C@@H]4O[C@H](C(=O)O)[C@@H](O)[C@H](O)[C@H]4O)c(CC(=O)CCOCCOCCOCCOCCNC(=O)OC[C@@H]4[C@@H]5CCC#CCC[C@@H]54)c3)c3cc(OCCCCCOc4cc5c(cc4OC)C(=O)N4CC(=C)C[C@H]4[C@H](O)N5C(=O)OCc4ccc(O[C@@H]5O[C@H](C(=O)O)[C@@H](O)[C@H](O)[C@H]5O)cc4)c(OC)cc3C(=O)N2C1]

To a mixture of 2-2f (18.7 mg, 0.012 mmol) and Int-4 (10.5 mg, 0.036 mmol) in DMF (2 mL) was added DIPEA (4.7 mg, 0.036 mmol). The mixture was stirred at r.t. for 30 min. The mixture was purified by prep-HPLC (0.1% FA) (Method: column:XBridge Prep C18 OBD 5 μm 19*150 mm; Mobile phase: A—water (0.1% formic acid): B-acetonitrile; Flow rate: 20 mL/min) to give 2-2 (11.9 mg, 57.9% yield) as a white solid. MS (ESI) m/z: 1733.8 [M+Na]⁺.

### Example 2-4

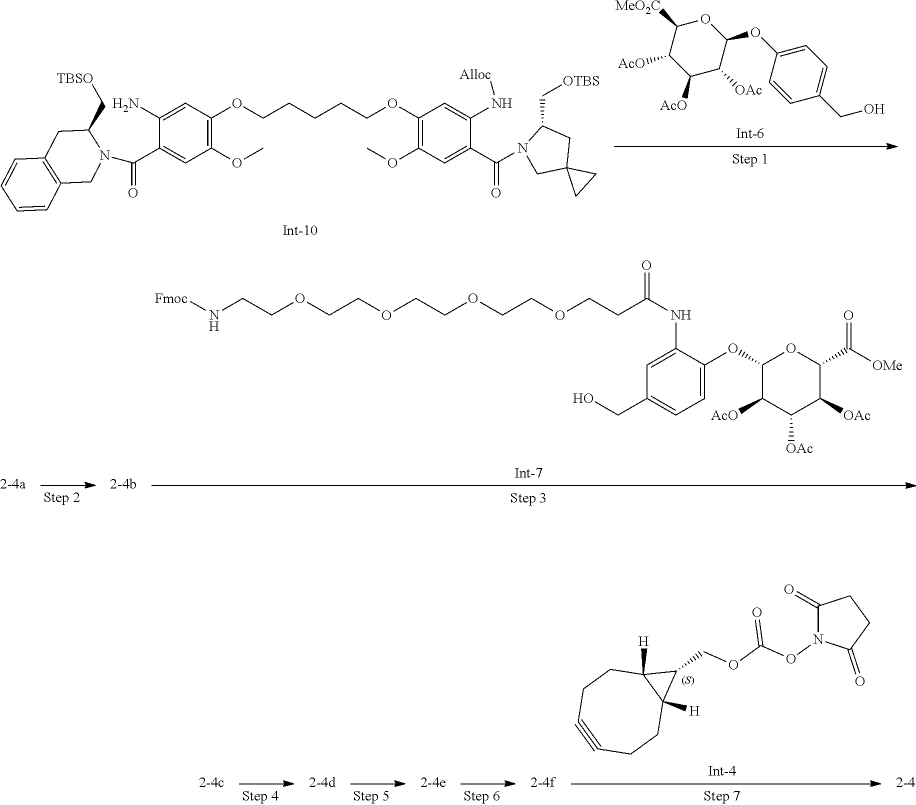

### Step 1: (2S,3R,4S,5S,6S)-2-(4-((((5-((5-(5-(((allyloxy)carbonyl)amino)-4-((S)-6-(((tert-butyldimethylsilyl)oxy)methyl)-5-azaspiro[2.4]heptane-5-carbonyl)-2-methoxyphenoxy)pentyl)oxy)-2-((S)-3-(((tert-butyldimethylsilyl)oxy)methyl)-1,2,3,4-tetrahydroisoquinoline-2-carbonyl)-4-methoxyphenyl)carbamoyl)oxy)methyl)phenoxy)-6-(methoxycarbonyl)tetrahydro-2H-pyran-3,4,5-triyl triacetate (2-4a)

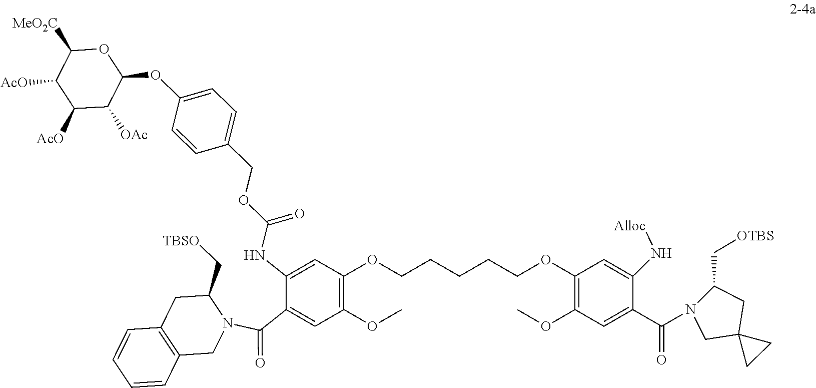

To a 0° C. solution of Int-10 (100 mg, 0.100 mmol), 4 Å Molecular Sieves (100 mg) and THF (3 mL) were added triphosgene (12 mg, 0.040 mmol) and Et₃N (31 μL, 0.22 mmol). The mixture was stirred at 0° C. for 10 min under N₂. A solution of Int-6 (57 mg, 0.13 mmol), Et₃N (21 μL, 0.15 mmol), dibutyltin dilaurate (6.3 mg, 0.010 mmol) in THF (1 mL) was added to the above reaction mixture. The mixture was stirred at r.t. overnight under N₂. The mixture was filtrated. The filterate was concentrated under vacuum to give a residue which was purified by silica gel column chromatography (Petroleum ether/EtOAc=20/80) to afford 2-4a (140 mg, 94.0% yield) as a white solid. MS (ESI) m/z: 1490.2 [M+H]⁺.

### Step 2: (2S,3R,4S,5S,6S)-2-(4-((((5-((5-(5-amino-4-((S)-6-(((tert-butyldimethylsilyl)oxy)methyl)-5-azaspiro[2.4]heptane-5-carbonyl)-2-methoxyphenoxy)pentyl)oxy)-2-((S)-3-(((tert-butyldimethylsilyl)oxy)methyl)-1,2,3,4-tetrahydroisoquinoline-2-carbonyl)-4-methoxyphenyl)carbamoyl)oxy)methyl)phenoxy)-6-(methoxycarbonyl)tetrahydro-2H-pyran-3,4,5-triyl triacetate (2-4b)

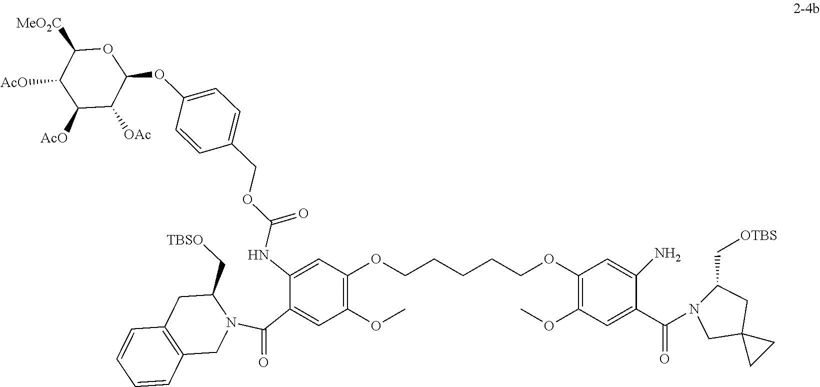 [SMILES: COC(=O)[C@H]1O[C@@H](Oc2ccc(COC(=O)Nc3cc(OCCCCCOc4cc(N)c(C(=O)N5CC6(CC6)C[C@H]5CO[Si](C)(C)C(C)(C)C)cc4OC)c(OC)cc3C(=O)N3Cc4ccccc4C[C@H]3CO[Si](C)(C)C(C)(C)C)cc2)[C@H](OC(C)=O)[C@@H](OC(C)=O)[C@@H]1OC(C)=O]

To a solution of 2-4a (140 mg, 0.095 mmol), pyrrolidine (20 μL, 0.24 mmol) and CH₂Cl₂ (5 mL) were added Pd(PPh₃)₄ (11 mg, 0.0095 mmol) under N₂. The mixture was stirred at r.t. for 1 h under N₂. The mixture was concentrated under vacuum to give a residue which was purified by silica gel column chromatography (CH₂Cl₂/MeOH=95/5) to afford 2-4b (98 mg, 74.8% yield) as a white solid. MS (ESI) m/z: 1383.9 [M+H].

### Step 3: (2S,3R,4S,5S,6S)-2-(2-(1-(9H-fluoren-9-yl)-3-oxo-2,7,10,13,16-pentaoxa-4-azanonadecan-19-amido)-4-((((5-((5-(4-((S)-3-(((tert-butyldimethylsilyl)oxy)methyl)-1,2,3,4-tetrahydroisoquinoline-2-carbonyl)-2-methoxy-5-((((4-(((2S,3R,4S,5S,6S)-3,4,5-triacetoxy-6-(methoxy carbonyl)tetrahydro-2H-pyran-2-yl)oxy)benzyl)oxy)carbonyl)amino)phenoxy)pentyl)oxy)-2-((S)-6-(((tert-butyldimethylsilyl)oxy)methyl)-5-azaspiro[2.4]heptane-5-carbonyl)-4-methoxyphenyl)carbamoyl)oxy)methyl)phenoxy)-6-(methoxycarbonyl)tetrahydro-2H-pyran-3,4,5-triyl triacetate (2-4c)

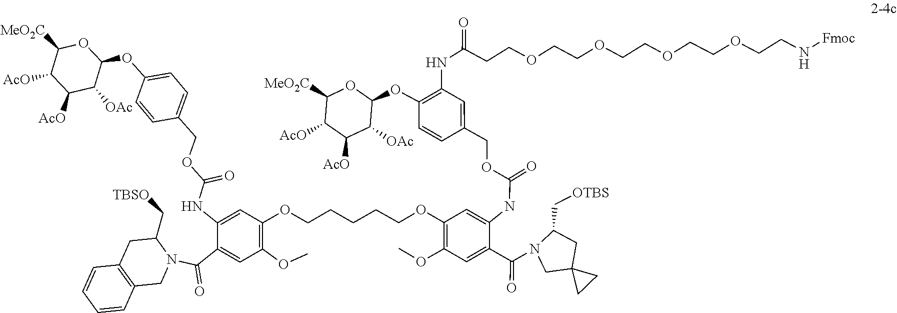

Compound 2-4c (210 mg, 75.0% yield) was synthesized according to the synthetic procedure of step 1 of example 2-4. MS (ESI) m/z: 2334.3 [M+H]⁺.

### Step 4: (2S,3R,4S,5S,6S)-2-(4-((((5-((5-(5-amino-4-((S)-6-(((tert-butyldimethylsilyl)oxy)methyl)-5-azaspiro[2.4]heptane-5-carbonyl)-2-methoxyphenoxy)pentyl)oxy)-2-((S)-3-(((tert-butyldimethylsilyl)oxy)methyl)-1,2,3,4-tetrahydroisoquinoline-2-carbonyl)-4-methoxyphenyl)carbamoyl)oxy)methyl)phenoxy)-6-(methoxycarbonyl)tetrahydro-2H-pyran-3,4,5-triyl triacetate (2-4d)

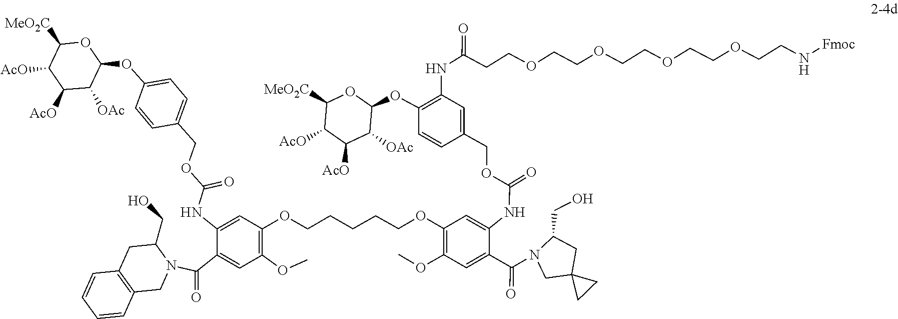

To a mixed solution of 2-4c (210 mg, 0.0900 mmol) in THF (5 mL) and H₂O (250 μL) was added PTSA (41 mg, 0.22 mmol). The mixture was stirred at r.t. for 3 h. The mixture was concentrated under vacuum to give a residue, which was purified by silica gel column chromatography (CH₂Cl₂/MeOH=95/5) to afford 2-4d (110 mg, 58.2% yield) as a white solid. MS (ESI) m/z: 2106.3 [M+H]⁺.

### Step 5: (2S,3R,4S,5S,6S)-2-(2-(1-(9H-fluoren-9-yl)-3-oxo-2,7,10,13,16-pentaoxa-4-azanonadecan-19-amido)-4-((((11S)-11-hydroxy-8-((5-(((6S)-6-hydroxy-2-methoxy-14-oxo-5-(((4-(((2S,3R,4S,5S,6S)-3,4,5-triacetoxy-6-(methoxycarbonyl)tetrahydro-2H-pyran-2-yl)oxy)benzyl)oxy)carbonyl)-5,6,6a,7,12,14-hexahydrobenzo[5,6][1,4]diazepino[1,2-b]isoquinolin-3-yl)oxy)pentyl)oxy)-7-methoxy-5-oxo-5,10,11,11a-tetrahydro-1H,3H-spiro[benzo[e]pyrrolo[1,2-a][1,4]diazepine-2,1′-cyclopropane]-10-carbonyl)oxy)methyl)phenoxy)-6-(methoxycarbonyl)tetrahydro-2H-pyran-3,4,5-triyl triacetate (2-4e)

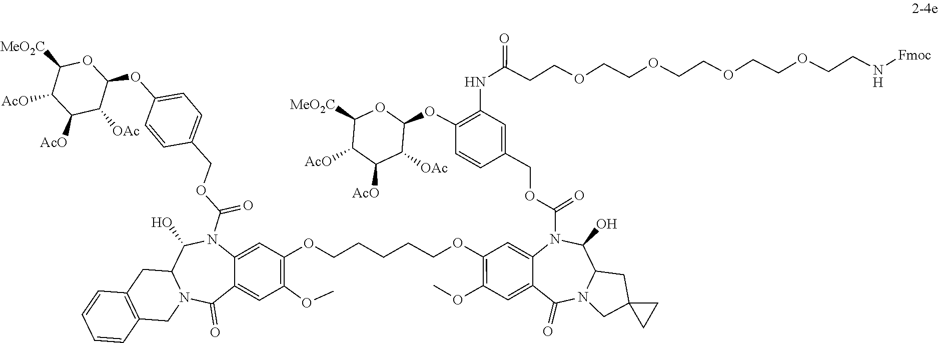

To a 0° C. solution of 2-4d (110 mg, 0.0052 mmol) in CH₂Cl₂ (5 mL) were added Dess-Martin periodinane (55 mg, 0.13 mmol). The mixture was stirred at r.t. for 2 h under N₂. The mixture was filtrated and the filtrate was washed with 3 M Na₂S₂O₃ and 3 M NaHCO₃. The organic phase was dried over Na₂SO₄, filtered and the filtrate concentrated under vacuum to give 2-4e (107 mg, 99.2% yield) as a white solid, which was used in the next step without further purification. MS (ESI) m/z: 2102.7 [M+H]⁺.

### Step 6: (2S,3S,4S,5R,6S)-6-(2-(1-amino-3,6,9,12-tetraoxapentadecan-15-amido)-4-((((11S,11aS)-8-((5-(((6S,6aS)-5-(((4-(((2S,3R,4S,5S,6S)-6-carboxy-3,4,5-trihydroxytetrahydro-2H-pyran-2-yl)oxy)benzyl)oxy)carbonyl)-6-hydroxy-2-methoxy-14-oxo-5,6,6a,7,12,14-hexahydrobenzo[5,6][1,4]diazepino[1,2-b]isoquinolin-3-yl)oxy)pentyl)oxy)-11-hydroxy-7-methoxy-5-oxo-5,10,11,11a-tetrahydro-1H,3H-spiro[benzo[e]pyrrolo[1,2-a][1,4]diazepine-2,1′-cyclopropane]-10-carbonyl)oxy)methyl)phenoxy)-3,4,5-trihydroxytetrahydro-2H-pyran-2-carboxylic acid (2-4f)

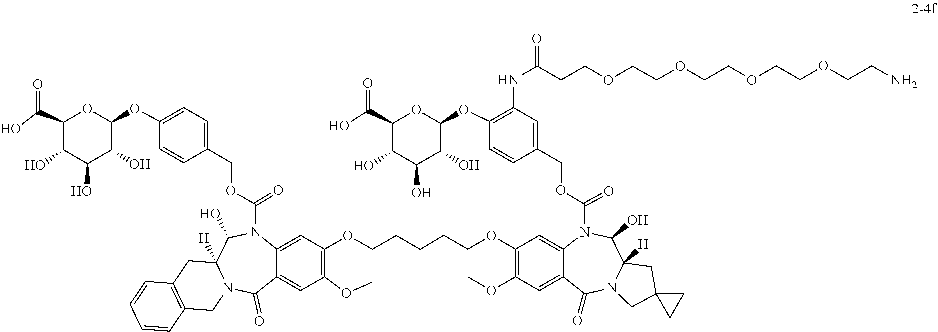 [SMILES: COc1cc2c(cc1OCCCCCOc1cc3c(cc1OC)C(=O)N1CC4(CC4)C[C@H]1[C@H](O)N3C(=O)OCc1ccc(O[C@@H]3O[C@H](C(=O)O)[C@@H](O)[C@H](O)[C@H]3O)c(NC(=O)CCOCCOCCOCCOCCN)c1)N(C(=O)OCc1ccc(O[C@@H]3O[C@H](C(=O)O)[C@@H](O)[C@H](O)[C@H]3O)cc1)[C@@H](O)[C@@H]1Cc3ccccc3CN1C2=O]

To a mixture of 2-4e (660 mg, 0.286 mmol) and Sat. LiBr (CH₃CN/H₂O=10:1, 10 mL) were added Et₃N (398 μL, 2.86 mmol). The mixture was stirred at r.t. for 1 h. The mixture was adjusted to pH 7 using AcOH and concentrated under vacuum to give a crude product. The afforded white form was re-dissolved in DMF (10 mL), then 4 Å Molecular Sieves (600 mg) and Et₂NH (592 μL, 5.72 mmol) was added. The mixture was stirred at r.t. for 1 h. The mixture was adjusted to pH 7 using AcOH and filtrated, and purified by prep-HPLC (Method: column:XBridge Prep C18 OBD 5 μm 19*150 mm; Mobile phase: A—water (0.1% formic acid): B-acetonitrile; Flow rate: 20 mL/min) to provide 2-4f (120 mg, 26.2% yield) as a white solid. MS (ESI) m/z: 1600.4 [M+H]⁺.

### Step 7: (2S,3S,4S,5R,6S)-6-(2-(1-((1R,8S,9s)-bicyclo[6.1.0]non-4-yn-9-yl)-3-oxo-2,7,10,13,16-pentaoxa-4-azanonadecan-19-amido)-4-((((11S,11aS)-8-((5-(((6S,6aS)-5-(((4-(((2S,3R,4S,5S,6S)-6-carboxy-3,4,5-trihydroxytetrahydro-2H-pyran-2-yl)oxy)benzyl)oxy)carbonyl)-6-hydroxy-2-methoxy-14-oxo-5,6,6a,7,12,14-hexahydrobenzo[5,6][1,4]diazepino[1,2-b]isoquinolin-3-yl)oxy)pentyl)oxy)-11-hydroxy-7-methoxy-5-oxo-5,10,11,11a-tetrahydro-1H,3H-spiro[benzo[e]pyrrolo[1,2-a][1,4]diazepine-2,1′-cyclopropane]-10-carbonyl)oxy)methyl)phenoxy)-3,4,5-trihydroxytetrahydro-2H-pyran-2-carboxylic acid (2-4)

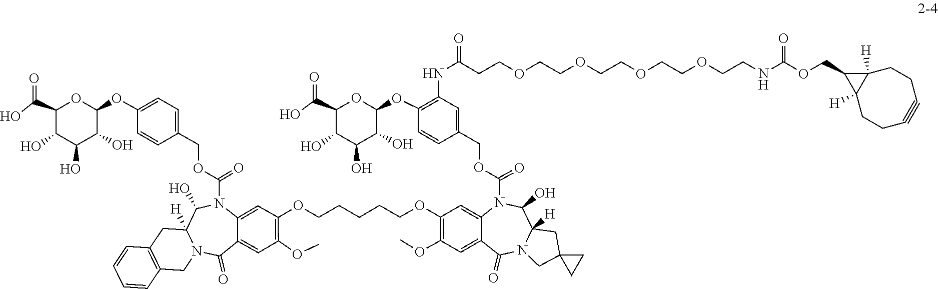 [SMILES: COc1cc2c(cc1OCCCCCOc1cc3c(cc1OC)C(=O)N1CC4(CC4)C[C@H]1[C@H](O)N3C(=O)OCc1ccc(O[C@@H]3O[C@H](C(=O)O)[C@@H](O)[C@H](O)[C@H]3O)c(NC(=O)CCOCCOCCOCCOCCNC(=O)OC[C@@H]3[C@@H]4CCC#CCC[C@@H]43)c1)N(C(=O)OCc1ccc(O[C@@H]3O[C@H](C(=O)O)[C@@H](O)[C@H](O)[C@H]3O)cc1)[C@@H](O)[C@@H]1Cc3ccccc3CN1C2=O]

To a mixture of 2-4f (10 mg, 0.0063 mmol), Int-4 (3.6 mg, 0.013 mmol) in DMF (2 mL) was added DIEA (7.7 μL, 0.039 mmol). The mixture was stirred at r.t. for 1 h. The mixture was adjusted to pH 7 using AcOH and filtrated, and purified by prep-HPLC (Method: column:XBridge Prep C18 OBD 5 μm 19*150 mm; Mobile phase: A—water (0.1% formic acid): B-acetonitrile; Flow rate: 20 mL/min) to provide 2-4 (4.2 mg, 37.2% yield) as a white solid. MS (ESI) m/z: 1797.8 [M+H]⁺.

### Example 2-6

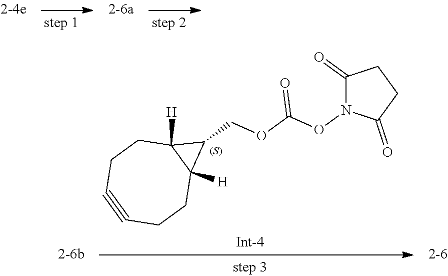

### Step 1: (2S,3R,4S,5S,6S)-2-(2-(1-(9H-fluoren-9-yl)-3-oxo-2,7,10,13,16-pentaoxa-4-azanonadecan-19-amido)-4-((((11S,11aS)-8-((5-(((6S,6aS)-2,6-dimethoxy-14-oxo-5-(((4-(((2S,3R,4S,5S,6S)-3,4,5-triacetoxy-6-(methoxycarbonyl)tetrahydro-2H-pyran-2-yl)oxy)benzyl)oxy)carbonyl)-5,6,6a,7,12,14-hexahydrobenzo[5,6][1,4]diazepino[1,2-b]isoquinolin-3-yl)oxy)pentyl)oxy)-7,11-dimethoxy-5-oxo-5,10,11,11a-tetrahydro-1H,3H-spiro[benzo[e]pyrrolo[1,2-a][1,4]diazepine-2,1′-cyclopropane]-10-carbonyl)oxy)methyl)phenoxy)-6-(methoxycarbonyl)tetrahydro-2H-pyran-3,4,5-triyl triacetate (2-6a)

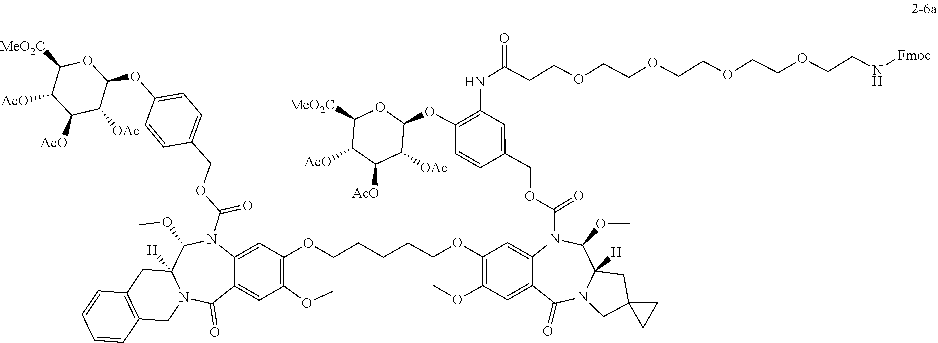

To a mixed solution of 2-4e (30 mg, 0.014 mmol) in MeOH (20 μL, 0.50 mmol) and THF (0.5 mL) were added TMSCl (18 μL, 0.14 mmol). The mixture was stirred at 65° C. for 2 h. The mixture was concentrated under vacuum to give a crude product, which was purified by silica gel column chromatography (CH₂Cl₂/MeOH=95/5) to afford 2-6a (25 mg, 83.3% yield) as a white solid. MS (ESI) m/z: 2130.4 [M+H].

### Step 2: (2S,3S,4S,5R,6S)-6-(2-(1-amino-3,6,9,12-tetraoxapentadecan-15-amido)-4-((((11S,11aS)-8-((5-(((6S,6aS)-5-(((4-(((2S,3R,4S,5S,6S)-6-carboxy-3,4,5-trihydroxytetrahydro-2H-pyran-2-yl)oxy)benzyl)oxy)carbonyl)-2,6-dimethoxy-14-oxo-5,6,6a,7,12,14-hexahydrobenzo[5,6][1,4]diazepino[1,2-b]isoquinolin-3-yl)oxy)pentyl)oxy)-7,11-dimethoxy-5-oxo-5,10,11,11a-tetrahydro-1H,3H-spiro[benzo[e]pyrrolo[1,2-a][1,4]diazepine-2,1′-cyclopropane]-10-carbonyl)oxy)methyl)phenoxy)-3,4,5-trihydroxytetrahydro-2H-pyran-2-carboxylic acid (2-6b)

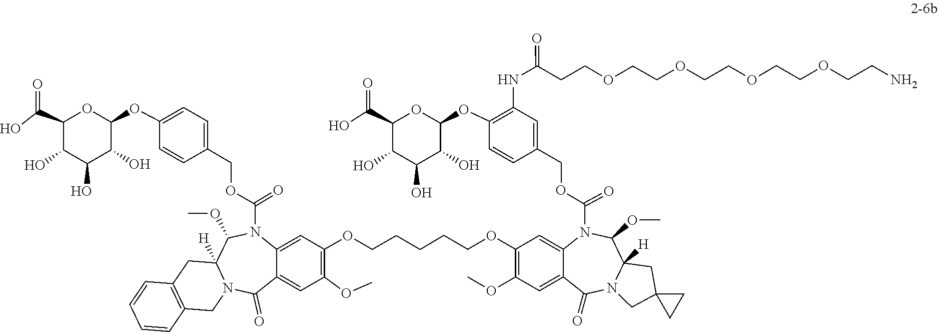 [SMILES: COc1cc2c(cc1OCCCCCOc1cc3c(cc1OC)C(=O)N1CC4(CC4)C[C@H]1[C@H](OC)N3C(=O)OCc1ccc(O[C@@H]3O[C@H](C(=O)O)[C@@H](O)[C@H](O)[C@H]3O)c(NC(=O)CCOCCOCCOCCOCCN)c1)N(C(=O)OCc1ccc(O[C@@H]3O[C@H](C(=O)O)[C@@H](O)[C@H](O)[C@H]3O)cc1)[C@@H](OC)[C@@H]1Cc3ccccc3CN1C2=O]

To a solution of 2-6a (5 mg, 0.0023 mmol) in H₂O (0.2 mL) and THF (0.2 mL) was added LiOH (0.6 mg, 0.023 mmol). The mixture was stirred at r.t. for 1 h. The mixture was adjusted to pH 7 using AcOH and concentrated under vacuum to give residue, which was purified using prep-HPLC (Method: column:XBridge Prep C18 OBD 5 μm 19*150 mm; Mobile phase: A—water (0.1% formic acid): B-acetonitrile; Flow rate: 20 mL/min) to provide 2-6b (2 mg, 54.1% yield) as a white solid. MS (ESI) m/z: 1628.2 [M+H]⁺.

### Step 3: (2S,3S,4S,5R,6S)-6-(2-(1-((1R,8S,9s)-bicyclo[6.1.0]non-4-yn-9-yl)-3-oxo-2,7,10,13,16-pentaoxa-4-azanonadecan-19-amido)-4-((((11S,11aS)-8-((5-(((6S,6aS)-5-(((4-(((2S,3R,4S,5S,6S)-6-carboxy-3,4,5-trihydroxytetrahydro-2H-pyran-2-yl)oxy)benzyl)oxy)carbonyl)-2,6-dimethoxy-14-oxo-5,6,6a,7,12,14-hexahydrobenzo[5,6][1,4]diazepino[1,2-b]isoquinolin-3-yl)oxy)pentyl)oxy)-7,11-dimethoxy-5-oxo-5,10,11,11a-tetrahydro-1H,3H-spiro[benzo[e]pyrrolo[1,2-a][1,4]diazepine-2,1′-cyclopropane]-10-carbonyl)oxy)methyl)phenoxy)-3,4,5-trihydroxytetrahydro-2H-pyran-2-carboxylic acid (2-6)

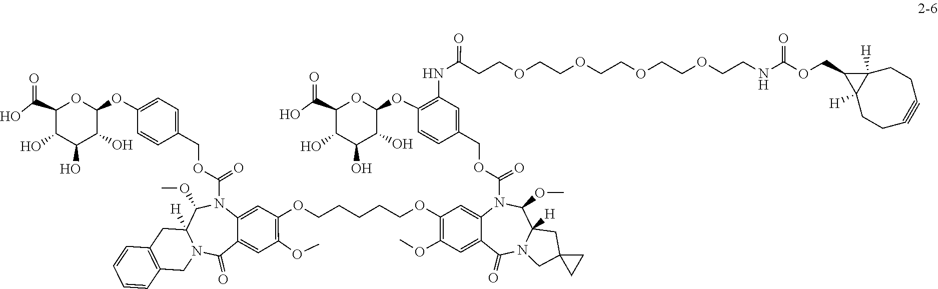 [SMILES: COc1cc2c(cc1OCCCCCOc1cc3c(cc1OC)C(=O)N1CC4(CC4)C[C@H]1[C@H](OC)N3C(=O)OCc1ccc(O[C@@H]3O[C@H](C(=O)O)[C@@H](O)[C@H](O)[C@H]3O)c(NC(=O)CCOCCOCCOCCOCCNC(=O)OC[C@@H]3[C@@H]4CCC#CCC[C@@H]43)c1)N(C(=O)OCc1ccc(O[C@@H]3O[C@H](C(=O)O)[C@@H](O)[C@H](O)[C@H]3O)cc1)[C@@H](OC)[C@@H]1Cc3ccccc3CN1C2=O]

2-6 (2.7 mg, 53.6% yield) was synthesized according to the synthetic procedure of step 7 of example 2-4. MS (ESI) m/z: 1803.9 [M+Na]⁺.

### Example 2-7

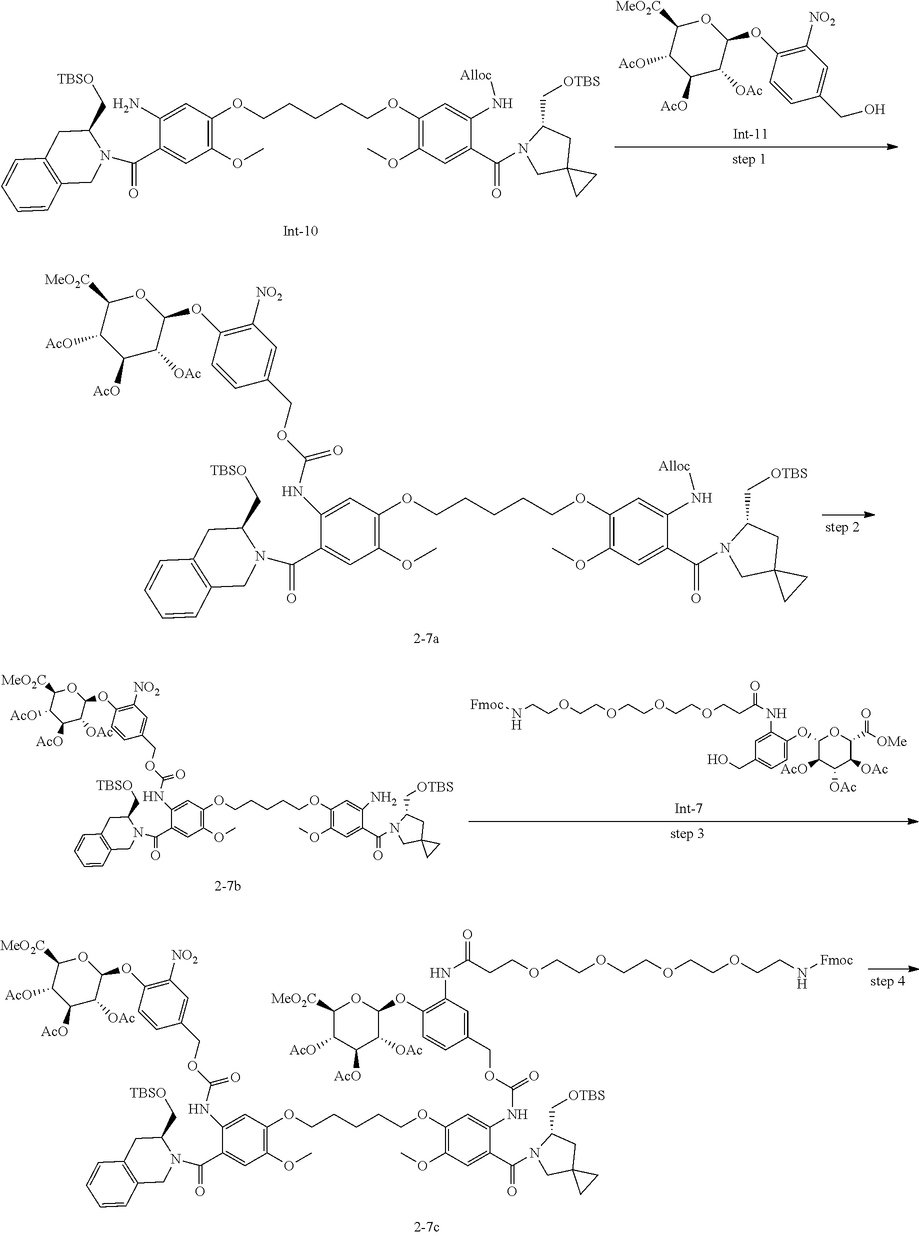

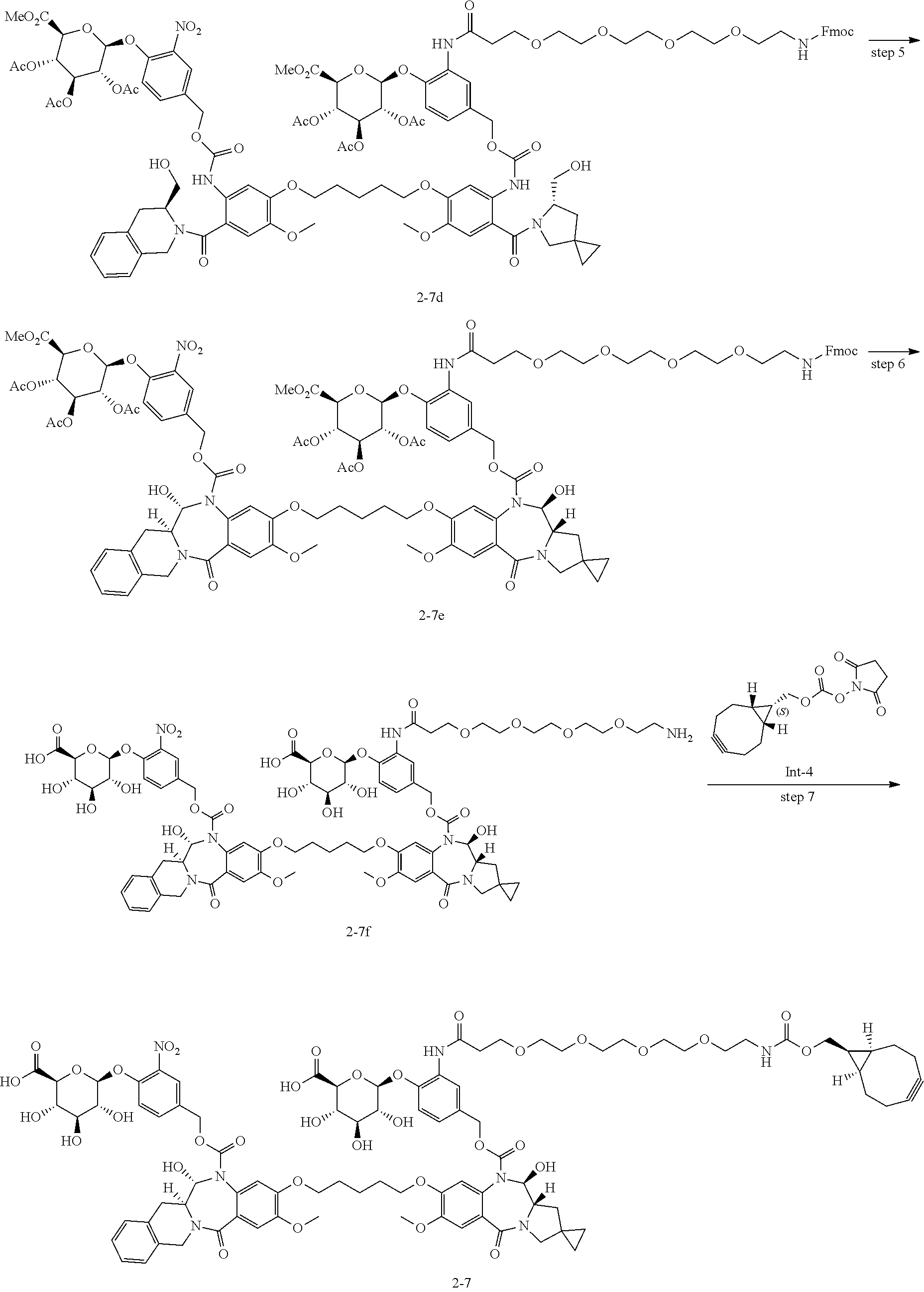

### Step 1: (2S,3R,4S,5S,6S)-2-(4-((((5-((5-(5-(((allyloxy)carbonyl)amino)-4-((S)-6-(((tert-butyldimethylsilyl)oxy)methyl)-5-azaspiro[2.4]heptane-5-carbonyl)-2-methoxyphenoxy)pentyl)oxy)-2-((S)-3-(((tert-butyldimethylsilyl)oxy)methyl)-1,2,3,4-tetrahydroisoquinoline-2-carbonyl)-4-methoxyphenyl)carbamoyl)oxy)methyl)-2-nitrophenoxy)-6-(methoxycarbonyl)tetrahydro-2H-pyran-3,4,5-triyl triacetate (2-7a)

Compound 2-7a (340 mg, 86.50% yield) was synthesized according to the synthetic procedure of step 1 of example 2-4. MS (ESI) m/z: 1513.1 [M+Na].

### Step 2: (2S,3R,4S,5S,6S)-2-(4-((((5-((5-(5-amino-4-((S)-6-(((tert-butyldimethylsilyl)oxy)methyl)-5-azaspiro[2.4]heptane-5-carbonyl)-2-methoxyphenoxy)pentyl)oxy)-2-((S)-3-(((tert-butyldimethylsilyl)oxy)methyl)-1,2,3,4-tetrahydroisoquinoline-2-carbonyl)-4-methoxyphenyl)carbamoyl)oxy)methyl)-2-nitrophenoxy)-6-(methoxycarbonyl)tetrahydro-2H-pyran-3,4,5-triyl triacetate (2-7b)

Compound 2-7b (127 mg, 55.0% yield) was synthesized according to the synthetic procedure of step 2 of example 2-4. MS (ESI) m/z: 1428.9 [M+H]+.

### Step 3: (2S,3R,4S,5S,6S)-2-(2-(1-(9H-fluoren-9-yl)-3-oxo-2,7,10,13,16-pentaoxa-4-azanonadecan-19-amido)-4-((((5-((5-(4-((S)-3-(((tert-butyldimethylsilyl)oxy)methyl)-1,2,3,4-tetrahydroisoquinoline-2-carbonyl)-2-methoxy-5-((((3-nitro-4-(((2S,3R,4S,5S,6S)-3,4,5-triacetoxy-6-(methoxy carbonyl)tetrahydro-2H-pyran-2-yl)oxy)benzyl)oxy)carbonyl)amino)phenoxy)pentyl)oxy)-2-((S)-6-(((tert-butyldimethylsilyl)oxy)methyl)-5-azaspiro[2.4]heptane-5-carbonyl)-4-methoxyphenyl)carbamoyl)oxy)methyl)phenoxy)-6-(methoxycarbonyl)tetrahydro-2H-pyran-3,4,5-triyl triacetate (2-7c)

Compound 2-7c (200 mg, 94.3% yield) was synthesized according to the synthetic procedure of step 3 of example 2-4. MS (ESI) m/z: 2379.3 [M+H]⁺.

### Step 4: (2S,3R,4S,5S,6S)-2-(2-(1-(9H-fluoren-9-yl)-3-oxo-2,7,10,13,16-pentaoxa-4-azanonadecan-19-amido)-4-((((5-((5-(4-((S)-3-(hydroxymethyl)-1,2,3,4-tetrahydroisoquinoline-2-carbonyl)-2-methoxy-5-((((3-nitro-4-(((2S,3R,4S,5S,6S)-3,4,5-triacetoxy-6-(methoxy carbonyl)tetrahydro-2H-pyran-2-yl)oxy)benzyl)oxy)carbonyl)amino)phenoxy)pentyl)oxy)-2-((S)-6-(hydroxymethyl)-5-azaspiro[2.4]heptane-5-carbonyl)-4-methoxyphenyl)carbamoyl)oxy)methyl)phenoxy)-6-(methoxycarbonyl)tetrahydro-2H-pyran-3,4,5-triyl triacetate (2-7d)

Compound 2-7d (160 mg, 89.2% yield) was synthesized according to the synthetic procedure of step 4 of example 2-4. MS (ESI) m/z: 2150.8 [M+H]⁺.

### Step 5: (2S,3R,4S,5S,6S)-2-(2-(1-(9H-fluoren-9-yl)-3-oxo-2,7,10,13,16-pentaoxa-4-azanonadecan-19-amido)-4-((((11S)-11-hydroxy-8-((5-(((6S)-6-hydroxy-2-methoxy-5-(((3-nitro-4-(((2S,3R,4S,5S,6S)-3,4,5-triacetoxy-6-(methoxycarbonyl)tetrahydro-2H-pyran-2-yl)oxy)benzyl)oxy)carbonyl)-14-oxo-5,6,6a,7,12,14-hexahydrobenzo[5,6][1,4]diazepino[1,2-b]isoquinolin-3-yl)oxy)pentyl)oxy)-7-methoxy-5-oxo-5,10,11,11a-tetrahydro-1H,3H-spiro[benzo[e]pyrrolo[1,2-a][1,4]diazepine-2,1′-cyclopropane]-10-carbonyl)oxy)methyl)phenoxy)-6-(methoxycarbonyl)tetrahydro-2H-pyran-3,4,5-triyl triacetate (2-7e)

Compound (2-7e (156 mg, 98.2% yield) was synthesized according to the synthetic procedure of step 5 of example 2-4. MS (ESI) m/z: 2329.3 [M+H]⁺.

### Step 6: (2S,3S,4S,5R,6S)-6-(2-(1-amino-3,6,9,12-tetraoxapentadecan-15-amido)-4-((((11S,11aS)-8-((5-(((6S,6aS)-5-(((4-(((2S,3R,4S,5S,6S)-6-carboxy-3,4,5-trihydroxytetrahydro-2H-pyran-2-yl)oxy)-3-nitrobenzyl)oxy)carbonyl)-6-hydroxy-2-methoxy-14-oxo-5,6,6a,7,12,14-hexahydrobenzo[5,6][1,4]diazepino[1,2-b]isoquinolin-3-yl)oxy)pentyl)oxy)-11-hydroxy-7-methoxy-5-oxo-5,10,11,11a-tetrahydro-1H,3H-spiro[benzo[e]pyrrolo[1,2-a][1,4]diazepine-2,1′-cyclopropane]-10-carbonyl)oxy)methyl)phenoxy)-3,4,5-trihydroxytetrahydro-2H-pyran-2-carboxylic acid (2-7f)

Compound 2-7f (20 mg, 16.3% yield) was synthesized according to the synthetic procedure of step 6 of example 2-4. MS (ESI) m/z: 1645.3 [M+H]⁺.

### Step 7: (2S,3S,4S,5R,6S)-6-(2-(1-((1R,8S,9s)-bicyclo[6.1.0]non-4-yn-9-yl)-3-oxo-2,7,10,13,16-pentaoxa-4-azanonadecan-19-amido)-4-((((11S,11aS)-8-((5-(((6S,6aS)-5-(((4-(((2S,3R,4S,5S,6S)-6-carboxy-3,4,5-trihydroxytetrahydro-2H-pyran-2-yl)oxy)-3-nitrobenzyl)oxy)carbonyl)-6-hydroxy-2-methoxy-14-oxo-5,6,6a,7,12,14-hexahydrobenzo[5,6][1,4]diazepino[1,2-b]isoquinolin-3-yl)oxy)pentyl)oxy)-11-hydroxy-7-methoxy-5-oxo-5,10,11,11a-tetrahydro-1H,3H-spiro[benzo[e]pyrrolo[1,2-a][1,4]diazepine-2,1′-cyclopropane]-10-carbonyl)oxy)methyl)phenoxy)-3,4,5-trihydroxytetrahydro-2H-pyran-2-carboxylic acid (2-7)

Compound 2-7 (11.1 mg, 48.7% yield) was synthesized according to the synthetic procedure of step 6 of example 2-4. MS (ESI) m/z: 1843.2 [M+Na]⁺.

### Example 2-8

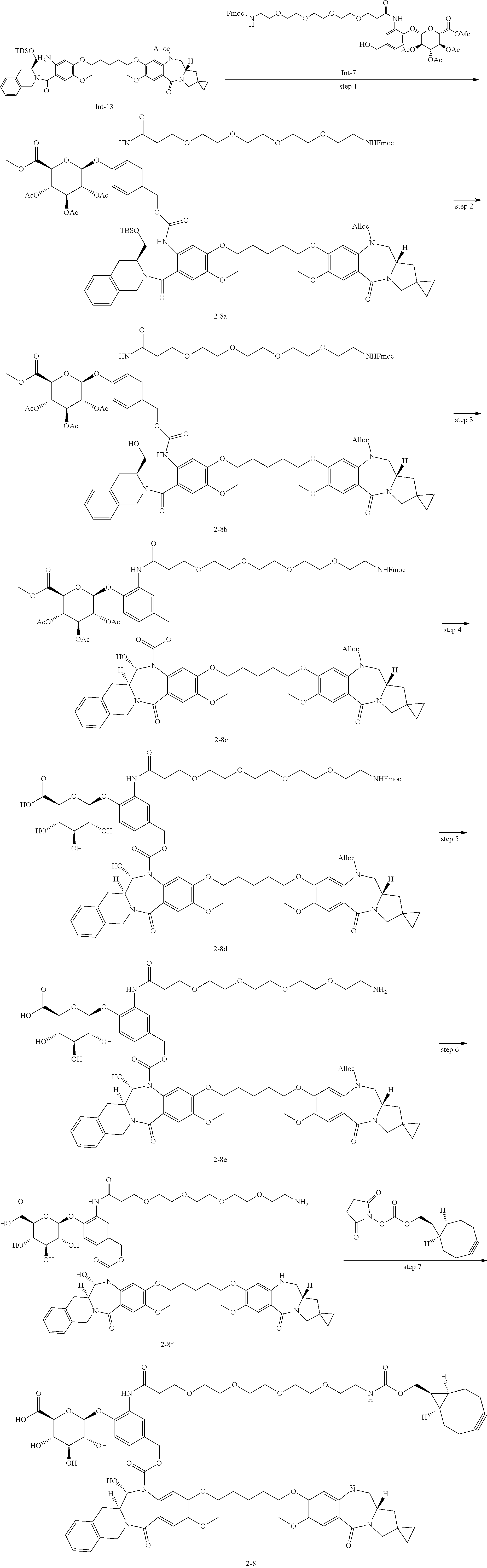

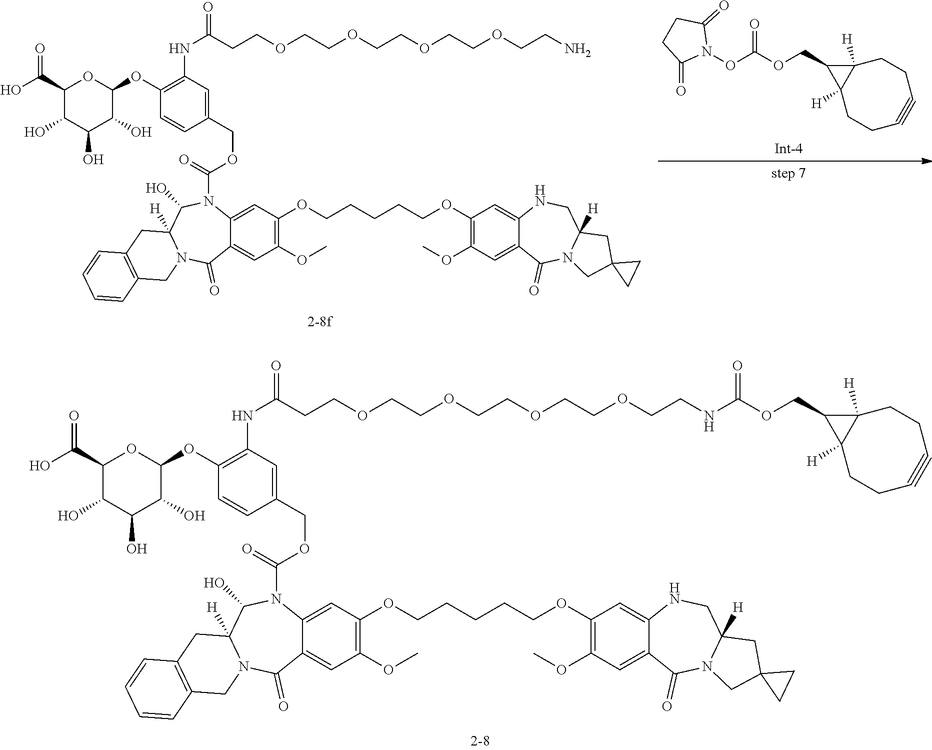 [SMILES: COc1cc2c(cc1OCCCCCOc1cc3c(cc1OC)C(=O)N1Cc4ccccc4C[C@H]1[C@H](O)N3C(=O)OCc1ccc(O[C@@H]3O[C@H](C(=O)O)[C@@H](O)[C@H](O)[C@H]3O)c(NC(=O)CCOCCOCCOCCOCCN)c1)NC[C@@H]1CC3(CC3)CN1C2=O ; O=C(OC[C@@H]1[C@@H]2CCC#CCC[C@@H]21)ON1C(=O)CCC1=O ; COc1cc2c(cc1OCCCCCOc1cc3c(cc1OC)C(=O)N1Cc4ccccc4C[C@H]1[C@H](O)N3C(=O)OCc1ccc(O[C@@H]3O[C@H](C(=O)O)[C@@H](O)[C@H](O)[C@H]3O)c(NC(=O)CCOCCOCCOCCOCCNC(=O)OC[C@@H]3[C@@H]4CCC#CCC[C@@H]43)c1)NC[C@@H]1CC3(CC3)CN1C2=O]

### Step 2: (2S,3R,4S,5S,6S)-2-(2-(1-(9H-fluoren-9-yl)-3-oxo-2,7,10,13,16-pentaoxa-4-azanonadecan-19-amido)-4-((((5-((5-(((S)-10-((allyloxy)carbonyl)-7-methoxy-5-oxo-5,10,11, a-tetrahydro-1H,3H-spiro[benzo[e]pyrrolo[1,2-a][1,4]diazepine-2,1′-cyclopropan]-8-yl)oxy)pentyl)oxy)-2-((S)-3-(hydroxymethyl)-1,2,3,4-tetrahydroisoquinoline-2-carbonyl)-4-methoxyphenyl)carbamoyl)oxy)methyl)phenoxy)-6-(methoxycarbonyl)tetrahydro-2H-pyran-3,4,5-triyl triacetate (2-8b)

Compound 2-8b (268 mg, 72% yield) was synthesized according to the synthetic procedure of step 2 of Ref-2-2. MS (ESI) m/z: 1705.8 [M+H]⁺.

### Step 3: (2S,3R,4S,5S,6S)-2-(2-(1-(9H-fluoren-9-yl)-3-oxo-2,7,10,13,16-pentaoxa-4-azanonadecan-19-amido)-4-((((6S,6aS)-3-((5-(((S)-10-((allyloxy)carbonyl)-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)pentyl)oxy)-6-hydroxy-2-methoxy-14-oxo-5,6,6a,7,12,14-hexahydrobenzo[5,6][1,4]diazepino[1,2-b]isoquinoline-5-carbonyl)oxy)methyl)phenoxy)-6-(methoxycarbonyl)tetrahydro-2H-pyran-3,4,5-triyl triacetate (2-8c)

Dess-Martin periodinane (70 mg, 0.16 mmol) was added slowly, portion-wise to a solution of 2-8b (267 mg, 0.16 mmol) in dry CH₂Cl₂ (5 mL) at 0° C. The reaction was then warmed to r.t. and stirred for 2 h. The reaction was quenched with sat. Na₂S203, followed by addition of sat. NaHCO₃ and water. The layers were separated, and the organic layer was washed with brine, dried over Na₂SO₄. The crude product was purified by silica gel column chromatography (CH₂Cl₂/EtOAc=93/7) to give 2-8c (218 mg, 82% yield). MS (ESI) m/z: 1703.8 [M+H]⁺.

### Step 4: (2S,3S,4S,5R,6S)-6-(2-(1-(9H-fluoren-9-yl)-3-oxo-2,7,10,13,16-pentaoxa-4-azanonadecan-19-amido)-4-((((6S,6aS)-3-((5-(((S)-10-((allyloxy)carbonyl)-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)pentyl)oxy)-6-hydroxy-2-methoxy-14-oxo-5,6,6a,7,12,14-hexahydrobenzo[5,6][1,4]diazepino[1,2-b]isoquinoline-5-carbonyl)oxy)methyl)phenoxy)-3,4,5-trihydroxytetrahydro-2H-pyran-2-carboxylic acid (2-8d)

LiBr (306 mg, 3.52 mmol) was dissolved in MeCN/H₂O (0.8/0.08 mL), then 2-8c (60 mg, 0.04 mmol) was added to the mixture. Et₃N (49 μL, 0.35 mmol) was added to the reaction at r.t. and the mixture was stirred at r.t. for 1 h. The reaction was complete by LCMS, the mixture was neutralized with AcOH (21 μL, 0.35 mmol) and concentrated. The residue was used directly in the next step without further purification. MS (ESI) m/z: 1563.7 [M+H]⁺.

### Step 5: (2S,3S,4S,5R,6S)-6-(4-((((6S,6aS)-3-((5-(((S)-10-((allyloxy)carbonyl)-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)pentyl)oxy)-6-hydroxy-2-methoxy-14-oxo-5,6,6a,7,12,14-hexahydrobenzo[5,6][1,4]diazepino[1,2-b]isoquinoline-5-carbonyl)oxy)methyl)-2-(1-amino-3,6,9,12-tetraoxapentadecan-15-amido)phenoxy)-3,4,5-trihydroxytetrahydro-2H-pyran-2-carboxylic acid (2-8e)

To a solution of crude 2-8d (55 mg, 0.032 mmol) in 2 mL DMF was added Et₂NH (67 μL, 0.65 mmol). The mixture was stirred at r.t. for 0.5 h. The mixture was concentrated to give the crude product, which was purified by prep-HPLC (Method: column:XBridge Prep C18 OBD 5 μm 19*150 mm; Mobile phase: A—water (0.1% formic acid): B-acetonitrile; Flow rate: 20 mL/min) to give 2-8e (28 mg, 37% yield) as a white solid. MS (ESI) m/z: 1341.7 [M+H]⁺.

### Step 6: (2S,3S,4S,5R,6S)-6-(2-(1-amino-3,6,9,12-tetraoxapentadecan-15-amido)-4-((((6S,6aS)-6-hydroxy-2-methoxy-3-((5-(((S)-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)pentyl)oxy)-14-oxo-5,6,6a,7,12,14-hexahydrobenzo[5,6][1,4]diazepino[1,2-b]isoquinoline-5-carbonyl)oxy)methyl)phenoxy)-3,4,5-trihydroxytetrahydro-2H-pyran-2-carboxylic acid (2-8f)

Pd(PPh₃)₄ (1.2 mg, 0.021 mmol) was added to a solution of 2-8e (28 mg, 0.021 mmol) and pyrrolidine (1.9 μL, 0.023 mmol) in CH₂Cl₂ (1 mL) at 0° C. The reaction mixture was allowed to stir for 1 h at 0° C. The reaction was neutralized with AcOH (1.3 μL, 0.023 mmol) and concentrated. The crude product was purified by prep-HPLC (Method: column:XBridge Prep C18 OBD 5 μm 19*150 mm; Mobile phase: A—water (0.1% formic acid): B-acetonitrile; Flow rate: 20 mL/min) to give 2-8f (16 mg, 61% yield) as a white solid. MS (ESI) m/z: 1257.7 [M+H]⁺.

### Step 7: (2S,3S,4S,5R,6S)-6-(2-(1-((1R,8S,9s)-bicyclo[6.1.0]non-4-yn-9-yl)-3-oxo-2,7,10,13,16-pentaoxa-4-azanonadecan-19-amido)-4-((((6S,6aS)-6-hydroxy-2-methoxy-3-((5-(((S)-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)pentyl)oxy)-14-oxo-5,6,6a,7,12,14-hexahydrobenzo[5,6][1,4]diazepino[1,2-b]isoquinoline-5-carbonyl)oxy)methyl)phenoxy)-3,4,5-trihydroxytetrahydro-2H-pyran-2-carboxylic acid (2-8)

DIEA (11.1 μL, 0.064 mmol) was added to the solution of 2-8f (16 mg, 0.013 mmol) and Int-4 (11.1 mmol, 0.038 mmol) in 1 mL DMF. The mixture was stirred at r.t. for 20 min. The mixture was purified by prep-HPLC (Method: column:XBridge Prep C18 OBD 5 μm 19*150 mm; Mobile phase: A—water (0.1% formic acid): B-acetonitrile; Flow rate: 20 mL/min) to give 2-8 (5.1 mg, 28% yield) as a white solid. MS (ESI) m/z: 1433.8 [M+H]⁺.

### Example 2-10

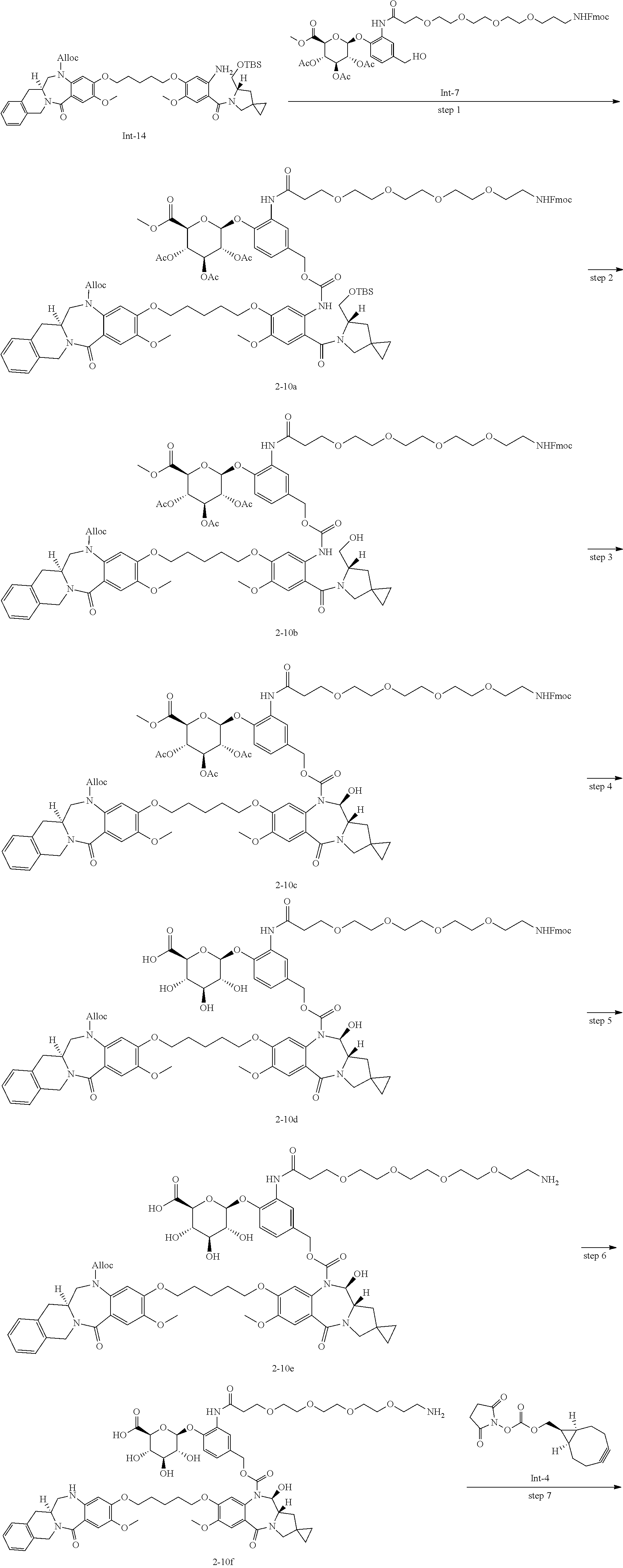

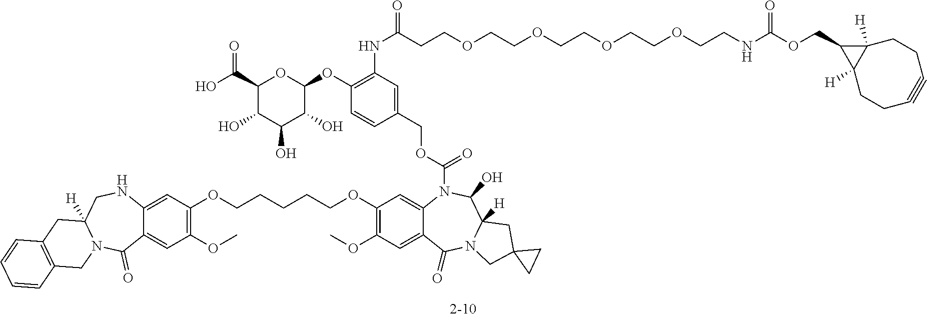 [SMILES: COc1cc2c(cc1OCCCCCOc1cc3c(cc1OC)C(=O)N1CC4(CC4)C[C@H]1[C@H](O)N3C(=O)OCc1ccc(O[C@@H]3O[C@H](C(=O)O)[C@@H](O)[C@H](O)[C@H]3O)c(NC(=O)CCOCCOCCOCCOCCNC(=O)OC[C@@H]3[C@@H]4CCC#CCC[C@@H]43)c1)NC[C@@H]1Cc3ccccc3CN1C2=O]

### Step 1 (2S,3R,4S,5S,6S)-2-(2-(1-(9H-fluoren-9-yl)-3-oxo-2,7,10,13,16-pentaoxa-4-azanonadecan-19-amido)-4-((((5-((5-(((S)-5-((allyloxy) carbonyl)-2-methoxy-14-oxo-5,6,6a, 7,12,14-hexahydrobenzo[5,6][1,4]diazepino[1,2-b]isoquinolin-3-yl)oxy) pentyl)oxy)-2-((S)-6-(((tert-butyldimethylsilyl)oxy) methyl)-5-azaspiro[2.4]heptane-5-carbonyl)-4-methoxyphenyl) carbamoyl)oxy) methyl) phenoxy)-6-(methoxycarbonyl)tetrahydro-2H-pyran-3,4,5-triyl triacetate (2-10a)

Int-14 (200 mg, 0.23 mmol) was dissolved in dry THF (2 mL), 4 Å molecular sieves (200 mg) was added. The mixture was cooled to 0° C., then triphosgene (24.6 mg, 0.083 mmol) was added, followed by addition of Et₃N (71.1 μL, 0.5 mmol). The mixture was stirred at 0° C. for 10 min under a nitrogen atmosphere. Int-7 (234.1 mg, 0.25 mmol), Et₃N (48.5 μL, 0.345 mmol) and dibutyltin dilaurate (27.3 μL, 0.046 mmol) were mixed in dry THF (1 mL), and then this solution was gradually added to the above reaction solution at 0° C. The reaction solution was warmed to r.t. and stirred for 2 h. The solvent was concentrated, and then purified by column chromatography (MeOH/CH₂Cl₂=5/95) to obtain 2-10a as a light-yellow solid (365 mg, 87.2% yield). MS (ESI) m/z: 1821.1 [M+H]⁺.

### Step 2 (2S,3R,4S,5S,6S)-2-(2-(1-(9H-fluoren-9-yl)-3-oxo-2,7,10,13,16-pentaoxa-4-azanonadecan-19-amido)-4-((((5-((5-(((S)-5-((allyloxy) carbonyl)-2-methoxy-14-oxo-5,6,6a, 7,12,14-hexahydrobenzo[5,6][1,4]diazepino[1,2-b]isoquinolin-3-yl)oxy) pentyl)oxy)-2-((S)-6-(hydroxymethyl)-5-azaspiro[2.4]heptane-5-carbonyl)-4-methoxyphenyl)carbamoyl)oxy)methyl)phenoxy)-6-(methoxycarbonyl)tetrahydro-2H-pyran-3,4,5-triyl triacetate (2-10b)

2-10a (365 mg, 0.2 mmol) was dissolved in THF/H₂O (3 mL/60 μL, 50/1), TsOH-H₂O (22.9 mg, 0.12 mmol) was added, and the mixture was stirred at r.t. for 2 h. The solvent was then removed by evaporation, the residue was purified by column chromatography (MeOH/EtOAc=2/98) to obtain 2-10b as a light-yellow solid (300 mg, 87.7% yield). MS (ESI) m/z: 1705.7 [M+H]⁺.

### Step 3: (2S,3R,4S,5S,6S)-2-(2-(1-(9H-fluoren-9-yl)-3-oxo-2,7,10,13,16-pentaoxa-4-azanonadecan-19-amido)-4-((((11S,11aS)-8-((5-(((S)-5-((allyloxy)carbonyl)-2-methoxy-14-oxo-5,6,6a,7,12,14-hexahydrobenzo[5,6][1,4]diazepino[1,2-b]isoquinolin-3-yl)oxy)pentyl)oxy)-11-hydroxy-7-methoxy-5-oxo-5,10,11,11a-tetrahydro-1H,3H-spiro[benzo[e]pyrrolo[1,2-a][1,4]diazepine-2,1′-cyclopropane]-10-carbonyl)oxy)methyl)phenoxy)-6-(methoxycarbonyl)tetrahydro-2H-pyran-3,4,5-triyl triacetate (2-10c)

To a solution of 2-10b (300 mg, 0.18 mmol) in CH₂Cl₂ (1 mL) was added Dess-Martin periodinane (82 mg, 0.19 mmol) at r.t., and the mixture was stirred at r.t. for 3 h under a nitrogen atmosphere. The reaction solution was then diluted with CH₂Cl₂ (10 mL), washed with sat. Na₂S₂O₃ (5 mL) and brine (5 mL). The organic phase was collected and dried over anhydrous Na₂SO₄, filtered and concentrated under vacuum to give a crude product, which was purified by silica gel column chromatography (MeOH/CH₂Cl₂=5/95) obtain 2-10c as a yellow solid (270 mg, 90.1% yield). MS (ESI) m/z: 1726.2 [M+Na]⁺.

### Step 4: (2S,3S,4S,5R,6S)-6-(2-(1-(9H-fluoren-9-yl)-3-oxo-2,7,10,13,16-pentaoxa-4-azanonadecan-19-amido)-4-((((11S,11aS)-8-((5-(((S)-5-((allyloxy)carbonyl)-2-methoxy-14-oxo-5,6,6a,7,12,14-hexahydrobenzo[5,6][1,4]diazepino[1,2-b]isoquinolin-3-yl)oxy)pentyl)oxy)-11-hydroxy-7-methoxy-5-oxo-5,10,11,11a-tetrahydro-1H,3H-spiro[benzo[e]pyrrolo[1,2-a][1,4]diazepine-2,1′-cyclopropane]-10-carbonyl)oxy)methyl)phenoxy)-3,4,5-trihydroxytetrahydro-2H-pyran-2-carboxylic acid (2-10d)

To a solution of LiBr (277.6 mg, 3.2 mmol) in CH₃CN/H₂O (0.5 mL/50 μL) was added 2-10c (50 mg, 0.032 mmol), followed by addition of triethylamine (44.3 μL, 0.32 mmol) at r.t. The reaction mixture was stirred at r.t. for 1 h under a nitrogen atmosphere. The solvent was removed by evaporation to give crude product 2-10d as a yellow oil, which was directly used in the next step without further purification. MS (ESI) m/z: 1563.7 [M+H]⁺.

### Step 5: (2S,3S,4S,5R,6S)-6-(4-((((11S,11aS)-8-((5-(((S)-5-((allyloxy)carbonyl)-2-methoxy-14-oxo-5,6,6a,7,12,14-hexahydrobenzo[5,6][1,4]diazepino[1,2-b]isoquinolin-3-yl)oxy)pentyl)oxy)-11-hydroxy-7-methoxy-5-oxo-5,10,11,11a-tetrahydro-1H,3H-spiro[benzo[e]pyrrolo[1,2-a][1,4]diazepine-2,1′-cyclopropane]-10-carbonyl)oxy)methyl)-2-(1-amino-3,6,9,12-tetraoxapentadecan-15-amido)phenoxy)-3,4,5-trihydroxytetrahydro-2H-pyran-2-carboxylic acid (2-10e)

To the crude product of 2-10d in 1 mL DMF was added diethylamine (0.32 mmol, 33 μL) at 0° C. The reaction solution was warm to r.t. and stirred for 1 h. The reaction solution was neutralized with AcOH, and then purified by prep-HPLC (Method: column:XBridge Prep C18 OBD 5 μm 19*150 mm; Mobile phase: A—water (0.1% formic acid): B-acetonitrile; Flow rate: 20 mL/min) to provide 2-10e as a white solid after lyophilization (25 mg, 58% yield). MS (ESI) m/z: 1342.3 [M+H]⁺.

### Step 6: (2S,3S,4S,5R,6S)-6-(2-(1-amino-3,6,9,12-tetraoxapentadecan-15-amido)-4-((((11S,11aS)-11-hydroxy-7-methoxy-8-((5-(((S)-2-methoxy-14-oxo-5,6,6a,7,12,14-hexahydrobenzo[5,6][1,4]diazepino[1,2-b]isoquinolin-3-yl)oxy)pentyl)oxy)-5-oxo-5,10,11,11a-tetrahydro-1H,3H-spiro[benzo[e]pyrrolo[1,2-a][1,4]diazepine-2,1′-cyclopropane]-10-carbonyl)oxy)methyl)phenoxy)-3,4,5-trihydroxytetrahydro-2H-pyran-2-carboxylic acid (2-10f)

To a solution of 2-10e in 1 mL CH₂Cl₂ was added Pd(PPh₃)₄ (1 mg, cat.) and diethylamine (3.87 μL, 0.037 mmol) at 0° C. The reaction solution was stirred at 0° C. for 1 h. The reaction solution was neutralized with AcOH, and then purified by prep-HPLC (Method: column:XBridge Prep C18 OBD 5 μm 19*150 mm; Mobile phase: A—water (0.1% formic acid): B-acetonitrile; Flow rate: 20 mL/min) to provide 2-10f as a white solid after lyophilization (15 mg, 80% yield). MS (ESI) m/z: 1258.2 [M+H]⁺.

### Step 7: (2S,3S,4S,5R,6S)-6-(2-(1-((1R,8S,9s)-bicyclo[6.1.0]non-4-yn-9-yl)-3-oxo-2,7,10,13,16-pentaoxa-4-azanonadecan-19-amido)-4-((((11S,11aS)-11-hydroxy-7-methoxy-8-((5-(((S)-2-methoxy-14-oxo-5,6,6a,7,12,14-hexahydrobenzo[5,6][1,4]diazepino[1,2-b]isoquinolin-3-yl)oxy)pentyl)oxy)-5-oxo-5,10,11,11a-tetrahydro-1H,3H-spiro[benzo[e]pyrrolo[1,2-a][1,4]diazepine-2,1′-cyclopropane]-10-carbonyl)oxy)methyl)phenoxy)-3,4,5-trihydroxytetrahydro-2H-pyran-2-carboxylic acid (2-10)

To a solution of 2-10f (15 mg, 0.012 mmol) in 0.5 mL DMF was added Int-4 (7 mg, 0.024 mmol) and DIPEA (11.4 μL, 0.06 mmol) at 0° C. The reaction solution was stirred at 0° C. for 0.5 h. The reaction solution was neutralized with AcOH, and then purified by prep-HPLC (Method: column:XBridge Prep C18 OBD 5 μm 19*150 mm; Mobile phase: A—water (0.1% formic acid): B-acetonitrile; Flow rate: 20 mL/min) to provide 2-10 as a white solid after lyophilization (6.3 mg, 36.8% yield). MS (ESI) m/z: 1434.2 [M+H]⁺.

### Example 2-12

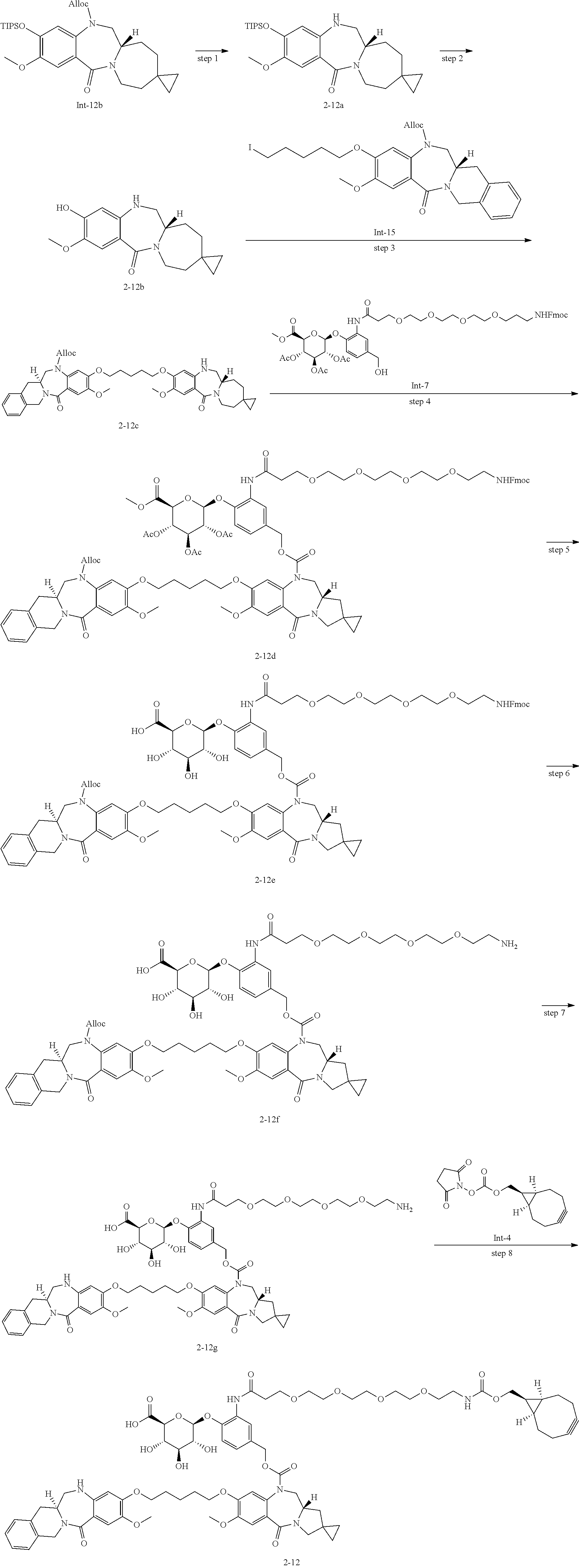

### Step 1: (S)-7-methoxy-8-((triisopropylsilyl)oxy)-1,10,11,11a-tetrahydro-3H,5H-spiro[lbenzo[e]pyrrolo[1,2-a][11,4]diazepine-2,1′-cyclopropan]-5-one (2-12a)

A solution of Int-12b (750 mg, 1.46 mmol) in CH₂Cl₂ (6 mL) was added pyrrolidine (0.3 mL, 3.65 mmol) and Pd(PPh₃)₄ (168.52 mg, 0.15 mmol) at r.t. The mixture was stirred at r.t. under N₂ for 30 min. Solvent was evaporated and the mixture was purified by silica gel column chromatography (A-Pet.ether; B-EtOAc) to afford product 2-12a (580 mg, 87.8% yield) as a white solid. MS (ESI) m/z: 431.2 [M+H]⁺.

### Step 2: (S)-8-hydroxy-7-methoxy-1,10,11,11a-tetrahydro-3H,5H-spiro[benzo[e]pyrrolo[1,2-a][1,4]diazepine-2,1′-cyclopropan]-5-one (2-12b)

To a mixed solution of compound 2-12a (580 mg, 1.35 mmol) in DMF (3 mL) and water (0.15 mL) was added LiOAc (133 mg, 2.02 mmol). The mixture was stirred at 20° C. for 1 h. The reaction mixture was diluted with EtOAc (20 mL) and washed with brine (15 mL*3). The organic layer was dried over Na₂SO₄. After filtration and evaporation, the residue was purified by silica gel column chromatography (eluent: A—CH₂Cl₂; B-MeOH) to afford 2-12b (350 mg, 89.98% yield) as a white solid. MS (ESI) m/z: 275.2 [M+H]⁺.

### Step 3: allyl (S)-2-methoxy-3-((5-(((S)-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)pentyl)oxy)-14-oxo-6,6a,7,12-tetrahydrobenzo[5s,6][1,4]diazepino[i1,2-b]isoquinoline-5(14H)-carboxylate (2-12c)

To a solution of 2-12b (90 mg, 0.33 mmol) and Int-15 (195 mg, 0.33 mmol) in DMF (2 mL) was added K₂CO₃ (91 mg, 0.66 mmol) at r.t. The mixture was stirred at r.t. for 20 h. The reaction mixture was diluted with EtOAc (20 mL) and washed with brine (10 mL*3). The organic layer was dried over Na₂SO₄, filtrated and concentrated to give the residue which was purified by silica gel column chromatography (A-Pet.ether; B-EtOAc) to afford 2-12c (150 mg, 59.0% yield) as an off-white solid. MS (ESI) m/z: 737.4 [M+H]⁺.

### Step 4: (2S,3R,4S,5S,6S)-2-(2-(1-(9H-fluoren-9-yl)-3-oxo-2,7,10,13,16-pentaoxa-4-azanonadecan-19-amido)-4-((((S)-8-((5-(((S)-5-((allyloxy)carbonyl)-2-methoxy-14-oxo-5,6,6a,7,12,14-hexahydrobenzo[5,6][1,4]diazepino[1,2-b]isoquinolin-3-yl)oxy)pentyl)oxy)-7-methoxy-5-oxo-5,10,11,11a-tetrahydro-1H,3H-spiro[benzo[e]pyrrolo[1,2-a][1,4]diazepine-2,1′-cyclopropane]-10-carbonyl)oxy)methyl)phenoxy)-6-(methoxycarbonyl)tetrahydro-2H-pyran-3,4,5-triyl triacetate (2-12d)

A solution of Int-7 (151 mg, 0.16 mmol) in CH₂Cl₂ (6 mL) was cooled to −90° C., triphosgene (12 mg, 0.04 mmol) in CH₂Cl₂ (2 mL) and pyridine (0.087 mL, 1.09 mmol) was added at −90° C. under N₂ protection and the temperature was maintained below −85° C. The mixture was stirred at −90° C. for 20 min, then 2-12c (100 mg, 0.14 mmol) in CH₂Cl₂ (2 mL) was added. The reaction was warmed to 0° C. and was stirred at 0° C. for 2 h. The reaction mixture was quenched with water (20 mL) and extracted with CH₂Cl₂ (30 mL*3). The organic layer was dried over Na₂SO₄ and concentrated to give the residue which was purified by silica gel column chromatography (A-Pet.ether; B-EtOAc) to afford 2-12d (150 mg, 49.1% yield) as an off-white solid. MS (ESI) m/z: 1688.3 [M+H]⁺.

### Step 5: (2S,3S,4S,5R,6S)-6-(2-(1-(9H-fluoren-9-yl)-3-oxo-2,7,10,13,16-pentaoxa-4-azanonadecan-19-amido)-4-((((S)-8-((5-(((S)-5-((allyloxy)carbonyl)-2-methoxy-14-oxo-5,6,6a,7,12,14-hexahydrobenzo[5,6][1,4]diazepino[1,2-b]isoquinolin-3-yl)oxy)pentyl)oxy)-7-methoxy-5-oxo-5,10,11,11a-tetrahydro-1H,3H-spiro[benzo[e]pyrrolo[1,2-a][1,4]diazepine-2,1′-cyclopropane]-10-carbonyl)oxy)methyl)phenoxy)-3,4,5-trihydroxytetrahydro-2H-pyran-2-carboxylic acid (2-12e)

To a solution of 12-2d (150 mg, 0.09 mmol) in MeCN (10 mL) and water (1 mL) was added LiBr (773 mg, 8.89 mmol) and Et₃N (0.12 mL, 0.89 mmol) at r.t. The mixture was stirred at r.t. for 2 h. The reaction mixture was acidified with AcOH (0.15 mL), and solvent was evaporated to give the residue 2-12e (crude product) which was used in the next step without further purification. MS (ESI) m/z: 1548.2 [M+H]⁺.

### Step 6: (2S,3S,4S,5R,6S)-6-(4-((((S)-8-((5-(((S)-5-((allyloxy)carbonyl)-2-methoxy-14-oxo-5,6,6a,7,12,14-hexahydrobenzo[5,6][1,4]diazepino[1,2-b]isoquinolin-3-yl)oxy)pentyl)oxy)-7-methoxy-5-oxo-5,10,11,11a-tetrahydro-1H,3H-spiro[benzo[e]pyrrolo[1,2-a][1,4]diazepine-2,1′-cyclopropane]-10-carbonyl)oxy)methyl)-2-(1-amino-3,6,9,12-tetraoxapentadecan-15-amido)phenoxy)-3,4,5-trihydroxytetrahydro-2H-pyran-2-carboxylic acid (2-12f)

To a solution of 2-12e (crude product) in DMF (5 mL) was added diethylamine (0.09 mL, 0.89 mmol) at r.t. The reaction mixture was stirred at r.t. for 1 h. LCMS showed the reaction was completed. The reaction mixture was acidified with AcOH (0.10 mL) and was purified by prep-HPLC (Method: column:XBridge Prep C18 OBD 5 μm 19*150 mm; Mobile phase: A—water (0.1% formic acid): B-acetonitrile; Flow rate: 20 mL/min) to give 2-12f (50 mg, 40.3% yield) as a white solid. MS (ESI) m/z: 1324.6 [M+H]⁺.

### Step 7: (2S,3S,4S,5R,6S)-6-(2-(1-amino-3,6,9,12-tetraoxapentadecan-15-amido)-4-((((S)-7-methoxy-8-((5-(((S)-2-methoxy-14-oxo-5,6,6a,7,12,14-hexahydrobenzo[5,6][1,4]diazepino[1,2-b]isoquinolin-3-yl)oxy)pentyl)oxy)-5-oxo-5,10,11,11a-tetrahydro-1H,3H-spiro[benzo[e]pyrrolo[1,2-a][1,4]diazepine-2,1′-cyclopropane]-10-carbonyl)oxy)methyl)phenoxy)-3,4,5-trihydroxytetrahydro-2H-pyran-2-carboxylic acid (2-12g)

To a solution of 2-12f (50 mg, 0.04 mmol) and in THF (3 mL) was added Dimedone (10 mg, 0.08 mmol) and Pd(PPh₃)₄ (4.36 mg, 0.04 mmol) at r.t. The mixture was stirred at r.t. under N₂ for 1 h. Solvent was evaporated, and the mixture was purified by prep-HPLC (Method: column:XBridge Prep C18 OBD 5 μm 19*150 mm; Mobile phase: A—water (0.1% formic acid): B-acetonitrile; Flow rate: 20 mL/min) to give 2-12g (30 mg, 60.1% yield) as a white solid. MS (ESI) m/z: 1241.5 [M+H]⁺.

### Step 8: (2S,3S,4S,5R,6S)-6-(2-(1-((1R,8S,9s)-bicyclo[6.1.0]non-4-yn-9-yl)-3-oxo-2,7,10,13,16-pentaoxa-4-azanonadecan-19-amido)-4-((((S)-7-methoxy-8-((5-(((S)-2-methoxy-14-oxo-5,6,6a,7,12,14-hexahydrobenzo[5,6][1,4]diazepino[1,2-b]isoquinolin-3-yl)oxy)pentyl)oxy)-5-oxo-5,10,11,11a-tetrahydro-1H,3H-spiro[benzo[e]pyrrolo[1,2-a][1,4]diazepine-2,1′-cyclopropane]-10-carbonyl)oxy)methyl)phenoxy)-3,4,5-trihydroxytetrahydro-2H-pyran-2-carboxylic acid (2-12)

To a solution of compound 2-12g (30 mg, 0.02 mmol) and in DMF (2 mL) was added Int-4 (7.39 mg, 0.03 mmol) and DIPEA (0.0082 mL, 0.05 mmol) at r.t. The mixture was stirred at r.t. for 2 h. The reaction mixture was purified by prep-HPLC (Method: column:XBridge Prep C18 OBD 5 μm 19*150 mm; Mobile phase: A—water (0.1% formic acid): B-acetonitrile; Flow rate: 20 mL/min) to give 2-12 (16.8 mg, 48.1% yield) as a white solid. MS (ESI) m/z: 1418.3 [M+H]⁺

### Example 2-14

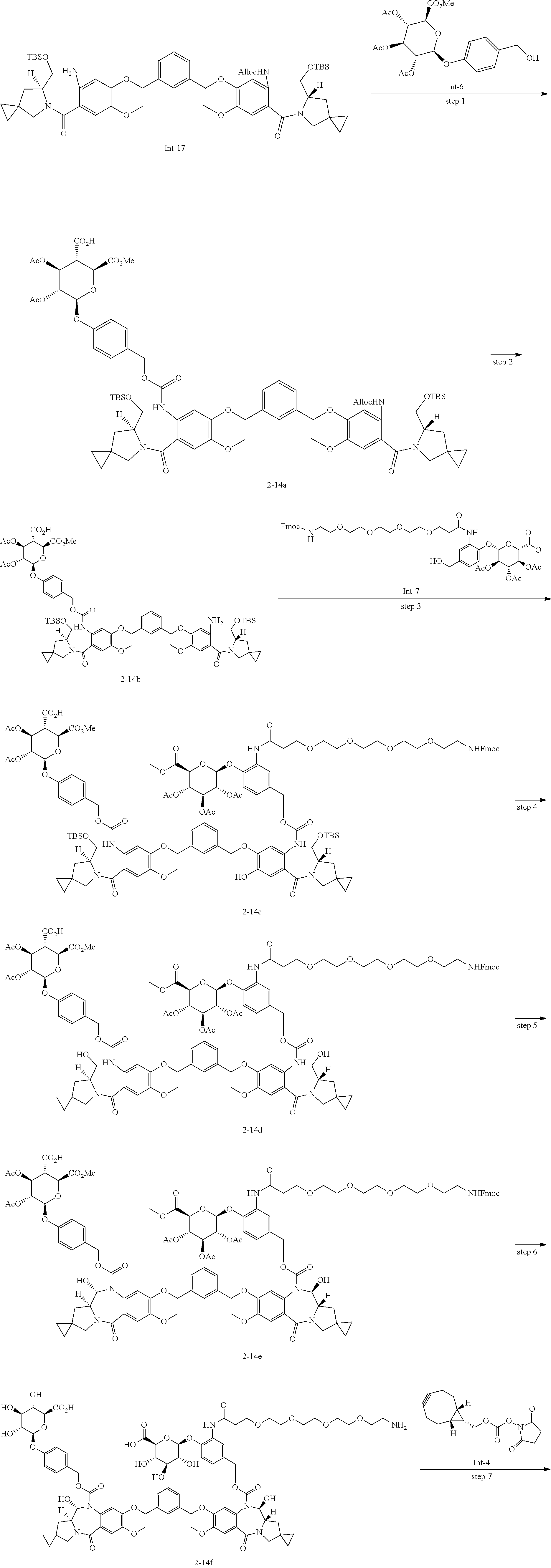

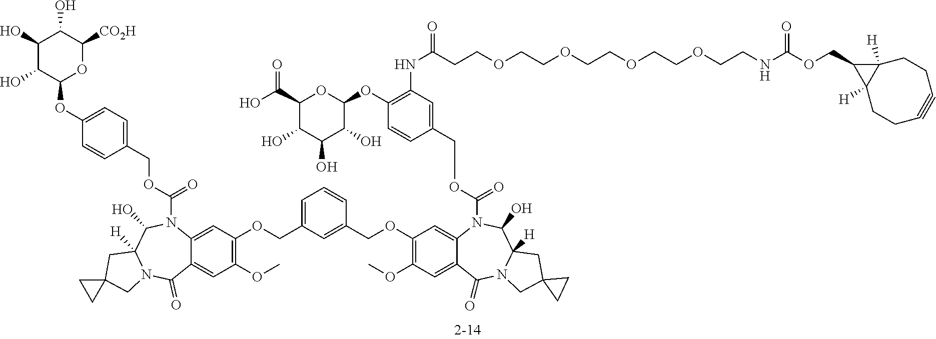 [SMILES: COc1cc2c(cc1OCc1cccc(COc3cc4c(cc3OC)C(=O)N3CC5(CC5)C[C@H]3[C@H](O)N4C(=O)OCc3ccc(O[C@@H]4O[C@H](C(=O)O)[C@@H](O)[C@H](O)[C@H]4O)c(NC(=O)CCOCCOCCOCCOCCNC(=O)OC[C@@H]4[C@@H]5CCC#CCC[C@@H]54)c3)c1)N(C(=O)OCc1ccc(O[C@@H]3O[C@H](C(=O)O)[C@@H](O)[C@H](O)[C@H]3O)cc1)[C@@H](O)[C@@H]1CC3(CC3)CN1C2=O]

### Step 1: (2S,3R,4S,5S,6S)-2-(4-((((5-((3-((5-(((allyloxy)carbonyl)amino)-4-((S)-6-(((tert-butyldimethylsilyl)oxy)methyl)-5-azaspiro[2.4]heptane-5-carbonyl)-2-methoxyphenoxy)methyl)benzyl)oxy)-2-((S)-6-(((tert-butyldimethylsilyl)oxy)methyl)-5-azaspiro[2.4]heptane-5-carbonyl)-4-methoxyphenyl)carbamoyl)oxy)methyl)phenoxy)-6-(methoxycarbonyl)tetrahydro-2H-pyran-3,4,5-triyl triacetate (2-14a)

To a mixture of Int-17 (340 mg, 0.34 mmol) and 4 Å molecular sieves (100 mg) in anhydrous THF (4 mL) were added triphosgene (38 mg, 0.13 mmol) and Et₃N (236 μL, 172 mg, 1.70 mmol) at 0° C., then stirred on ice-bath for 10 min. A mixture of Int-6 (180 mg, 0.41 mmol), Dibutyltin dilaurate (21 mg, 0.034 mmol) in TH (4 mL) was added. The mixture was stirred at r.t. overnight. After reaction was complete then purified by silica gel column chromatography (10 g, EtOAc/Petroleum=0/100-50150), fraction was concentrated under vacuum to give 2-14a (435.6 mg, 87.4% yield) as a white solid. MS (ESI) m/z: 1466.4 [M+H]⁺.

### Step 2: (2S,3R,4S,5S,6S)-2-(4-((((5-((3-((5-amino-4-((S)-6-(((tert-butyldimethylsilyl)oxy)methyl)-5-azaspiro[2.4]heptane-5-carbonyl)-2-methoxyphenoxy)methyl)benzyl)oxy)-2-((S)-6-(((tert-butyldimethylsilyl)oxy)methyl)-5-azaspiro[2.4]heptane-5-carbonyl)-4-methoxyphenyl)carbamoyl)oxy)methyl)phenoxy)-6-(methoxycarbonyl)tetrahydro-2H-pyran-3,4,5-triyl triacetate (2-14b)

To a mixture of 2-14a (435.6 mg, 0.30 mmol) in THF (5.0 mL) was added Dimedone (83 mg, 0.59 mmol) and Pd(PPh₃)₄ (34 mg, 0.030 mmol). The light-yellow mixture was degassed with N₂ balloon for three times then stirred at r.t. for 2 h under nitrogen atmosphere. The mixture was filtered, concentrated under vacuum, and purified by reserved phase chromatography (C18, MeCN/H₂O=20/80-100/0), fraction was concentrated under vacuum to give 2-14b (375 mg, 91.3% yield) as a white solid. MS (ESI) m/z: 1382.4 [M+H]⁺.

### Step 3: ((2S,3R,4S,5S,6S)-2-(2-(1-(9H-fluoren-9-yl)-3-oxo-2,7,10,13,16-pentaoxa-4-azanonadecan-19-amido)-4-((((5-((3-((4-((S)-6-(((tert-butyldimethylsilyl)oxy)methyl)-5-azaspiro[2.4]heptane-5-carbonyl)-2-methoxy-5-((((4-(((2S,3R,4S,5S,6S)-3,4,5-triacetoxy-6-(methoxy carbonyl)tetrahydro-2H-pyran-2-yl)oxy)benzyl)oxy)carbonyl)amino)phenoxy)methyl)benzyl)oxy)-2-((S)-6-(((tert-butyldimethylsilyl)oxy)methyl)-5-azaspiro[2.4]heptane-5-carbonyl)-4-methoxyphenyl)carbamoyl)oxy)methyl)phenoxy)-6-(methoxycarbonyl)tetrahydro-2H-pyran-3,4,5-triyl triacetate (2-14c)

To a mixture of 2-14b (224 mg, 0.16 mmol) and 4 Å molecular sieves (100 mg) in anhydrous THF (3 mL) were added triphosgene (18 mg, 0.062 mmol) and Et₃N (113 μL, 82 mg, 0.81 mmol) at 0° C., then stirred on ice-bath for 10 min. A mixture of Int-7 (180 mg, 0.20 mmol), Dibutyltin dilaurate (10 mg, 0.016 mmol) in THF (2 mL) was added to the above solution. The mixture was stirred at r.t. overnight. The mixture was filtered, concentrated under vacuum, the crude was purified by reserved phase chromatography (C18, MeCN/H₂O=20/80-100/0), fraction was concentrated under vacuum to give 2-14c (311 mg, 82.3% yield) as a white solid. MS (ESI) m/z: 2332.5 [M+H]⁺.

### Step 4: (2S,3R,4S,5S,6S)-2-(2-(1-(9H-fluoren-9-yl)-3-oxo-2,7,10,13,16-pentaoxa-4-azanonadecan-19-amido)-4-((((5-((3-((4-((S)-6-(hydroxymethyl)-5-azaspiro[2.4]heptane-5-carbonyl)-2-methoxy-5-((((4-(((2S,3R,4S,5S,6S)-3,4,5-triacetoxy-6-(methoxy carbonyl)tetrahydro-2H-pyran-2-yl)oxy)benzyl)oxy)carbonyl)amino)phenoxy)methyl)benzyl)oxy)-2-((S)-6-(hydroxymethyl)-5-azaspiro[2.4]heptane-5-carbonyl)-4-methoxyphenyl)carbamoyl)oxy)methyl)phenoxy)-6-(methoxycarbonyl)tetrahydro-2H-pyran-3,4,5-triyl triacetate (2-14d)

A solution of 2-14c (102.0 mg, 0.044 mmol) in AcOH/THF/H₂O (1 mL/0.6 mL/0.3 mL) was stirred at r.t. overnight. The mixture was diluted with EtOAc (30 mL), washed with brine (20 mL), dried over Na₂SO₄, filtered and the filtrate was concentrated under vacuum to give a residue then purified by silica gel column chromatography (MeOH/CH₂Cl₂=0/100-3/97), fraction was concentrated under vacuum to give 2-14d (90 mg, 97.8% yield) as a white solid. MS (ESI) m/z: 2104.6 [M+H]⁺.

### Step 5: (2S,3R,4S,5S,6S)-2-(2-(1-(9H-fluoren-9-yl)-3-oxo-2,7,10,13,16-pentaoxa-4-azanonadecan-19-amido)-4-((((11S,11aS)-11-hydroxy-8-((3-((((11S,11aS)-11-hydroxy-7-methoxy-5-oxo-10-(((4-(((2S,3R,4S,5S,6S)-3,4,5-triacetoxy-6-(methoxycarbonyl)tetrahydro-2H-pyran-2-yl)oxy)benzyl)oxy)carbonyl)-5,10,11,11a-tetrahydro-1H,3H-spiro[benzo[e]pyrrolo[1,2-a][1,4]diazepine-2,1′-cyclopropan]-8-yl)oxy)methyl)benzyl)oxy)-7-methoxy-5-oxo-5,10,11,11a-tetrahydro-1H,3H-spiro[benzo[e]pyrrolo[1,2-a][1,4]diazepine-2,1′-cyclopropane]-10-carbonyl)oxy)methyl)phenoxy)-6-(methoxycarbonyl)tetrahydro-2H-pyran-3,4,5-triyl triacetate (2-14e)

To a solution of 2-14d (90 mg, 0.043 mmol) in CH₂Cl₂ (3 mL) was added Dess-Martin periodinane (42 mg, 0.098 mmol) then stirred at r.t. for 2 h. The reaction mixture was quenched with sat. Na₂S₂O₃/sat·NaHCO₃H₂O (1:1:1, 15 mL), extracted with CH₂Cl₂ (20 mL*3). Combined organic layers were washed with brine (20 mL*3), dried over Na₂SO₄, filtered and the filtrate was concentrated under vacuum to give 2-14e (87.0 mg, crude) as a yellow solid. MS (ESI) m/z: 2100.6 [M+H]⁺.

### Step 6: (2S,3S,4S,5R,6S)-6-(2-(1-amino-3,6,9,12-tetraoxapentadecan-15-amido)-4-((((11S,11aS)-8-((3-((((11S,11aS)-10-(((4-(((2S,3R,4S,5S,6S)-6-carboxy-3,4,5-trihydroxytetrahydro-2H-pyran-2-yl)oxy)benzyl)oxy)carbonyl)-11-hydroxy-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)methyl)benzyl)oxy)-11-hydroxy-7-methoxy-5-oxo-5,10,11,11a-tetrahydro-1H,3H-spiro[benzo[e]pyrrolo[1,2-a][1,4]diazepine-2,1′-cyclopropane]-10-carbonyl)oxy)methyl)phenoxy)-3,4,5-trihydroxytetrahydro-2H-pyran-2-carboxylic acid (2-14f)

To a solution of 2-14e (87 mg, crude) were added a mixture of LiBr (360.0 mg, 4.143 mmol) in MeCN/H₂O (0.9 mL/0.3 mL) and Et₃N (31 μL, 23.06 mmol). The mixture was stirred at r.t. for 5 h. The mixture was acidified with AcOH to pH 5. The mixture was concentrated under vacuum to give crude product, which was re-dissolved in DMF (2 mL). Diethylamine (214 μL, 2.071 mmol) was added and the mixture was stirred at r.t. for 1 h. The mixture was acidified with AcOH, filtrated, purified by prep-HPLC (0.1% FA) (Method: column:XBridge Prep C18 OBD 5 μm 19*150 mm; Mobile phase: A—water (0.1% formic acid): B-acetonitrile; Flow rate: 20 mL/min), fraction was concentrated under vacuum to give 2-14f (45 mg, 68.0%) as a white solid. MS (ESI) m/z: 1598.3 [M+H]⁺.

### Step 7: (2S,3S,4S,5R,6S)-6-(2-(1-((1R,8S,9s)-bicyclo[6.1.0]non-4-yn-9-yl)-3-oxo-2,7,10,13,16-pentaoxa-4-azanonadecan-19-amido)-4-((((11S,11aS)-8-((3-((((11S,11aS)-10-(((4-(((2S,3R,4S,5S,6S)-6-carboxy-3,4,5-trihydroxytetrahydro-2H-pyran-2-yl)oxy)benzyl)oxy)carbonyl)-11-hydroxy-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)methyl)benzyl)oxy)-11-hydroxy-7-methoxy-5-oxo-5,10,11,11a-tetrahydro-1H,3H-spiro[benzo[e]pyrrolo[1,2-a][1,4]diazepine-2,1′-cyclopropane]-10-carbonyl)oxy)methyl)phenoxy)-3,4,5-trihydroxytetrahydro-2H-pyran-2-carboxylic (2-14)

To a mixture of 2-14f (45 mg, 0.028 mmol) and Int-4 (12 mg, 0.042 mmol) in DMF (1.5 mL) was added DIPEA (5 μL, 4 mg, 0.028 mmol). The mixture was stirred at r.t. for 15 min. After 2-14f was fully consumed, the mixture was purified by prep-HPLC (0.1% FA) (Method: column:XBridge Prep C18 OBD 5 μm 19*150 mm; Mobile phase: A—water (0.1% formic acid): B-acetonitrile; Flow rate: 20 mL/min) to give 2-14 (15.3 mg, 30.6% yield) as a white solid. MS (ESI) m/z: 1774.8[M+H]⁺.

### Example 2-16

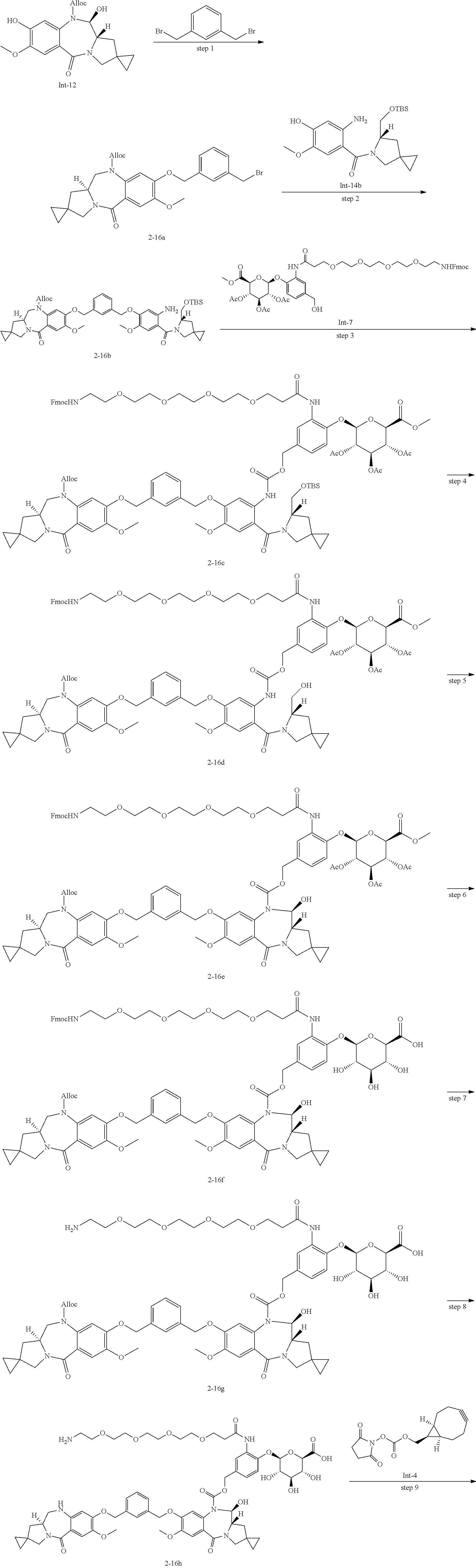

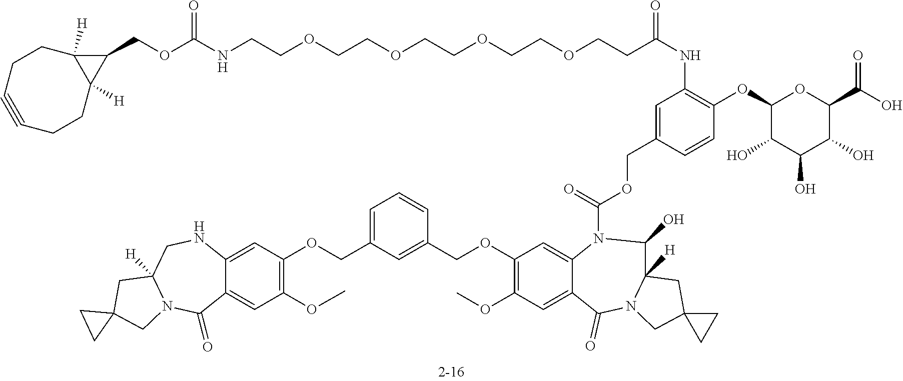 [SMILES: COc1cc2c(cc1OCc1cccc(COc3cc4c(cc3OC)C(=O)N3CC5(CC5)C[C@H]3[C@H](O)N4C(=O)OCc3ccc(O[C@H]4O[C@@H](C(=O)O)[C@H](O)[C@@H](O)[C@@H]4O)c(NC(=O)CCOCCOCCOCCOCCNC(=O)OC[C@@H]4[C@@H]5CCC#CCC[C@@H]54)c3)c1)NC[C@@H]1CC3(CC3)CN1C2=O]

### Step 1: Allyl (S)-8-((3-(bromomethyl)benzyl) oxy)-7-methoxy-5-oxo-11,11a-dihydro-1H,3H-spiro[benzo[e]pyrrolo[1,2-a][1,4]diazepine-2,1′-cyclopropane]-10 (5H)-carboxylate (2-16a)

To a solution of Int-12 (162 mg, 0.45 mmol) and 1,3-bis(bromomethyl)benzene (596 mg, 2.26 mmol) in DMF (1.5 mL) were added K₂CO₃ (94 mg, 0.68 mmol) at r.t. After addition, the reaction mixture was reacted at r.t. for 2 h. The mixture was diluted with EtOAc (15 mL). The organic layer was washed with sat. NH₄Cl aq. (15 mL), H₂O (15 mL), and brine (15 mL), dried over anhydrous Na₂SO₄, filtered and concentrated under vacuum to give a residue which was purified by silica gel column chromatography (A-Petroleum ether; B-EtOAc) to provide 2-16a (195 mg, 79.5% yield) as a white solid. MS (ESI) m/z: 541.3 [M+H]⁺.

### Step 2: Allyl (S)-8-((3-((5-amino-4-((S)-6-(((tert-butyldimethylsilyl)oxy)methyl)-5-azaspiro[2.4]heptane-5-carbonyl)-2-methoxyphenoxy)methyl)benzyl)oxy)-7-methoxy-5-oxo-11,11a-dihydro-1H,3H-spiro[benzo[e]pyrrolo[1,2-a][1,4]diazepine-2,1′-cyclopropane]-10(5H)-carboxylate (2-16b)

To a solution of Int-14b (243 mg, 0.60 mmol) in DMF (6.5 mL) were added t-BuOK (69 mg, 0.60 mmol) on ice-bath and stirred for 0.5 h. Then 2-16a (295 mg, 0.54 mmol) was added at this temperature and stirred for another 0.5 h. The mixture was quenched with sat. NH₄Cl aq. (10 mL). The aqueous layer was extracted with EtOAc (15 mL*3). The combined organic layers were washed with H₂O (20 mL), brine (20 mL), dried over anhydrous Na₂SO₄, filtered and concentrated under vacuum to give a residue which was purified by silica gel column chromatography (A-Petroleum ether; B-EtOAc) to provide 2-16b (376.3 mg, 79.6% yield) as a pale-yellow solid. MS (ESI) m/z: 868.1 [M+H].

### Step 3: (2R,3S,4R,5R,6R)-2-(2-(1-(9H-Fluoren-9-yl)-3-oxo-2,7,10,13,16-pentaoxa-4-azanonadecan-19-amido)-4-((((5-((3-((((S)-10-((allyloxy)carbonyl)-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)methyl)benzyl)oxy)-2-((S)-6-(((tert-butyldimethylsilyl)oxy)methyl)-5-azaspiro[2.4]heptane-5-carbonyl)-4-methoxyphenyl)carbamoyl)oxy)methyl)phenoxy)-6-(methoxycarbonyl)tetrahydro-2H-pyran-3,4,5-triyl triacetate (2-16c)

Compound 2-16c (625 mg, 79.2% yield) was synthesized according to the synthetic procedure of step 1 of example Ref-2-2. MS (ESI) m/z: 1818.0 [M+H].

### Step 4: (2R,3S,4R,5R,6R)-2-(2-(1-(9H-fluoren-9-yl)-3-oxo-2,7,10,13,16-pentaoxa-4-azanonadecan-19-amido)-4-((((5-((3-((((S)-10-((allyloxy)carbonyl)-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)methyl)benzyl)oxy)-2-((S)-6-(hydroxymethyl)-5-azaspiro[2.4]heptane-5-carbonyl)-4-methoxyphenyl)carbamoyl)oxy)methyl)phenoxy)-6-(methoxycarbonyl)tetrahydro-2H-pyran-3,4,5-triyl triacetate (2-16d)

Compound 2-16d (568 mg, 96.9% yield) was synthesized according to the synthetic procedure of step 2 of example 2-8. MS (ESI) m/z: 1703.8 [M+H]⁺.

### Step 5: (2R,3S,4R,5R,6R)-2-(2-(1-(9H-Fluoren-9-yl)-3-oxo-2,7,10,13,16-pentaoxa-4-azanonadecan-19-amido)-4-((((11S,11aS)-8-((3-((((S)-10-((allyloxy)carbonyl)-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)methyl)benzyl)oxy)-11-hydroxy-7-methoxy-5-oxo-5,10,11,11a-tetrahydro-1H,3H-spiro[benzo[e]pyrrolo[1,2-a][1,4]diazepine-2,1′-cyclopropane]-10-carbonyl)oxy)methyl)phenoxy)-6-(methoxycarbonyl)tetrahydro-2H-pyran-3,4,5-triyl triacetate (2-16e)

Compound 2-16e (200 mg, 99.9% yield) was synthesized according to the synthetic procedure of step 3 of example 2-8. MS (ESI) m/z: 1702.1 [M+H]⁺.

### Step 6: (2R,3R,4R,5S,6R)-6-(2-(1-(9H-fluoren-9-yl)-3-oxo-2,7,10,13,16-pentaoxa-4-azanonadecan-19-amido)-4-((((11S,11aS)-8-((3-((((S)-10-((allyloxy)carbonyl)-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)methyl)benzyl)oxy)-11-hydroxy-7-methoxy-5-oxo-5,10,11,11a-tetrahydro-1H,3H-spiro[benzo[e]pyrrolo[1,2-a][1,4]diazepine-2,1′-cyclopropane]-10-carbonyl)oxy)methyl)phenoxy)-3,4,5-trihydroxytetrahydro-2H-pyran-2-carboxylic acid (2-16f)

Compound 2-16f (crude, 0.12 mmol) was synthesized according to the synthetic procedure of step 4 of example 2-8. MS (ESI) m/z: 1562.4 [M+H]⁺.

### Step 7: (2R,3R,4R,5S,6R)-6-(4-((((11S,11aS)-8-((3-((((S)-10-((Allyloxy)carbonyl)-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)methyl)benzyl)oxy)-11-hydroxy-7-methoxy-5-oxo-5,10,11,11a-tetrahydro-1H,3H-spiro[benzo[e]pyrrolo[1,2-a][1,4]diazepine-2,1′-cyclopropane]-10-carbonyl)oxy)methyl)-2-(1-amino-3,6,9,12-tetraoxapentadecan-15-amido)phenoxy)-3,4,5-trihydroxytetrahydro-2H-pyran-2-carboxylic acid (2-16g)

Compound 2-16g (85 mg, 54.10% over 2 steps) was synthesized according to the synthetic procedure of step 5 of example 2-8. MS (ESI) m/z: 1339.8 [M+H]⁺.

### Step 8: (2R,3R,4R,5S,6R)-6-(2-(1-Amino-3,6,9,12-tetraoxapentadecan-15-amido)-4-((((11S,11aS)-11-hydroxy-7-methoxy-8-((3-((((S)-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)methyl)benzyl)oxy)-5-oxo-5,10,11,11a-tetrahydro-1H,3H-spiro[benzo[e]pyrrolo[1,2-a][1,4]diazepine-2,1′-cyclopropane]-10-carbonyl)oxy)methyl)phenoxy)-3,4,5-trihydroxytetrahydro-2H-pyran-2-carboxylic acid (2-16h)

Compound 2-16h (41 mg, 51.1%) was synthesized according to the synthetic procedure of step 6 of example 2-8. MS (ESI) m/z: 1256.1 [M+H]⁺.

### Step 9: (2R,3R,4R,5S,6R)-6-(2-(1-((1R,8S,9s)-bicyclo[6.1.0]non-4-yn-9-yl)-3-oxo-2,7,10,13,16-pentaoxa-4-azanonadecan-19-amido)-4-((((11S,11aS)-11-hydroxy-7-methoxy-8-((3-((((S)-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)methyl)benzyl)oxy)-5-oxo-5,10,11,11a-tetrahydro-1H,3H-spiro[benzo[e]pyrrolo[1,2-a][1,4]diazepine-2,1′-cyclopropane]-10-carbonyl)oxy) methyl) phenoxy)-3,4,5-trihydroxytetrahydro-2H-pyran-2-carboxylic acid (2-16)

Compound 2-16 (17.1 mg, 50.0%) was synthesized according to the synthetic procedure of step 6 of example Ref-2-2. MS (ESI) m/z: 1432.1 [M+H]⁺.

### Example 2-18

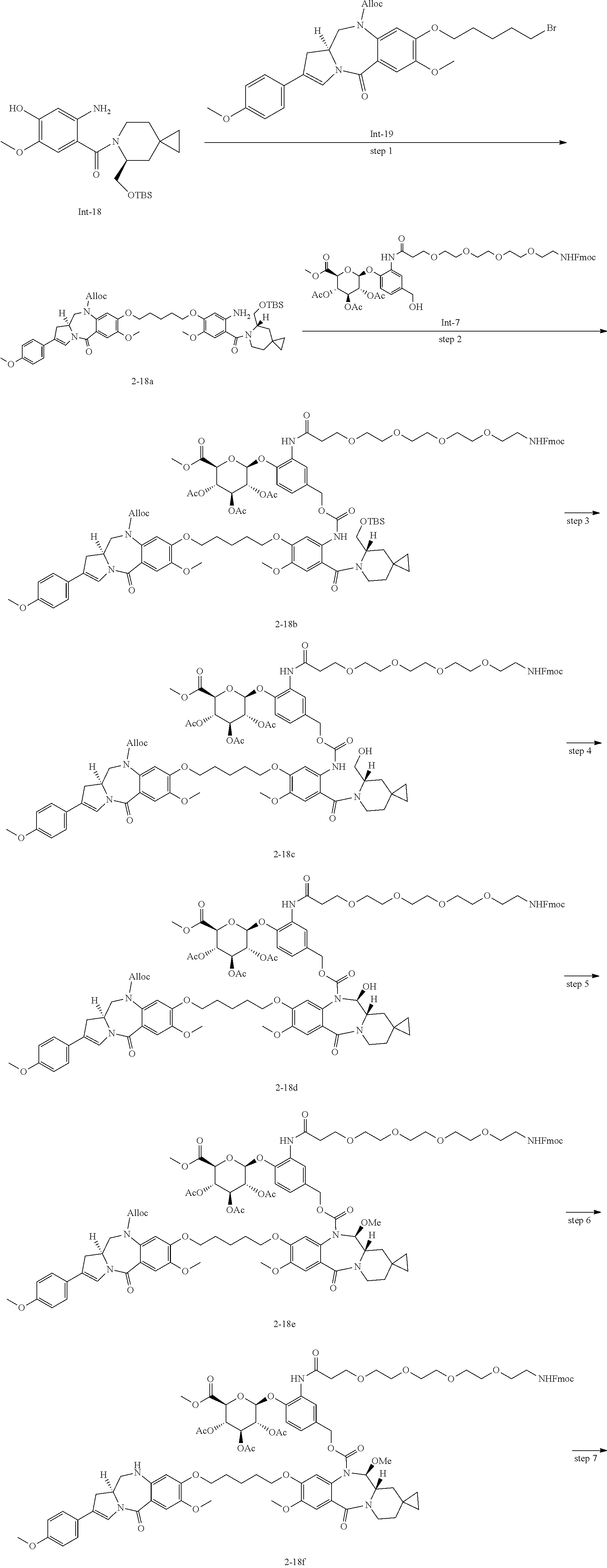

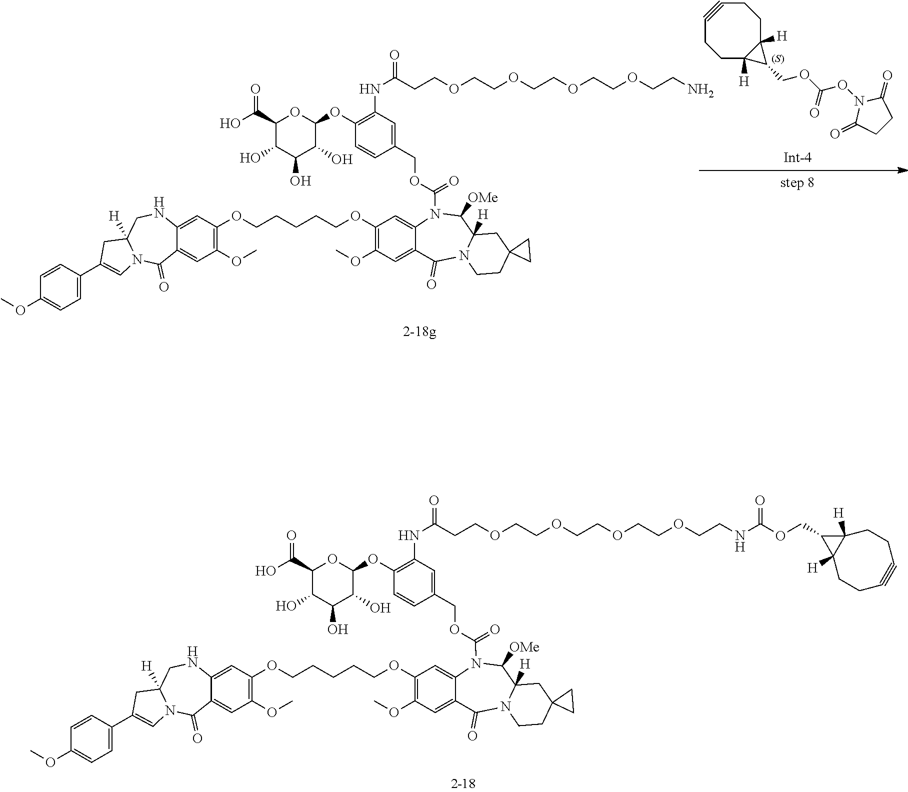

### Step 1: allyl(S)-8-((5-(5-amino-4-((S)-5-(((tert-butyldimethylsilyl)oxy) methyl)-6-azaspiro[2.5]octane-6-carbonyl)-2-methoxyphenoxy) pentyl)oxy)-7-methoxy-2-(4-methoxyphenyl)-5-oxo-11,11a-dihydro-1H-benzo[e]pyrrolo[1,2-a][1,4]diazepine-10 (5H)-carboxylate (2-18a)

To the solution of Int-18 (150 mg, 0.36 mmol) and Int-19 (213 mg, 0.36 mmol) in DMSO (3 mL) was added K₂CO₃ (100 mg, 0.71 mmol) under N₂ atmosphere, stirred at 80° C. for 20 min. The solution was added to water (50 mL), extracted with EtOAc (20 mL*3), washed with brine (20 mL*2). The organic phase was concentrated and purified by flash column chromatography (petroleum ether/EtOAc=25/75) to give 2-18a as an off-white solid (277 mg, 84% yield). MS (ESI) m/z: 925.7 [M+H]⁺.

### Step 2: (2S,3R,4S,5S,6S)-2-(2-(1-(9H-fluoren-9-yl)-3-oxo-2,7,10,13,16-pentaoxa-4-azanonadecan-19-amido)-4-((((5-((5-(((S)-10-((allyloxy) carbonyl)-7-methoxy-2-(4-methoxyphenyl)-5-oxo-5,10,11,11a-tetrahydro-1H-benzo[e]pyrrolo[1,2-a][1,4]diazepin-8-yl)oxy)pentyl)oxy)-2-((S)-5-(((tert-butyldimethylsilyl)oxy)methyl)-6-azaspiro[2.5]octane-6-carbonyl)-4-methoxyphenyl)carbamoyl)oxy)methyl)phenoxy)-6-(methoxycarbonyl)tetrahydro-2H-pyran-3,4,5-triyl triacetate (2-18b)

To the solution of 2-18a (186 mg, 0.2 mmol) in dry THF (2 mL) was added the solution of triphosgene (22.5 mg, 0.07 mmol) in dry THF (300 μL) under N₂ atmosphere at 0° C., followed by the addition of Et₃N (43 μL, 0.3 mmol), stirred at r.t. for 10 min. To the solution was added the solution of Int-7 (199 mg, 0.2 mmol), dibutyltin dilaurate (12 μL, 0.02 mmol) and Et₃N (43 μL, 0.3 mmol) in dry THF (1 mL) with 4 Å MS dried, stirred at r.t. for 1 h. The solution was filtered and washed with sat. NH₄Cl (5 mL) and water (5 mL), extracted with EtOAc (10 mL*3), concentrated, purified by flash column chromatography (10% MeOH in CH₂Cl₂/CH₂Cl₂=30/70) to give the title compound 2-18b as a yellow solid (343 mg, 91% yield). MS (ESI) m/z: 1876.4 [M+H]⁺.

### Step 3: (2S,3R,4S,5S,6S)-2-(2-(1-(9H-fluoren-9-yl)-3-oxo-2,7,10,13,16-pentaoxa-4-azanonadecan-19-amido)-4-((((5-((5-(((S)-10-((allyloxy)carbonyl)-7-methoxy-2-(4-methoxyphenyl)-5-oxo-5,10,11,11a-tetrahydro-1H-benzo[e]pyrrolo[1,2-a][1,4]diazepin-8-yl)oxy)pentyl)oxy)-2-((S)-5-(hydroxymethyl)-6-azaspiro[2.5]octane-6-carbonyl)-4-methoxyphenyl)carbamoyl)oxy)methyl)phenoxy)-6-(methoxycarbonyl)tetrahydro-2H-pyran-3,4,5-triyl triacetate (2-18c)

To the solution of 2-18b (343 mg, 0.18 mmol) in THF/H₂O (2.1/0.7 mL) was added PTSA (35.5 mg, 0.18 mmol) under N₂ atmosphere, stirred at r.t. for 5 h. The solution was added to sat. NaHCO₃ (10 mL), extracted with EtOAc (10 mL*3), concentrated, purified by flash column chromatography (10% MeOH in CH₂Cl₂/CH₂Cl₂=30/70) to give 2-18c as an off-white solid (262 mg, 81.3% yield). MS (ESI) m/z: 1762.3 [M+H]⁺.

### Step 4: (2S,3R,4S,5S,6S)-2-(2-(1-(9H-fluoren-9-yl)-3-oxo-2,7,10,13,16-pentaoxa-4-azanonadecan-19-amido)-4-((((6S,6aS)-3-((5-(((S)-10-((allyloxy)carbonyl)-7-methoxy-2-(4-methoxyphenyl)-5-oxo-5,10,11,11a-tetrahydro-1H-benzo[e]pyrrolo[1,2-a][1,4]diazepin-8-yl)oxy)pentyl)oxy)-6-hydroxy-2-methoxy-12-oxo-5,6a,7,9,10,12-hexahydro-6H-spiro[benzo[e]pyrido[1,2-a][1,4]diazepine-8,1′-cyclopropane]-5-carbonyl)oxy)methyl)phenoxy)-6-(methoxycarbonyl)tetrahydro-2H-pyran-3,4,5-triyl triacetate (2-18d)

To the solution of 2-18c (260 mg, 0.15 mmol) in CH₂Cl₂ (2 mL) was added Dess-Martin Periodinane (70 mg, 0.16 mmol) at 0° C., stirred at r.t. for 1.5 h. Dess-Martin Periodinane (70 mg, 0.16 mmol) was added at 0° C. again, stirred at r.t. for another 30 min. To the solution was added sat. Na₂S203 (5 mL) and sat. NaHCO₃ (5 mL), extracted with EtOAc (5 mL*3), concentrated, purified by flash column chromatography (10% MeOH in CH₂Cl₂/CH₂Cl₂=30/70) to give the mixture (aldehyde-form and ring-close-form products, 256 mg). To the solution of the mixture (256 mg, 0.14 mmol) in dry CH₂Cl₂ (4 mL) was added AcOH (170 μL, 2.9 mmol) under N₂ atmosphere, stirred at r.t. for 16 h. The solution was concentrated and purified by flash column chromatography (10% MeOH in CH₂Cl₂/CH₂Cl₂=30/70) to give 2-18d as a pale-yellow solid (180 mg, 69% yield). MS (ESI) m/z: 1760.3 [M+H]⁺.

### Step 5: (2S,3R,4S,5S,6S)-2-(2-(1-(9H-fluoren-9-yl)-3-oxo-2,7,10,13,16-pentaoxa-4-azanonadecan-19-amido)-4-((((6S,6aS)-3-((5-(((S)-10-((allyloxy)carbonyl)-7-methoxy-2-(4-methoxyphenyl)-5-oxo-5,10,11,11a-tetrahydro-1H-benzo[e]pyrrolo[1,2-a][1,4]diazepin-8-yl)oxy)pentyl)oxy)-2,6-dimethoxy-12-oxo-5,6a,7,9,10,12-hexahydro-6H-spiro[benzo[e]pyrido[1,2-a][1,4]diazepine-8,1′-cyclopropane]-5-carbonyl)oxy)methyl)phenoxy)-6-(methoxycarbonyl)tetrahydro-2H-pyran-3,4,5-triyl triacetate (2-18e)

To the solution of 2-18d (100 mg, 0.056 mmol) in dry THF (1 mL) were added TMSCl (74 μL, 0.57 mmol) and dry MeOH (82 μL, 1.99 mmol) under N₂ atmosphere, stirred at r.t. for 30 min and then at 50° C. for 90 min. The solution was concentrated and purified by flash column chromatography (10% MeOH in CH₂Cl₂/CH₂Cl₂=30/70) to give 2-18e as a yellow solid (37.5 mg, 37% yield). MS (ESI) m/z: 1774.3 [M+H]⁺.

### Step 6: (2S,3R,4S,5S,6S)-2-(2-(1-(9H-fluoren-9-yl)-3-oxo-2,7,10,13,16-pentaoxa-4-azanonadecan-19-amido)-4-((((6S,6aS)-2,6-dimethoxy-3-((5-(((S)-7-methoxy-2-(4-methoxyphenyl)-5-oxo-5,10,11,11a-tetrahydro-1H-benzo[e]pyrrolo[1,2-a][1,4]diazepin-8-yl)oxy)pentyl)oxy)-12-oxo-5,6a,7,9,10,12-hexahydro-6H-spiro[benzo[e]pyrido[1,2-a][1,4]diazepine-8,1′-cyclopropane]-5-carbonyl)oxy)methyl)phenoxy)-6-(methoxycarbonyl)tetrahydro-2H-pyran-3,4,5-triyl triacetate (2-18f)

To the mixture of 2-18e (49 mg, 0.028 mmol), dimedone (4.1 mg, 0.029 mmol) and Pd(PPh₃)₄ (0.8 mg, 0.7 μmol) were added dry THF (0.6 mL) and dry MeOH (0.2 mL) under N₂ atmosphere, stirred at r.t. for 30 min. The solution was concentrated and purified by flash column chromatography (10% MeOH in CH₂Cl₂/CH₂Cl₂=30/70) to give 2-18f as a yellow solid (28 mg, 60% yield). MS (ESI) m/z: 1690.3 [M+H]⁺.

### Step 7: (2S,3S,4S,5R,6S)-6-(2-(1-amino-3,6,9,12-tetraoxapentadecan-15-amido)-4-((((6S,6aS)-2,6-dimethoxy-3-((5-(((S)-7-methoxy-2-(4-methoxyphenyl)-5-oxo-5,10,11,11a-tetrahydro-1H-benzo[e]pyrrolo[1,2-a][1,4]diazepin-8-yl)oxy)pentyl)oxy)-12-oxo-5,6a,7,9,10,12-hexahydro-6H-spiro[benzo[e]pyrido[1,2-a][1,4]diazepine-8,1′-cyclopropane]-5-carbonyl)oxy)methyl)phenoxy)-3,4,5-trihydroxytetrahydro-2H-pyran-2-carboxylic acid (2-18g)

To the solution of 2-18f (28 mg, 0.017 mmol) in THF (0.3 mL) was added the solution of LiOH in water (4 mg, 0.17 mmol, 2 M, 83 μL) at 0° C., stirred at 0° C. for 30 min and then at r.t. for 2 h. AcOH (20 μL) was added to the solution. The solution was purified by prep-HPLC (Column: Sunfire Prep C18 OBD™ 5 μm, 19*150 mm; Mobile phase A: 0.1% FA in water, B: MeCN; Gradient: 20%-55% B; Flow rate: 20 mL/min), lyophilized to give 2-18g as a white solid (9 mg, 41% yield). MS (ESI) m/z: 1328.0 [M+H]⁺.

### Step 8: (2S,3S,4S,5R,6S)-6-(2-(1-((1R,8S,9s)-bicyclo[6.1.0]non-4-yn-9-yl)-3-oxo-2,7,10,13,16-pentaoxa-4-azanonadecan-19-amido)-4-((((6S,6aS)-2,6-dimethoxy-3-((5-(((S)-7-methoxy-2-(4-methoxyphenyl)-5-oxo-5,10,11,11a-tetrahydro-H-benzo[e]pyrrolo[1,2-a][1,4]diazepin-8-yl)oxy)pentyl)oxy)-12-oxo-5,6a,7,9,10,12-hexahydro-6H-spiro[benzo[e]pyrido[1,2-a][1,4]diazepine-8,1′-cyclopropane]-5-carbonyl)oxy)methyl)phenoxy)-3,4,5-trihydroxytetrahydro-2H-pyran-2-carboxylic acid (2-18)

To the solution of 2-18g (9 mg, 7 μmol) and Int-4 (2 mg, 7 μmol) in dry THF (0.5 mL) was added DIEA (4 μL, 20 μmol), stirred at r.t. for 10 min. The solution was purified by prep-HPLC (Column: Sunfire Prep C18 OBD™ 5 μm, 19*150 mm; Mobile phase A: 0.1% FA in water, B: MeCN; Gradient: 50%-80%-95% B; Flow rate: 20 mL/min), lyophilized to give the compound 2-18 as an off-white solid (5.8 mg, 56.9% yield). MS (ESI) m/z: 1504.1 [M+H]⁺.

### Example 2-20

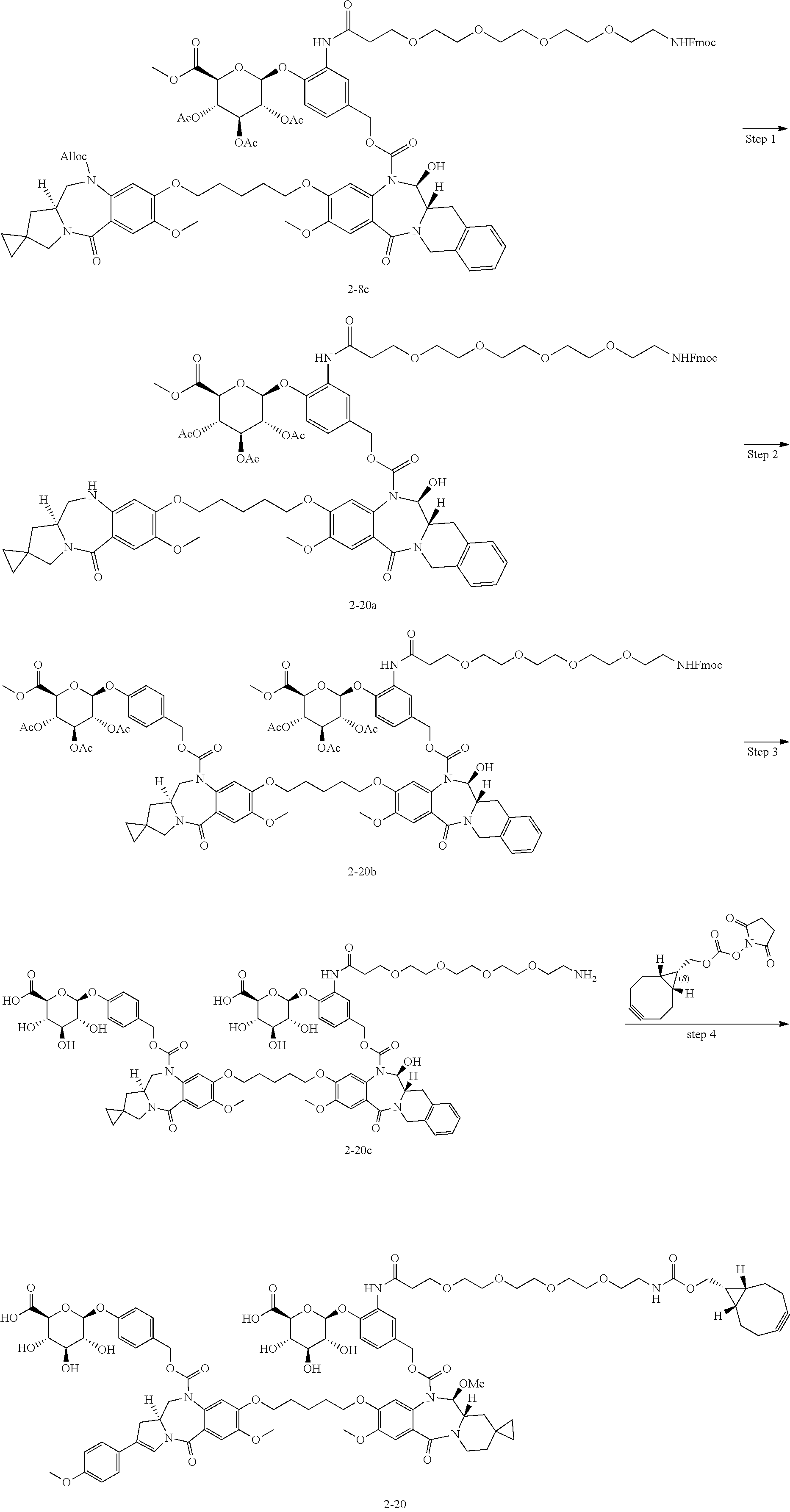

### Step 1: (2S,3R,4S,5S,6S)-2-(2-(1-(9H-fluoren-9-yl)-3-oxo-2,7,10,13,16-pentaoxa-4-azanonadecan-19-amido)-4-((((6S,6aS)-6-hydroxy-2-methoxy-3-((5-(((S)-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)pentyl)oxy)-14-oxo-5,6,6a,7,12,14-hexahydrobenzo[5,6][1,4]diazepino[1,2-b]isoquinoline-5-carbonyl)oxy)methyl)phenoxy)-6-(methoxycarbonyl)tetrahydro-2H-pyran-3,4,5-triyl triacetate (2-20a)

To a solution of 2-8c (80 mg, 0.048 mmol) in 2 mL CH₂Cl₂ was added Pd(PPh₃)₄ (1 mg, cat.) and dimedone (13 mg, 0.094 mmol) at 0° C. The reaction solution was stirred at r.t. for 2 h. The organic solvent was removed by evaporation. The crude product was purified by silica gel column chromatography (MeOH/CH₂Cl₂=10/90) obtain 2-20a as a white solid (60 mg, 79% yield). MS (ESI) m/z: 1260.0 [M+H]⁺.

### Step 2: (2S,3R,4S,5S,6S)-2-(2-(1-(9H-fluoren-9-yl)-3-oxo-2,7,10,13,16-pentaoxa-4-azanonadecan-19-amido)-4-((((6S,6aS)-6-hydroxy-2-methoxy-3-((5-(((S)-7-methoxy-5-oxo-10-(((4-(((2S,3R,4S,5S,6S)-3,4,5-triacetoxy-6-(methoxycarbonyl)tetrahydro-2H-pyran-2-yl)oxy)benzyl)oxy)carbonyl)-5,10,11,11a-tetrahydro-1H,3H-spiro[benzo[e]pyrrolo[1,2-a][1,4]diazepine-2,1′-cyclopropan]-8-yl)oxy)pentyl)oxy)-14-oxo-5,6,6a,7,12,14-hexahydrobenzo[5,6][1,4]diazepino[1,2-b]isoquinoline-5-carbonyl)oxy)methyl)phenoxy)-6-(methoxycarbonyl)tetrahydro-2H-pyran-3,4,5-triyl triacetate (2-20b)

Int-6 (60 mg, 0.04 mmol) was dissolved in dry CH₂Cl₂ (1 mL), 4 Å molecular sieves (100 mg) was added, followed by addition of dry pyridine (16.1 μL, 0.2 mmol). The mixture was cooled to −78° C., then triphosgene (4.3 mg, 0.014 mmol) in 0.5 mL CH₂Cl₂ was added dropwise. The mixture was stirred at −78° C. for 15 min under a nitrogen atmosphere. 2-20a (17.6 mg, 0.04 mmol) in dry CH₂Cl₂ (1 mL) was added. The reaction solution was stirred at 0° C. for 1 h. The organic solvent was removed by evaporation. The crude product was purified by silica gel column chromatography (MeOH/CH₂Cl_{2=15/85}) obtain 2-20b as a light-yellow solid (79 mg, 95% yield). MS (ESI) m/z: 2086.8 [M+H]⁺.

### Step 3: (2S,3S,4S,5R,6S)-6-(2-(1-amino-3,6,9,12-tetraoxapentadecan-15-amido)-4-((((6S,6aS)-3-((5-(((S)-10-(((4-(((2S,3R,4S,5S,6S)-6-carboxy-3,4,5-trihydroxytetrahydro-2H-pyran-2-yl)oxy)benzyl)oxy)carbonyl)-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)pentyl)oxy)-6-hydroxy-2-methoxy-14-oxo-5,6,6a,7,12,14-hexahydrobenzo[5,6][1,4]diazepino[1,2-b]isoquinoline-5-carbonyl)oxy)methyl)phenoxy)-3,4,5-trihydroxytetrahydro-2H-pyran-2-carboxylic acid (2-20c)

To a solution of sat. LiBr (1 mL) in CH₃CN/H₂O (10/1, v v) and triethylamine (0.2 mmol, 27 μL) was added 2-20b (80 mg, 0.04 mmol). The reaction mixture was stirred at r.t. for 4 h under N₂ atmosphere. The solvent was removed by evaporation to give crude product as a yellow solid, which was directly used in the next step without further purification.

To the above crude product in 1 mL DMF was added diethylamine (0.38 mmol, 40 μL) at 0° C. The reaction solution stirred at r.t. for 1 h. The reaction solution was neutralized with AcOH, and then purified by prep-HPLC (Method: column:XBridge Prep C18 OBD 5 μm 19*150 mm; Mobile phase: A—water (0.1% formic acid): B-acetonitrile; Flow rate: 20 mL/min) to provide 2-20c as a white solid after lyophilization (30 mg, 50% yield). MS (ESI) m/z: 1584.1 [M+H]⁺.

### Step 4: (2S,3S,4S,5R,6S)-6-(2-(1-((1R,8S,9s)-bicyclo[6.1.0]non-4-yn-9-yl)-3-oxo-2,7,10,13,16-pentaoxa-4-azanonadecan-19-amido)-4-((((6S,6aS)-3-((5-(((S)-10-(((4-(((2S,3R,4S,5S,6S)-6-carboxy-3,4,5-trihydroxytetrahydro-2H-pyran-2-yl)oxy)benzyl)oxy)carbonyl)-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)pentyl)oxy)-6-hydroxy-2-methoxy-14-oxo-5,6,6a,7,12,14-hexahydrobenzo[5,6][1,4]diazepino[1,2-b]isoquinoline-5-carbonyl)oxy)methyl)phenoxy)-3,4,5-trihydroxytetrahydro-2H-pyran-2-carboxylic acid (2-20)

To a solution of 2-20c (30 mg, 0.019 mmol) in 1 mL DMF was added Int-4 (11 mg, 0.038 mmol) and DIPEA (18.2 μL, 0.095 mmol) at 0° C. The reaction solution was stirred at 0° C. for 1 h. The reaction solution was neutralized with AcOH, and then purified by prep-HPLC (Method: column:XBridge Prep C18 OBD 5 μm 19*150 mm; Mobile phase: A—water (0.1% formic acid): B-acetonitrile; Flow rate: 20 mL/min) to provide 2-20 as a white solid after lyophilization (14.7 mg, 42.6% yield). MS (ESI) m/z: 1759.94 [M+H]⁺.

### Example 2-22

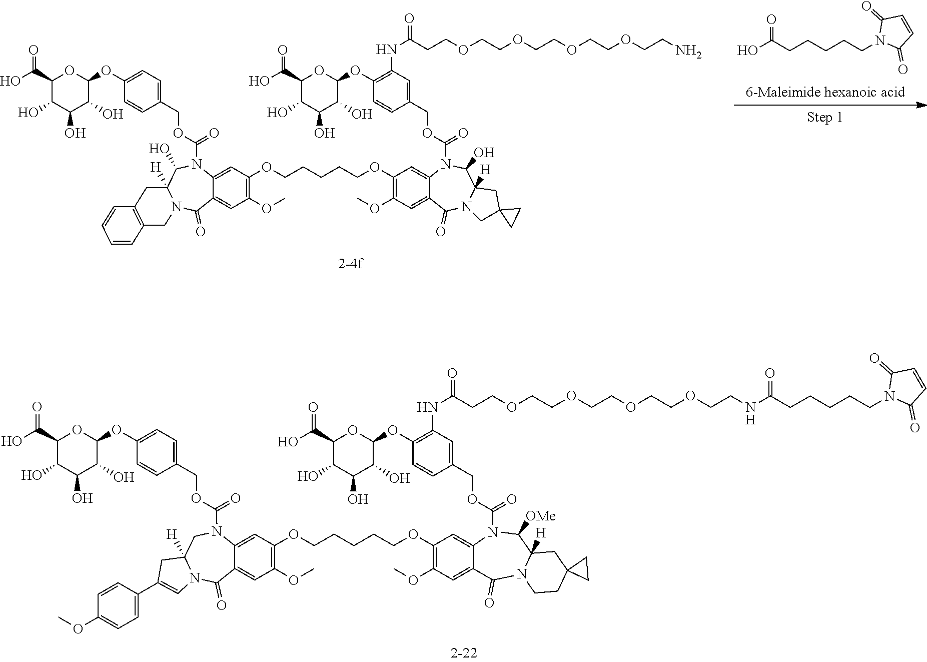

### Step 1: (2S,3S,4S,5R,6S)-6-(4-((((6S,6aS)-3-((5-(((11S,11aS)-10-(((4-(((2S,3R,4S,5S,6S)-6-carboxy-3,4,5-trihydroxytetrahydro-2H-pyran-2-yl)oxy)-3-(22-(2,5-dioxo-2,5-dihydro-1H-pyrrol-1-yl)-17-oxo-4,7,10,13-tetraoxa-16-azadocosanamido)benzyl)oxy)carbonyl)-11-hydroxy-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)pentyl)oxy)-6-hydroxy-2-methoxy-14-oxo-5,6,6a,7,12,14-hexahydrobenzo[5,6][1,4]diazepino[1,2-b]isoquinoline-5-carbonyl)oxy)methyl)phenoxy)-3,4,5-trihydroxytetrahydro-2H-pyran-2-carboxylic acid (2-22)

To a mixture of 6-maleimide hexanoic acid (5.3 mg, 0.025 mmol), HATU (7.1 mg, 0.019 mmol) in DMF (0.5 mL) were added DIEA (8.9 μL, 0.05 mmol) at r.t. After stirred for 10 min, 2-4f (20 mg, 0.0125 mmol) was added to the mixture. The mixture was stirred at r.t. for 1 h. The mixture was adjusted to pH 7 using AcOH and filtrated, and the filtrate was purified using prep-HPLC (Method: column:XBridge Prep C18 OBD 5 μm 19*150 mm; Mobile phase: A—water (0.1% formic acid): B-acetonitrile; Flow rate: 20 mL/min) to provide 2-22 (13.6 mg, 60.7% yield) as a white solid. MS (ESI) m/z: 1792.7 [M+H].

### Example 2-23

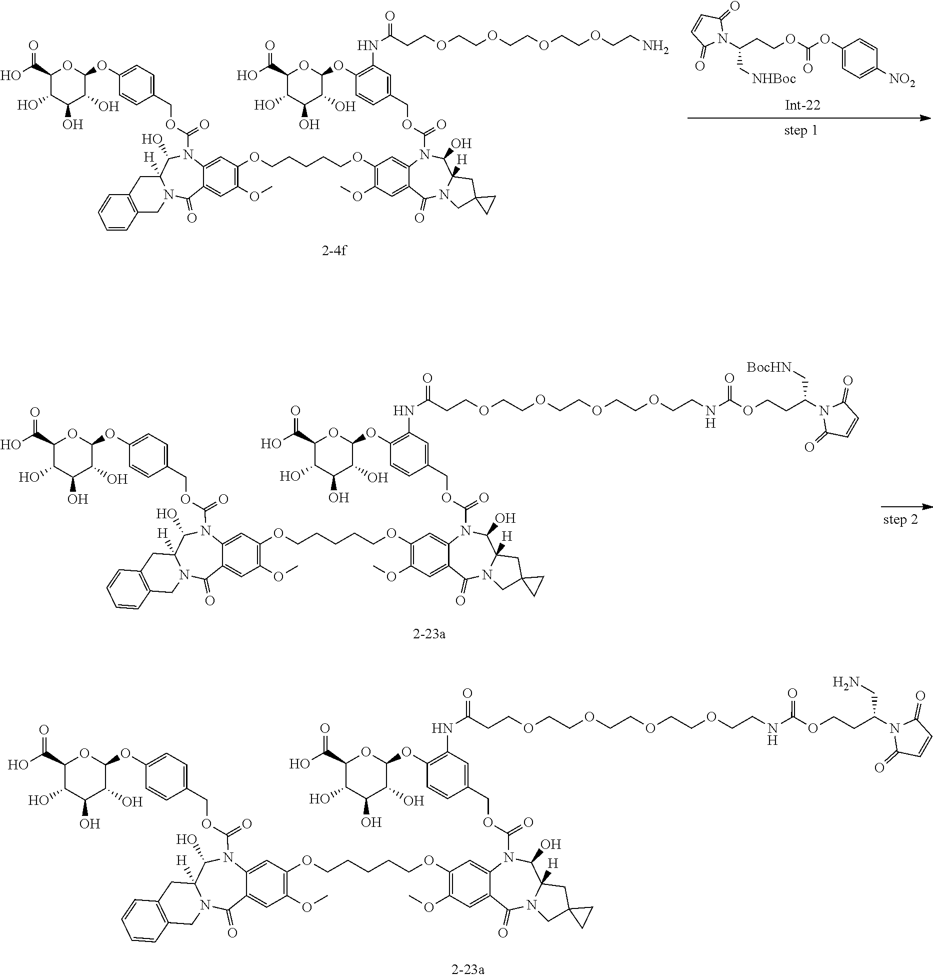

### Step 1: (2S,3S,4S,5R,6S)-6-(4-((((6S,6aS)-3-((5-(((11S,11aS)-10-(((4-(((2S,3R,4S,5S,6S)-6-carboxy-3,4,5-trihydroxytetrahydro-2H-pyran-2-yl)oxy)-3-((R)-7-(2,5-dioxo-2,5-dihydro-1H-pyrrol-1-yl)-2,2-dimethyl-4,11-dioxo-3,10,15,18,21,24-hexaoxa-5,12-diazaheptacosan-27-amido)benzyl)oxy)carbonyl)-11-hydroxy-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)pentyl)oxy)-6-hydroxy-2-methoxy-14-oxo-5,6,6a,7,12,14-hexahydrobenzo[5,6][1,4]diazepino[1,2-b]isoquinoline-5-carbonyl)oxy)methyl)phenoxy)-3,4,5-trihydroxytetrahydro-2H-pyran-2-carboxylic acid (2-23a)

To a mixture of 2-4f (47 mg, 0.030 mmol), Int-22 (16 mg, 0.037 mmol) in DMF (1 mL) was added DIEA (16 μL, 0.089 mmol). The mixture was stirred at r.t. for 3 h. The mixture was adjusted to pH=7 using AcOH and filtrated, and the filtrate purified using prep-HPLC (Method: column:XBridge Prep C18 OBD 5 μm 19*150 mm; Mobile phase: A—water (0.1% formic acid): B-acetonitrile; Flow rate: 20 mL/min) to provide 2-23a (35 mg, 62.5% yield) as a white solid. MS (ESI) m/z: 1910.0 [M+H]⁺.

### Step 2: (2S,3S,4S,5R,6S)-6-(2-((R)-22-amino-21-(2,5-dioxo-2,5-dihydro-1H-pyrrol-1-yl)-17-oxo-4,7,10,13,18-pentaoxa-16-azadocosanamido)-4-((((11S,11aS)-8-((5-(((6S,6aS)-5-(((4-(((2S,3R,4S,5S,6S)-6-carboxy-3,4,5-trihydroxytetrahydro-2H-pyran-2-yl)oxy)benzyl)oxy)carbonyl)-6-hydroxy-2-methoxy-14-oxo-5,6,6a,7,12,14-hexahydrobenzo[5,6][1,4]diazepino[1,2-b]isoquinolin-3-yl)oxy)pentyl)oxy)-11-hydroxy-7-methoxy-5-oxo-5,10,11,11a-tetrahydro-1H,3H-spiro[benzo[e]pyrrolo[1,2-a][1,4]diazepine-2,1′-cyclopropane]-10-carbonyl)oxy)methyl)phenoxy)-3,4,5-trihydroxytetrahydro-2H-pyran-2-carboxylic acid (2-23)

To a mixture of 2-23a (20 mg, 0.010 mmol) in CH₂Cl₂ (2 mL) and H₂O (200 μL) at 0° C. was added TFA (1 mL). The mixture was stirred at 0° C. for 2 h. The mixture was concentrated under vacuum to give a residue which was purified using prep-HPLC (Method: column:XBridge Prep C18 OBD 5 μm 19*150 mm; Mobile phase: A—water (0.1% formic acid): B-acetonitrile; Flow rate: 20 mL/min) to provide 2-23 (8.9 mg, 47.1% yield) as a white solid. MS (ESI) m/z: 1810.2 [M+H]⁺.

### Example 2-24

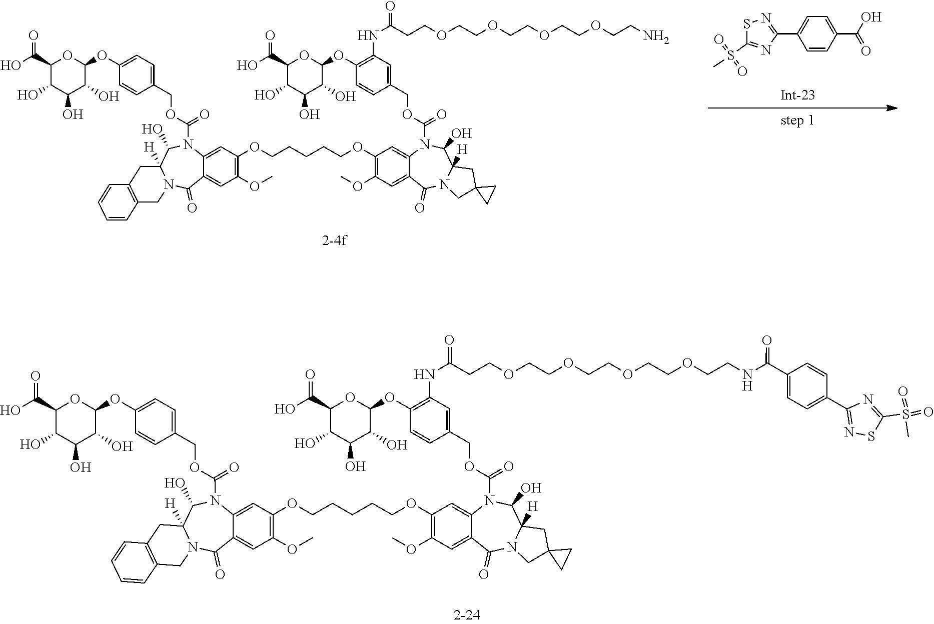

### Step 1: (2S,3S,4S,5R,6S)-6-(4-((((6S,6aS)-3-((5-(((11S,11aS)-10-(((4-(((2S,3R,4S,5S,6S)-6-carboxy-3,4,5-trihydroxytetrahydro-2H-pyran-2-yl)oxy)-3-(1-(4-(5-(methylsulfonyl)-1,2,4-thiadiazol-3-yl)phenyl)-1-oxo-5,8,11,14-tetraoxa-2-azaheptadecan-17-amido)benzyl)oxy)carbonyl)-11-hydroxy-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)pentyl)oxy)-6-hydroxy-2-methoxy-14-oxo-5,6,6a,7,12,14-hexahydrobenzo[5,6][1,4]diazepino[1,2-b]isoquinoline-5-carbonyl)oxy)methyl)phenoxy)-3,4,5-trihydroxytetrahydro-2H-pyran-2-carboxylic acid (2-24)

Compound 2-24 (7.4 mg, 31.8% yield) was synthesized according to the synthetic procedure of step 1 of example 2-22. MS (ESI) m/z: 1865.5 [M+H]⁺.

### Example 2-25

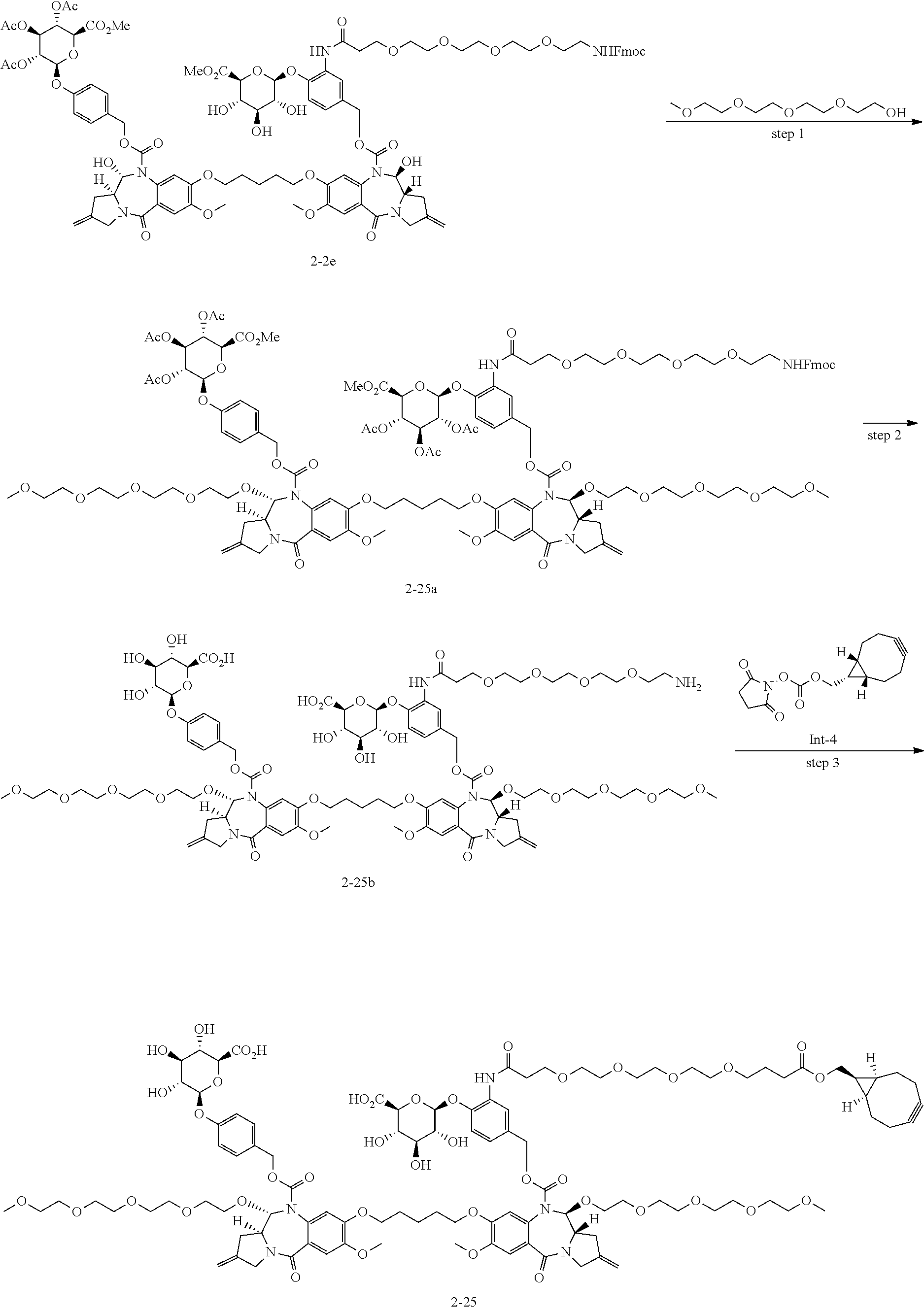

### Step 1: (2S,3R,4S,5S,6S)-2-(4-((((11S,11aS)-11-((2,5,8,11-tetraoxatridecan-13-yl)oxy)-8-((5-(((11S,11aS)-11-((2,5,8,11-tetraoxatridecan-13-yl)oxy)-10-(((3-(1-(9H-fluoren-9-yl)-3-oxo-2,7,10,13,16-pentaoxa-4-azanonadecan-19-amido)-4-(((2S,3R,4S,5S,6S)-3,4,5-triacetoxy-6-(methoxycarbonyl)tetrahydro-2H-pyran-2-yl)oxy)benzyl)oxy)carbonyl)-7-methoxy-2-methylene-5-oxo-2,3,5,10,11,11a-hexahydro-1H-benzo[e]pyrrolo[1,2-a][1,4]diazepin-8-yl)oxy)pentyl)oxy)-7-methoxy-2-methylene-5-oxo-2,3,5,10,11,11a-hexahydro-1H-benzo[e]pyrrolo[1,2-a][1,4]diazepine-10-carbonyl)oxy)methyl)phenoxy)-6-(methoxycarbonyl)tetrahydro-2H-pyran-3,4,5-triyl triacetate (2-25a)

To a solution of 2-2e (90 mg, 0.044 mmol) and 2,5,8,11-tetraoxatridecan-13-ol (322 mg, 1.55 mmol) in THF (0.9 mL) was added TMSCl (48 mg, 0.44 mmol) on ice-bath, after addition the reaction was stirred at 65° C. for 5 h. The mixture was quenched with H₂O (5 mL). The aqueous layer was extracted with EtOAc (10 mL*3). The combined organic layers were washed with brine (20 mL), dried over anhydrous Na₂SO₄, filtered and concentrated under vacuum to give a residue which was purified by silica gel column chromatography (A-CH₂Cl₂; B-MeOH) to provide 2-25a (86.7 mg, 81.2%) as a pale-yellow oil. MS (ESI) m/z: 2418.1 [M+H]⁺.

### Step 2: (2S,3S,4S,5R,6S)-6-(4-((((11S,11aS)-11-((2,5,8,11-Tetraoxatridecan-13-yl)oxy)-8-((5-(((11S,11aS)-11-((2,5,8,11-tetraoxatridecan-13-yl)oxy)-10-(((3-(1-amino-3,6,9,12-tetraoxapentadecan-15-amido)-4-(((2S,3R,4S,5S,6S)-6-carboxy-3,4,5-trihydroxytetrahydro-2H-pyran-2-yl)oxy)benzyl)oxy)carbonyl)-7-methoxy-2-methylene-5-oxo-2,3,5,10,11,11a-hexahydro-1H-benzo[e]pyrrolo[1,2-a][1,4]diazepin-8-yl)oxy)pentyl)oxy)-7-methoxy-2-methylene-5-oxo-2,3,5,10,11,11a-hexahydro-1H-benzo[e]pyrrolo[1,2-a][1,4]diazepine-10-carbonyl)oxy)methyl)phenoxy)-3,4,5-trihydroxytetrahydro-2H-pyran-2-carboxylic acid (2-25b)

To a mixed solution of 2-25a (86.7 mg, 0.036 mmol) in THF-H₂O (1:1, 6 mL) were added LiOH (8.6 mg, 0.36 mmol) at r.t., after addition the reaction was stirred at this temperature for 2 h. After complete reaction, the mixture was quenched with AcOH (22 mg, 0.36 mmol) and concentrated under vacuum to give a residue which was diluted by DMF (3 mL). The crude product was purified using prep-HPLC (Method: column:XBridge Prep C18 OBD 5 μm 19*150 mm; Mobile phase: A—water (0.1% formic acid): B-acetonitrile; Flow rate: 20 mL/min) to provide 2-25b (17 mg, 25.3% yield) as a white solid. MS (ESI) m/z: 1916.2 [M+H]⁺.

### Step 3: (2S,3S,4S,5R,6S)-6-(4-((((11S,11aS)-11-((2,5,8,11-tetraoxatridecan-13-yl)oxy)-8-((5-(((11S,11aS)-11-((2,5,8,11-tetraoxatridecan-13-yl)oxy)-10-(((3-(1-((1R,8S,9s)-bicyclo[6.1.0]non-4-yn-9-yl)-3-oxo-2,7,10,13,16-pentaoxa-4-azanonadecan-19-amido)-4-(((2S,3R,4S,5S,6S)-6-carboxy-3,4,5-trihydroxytetrahydro-2H-pyran-2-yl)oxy)benzyl)oxy)carbonyl)-7-methoxy-2-methylene-5-oxo-2,3,5,10,11,11a-hexahydro-1H-benzo[e]pyrrolo[1,2-a][1,4]diazepin-8-yl)oxy)pentyl)oxy)-7-methoxy-2-methylene-5-oxo-2,3,5,10,11,11a-hexahydro-1H-benzo[e]pyrrolo[1,2-a][1,4]diazepine-10-carbonyl)oxy)methyl)phenoxy)-3,4,5-trihydroxytetrahydro-2H-pyran-2-carboxylic acid (2-25)

Compound 2-25 (13 mg, 71.6%) was synthesized according to the synthetic procedure of step 6 of example Ref-2-2. MS (ESI) m/z: 2092.4 [M+H].

### Example 2-29

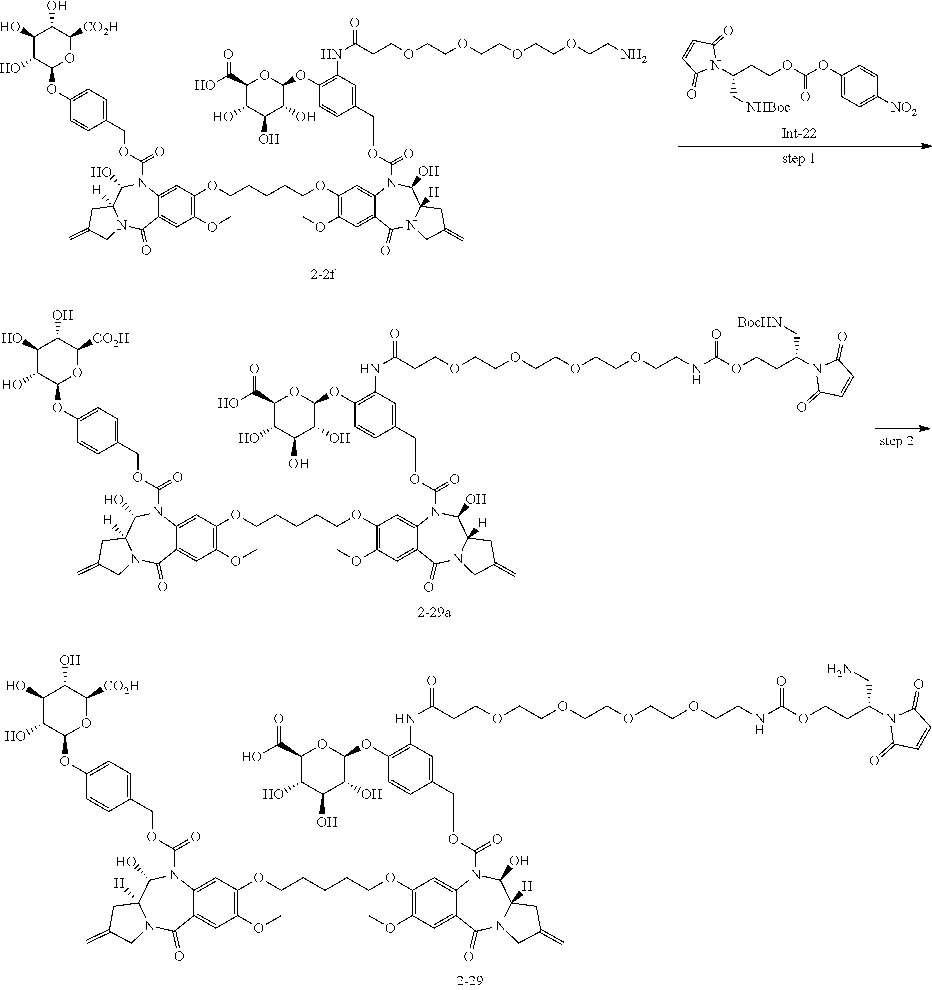

### Step 1: (2S,3S,4S,5R,6S)-6-(4-((((11S,11aS)-8-((5-(((11S,11aS)-10-(((4-(((2S,3R,4S,5S,6S)-6-carboxy-3,4,5-trihydroxytetrahydro-2H-pyran-2-yl)oxy)-3-((R)-7-(2,5-dioxo-2,5-dihydro-1H-pyrrol-1-yl)-2,2-dimethyl-4,11-dioxo-3,10,15,18,21,24-hexaoxa-5,12-diazaheptacosan-27-amido)benzyl)oxy)carbonyl)-11-hydroxy-7-methoxy-2-methylene-5-oxo-2,3,5,10,11,11a-hexahydro-1H-benzo[e]pyrrolo[1,2-a][1,4]diazepin-8-yl)oxy)pentyl)oxy)-11-hydroxy-7-methoxy-2-methylene-5-oxo-2,3,5,10,11,11a-hexahydro-1H-benzo[e]pyrrolo[1,2-a][1,4]diazepine-10-carbonyl)oxy)methyl)phenoxy)-3,4,5-trihydroxytetrahydro-2H-pyran-2-carboxylic acid (2-29a)

To a mixture of 2-2f (15 mg, 0.0099 mmol), Int-22 (9 mg, 0.02 mmol) in DMF (2 mL) was added DIEA (6 μL, 0.03 mmol). The mixture was stirred at r.t. for 3 h. The mixture was adjusted to pH=7 using AcOH and filtrated, the filtrate purified using prep-HPLC (Method: column:XBridge Prep C18 OBD 5 μm 19*150 mm; Mobile phase: A—water (0.1% formic acid): B-acetonitrile; Flow rate: 20 mL/min) to provide 2-29a (12 mg, 66.7% yield) as a white solid. MS (ESI) m/z: 1846.4 [M+H]⁺.

### Step 2: (2S,3S,4S,5R,6S)-6-(2-((R)-22-amino-21-(2,5-dioxo-2,5-dihydro-1H-pyrrol-1-yl)-17-oxo-4,7,10,13,18-pentaoxa-16-azadocosanamido)-4-((((11S,11aS)-8-((5-(((11S,11aS)-10-(((4-(((2S,3R,4S,5S,6S)-6-carboxy-3,4,5-trihydroxytetrahydro-2H-pyran-2-yl)oxy)benzyl)oxy)carbonyl)-11-hydroxy-7-methoxy-2-methylene-5-oxo-2,3,5,10,11,11a-hexahydro-1H-benzo[e]pyrrolo[1,2-a][1,4]diazepin-8-yl)oxy)pentyl)oxy)-11-hydroxy-7-methoxy-2-methylene-5-oxo-2,3,5,10,11,11a-hexahydro-1H-benzo[e]pyrrolo[1,2-a][1,4]diazepine-10-carbonyl)oxy)methyl)phenoxy)-3,4,5-trihydroxytetrahydro-2H-pyran-2-carboxylic acid (2-29)

To a mixture of 2-29a (12 mg, 0.0065 mmol) in CH₂Cl₂ (2 mL) and H₂O (200 μL) at 0° C. was added TFA (400 μL). The mixture was stirred at r.t. for 1 h. The mixture was concentrated under vacuum to give a residue, which was purified using prep-HPLC (Method: column:XBridge Prep C18 OBD 5 μm 19*150 mm; Mobile phase: A—water (0.1% formic acid): B-acetonitrile; Flow rate: 20 mL/min) to provide 2-29 (7.0 mg, 61.9% yield) as a white solid. MS (ESI) m/z: 1745.6 [M+H]⁺.

### Example 2-34

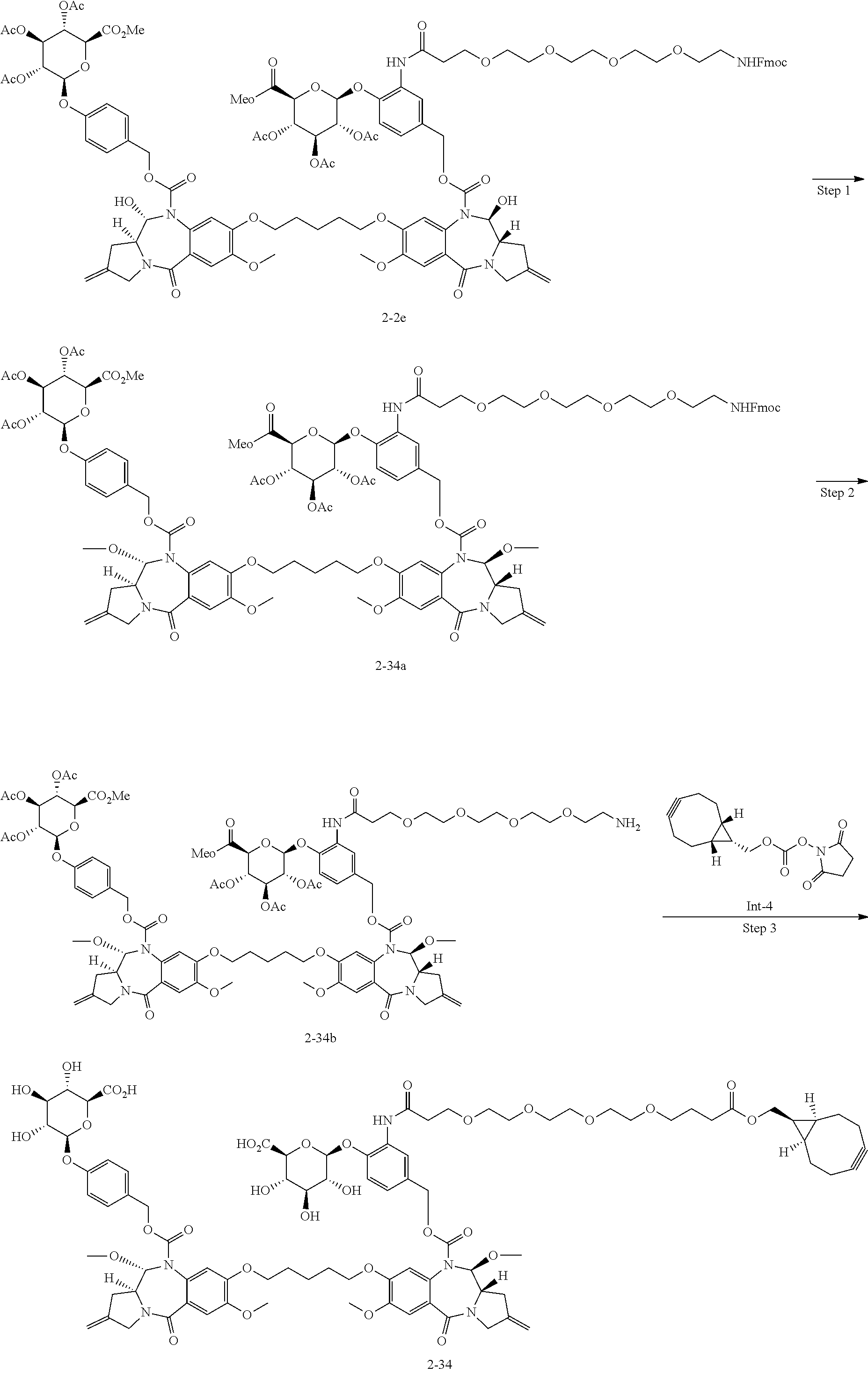

### Step 1: (2S,3R,4S,5S,6S)-2-(2-(1-(9H-fluoren-9-yl)-3-oxo-2,7,10,13,16-pentaoxa-4-azanonadecan-19-amido)-4-((((11S,11aS)-8-((5-(((11S,11aS)-7,11-dimethoxy-2-methylene-5-oxo-10-(((4-(((2S,3R,4S,5S,6S)-3,4,5-triacetoxy-6-(methoxycarbonyl)tetrahydro-2H-pyran-2-yl)oxy)benzyl)oxy)carbonyl)-2,3,5,10,11,11a-hexahydro-H-benzo[e]pyrrolo[1,2-a][1,4]diazepin-8-yl)oxy)pentyl)oxy)-7,11-dimethoxy-2-methylene-5-oxo-2,3,5,10,11,11a-hexahydro-1H-benzo[e]pyrrolo[1,2-a][1,4]diazepine-10-carbonyl)oxy)methyl)phenoxy)-6-(methoxycarbonyl)tetrahydro-2H-pyran-3,4,5-triyl triacetate (2-34a)

To a solution of 2-2e (50 mg, 0.025 mmol), MeOH (35 μL, 0.86 mmol) and THF (5 mL) were added TMSCl (31 μL, 0.25 mmol). The mixture was stirred for 4 h at 65° C. The mixture was concentrated under vacuum to give a residue which was purified by silica gel column chromatography (Petroleum ether/EtOAc=30/70) to afford 2-34a (50 mg, 98.0% yield) as a yellow solid. MS (ESI) m/z: 2066.6 [M+H]⁺.

### Step 2: (2S,3S,4S,5R,6S)-6-(2-(1-amino-3,6,9,12-tetraoxapentadecan-15-amido)-4-((((11S,11aS)-8-((5-(((11S,11aS)-10-(((4-(((2S,3R,4S,5S,6S)-6-carboxy-3,4,5-trihydroxytetrahydro-2H-pyran-2-yl)oxy)benzyl)oxy)carbonyl)-7,11-dimethoxy-2-methylene-5-oxo-2,3,5,10,11,11a-hexahydro-1H-benzo[e]pyrrolo[1,2-a][1,4]diazepin-8-yl)oxy)pentyl)oxy)-7,11-dimethoxy-2-methylene-5-oxo-2,3,5,10,11,11a-hexahydro-1H-benzo[e]pyrrolo[1,2-a][1,4]diazepine-10-carbonyl)oxy)methyl)phenoxy)-3,4,5-trihydroxytetrahydro-2H-pyran-2-carboxylic acid (2-34b)

To a solution of 2-34a (50 mg, 0.0024 mmol), H₂O (2 mL) in THF (2 mL) was added LiOH (8 mg, 0.34 mmol). The mixture was stirred at r.t. for 1 h. The mixture was adjusted to pH 7 using AcOH and concentrated under vacuum to give residue, which was purified using prep-HPLC (Method: column:XBridge Prep C18 OBD 5 μm 19*150 mm; Mobile phase: A—water (0.1% formic acid): B-acetonitrile; Flow rate: 20 mL/min) to provide 2-34b (10 mg, 27.0% yield) as a white solid. MS (ESI) m/z: 1563.9 [M+H]⁺.

### Step 3: (2S,3S,4S,5R,6S)-6-(2-(1-((1R,8S,9s)-bicyclo[6.1.0]non-4-yn-9-yl)-3-oxo-2,7,10,13,16-pentaoxa-4-azanonadecan-19-amido)-4-((((11 S,11aS)-8-((5-(((11S,11aS)-10-(((4-(((2S,3R,4S,5S,6S)-6-carboxy-3,4,5-trihydroxytetrahydro-2H-pyran-2-yl)oxy)benzyl)oxy)carbonyl)-7,11-dimethoxy-2-methylene-5-oxo-2,3,5,10,11,11a-hexahydro-H-benzo[e]pyrrolo[1,2-a][1,4]diazepin-8-yl)oxy)pentyl)oxy)-7,11-dimethoxy-2-methylene-5-oxo-2,3,5,10,11,11a-hexahydro-TH-benzo[e]pyrrolo[1,2-a][1,4]diazepine-10-carbonyl)oxy)methyl)phenoxy)-3,4,5-trihydroxytetrahydro-2H-pyran-2-carboxylic acid (2-34)

To a mixture of 2-34b (10 mg, 0.0064 mmol), Int-4 (4 mg, 0.013 mmol) in DMF (2 mL) was added DIEA (6 μL, 0.03 mmol). The mixture was stirred at r.t. for 1 h. The mixture was adjusted to pH 7 using AcOH and filtrated and the filter purified using prep-HPLC (Method: column:XBridge Prep C18 OBD 5 μm 19*150 mm; Mobile phase: A—water (0.1% formic acid): B-acetonitrile; Flow rate: 20 mL/min) to provide 2-34 (6.0 mg, 54.1% yield) as a white solid. MS (ESI) m/z: 1740.1 [M+H]⁺.

### Example 2-35

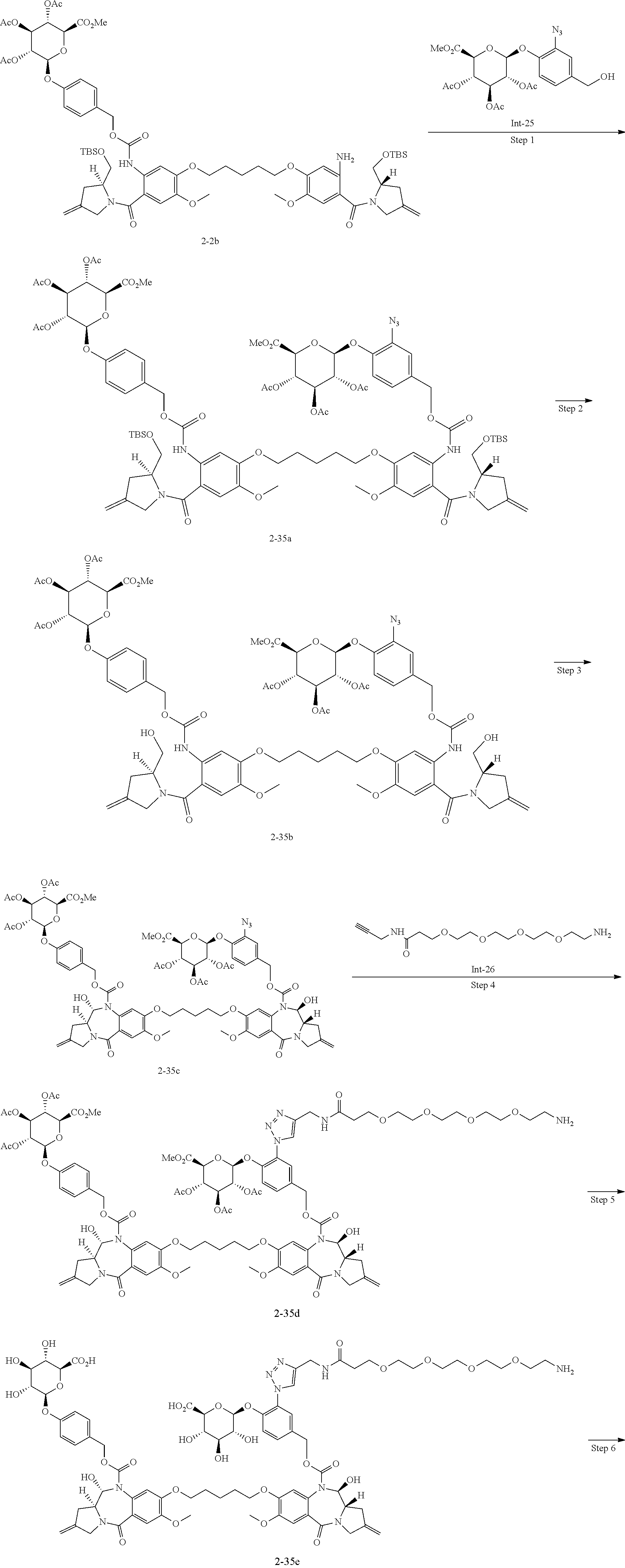

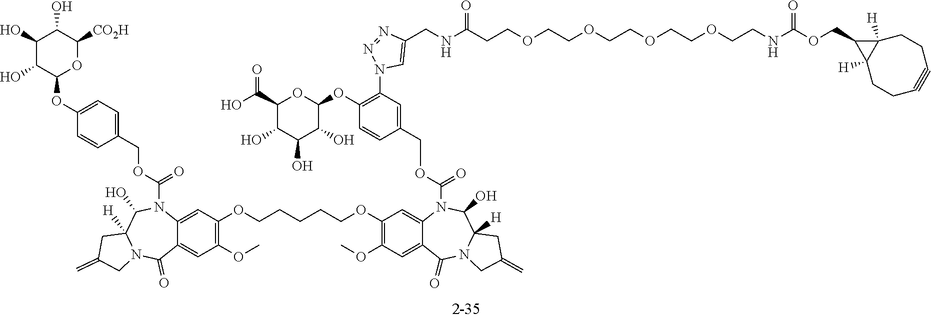 [SMILES: C=C1C[C@H]2[C@H](O)N(C(=O)OCc3ccc(O[C@@H]4O[C@H](C(=O)O)[C@@H](O)[C@H](O)[C@H]4O)cc3)c3cc(OCCCCCOc4cc5c(cc4OC)C(=O)N4CC(=C)C[C@H]4[C@H](O)N5C(=O)OCc4ccc(O[C@@H]5O[C@H](C(=O)O)[C@@H](O)[C@H](O)[C@H]5O)c(-n5cc(CNC(=O)CCOCCOCCOCCOCCNC(=O)OC[C@@H]6[C@@H]7CCC#CCC[C@@H]76)nn5)c4)c(OC)cc3C(=O)N2C1]

### Step 1: (2S,3R,4S,5S,6S)-2-(2-azido-4-((((2-((S)-2-(((tert-butyldimethylsilyl)oxy)methyl)-4-methylenepyrrolidine-1-carbonyl)-5-((5-(4-((S)-2-(((tert-butyldimethylsilyl)oxy)methyl)-4-methylenepyrrolidine-1-carbonyl)-2-methoxy-5-((((4-(((2S,3R,4S,5S,6S)-3,4,5-triacetoxy-6-(methoxy carbonyl)tetrahy dro-2H-pyran-2-yl)oxy)benzyl)oxy)carbonyl)amino)phenoxy)pentyl)oxy)-4-methoxyphenyl)carbamoyl)oxy)methyl)phenoxy)-6-(methoxycarbonyl)tetrahydro-2H-pyran-3,4,5-triyl triacetate (2-35a)

2-2b (100 mg, 0.076 mmol) was dissolved in dry THF (1 mL), 4 Å molecular sieves (100 mg) was added. The mixture was cooled to 0° C., then triphosgene (8.11 mg, 0.027 mmol) was added, followed by addition of triethylamine (23.43 μL, 0.167 mmol). The mixture was stirred at 0° C. for 15 min under a nitrogen atmosphere. Int-25 (38.3 mg, 0.08 mmol), triethylamine (16 μL, 0.114 mmol) and dibutyltin dilaurate 9 μL, 0.015 mmol) was mixed in dry THF (1 mL), and then this solution was gradually added to the above reaction solution at 0° C. The reaction solution was warm to r.t. and stirred for 2 h. The reaction solution was concentrated, and then purified by column chromatography (Petroleum ether/EtOAc=40/60) to obtain 2-35a as a white solid (135 mg, 97% yield). MS (ESI) m/z: 1827.4 [M+H]⁺.

### Step 2: (2S,3R,4S,5S,6S)-2-(2-azido-4-((((2-((S)-2-(hydroxymethyl)-4-methylenepyrrolidine-1-carbonyl)-5-((5-(4-((S)-2-(hydroxymethyl)-4-methylenepyrrolidine-1-carbonyl)-2-methoxy-5-((((4-(((2S,3R,4S,5S,6S)-3,4,5-triacetoxy-6-(methoxy carbonyl)tetrahydro-2H-pyran-2-yl)oxy)benzyl)oxy)carbonyl)amino)phenoxy)pentyl)oxy)-4-methoxyphenyl)carbamoyl)oxy)methyl)phenoxy)-6-(methoxycarbonyl)tetrahydro-2H-pyran-3,4,5-triyl triacetate (2-35b)

Compound 2-35a (135 mg, 0.074 mmol) was dissolved in THF/H₂O (1 mL/20 μL, 50/1), TsOH-H₂O (28 mg, 0.148 mmol) was added, and the mixture was stirred at r.t. for 12 h. The solvent was then removed by evaporation, the residue was purified by column chromatography (MeOH/CH₂Cl₂=6/94) to obtain 2-35b as a white solid (83 mg, 70.26% yield). MS (ESI) m/z: 1599.3 [M+H]⁺.

### Step 3: (2S,3R,4S,5S,6S)-2-(2-azido-4-((((11S,11aS)-11-hydroxy-8-((5-(((11S,11aS)-11-hydroxy-7-methoxy-2-methylene-5-oxo-10-(((4-(((2S,3R,4S,5S,6S)-3,4,5-triacetoxy-6-(methoxycarbonyl)tetrahydro-2H-pyran-2-yl)oxy)benzyl)oxy)carbonyl)-2,3,5,10,11,11a-hexahydro-TH-benzo[e]pyrrolo[1,2-a][1,4]diazepin-8-yl)oxy)pentyl)oxy)-7-methoxy-2-methylene-5-oxo-2,3,5,10,11,11a-hexahydro-TH-benzo[e]pyrrolo[1,2-a][1,4]diazepine-10-carbonyl)oxy)methyl)phenoxy)-6-(methoxycarbonyl)tetrahydro-2H-pyran-3,4,5-triyl triacetate (2-35c)

To a solution of 2-35b (80 mg, 0.05 mmol) in CH₂Cl₂ (1 mL) was added Dess-Martin periodinane (51 mg, 0.12 mmol) at r.t., and the mixture was stirred at r.t. for 12 h under a nitrogen atmosphere. The reaction solution was then diluted with CH₂Cl₂ (10 mL), washed with sat. Na₂S₂O₃ (1 mL) and brine (5 mL). The organic phase was collected and dried over anhydrous Na₂SO₄, filtered and concentrated under vacuum to give a residue which was purified by silica gel column chromatography (MeOH/CH₂Cl_{2=6/94}) obtain 2-35c as a white solid (65 mg, 81% yield). MS (ESI) m/z: 1595.2 [M+H]⁺.

### Step 4: (2S,3R,4S,5S,6S)-2-(2-(4-(17-amino-3-oxo-6,9,12,15-tetraoxa-2-azaheptadecyl)-1H-1,2,3-triazol-1-yl)-4-((((11S,11aS)-11-hydroxy-8-((5-(((11S,11aS)-11-hydroxy-7-methoxy-2-methylene-5-oxo-10-(((4-(((2S,3R,4S,5S,6S)-3,4,5-triacetoxy-6-(methoxycarbonyl)tetrahydro-2H-pyran-2-yl)oxy)benzyl)oxy)carbonyl)-2,3,5,10,11,11a-hexahydro-1H-benzo[e]pyrrolo[1,2-a][1,4]diazepin-8-yl)oxy)pentyl)oxy)-7-methoxy-2-methylene-5-oxo-2,3,5,10,11,11a-hexahydro-1H-benzo[e]pyrrolo[1,2-a][1,4]diazepine-10-carbonyl)oxy)methyl)phenoxy)-6-(methoxycarbonyl)tetrahydro-2H-pyran-3,4,5-triyl triacetate (2-35d)

To a solution of 2-35c (65 mg, 0.041 mmol) and Int-26 (13.6 mg, 0.045 mmol) in 1 mL DMF was added DIPEA (15 μL, 0.082 mmol) at 0° C., followed by addition of cat. amount of CuI. The mixture was degassed with N₂ for 10 min and then stirred under a nitrogen atmosphere for 1 h. The solvent was then removed by evaporation, the residue was purified by column chromatography (MeOH/CH₂Cl₂=10/90) to obtain 2-35d as a white solid (70 mg, 90.5% yield). MS (ESI) m/z: 1897.1 [M+H]⁺.

### Step 5: (2S,3S,4S,5R,6S)-6-(2-(4-(17-amino-3-oxo-6,9,12,15-tetraoxa-2-azaheptadecyl)-1H-1,2,3-triazol-1-yl)-4-((((11S,11aS)-8-((5-(((11S,11aS)-10-(((4-(((2S,3R,4S,5S,6S)-6-carboxy-3,4,5-trihydroxytetrahydro-2H-pyran-2-yl)oxy)benzyl)oxy)carbonyl)-11-hydroxy-7-methoxy-2-methylene-5-oxo-2,3,5,10,11,11a-hexahydro-1H-benzo[e]pyrrolo[1,2-a][1,4]diazepin-8-yl)oxy)pentyl)oxy)-11-hydroxy-7-methoxy-2-methylene-5-oxo-2,3,5,10,11,11a-hexahydro-1H-benzo[e]pyrrolo[1,2-a][1,4]diazepine-10-carbonyl)oxy)methyl)phenoxy)-3,4,5-trihydroxytetrahydro-2H-pyran-2-carboxylic acid (2-35e)

To a solution of sat. LiBr (1 mL) in CH₃CN/H₂O (10/1, v v) and triethylamine (0.32 mmol, 45 μL) was added 2-35d (30 mg, 0.016 mmol). The reaction mixture was stirred at r.t. for 1 h under a nitrogen atmosphere. The solvent was removed by evaporation to give crude product as a yellow solid, and then purified by prep-HPLC (Method: column:XBridge Prep C18 OBD 5 μm 19*150 mm; Mobile phase: A—water (0.1% formic acid): B-acetonitrile; Flow rate: 20 mL/min) to provide 2-35e as a white solid after lyophilization (9 mg, 35% yield). MS (ESI) m/z: 1617.4 [M+H]⁺.

### Step 6: (2S,3S,4S,5R,6S)-6-(2-(4-(1-((1R,8S,9s)-bicyclo[6.1.0]non-4-yn-9-yl)-3,19-dioxo-2,7,10,13,16-pentaoxa-4,20-diazahenicosan-21-yl)-1H-1,2,3-triazol-1-yl)-4-((((11S,11aS)-8-((5-(((11S,11aS)-10-(((4-(((2S,3R,4S,5 S,6S)-6-carboxy-3,4,5-trihydroxytetrahydro-2H-pyran-2-yl)oxy)benzyl)oxy)carbonyl)-11-hydroxy-7-methoxy-2-methylene-5-oxo-2,3,5,10,11,11a-hexahydro-11H-benzo[e]pyrrolo[1,2-a][1,4]diazepin-8-yl)oxy)pentyl)oxy)-11 I-hydroxy-7-methoxy-2-methylene-5-oxo-2,3,5,10,11,11a-hexahydro-1H-benzo[e]pyrrolo[1,2-a][1,4]diazepine-10-carbonyl)oxy)methyl)phenoxy)-3,4,5-trihydroxytetrahydro-2H-pyran-2-carboxylic acid (2-35)

To a solution of 2-35e (9 mg, 0.003 mmol) in 0.5 mL DMF was added Int-4 (3.3 mg, 0.011 mmol) and DIPEA (5.4 μL, 0.028 mmol) at 0° C. The reaction solution was stirred at 0° C. for 1 h. The reaction solution was neutralized with AcOH, and then purified by prep-HPLC (Method: column:XBridge Prep C18 OBD 5 μm 19*150 mm; Mobile phase: A—water (0.1% formic acid): B-acetonitrile; Flow rate: 20 mL/min) to provide 2-35 as a white solid after lyophilization (5.3 mg, 53% yield). MS (ESI) m/z: 1792.9 [M+H]⁺.

The foregoing conjugator-linker-payload compounds are summarized below in Table 1.

TABLE 1
Conjugated-linker-payloads

| Com- | |
|---|---|
| pound | |
| No. | Conjugator-Linker-Payload Structure |
| Ref- 2-1 | 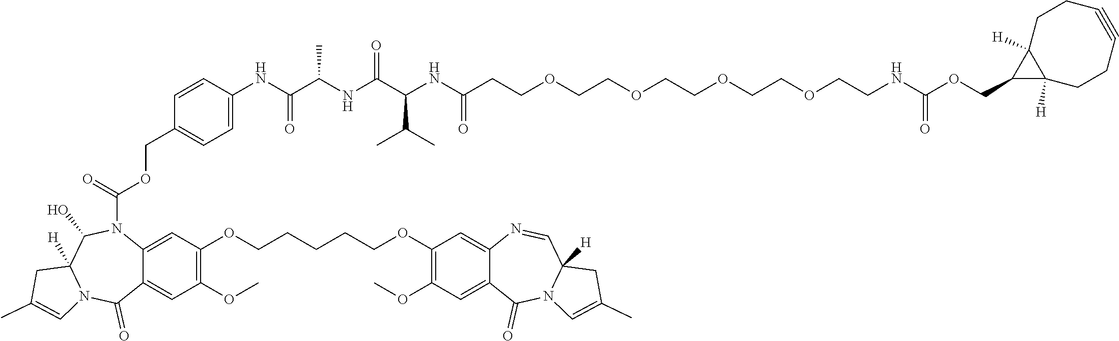 [SMILES: COc1cc2c(cc1OCCCCCOc1cc3c(cc1OC)C(=O)N1C=C(C)C[C@H]1[C@H](O)N3C(=O)OCc1ccc(NC(=O)[C@H](C)NC(=O)[C@@H](NC(=O)CCOCCOCCOCCOCCNC(=O)OC[C@@H]3[C@@H]4CCC#CCC[C@@H]43)C(C)C)cc1)N=C[C@@H]1CC(C)=CN1C2=O] |
| Ref- 2-2 | 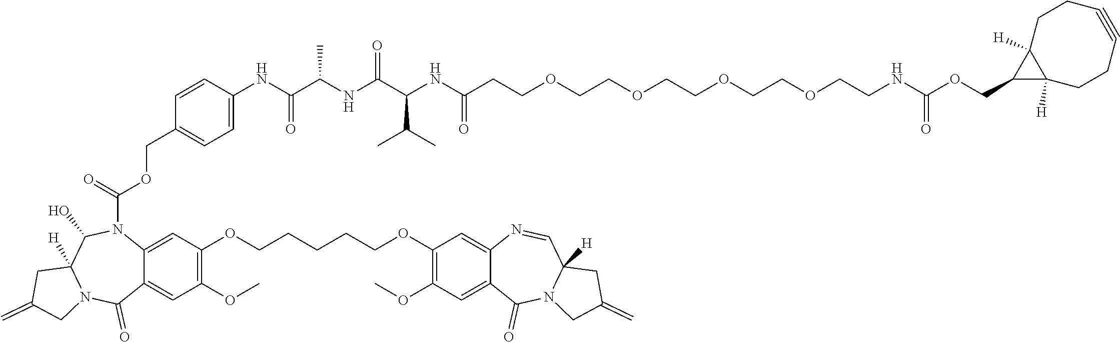 [SMILES: C=C1C[C@H]2C=Nc3cc(OCCCCCOc4cc5c(cc4OC)C(=O)N4CC(=C)C[C@H]4[C@H](O)N5C(=O)OCc4ccc(NC(=O)[C@H](C)NC(=O)[C@@H](NC(=O)CCOCCOCCOCCOCCNC(=O)OC[C@@H]5[C@@H]6CCC#CCC[C@@H]65)C(C)C)cc4)c(OC)cc3C(=O)N2C1] |
| 2-1 | 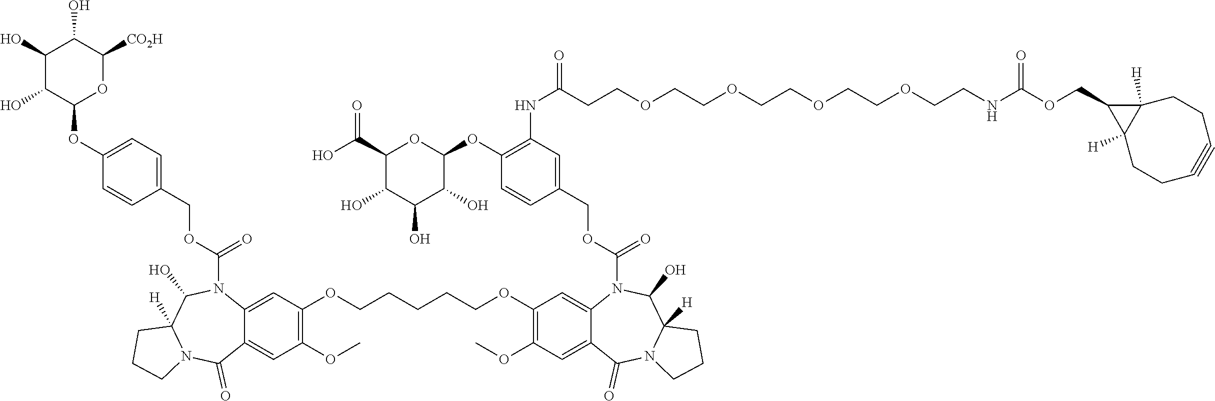 [SMILES: COc1cc2c(cc1OCCCCCOc1cc3c(cc1OC)C(=O)N1CCC[C@H]1[C@H](O)N3C(=O)OCc1ccc(O[C@@H]3O[C@H](C(=O)O)[C@@H](O)[C@H](O)[C@H]3O)c(NC(=O)CCOCCOCCOCCOCCNC(=O)OC[C@@H]3[C@@H]4CCC#CCC[C@@H]43)c1)N(C(=O)OCc1ccc(O[C@@H]3O[C@H](C(=O)O)[C@@H](O)[C@H](O)[C@H]3O)cc1)[C@@H](O)[C@@H]1CCCN1C2=O] |
| 2-2 | 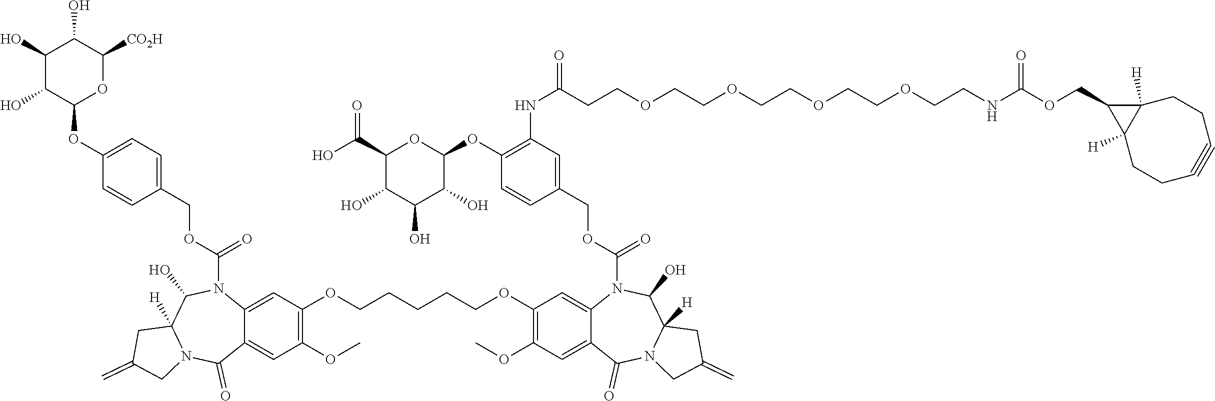 [SMILES: C=C1C[C@H]2[C@H](O)N(C(=O)OCc3ccc(O[C@@H]4O[C@H](C(=O)O)[C@@H](O)[C@H](O)[C@H]4O)cc3)c3cc(OCCCCCOc4cc5c(cc4OC)C(=O)N4CC(=C)C[C@H]4[C@H](O)N5C(=O)OCc4ccc(O[C@@H]5O[C@H](C(=O)O)[C@@H](O)[C@H](O)[C@H]5O)c(NC(=O)CCOCCOCCOCCOCCNC(=O)OC[C@@H]5[C@@H]6CCC#CCC[C@@H]65)c4)c(OC)cc3C(=O)N2C1] |
| 2-4 | 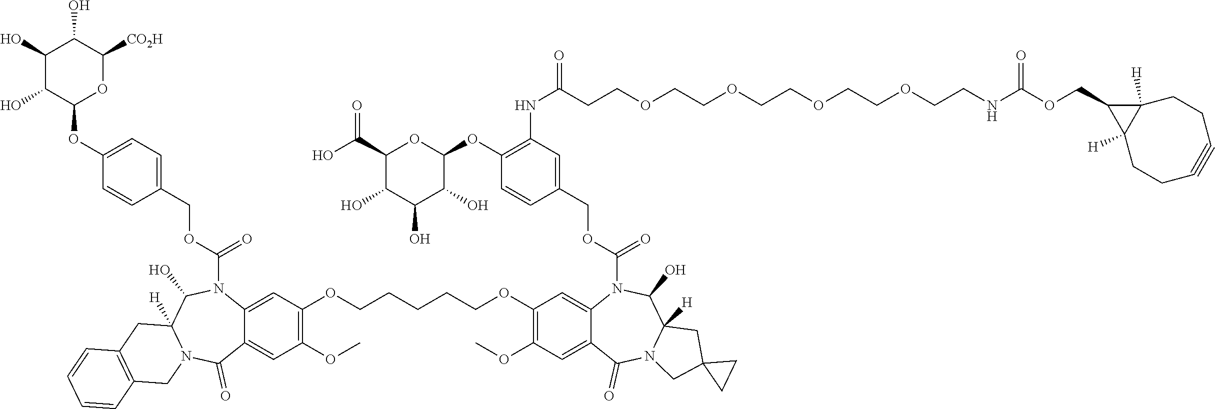 [SMILES: COc1cc2c(cc1OCCCCCOc1cc3c(cc1OC)C(=O)N1CC4(CC4)C[C@H]1[C@H](O)N3C(=O)OCc1ccc(O[C@@H]3O[C@H](C(=O)O)[C@@H](O)[C@H](O)[C@H]3O)c(NC(=O)CCOCCOCCOCCOCCNC(=O)OC[C@@H]3[C@@H]4CCC#CCC[C@@H]43)c1)N(C(=O)OCc1ccc(O[C@@H]3O[C@H](C(=O)O)[C@@H](O)[C@H](O)[C@H]3O)cc1)[C@@H](O)[C@@H]1Cc3ccccc3CN1C2=O] |
| 2-6 | 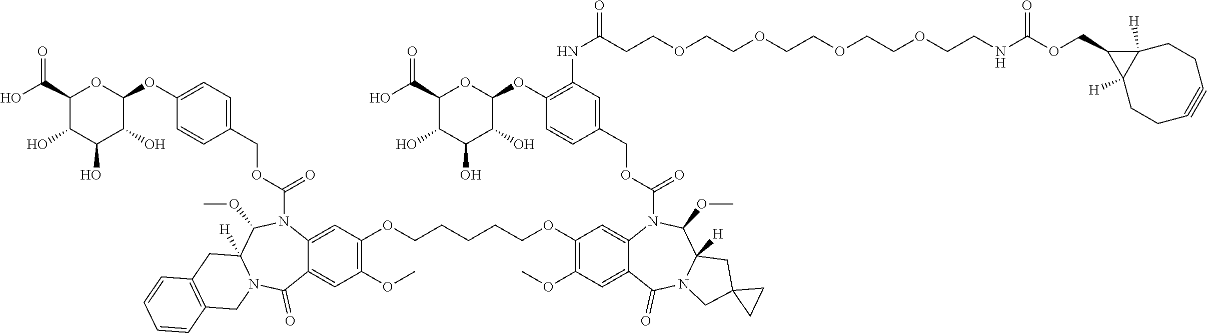 [SMILES: COc1cc2c(cc1OCCCCCOc1cc3c(cc1OC)C(=O)N1CC4(CC4)C[C@H]1[C@H](OC)N3C(=O)OCc1ccc(O[C@@H]3O[C@H](C(=O)O)[C@@H](O)[C@H](O)[C@H]3O)c(NC(=O)CCOCCOCCOCCOCCNC(=O)OC[C@@H]3[C@@H]4CCC#CCC[C@@H]43)c1)N(C(=O)OCc1ccc(O[C@@H]3O[C@H](C(=O)O)[C@@H](O)[C@H](O)[C@H]3O)cc1)[C@@H](OC)[C@@H]1Cc3ccccc3CN1C2=O] |
| 2-7 | 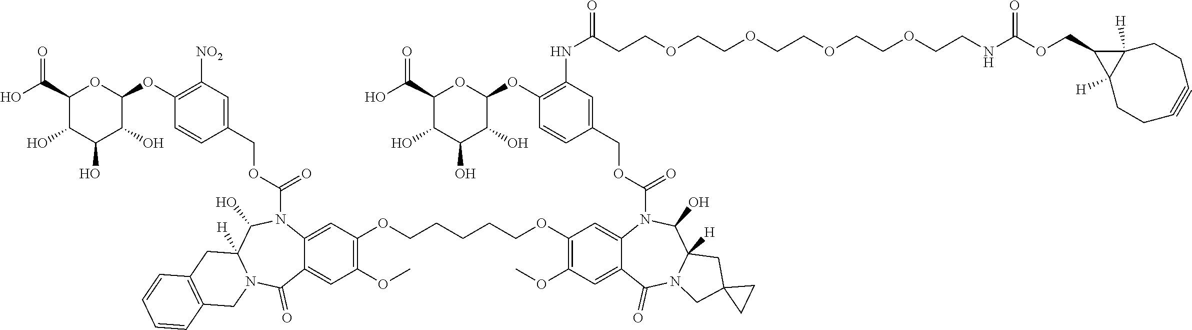 [SMILES: COc1cc2c(cc1OCCCCCOc1cc3c(cc1OC)C(=O)N1CC4(CC4)C[C@H]1[C@H](O)N3C(=O)OCc1ccc(O[C@@H]3O[C@H](C(=O)O)[C@@H](O)[C@H](O)[C@H]3O)c(NC(=O)CCOCCOCCOCCOCCNC(=O)OC[C@@H]3[C@@H]4CCC#CCC[C@@H]43)c1)N(C(=O)OCc1ccc(O[C@@H]3O[C@H](C(=O)O)[C@@H](O)[C@H](O)[C@H]3O)c([N+](=O)[O-])c1)[C@@H](O)[C@@H]1Cc3ccccc3CN1C2=O] |
| 2-8 | 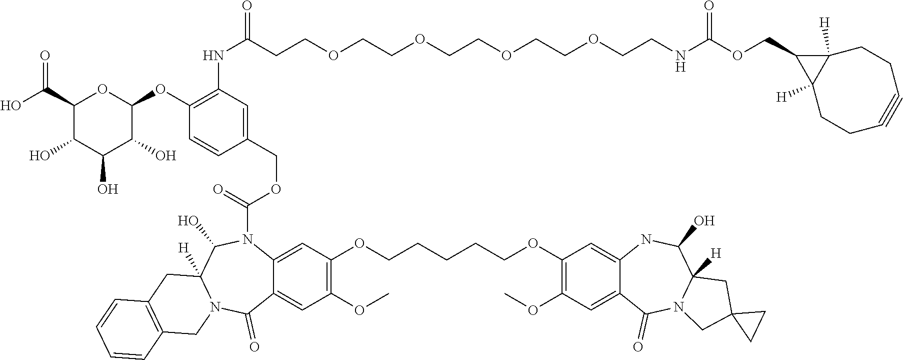 [SMILES: COc1cc2c(cc1OCCCCCOc1cc3c(cc1OC)C(=O)N1Cc4ccccc4C[C@H]1[C@H](O)N3C(=O)OCc1ccc(O[C@@H]3O[C@H](C(=O)O)[C@@H](O)[C@H](O)[C@H]3O)c(NC(=O)CCOCCOCCOCCOCCNC(=O)OC[C@@H]3[C@@H]4CCC#CCC[C@@H]43)c1)N[C@@H](O)[C@@H]1CC3(CC3)CN1C2=O] |
| 2-10 | 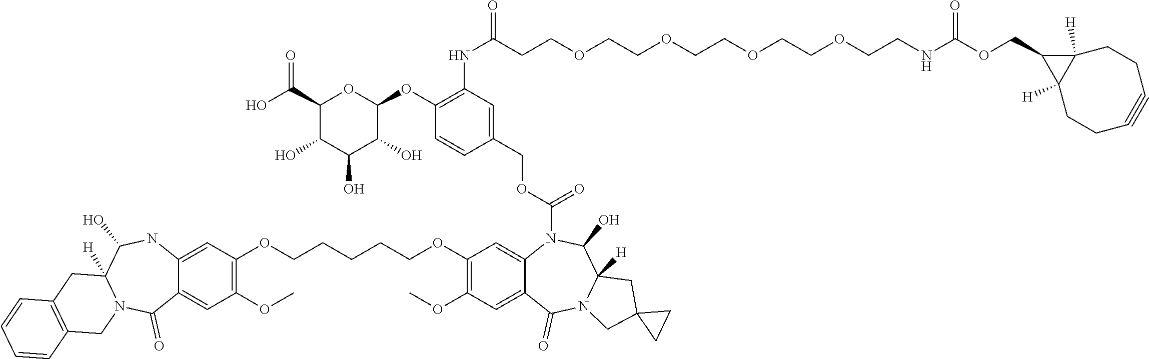 [SMILES: COc1cc2c(cc1OCCCCCOc1cc3c(cc1OC)C(=O)N1CC4(CC4)C[C@H]1[C@H](O)N3C(=O)OCc1ccc(O[C@@H]3O[C@H](C(=O)O)[C@@H](O)[C@H](O)[C@H]3O)c(NC(=O)CCOCCOCCOCCOCCNC(=O)OC[C@@H]3[C@@H]4CCC#CCC[C@@H]43)c1)N[C@@H](O)[C@@H]1Cc3ccccc3CN1C2=O] |
| 2-12 | 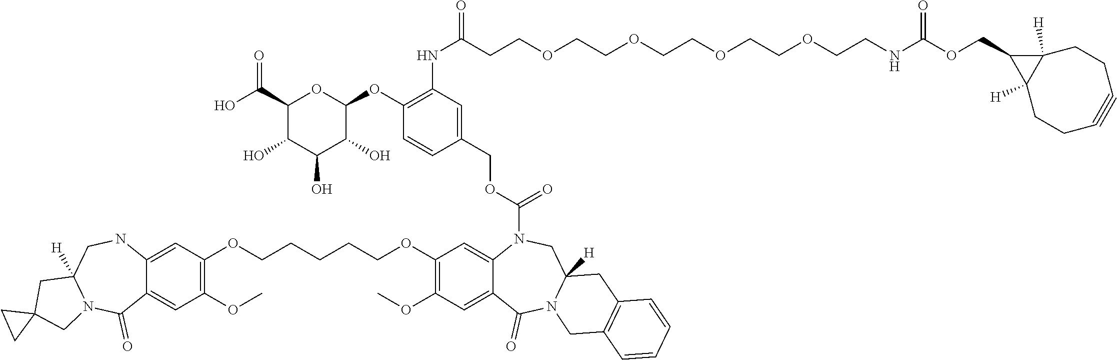 [SMILES: COc1cc2c(cc1OCCCCCOc1cc3c(cc1OC)C(=O)N1Cc4ccccc4C[C@H]1CN3C(=O)OCc1ccc(O[C@@H]3O[C@H](C(=O)O)[C@@H](O)[C@H](O)[C@H]3O)c(NC(=O)CCOCCOCCOCCOCCNC(=O)OC[C@@H]3[C@@H]4CCC#CCC[C@@H]43)c1)NC[C@@H]1CC3(CC3)CN1C2=O] |
| 2-14 | 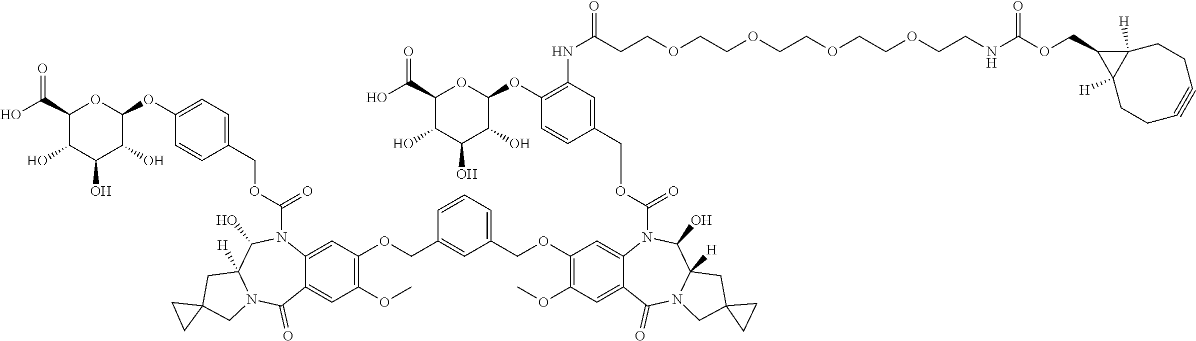 [SMILES: COc1cc2c(cc1OCc1cccc(COc3cc4c(cc3OC)C(=O)N3CC5(CC5)C[C@H]3[C@H](O)N4C(=O)OCc3ccc(O[C@@H]4O[C@H](C(=O)O)[C@@H](O)[C@H](O)[C@H]4O)c(NC(=O)CCOCCOCCOCCOCCNC(=O)OC[C@@H]4[C@@H]5CCC#CCC[C@@H]54)c3)c1)N(C(=O)OCc1ccc(O[C@@H]3O[C@H](C(=O)O)[C@@H](O)[C@H](O)[C@H]3O)cc1)[C@@H](O)[C@@H]1CC3(CC3)CN1C2=O] |
| 2-16 | 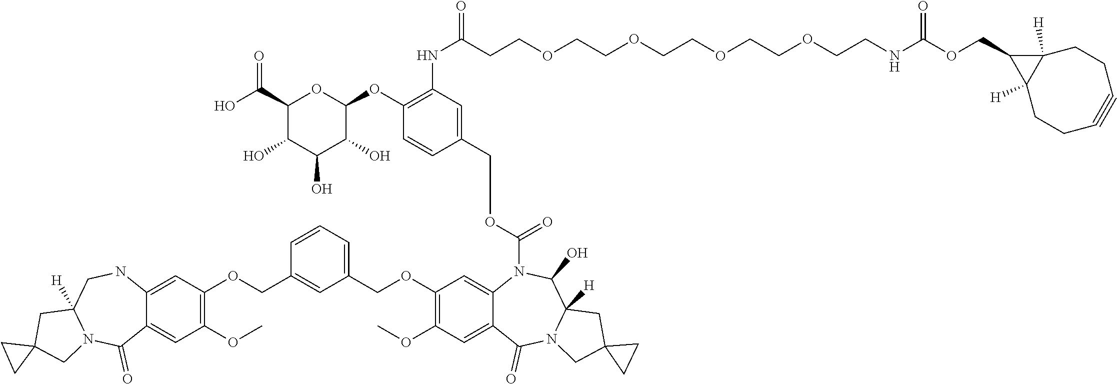 [SMILES: COc1cc2c(cc1OCc1cccc(COc3cc4c(cc3OC)C(=O)N3CC5(CC5)C[C@H]3[C@H](O)N4C(=O)OCc3ccc(O[C@@H]4O[C@H](C(=O)O)[C@@H](O)[C@H](O)[C@H]4O)c(NC(=O)CCOCCOCCOCCOCCNC(=O)OC[C@@H]4[C@@H]5CCC#CCC[C@@H]54)c3)c1)NC[C@@H]1CC3(CC3)CN1C2=O] |
| 2-18 | 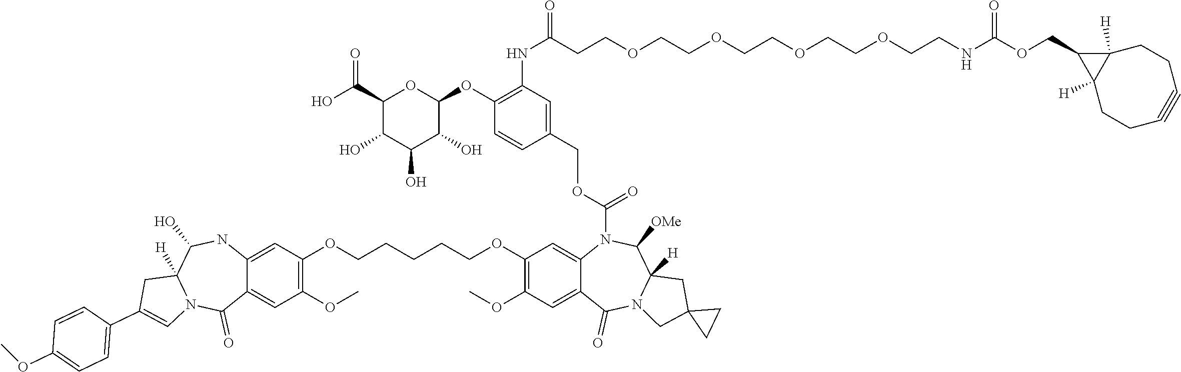 [SMILES: COc1ccc(C2=CN3C(=O)c4cc(OC)c(OCCCCCOc5cc6c(cc5OC)C(=O)N5CC7(CC7)C[C@H]5[C@H](OC)N6C(=O)OCc5ccc(O[C@@H]6O[C@H](C(=O)O)[C@@H](O)[C@H](O)[C@H]6O)c(NC(=O)CCOCCOCCOCCOCCNC(=O)OC[C@@H]6[C@@H]7CCC#CCC[C@@H]76)c5)cc4N[C@@H](O)[C@@H]3C2)cc1] |
| 2-20 | 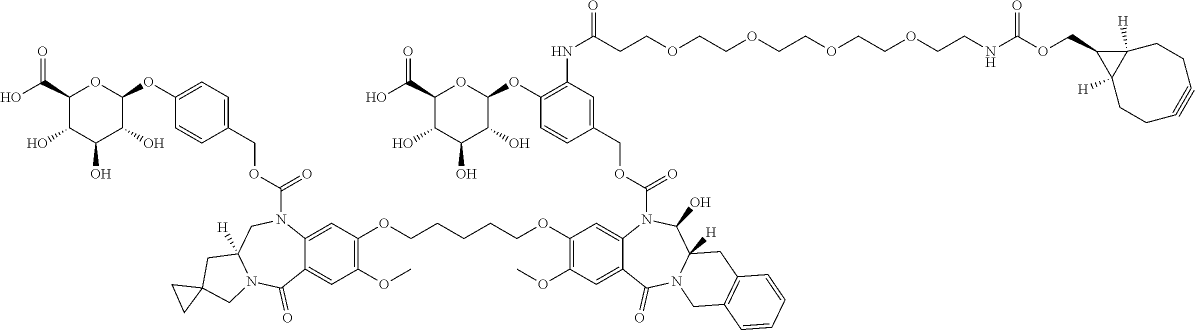 [SMILES: COc1cc2c(cc1OCCCCCOc1cc3c(cc1OC)C(=O)N1Cc4ccccc4C[C@H]1[C@H](O)N3C(=O)OCc1ccc(O[C@@H]3O[C@H](C(=O)O)[C@@H](O)[C@H](O)[C@H]3O)c(NC(=O)CCOCCOCCOCCOCCNC(=O)OC[C@@H]3[C@@H]4CCC#CCC[C@@H]43)c1)N(C(=O)OCc1ccc(O[C@@H]3O[C@H](C(=O)O)[C@@H](O)[C@H](O)[C@H]3O)cc1)C[C@@H]1CC3(CC3)CN1C2=O] |
| 2-22 | 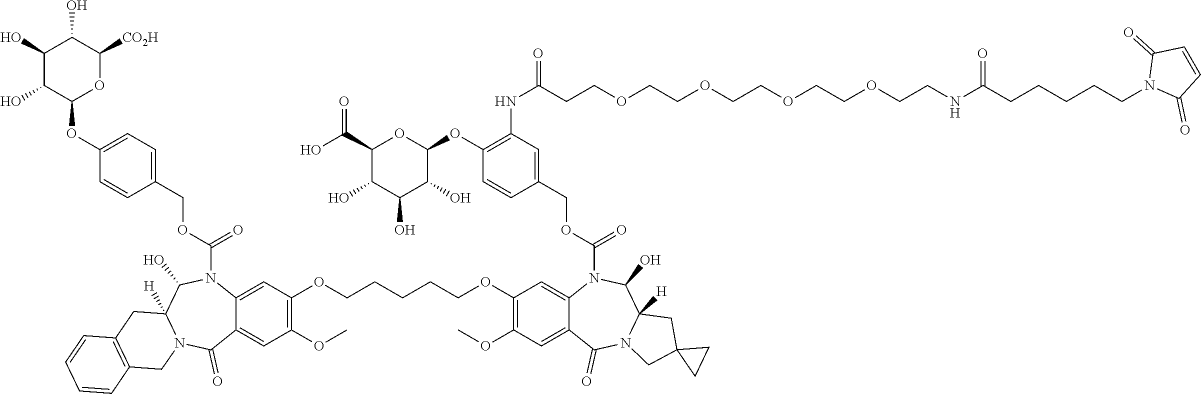 [SMILES: COc1cc2c(cc1OCCCCCOc1cc3c(cc1OC)C(=O)N1CC4(CC4)C[C@H]1[C@H](O)N3C(=O)OCc1ccc(O[C@@H]3O[C@H](C(=O)O)[C@@H](O)[C@H](O)[C@H]3O)c(NC(=O)CCOCCOCCOCCOCCNC(=O)CCCCCN3C(=O)C=CC3=O)c1)N(C(=O)OCc1ccc(O[C@@H]3O[C@H](C(=O)O)[C@@H](O)[C@H](O)[C@H]3O)cc1)[C@@H](O)[C@@H]1Cc3ccccc3CN1C2=O] |
| 2-23 | 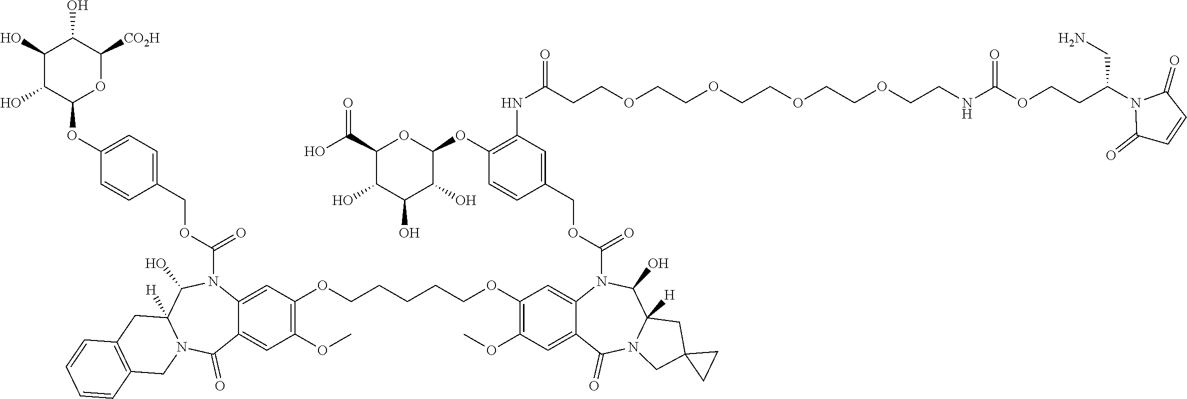 [SMILES: COc1cc2c(cc1OCCCCCOc1cc3c(cc1OC)C(=O)N1CC4(CC4)C[C@H]1[C@H](O)N3C(=O)OCc1ccc(O[C@@H]3O[C@H](C(=O)O)[C@@H](O)[C@H](O)[C@H]3O)c(NC(=O)CCOCCOCCOCCOCCNC(=O)OCC[C@H](CN)N3C(=O)C=CC3=O)c1)N(C(=O)OCc1ccc(O[C@@H]3O[C@H](C(=O)O)[C@@H](O)[C@H](O)[C@H]3O)cc1)[C@@H](O)[C@@H]1Cc3ccccc3CN1C2=O] |
| 2-24 | 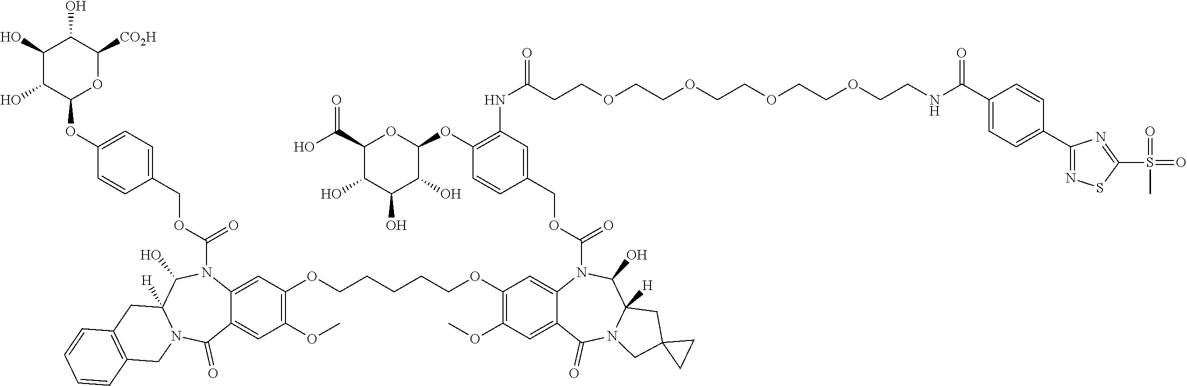 [SMILES: COc1cc2c(cc1OCCCCCOc1cc3c(cc1OC)C(=O)N1CC4(CC4)C[C@H]1[C@H](O)N3C(=O)OCc1ccc(O[C@@H]3O[C@H](C(=O)O)[C@@H](O)[C@H](O)[C@H]3O)c(NC(=O)CCOCCOCCOCCOCCNC(=O)c3ccc(-c4nsc(S(C)(=O)=O)n4)cc3)c1)N(C(=O)OCc1ccc(O[C@@H]3O[C@H](C(=O)O)[C@@H](O)[C@H](O)[C@H]3O)cc1)[C@@H](O)[C@@H]1Cc3ccccc3CN1C2=O] |
| 2-25 | 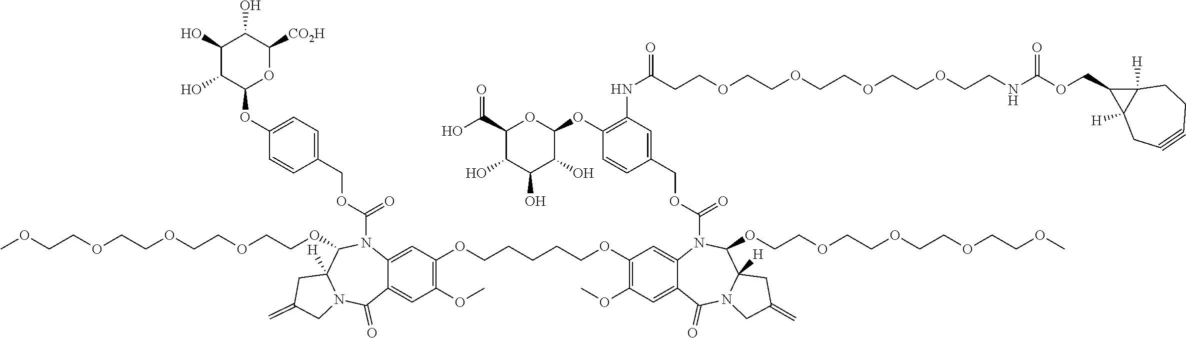 [SMILES: C=C1C[C@H]2[C@H](OCCOCCOCCOCCOC)N(C(=O)OCc3ccc(O[C@@H]4O[C@H](C(=O)O)[C@@H](O)[C@H](O)[C@H]4O)cc3)c3cc(OCCCCCOc4cc5c(cc4OC)C(=O)N4CC(=C)C[C@H]4[C@H](OCCOCCOCCOCCOC)N5C(=O)OCc4ccc(O[C@@H]5O[C@H](C(=O)O)[C@@H](O)[C@H](O)[C@H]5O)c(NC(=O)CCOCCOCCOCCOCCNC(=O)OC[C@@H]5[C@@H]6CC#CCC[C@@H]65)c4)c(OC)cc3C(=O)N2C1] |
| 2-29 | 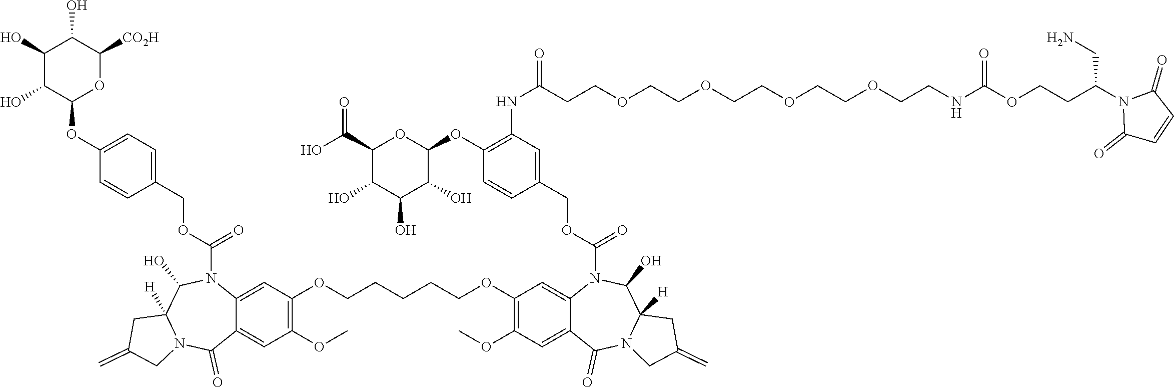 [SMILES: C=C1C[C@H]2[C@H](O)N(C(=O)OCc3ccc(O[C@@H]4O[C@H](C(=O)O)[C@@H](O)[C@H](O)[C@H]4O)cc3)c3cc(OCCCCCOc4cc5c(cc4OC)C(=O)N4CC(=C)C[C@H]4[C@H](O)N5C(=O)OCc4ccc(O[C@@H]5O[C@H](C(=O)O)[C@@H](O)[C@H](O)[C@H]5O)c(NC(=O)CCOCCOCCOCCOCCNC(=O)OCC[C@H](CN)N5C(=O)C=CC5=O)c4)c(OC)cc3C(=O)N2C1] |
| 2-34 | 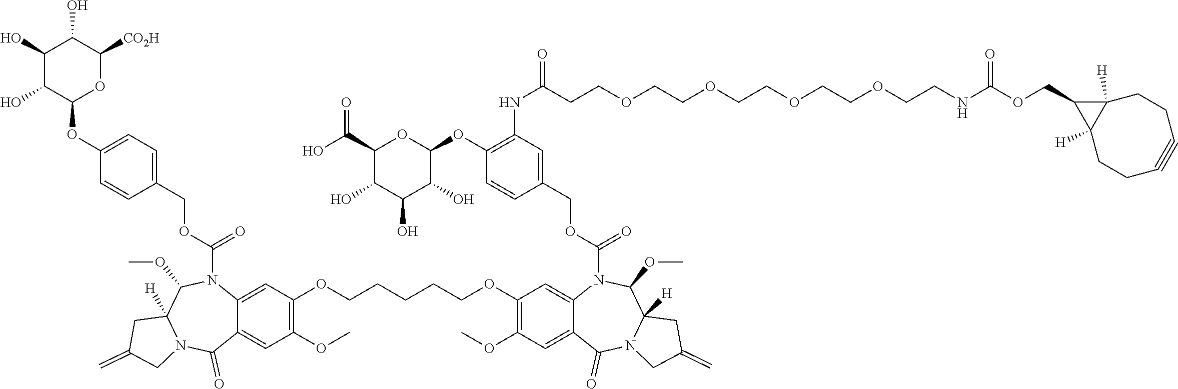 [SMILES: C=C1C[C@H]2[C@H](OC)N(C(=O)OCc3ccc(O[C@@H]4O[C@H](C(=O)O)[C@@H](O)[C@H](O)[C@H]4O)cc3)c3cc(OCCCCCOc4cc5c(cc4OC)C(=O)N4CC(=C)C[C@H]4[C@H](OC)N5C(=O)OCc4ccc(O[C@@H]5O[C@H](C(=O)O)[C@@H](O)[C@H](O)[C@H]5O)c(NC(=O)CCOCCOCCOCCOCCNC(=O)OC[C@@H]5[C@@H]6CCC#CCC[C@@H]65)c4)c(OC)cc3C(=O)N2C1] |
| 2-35 | 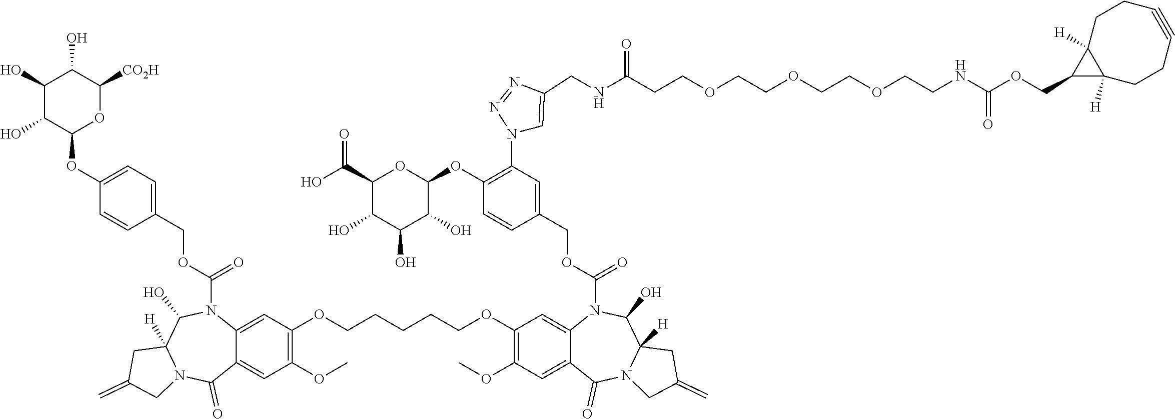 [SMILES: C=C1C[C@H]2[C@H](O)N(C(=O)OCc3ccc(O[C@@H]4O[C@H](C(=O)O)[C@@H](O)[C@H](O)[C@H]4O)cc3)c3cc(OCCCCCOc4cc5c(cc4OC)C(=O)N4CC(=C)C[C@H]4[C@H](O)N5C(=O)OCc4ccc(O[C@@H]5O[C@H](C(=O)O)[C@@H](O)[C@H](O)[C@H]5O)c(-n5cc(CNC(=O)CCOCCOCCOCCNC(=O)OC[C@@H]6[C@@H]7CCC#CCC[C@@H]76)nn5)c4)c(OC)cc3C(=O)N2C1] |

### Example 2: Antibody Drug Conjugate Preparation and Characterization

DAR2 antibody drug conjugate preparation. Anti-CD74 antibody STRO-001 or isotype antibody CB6 (either, “mAb1”) in reaction buffer (with concentration 0.5-25 mg/mL, 50 mM Tris-HCl buffer pH 7.0-8.5) was incubated with 1/2000-1/500 w/w (EndoS2/mAb weight ratio) endoS2 under reaction temperature (0-40° C.) for 1-24 h. 2-40 eq. UDP-GalNAz (20 mM) and 0.1 w/w %-10 w/w % (GalT/mAb weight ratio) β1,4-GalT were added into the reaction mixture and incubated in reaction buffer (50 mM Tris-HCl buffer pH 7.0-8.5, 20 mM MnCl₂) for 8-24 hours at reaction temperature (0-40° C.). The reaction mixture was purified with protein A resin to give the mAb1-GalNAz.

Organic solvent (e.g., DMSO, DMF, DMA, PG, acetonitrile, 0-25% v/v) and linker-payload stock (10-25 eq., 10 mM stock in organic solvent) were added stepwise in reaction buffer (PBS buffer pH 7.0-8.5) with mAb1-GalNAz (1-20 mg/mL) at 0-25° C. for 0.5-24 h. The solution was submitted to buffer exchange (spin desalting column, ultrafiltration, and dialysis) into storage buffer (for example, pH 5.5-6.5 histidine acetate buffer, with optional additive such as sucrose, trehalose, tween 20, 60, 80).

Synthesized ADCs are shown below in Table 2. Ab was STRO-001 for all ADCs except Isotype ADC, in which Ab was CB6.

ADC characterization. ADCs were characterized via the following analytical methods. SEC purity of all ADCs were >95% purity.

### Drug to Antibody Ratio (DAR) Determination by LCMS or HIC Methods

LCMS method: LC-MS analysis was carried out under the following measurement conditions:
LC-MS system: Vanquish Flex UHPLC and Orbitrap Exploris 240 Mass Spectrometer
Column: MAbPac™ RP, 2.1*50 mm, 4 μm, 1,500 Å, Thermo Scientific™
Column temperature: 80° C.
Mobile phase A: 0.1% formic acid (FA) aqueous solution
Mobile phase B: Acetonitrile solution containing 0.1% formic acid (FA)
Gradient program: 25% B-25% B (0 min-2 min), 25% B-50% B (2 min-18 min), 50% B-90% B (18 min-18.1 min), 90% B-90% B (18.1 min-20 min), 90% B-25% B (20 min-20.1 min), 25% B-25% B (20.1 min-25 min)
Injected sample amount: 1 μg
MS parameters: Intact and denaturing MS data were acquired in HMR mode at setting of R=15 k and deconvolved using the ReSpect™ algorithm and Sliding Window integration in Thermo Scientific™ BioPharma Finder™ 4.0 software.

HIC method: HPLC analysis was carried out under the following measurement conditions:
HPLC system: Waters ACQUITY ARC HPLC System
Detector: measurement wavelength: 280 nm
Column: Tosoh Bioscience 4.6 μm ID×3.5 cm, 2.5 μm butyl-nonporous resin column
Column temperature: 25° C.
Mobile phase A: 1.5 M ammonium sulfate, 50 mM phosphate buffer, pH 7.0
Mobile phase B: 50 mM phosphate buffer, 25% (V/V) isopropanol, pH 7.0
Gradient program: 0% B-0% B (0 min-2 min), 0% B-100% B (2 min-15 min), 100% B-100% B (15 min-16 min), 100% B-0% B (16 min-17 min), 0% B-0% B (17 min-20 min)
Injected sample amount: 20 μg

### SEC Method to Determine ADC Purity

HPLC analysis was carried out under the following measurement conditions:
HPLC system: Waters H-Class UPLC System
Detector: measurement wavelength: 280 nm
Column: ACQUITY UPLC BEH200 SEC 1.7 um 4.6×150 mm, Waters
Column temperature: room temperature
Mobile phase A: 200 mM phosphate buffer, 250 mM potassium chloride, 15% isopropyl alcohol, pH 7.0
Gradient program: under 10 min isocratic elutions with a flow rate of 0.3 mL/min
Injected sample amount: 20 μg

ADC hydrophobicity evaluation by HIC method: An ADC with a higher hydrophobic property would appear with a later retention time from HIC (hydrophobicity interaction column) chromatography. The DAR2 peak was used as a reference.

HPLC analysis was carried out under the following measurement conditions:

### Method 1

HPLC system: Waters ACQUITY ARC HPLC System
Detector: measurement wavelength: 280 nm
Column: Tosoh Bioscience 4.6 μm ID×3.5 cm, 2.5 μm butyl-nonporous resin column
Column temperature: 25° C.
Mobile phase A: 1.5 M ammonium sulfate, 50 mM phosphate buffer, pH 7.0
Mobile phase B: 50 mM Phosphate buffer, 25% (V/V) isopropanol, pH 7.0
Gradient program: 0% B-0% B (0 min-2 min), 0% B-100% B (2 min-15 min), 100% B-100% B (15 min-16 min), 100% B-0% B (16 min-17 min), 0% B-0% B (17 min-20 min)
Injected sample amount: 20 μg

### Method 2

HPLC system: Waters ACQUITY ARC HPLC System
Detector: measurement wavelength: 280 nm
Column: MABPac HIC-10, 5 μm, 4.6×10 mm (Thermo)
Column temperature: 25° C.
Mobile phase A: 1.5 M ammonium sulfate, 50 mM sodium phosphate, pH 7.0
Mobile phase B: 50 mM sodium phosphate, pH 7.0
Gradient program: 20% B-20% B (0 min-1 min), 0% B-0% B (1 min-35 min), 20% B-20% B (35 min-40 min)
Flow rate: 0.5 mL/min

Sample preparation: The sample was diluted with initial mobile phase to 0.5 mg/mL.

TABLE 2
ADC Structures

| ADC | |
|---|---|
| No. | ADC Structure |
| ADC3- A | 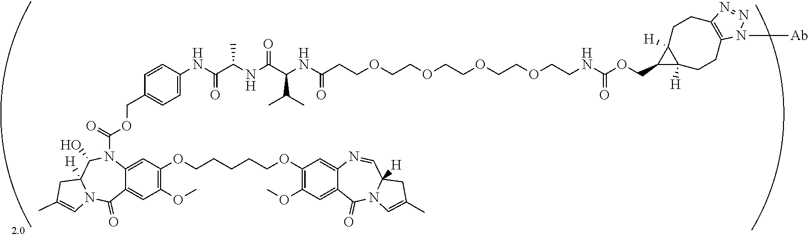 |
| ADC3- B | 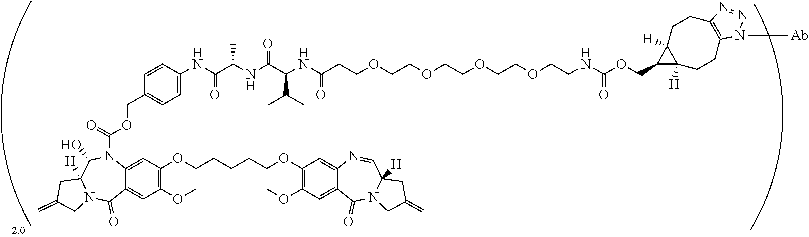 |
| ADC3- 1 | 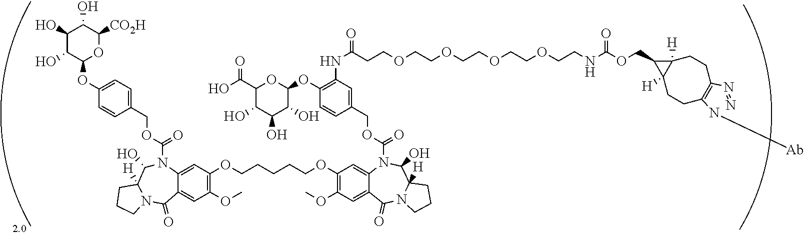 |
| ADC3- 2 | 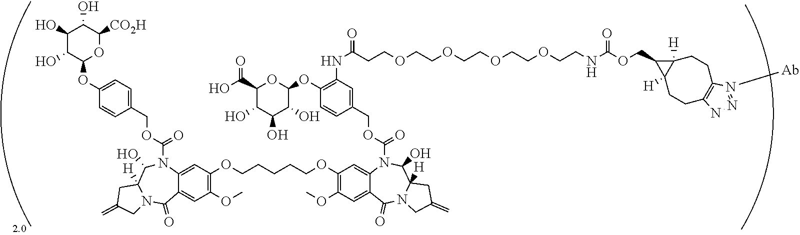 |
| ADC3- 3 | 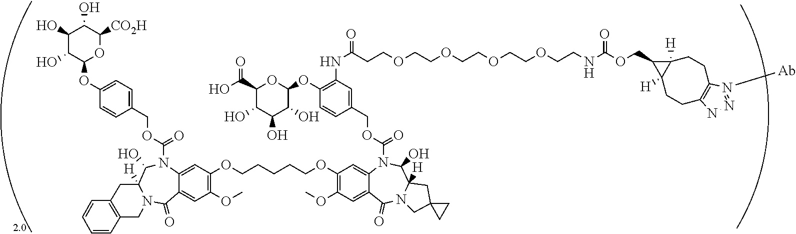 |
| ADC3- 4 | 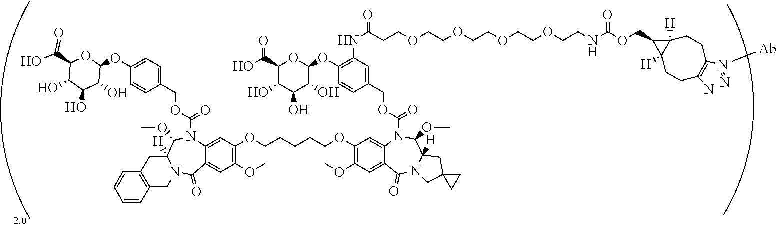 |
| ADC3- 5 | 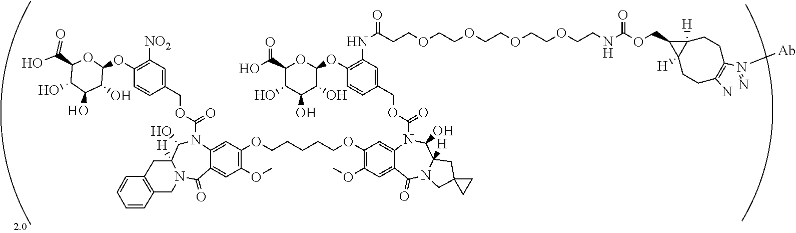 |
| ADC3- 6 | 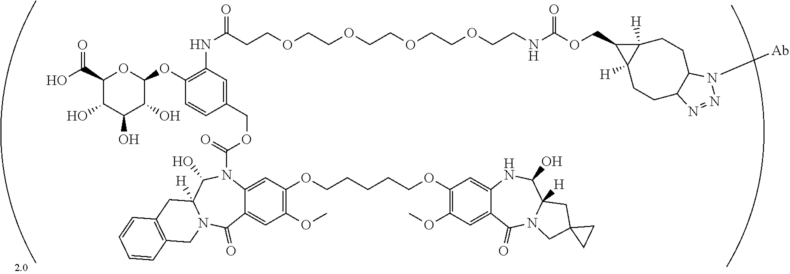 |
| ADC3- 7 | 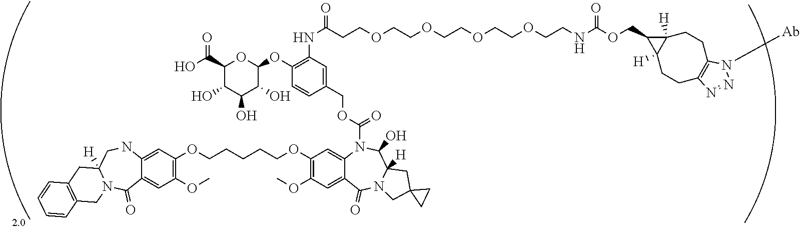 |
| ADC3- 8 | 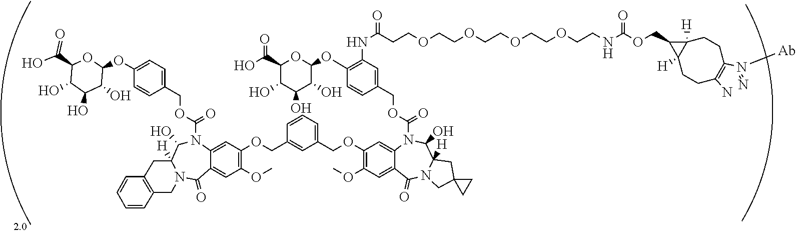 |
| ADC3- 9 | 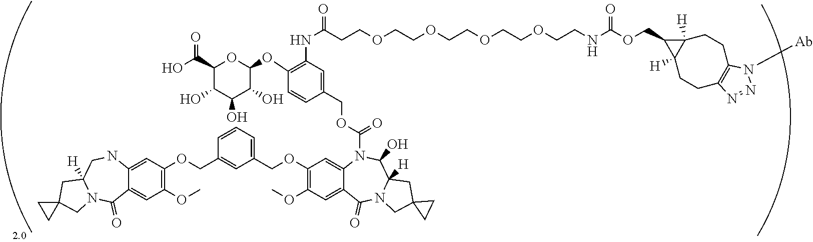 |
| ADC3- 10 | 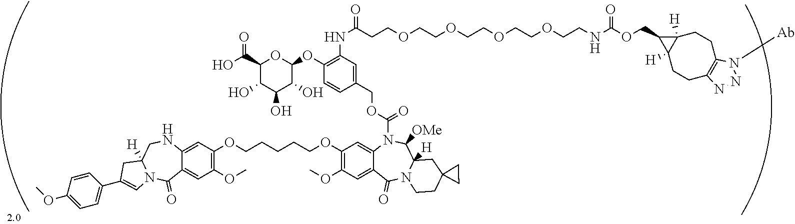 |
| ADC3- 11 | 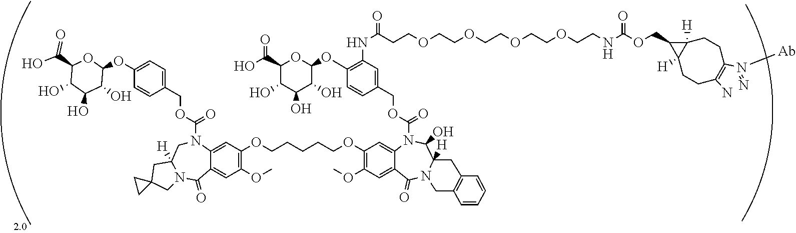 |
| ADC3- 12 | 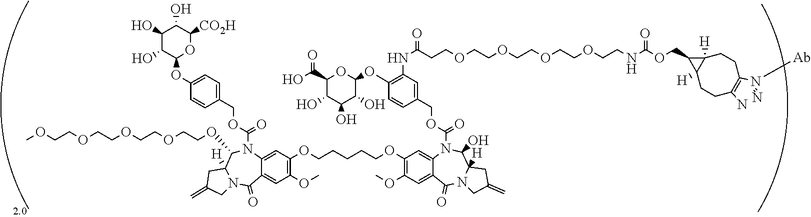 |
| ADC3- 13 | 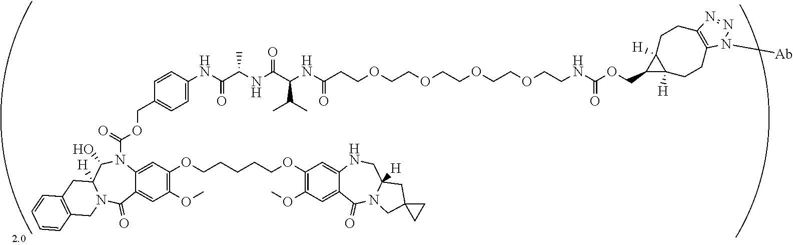 |
| ADC3- 14 | 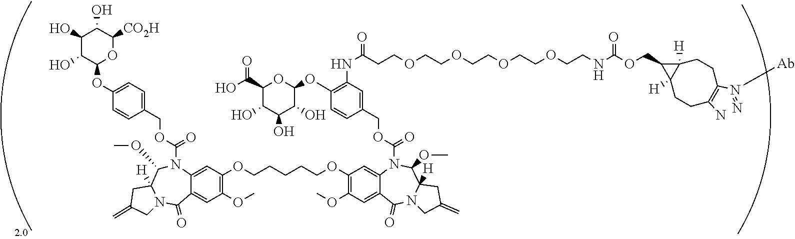 |
| ADC3- 15 | 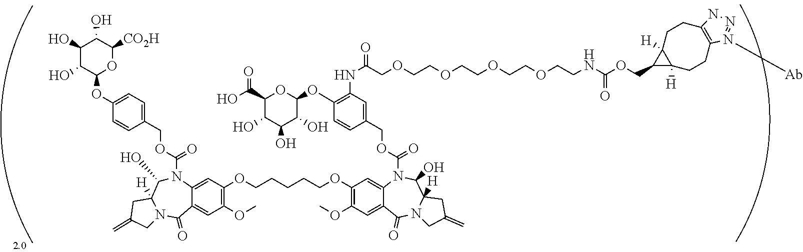 |
| Isotype ADC | 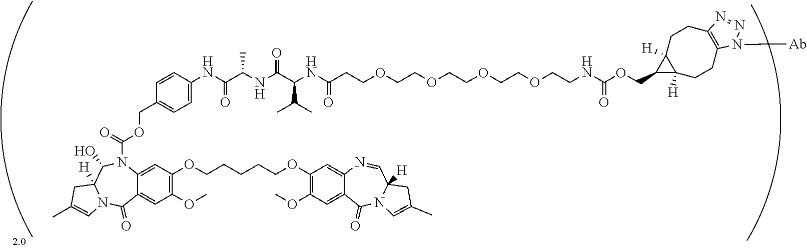 |

CD74 antibody STRO-001

Light chain sequence
(SEQ ID NO: 1)
DIQMTQSPSSVSASVGDRVTITCRASQGIGSWLAWYQQKPGKAPKLLIY
AADRLQSGVPSRFSGSGSGTDFTLTISSLQPEDFATYYCQQYHTYPLTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACE
VTHQGLSSPVTKSFNRGEC
Heavy chain sequence
(SEQ ID NO: 2)
QVQLVESGGGVVQPGRSLRLSCAASGFNFSDYGMHWVRQAPGKGLEWVA
VIWYDGSISYYADSVKGRFTISRDNSKNTLYLQMNSLRAEDTAVYYCAR
GGTVEHGAVYGTDVWGQGTTVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNH
YTQKSLSLSPGK
Anti-SARS-CoV-2 antibody CB6 (isotype antibody)
Light chain sequence
(SEQ ID NO: 3)
DIVMTQSPSSLSASVGDRVTITCRASQSISRYLNWYQQKPGKAPKLLIY
AASSLQSGVPSRFSGSGSGTDFTLTISSLQPEDFATYYCQQSYSTPPEY
TFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYAC
EVTHQGLSSPVTKSFNRGEC
Heavy chain sequence
(SEQ ID NO: 4)
EVQLVESGGGLVQPGGSLRLSCAASGFTVSSNYMSWVRQAPGKGLEWVS
VIYSGGSTFYADSVKGRFTISRDNSMNTLFLQMNSLRAEDTAVYYCARV
LPMYGDYLDYWGQGTLVTVSSASTKGPSVFPLAPSSKSTSGGTAALGCL
VKDYFPEPVTVSWNSGALTSGVHTFPAVLQSSGLYSLSSVVTVPSSSLG
TQTYICNVNHKPSNTKVDKKVEPKSCDKTHTCPPCPAPPAAGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALAAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPGK

### Example 3: ADC Direct Killing in NOMO-1 and K562 Cancer Cell Lines

### Cell Lines

NOMO-1 (JCRB, IF050474). NOMO-1 (purchased from JCRB) is a cell line exhibiting hemo-lymphocytic morphology. The base medium is ATCC-formulated RPMI-1640 Medium, Catalog No. ATCC 30-2001. To make the complete growth medium, fetal bovine serum was added to a final concentration of 10% (Gibco, 10099-141C). The cell line was grown in a humidified 5% C02 atmosphere at 37° C., and was regularly tested for the presence of Mycoplasma with MycoAlert™ PLUS Mycoplasma Detection Kit (Lonza, LT07-710). NOMO-1 is a CD74-positive cell line (Table 3).

K562 (ATCC, CCL-243). K562 (purchased from ATCC) is a cell line exhibiting lymphoblast morphology. The base medium for K562 is ATCC-formulated Iscove's Modified Dulbecco's Medium, Catalog No. 30-2005. To make the complete growth medium, fetal bovine serum was added to a final concentration of 10% (Gibco, 10099-141C). The cell line was grown in a humidified 5% CO₂ atmosphere at 37° C., and was regularly tested for the presence of mycoplasma with MycoAlert™ PLUS Mycoplasma Detection Kit (Lonza, LT07-710). K562 is a CD74-negative cell line (Table 3).

TABLE 3
CD74 expression level

| | Molecular No. |
|---|---|
| Cell line | (QSC kit) |
| NOMO-1 | 18929 |
| K562 | 982 |

ADC direct killing. ADC direct killing was assessed in NOMO-1(CD74⁺) and K562 (CD74⁻) cancer lines. Cells were seeded (NOMO-1 at 6E3/well and K562 at 3E3/well) into 96-well plates (Greiner: 655090), 100 μl/well (including 100 μg/ml Fc blocker for NOMO-1 cells), and incubated at 37° C., 5% CO₂, for 2 h. Fresh growth medium was added containing varying concentrations of ADCs, 50 μl/well, and incubated at 37° C., 5% CO₂, for 6 days. The cell viability was detected by Cell Titer-Glo (Promega, G7573), 70 μl/well. The plates were allowed to incubate at room temperature for 10 minutes to stabilize the luminescent signal. The plates were analyzed with a microplate reader.

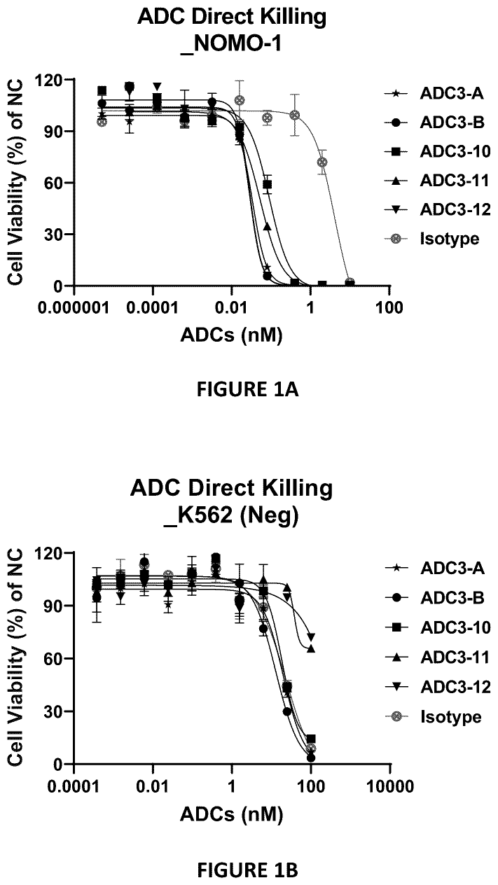

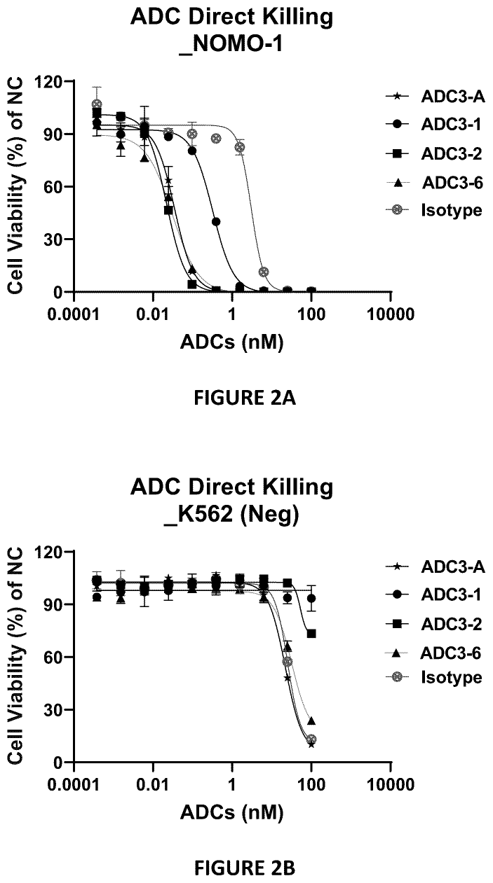

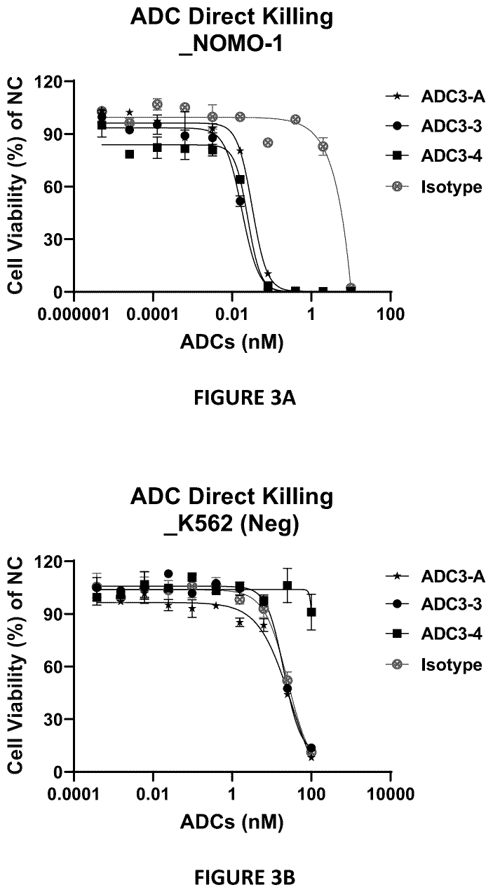

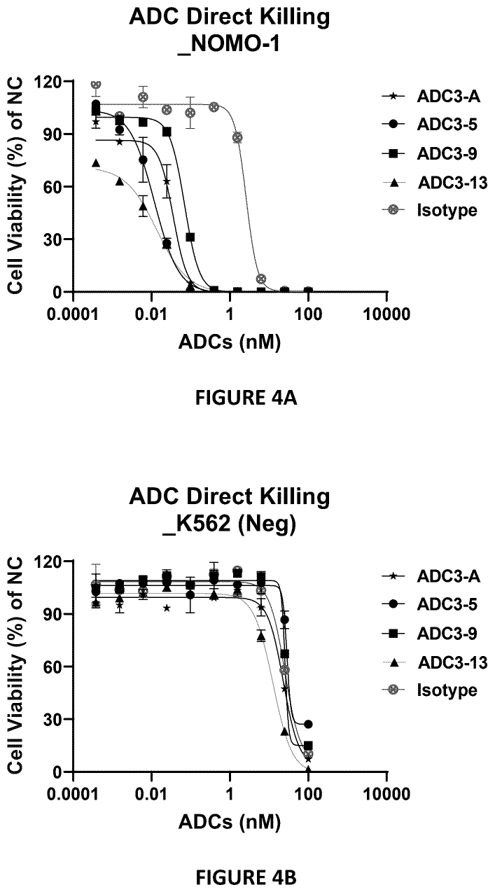

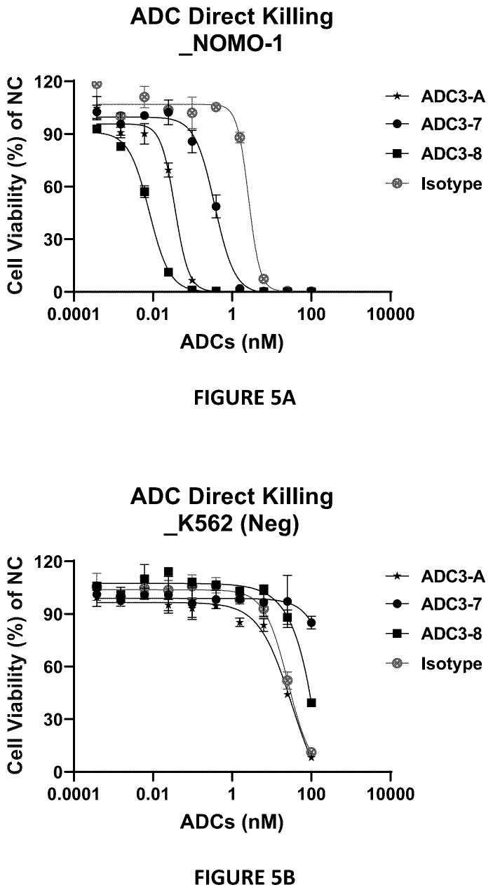

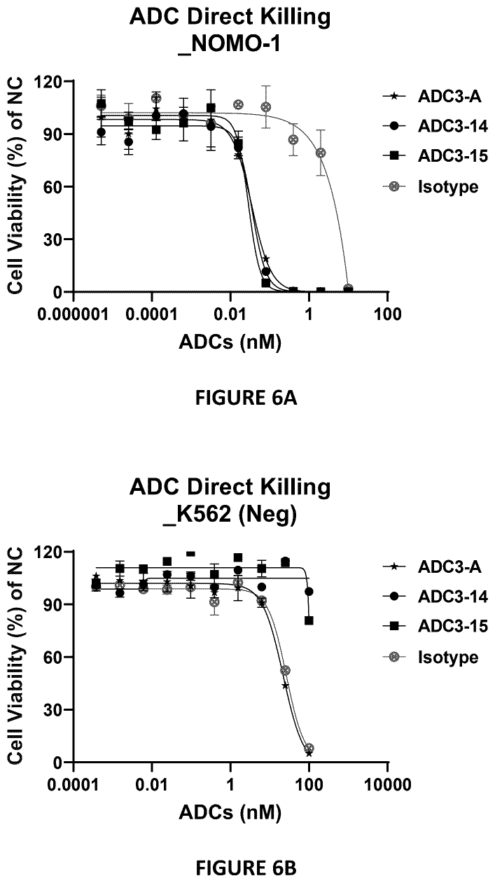

ADC direct cellular killing data are presented in Tables 4 to 9 and FIGS. 1A to 6B.

TABLE 4
ADC direct cellular killing

| | |
|---|---|
| NOMO-1 | K562 |

| | Emax | EC50 | Emax | EC50 |
|---|---|---|---|---|
| ADC | (%) | (nM) | (%) | (nM) |
| ADC3-A | 99.9 | 0.033 | 93.2 | 18.2 |
| ADC3-B | 99.9 | 0.030 | 96.4 | 13.3 |
| ADC3-10 | 99.7 | 0.094 | 85.7 | 19.4 |
| ADC3-11 | 99.9 | 0.053 | 34.2 | >100 |
| ADC3-12 | 99.8 | 0.030 | 28.1 | >100 |
| Isotype ADC | 98.1 | 3.004 | 91.2 | 19.3 |

TABLE 5
ADC direct cellular killing

| | |
|---|---|
| NOMO-1 | K562 |

| | Emax | EC50 | Emax | EC50 |
|---|---|---|---|---|
| ADC | (%) | (nM) | (%) | (nM) |
| ADC3-A | 100.0 | 0.033 | 89.8 | 21.7 |
| ADC3-1 | 99.9 | 0.291 | 6.6 | >100 |
| ADC3-2 | 100.0 | 0.022 | 26.6 | >100 |
| ADC3-6 | 100.0 | 0.025 | 76.1 | 27.9 |
| Isotype ADC | 99.9 | 2.930 | 87.0 | 25.3 |

TABLE 6
ADC direct cellular killing

| | |
|---|---|
| NOMO-1 | K562 |

| | Emax | EC50 | Emax | EC50 |
|---|---|---|---|---|
| ADC | (%) | (nM) | (%) | (nM) |
| ADC3-A | 99.9 | 0.031 | 91.9 | 18.9 |
| ADC3-3 | 99.9 | 0.016 | 86.4 | 20.8 |
| ADC3-4 | 99.8 | 0.021 | 9.0 | >100 |
| Isotype ADC | 97.8 | 5.217 | 88.9 | 22.9 |

TABLE 7
ADC direct cellular killing

| | |
|---|---|
| NOMO-1 | K562 |

| | Emax | EC50 | Emax | EC50 |
|---|---|---|---|---|
| ADC | (%) | (nM) | (%) | (nM) |
| ADC3-A | 100.0 | 0.031 | 92.7 | 22.2 |
| ADC3-5 | 100.0 | 0.012 | 72.8 | 29.3 |
| ADC3-9 | 100.0 | 0.069 | 84.9 | 26.1 |
| ADC3-13 | 100.0 | 0.006 | 98.7 | 12.5 |
| Isotype ADC | 99.9 | 2.719 | 89.9 | 26.3 |

TABLE 8
ADC direct cellular killing

| | |
|---|---|
| NOMO-1 | K562 |

| | Emax | EC50 | Emax | EC50 |
|---|---|---|---|---|
| ADC | (%) | (nM) | (%) | (nM) |
| ADC3-A | 100.0 | 0.034 | 91.9 | 18.9 |
| ADC3-7 | 99.9 | 0.361 | 14.9 | >100 |
| ADC3-8 | 100.0 | 0.007 | 60.6 | 47.2 |
| Isotype ADC | 99.9 | 2.741 | 88.9 | 22.9 |

TABLE 9
ADC direct cellular killing

| | |
|---|---|
| NOMO-1 | K562 |

| | Emax | EC50 | Emax | EC50 |
|---|---|---|---|---|
| ADC | (%) | (nM) | (%) | (nM) |
| ADC3-A | 99.9 | 0.034 | 94.9 | 20.5 |
| ADC3-14 | 99.9 | 0.033 | 2.7 | >100 |
| ADC3-15 | 99.9 | 0.029 | 19.1 | >100 |
| Isotype ADC | 98.2 | 4.473 | 92.0 | 24.1 |

Although the foregoing disclosure has been presented in some detail by way of illustration and example for purposes of clarity of understanding, it is apparent to those skilled in the art that certain minor changes and modifications will be practiced. Therefore, the description and examples should not be construed as limiting.

It is to be understood that, if any publication is referred to herein, such reference does not constitute an admission that the publication forms a part of the common general knowledge in the art in any country.

The disclosures of all non-patent publications, patents, patent applications, and published patent applications referred to herein are hereby incorporated herein by reference in their entireties.

What is claimed is:

1. A compound of Formula (I): 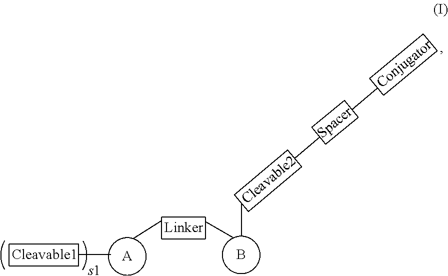
or a pharmaceutically acceptable salt, tautomer, solvate, stereoisomer, or isotopologue thereof, wherein:
each of ring A and ring B is, independently, one of formula (IIa), (IIb), (IIc), (IId), (IIe), (IIf), or (IIg): 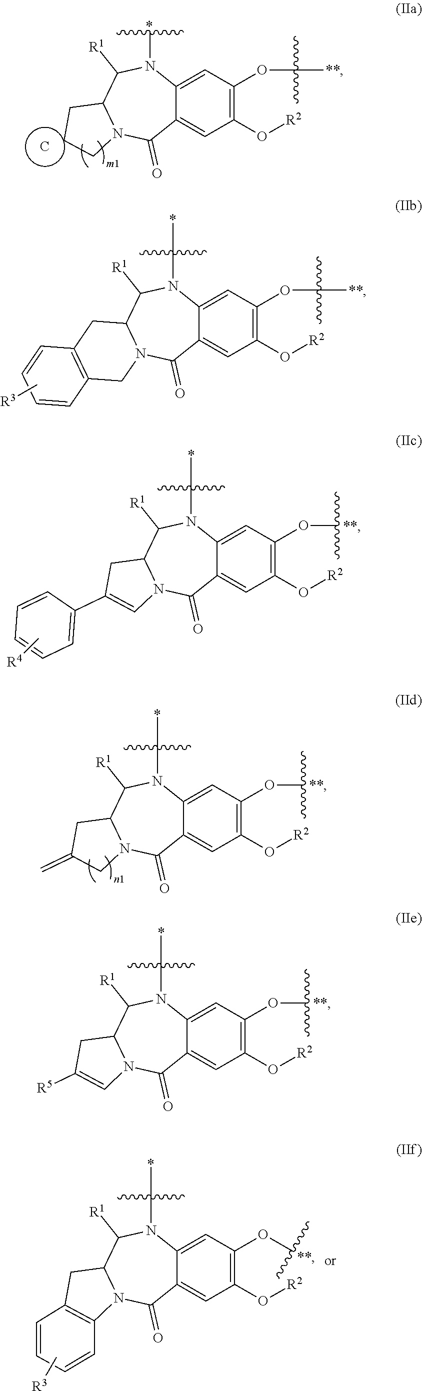 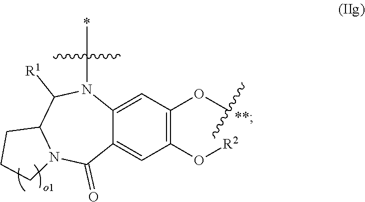
wherein:
each ring C is independently a cyclopropyl ring or a cyclobutyl ring;
each R¹ is independently H, OH, C₁₋₄ alkyl, C₁₋₄ alkoxyl, or —O—(CH₂CH₂O)ₜ₁—CH₃;
each R² is independently H or C₁₋₄ alkyl;
each R³ and R⁴ is, independently, H, NR^{a1}R^{b1}, OH, C₁₋₄ alkyl, C₁₋₄ alkoxyl, or aryl;
each of R^{a1} and R^{b1} is, independently, H or C₁₋₄ alkyl;
each R⁵ is independently H, C₁₋₄ alkyl, C₁₋₄ alkoxyl, or aryl;
each m1, n1, and o1 is, independently, 1 or 2;
t1 is 1, 2, 3, 4, 5, 6, 7, or 8;
marks the bond where ring A connects to Cleavable1 when s1 is 1 or to —H when s1 is 0, or where ring B connects to Cleavable2; and
** marks the bond where ring A or ring B connects to Linker;
Linker is —(CH₂)ᵣ—, —(CH₂)ₚ—X—(CH₂)_{q}—, or —(CH₂)ₚ—CH═CH—(CH₂)_{q}—; wherein:
each r, p, and q, is, independently, 1, 2, 3, 4, 5, 6, 7, or 8;
the sum of p and q is 2, 3, 4, 5, 6, 7, or 8;
X is NR⁶, NHC(═O), C(═O)NH, 0, S02, a substituted or unsubstituted aryl ring, substituted or unsubstituted heteroaryl ring, substituted or unsubstituted heterocyclic ring, or substituted or unsubstituted cyclic ring; and
R⁶ is H or C₁₋₄ alkyl;
Cleavable1, if present, has formula (VIa) or (VIc): 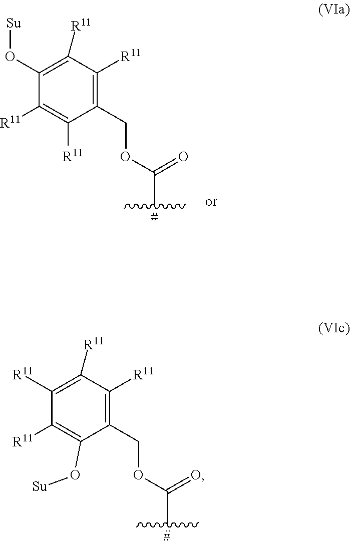
wherein:
Su is a sugar moiety;
each R¹¹ is, independently, hydrogen, halogen, substituted or unsubstituted C₁₋₄ alkyl, —CN, or —NO₂; and
# marks the bond where Cleavable1 connects to ring A;
s1 is 0 or 1;
Cleavable2 has formula (VIIa1), (VIIc1, (VIId1), or (VIIf1): 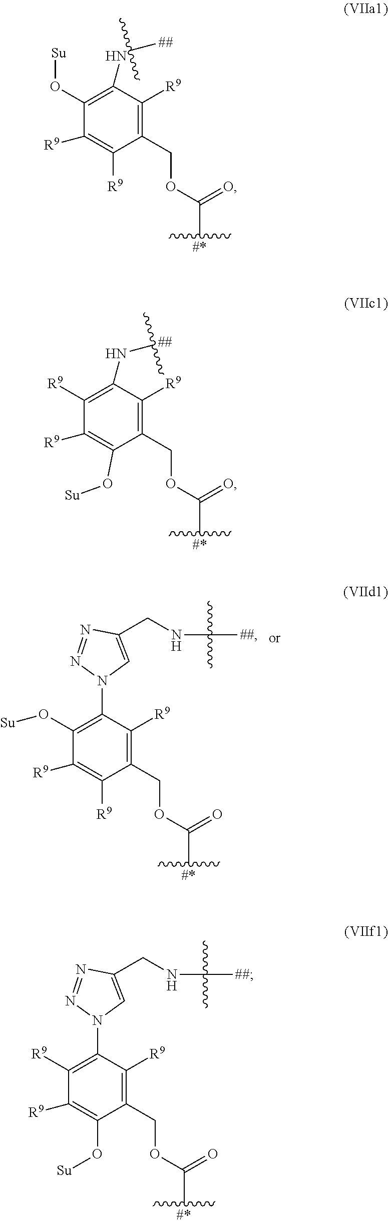
wherein:
Su is a sugar moiety;
each R⁹ is, independently, hydrogen, halogen, substituted or unsubstituted C₁₋₄ alkyl, —CN, or —NO₂;
#* marks the bond where Cleavable2 connects to ring B; and
## marks the bond where Cleavable2 connects to Spacer;
Spacer is a bond, ^{###}—NH—(CH₂CH₂O)ₘ₂—CH₂CH₂—C(═O)—, ^{###}—NH—(CH₂CH₂O)ₘ₂—CH₂—C(═O)—, ^{###}—(CH₂)ₘ₂—C(═O)—, ^{###}—CH₂—C(═O)—NH—(CH₂)ₘ₂—C(═O)—, ^{###}—(CH₂CH₂O)ₘ₂—CH₂CH₂—C(═O)—, ^{###}—CH[—(CH₂)ₘ₂—COOH]—C(═O)—, ^{###}—CH₂—C(═O)—NH—(CH₂)ₘ₂—C(═O)—NH—(CH₂)ₘ₂—C(═O)—, ^{###}—C(═O)—(CH₂)ₘ₂—C(═O)—, or ^{###}—NH—(CH₂)ₘ₂—C(═O)—; wherein:
each m2 is, independently, 1, 2, 3, 4, 6, 7, or 8; and
### marks the bond where Spacer connects to Conjugator; and
Conjugator has formula (III), (IV), or (V): 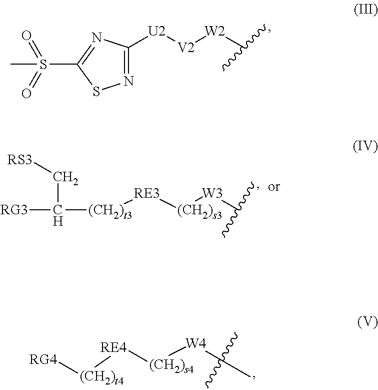
wherein:
U2 is a bond, heteroarylene, or arylene;
V2 is a bond or —C≡C—(CH₂)ₙ₂—;
n2 is an integer from 0 to 10 inclusive;
W2 is —C(═O)—, —NH—, or —O—;
RG3 is 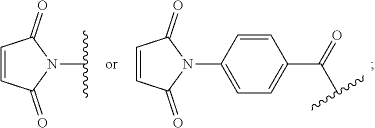 [SMILES: *N1C(=O)C=CC1=O ; *C(=O)c1ccc(N2C(=O)C=CC2=O)cc1]
RS3 is —NR^{a2}R^{b2};
each of R^{a2} and R^{b2} is, independently, H or substituted or unsubstituted C₁₋₄ alkyl;
RE3 is a bond, —O—, —OC(═O)—, —OC(═O)NR⁷—, —NHC(═O)NR⁷—, —OS(═O)₂NR⁷—, —NHS(═O)₂NR⁷—, or —OC(═O)NHS(═O)₂NR⁷;
R⁷ is H or substituted or unsubstituted C₁₋₄ alkyl;
W3 is —C(═O)—, —NH—, or —O—;
t3 is 1 or 2;
s3 is 0, 1, or 2;
RG4 is 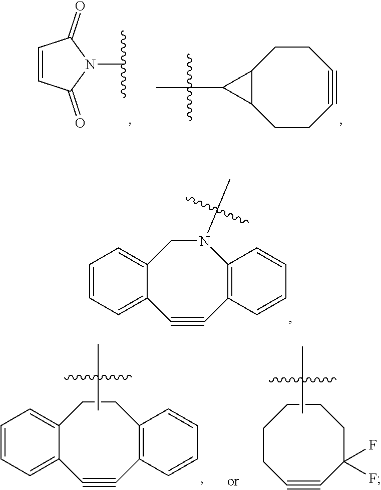
RE4 is a bond, —O—, —OC(═O)—, —OC(═O)NR—, —NHC(═O)NR—, —OS(═O)₂NR⁸—, —NHS(═O)₂NR⁸—, or —OC(═O)NHS(═O)₂NR⁸—;
R⁸ is H or substituted or unsubstituted C₁₋₄ alkyl;
W4 is —C(═O)—, —NH—, or —O—;
t4 is 1, 2, 3, 4, 5, 6, 7, or 8; and
s4 is 0, 1, or 2.

2. The compound of claim 1, wherein
Linker is —(CH₂)ᵣ or —(CH₂)ₚ—X—(CH₂)_{q}—;
ring A has formula (IIa), (IIb), (IIc), (IId), or (IIg); and
ring B has formula (IIa), (IIb), (IId), or (IIg).

3. The compound of claim 1 or claim 2, wherein
Linker is —(CH₂)ᵣ—, ring A has formula (IIg), and ring B has formula (IIg);
Linker is —(CH₂)ᵣ—, ring A has formula (IId), and ring B has formula (IId);
Linker is —(CH₂)ᵣ—, ring A has formula (IIb), and ring B has formula (IIa);
Linker is —(CH₂)ᵣ—, ring A has formula (IIa), and ring B has formula (IIb);
Linker is —(CH₂)ᵣ—, ring A has formula (IIa), and ring B has formula (IIa);
Linker is —(CH₂)ᵣ—, ring A has formula (IIc), and ring B has formula (IIa); or
Linker is —(CH₂)ₚ—X—(CH₂)_{q}—, ring A has formula (IIa), and ring B has formula (IIa).

4. The compound of any one of claims 1-3, wherein Linker is —(CH₂)ᵣ— and r is 5.

5. The compound of any one of claims 1-3, wherein Linker is —(CH₂)ₚ—X—(CH₂)_{q}—, X is an unsubstituted aryl ring, p is 1, and q is 1.

6. The compound of claim 5, wherein X is phenylene.

7. The compound of any one of claims 1-6, wherein
formula (IIa) is 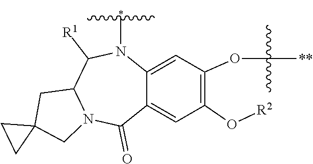
(IIa1);
formula (IId) is 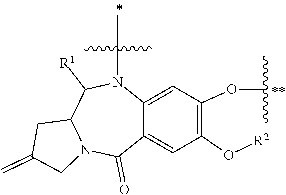
(IId1);
formula (IIg) is 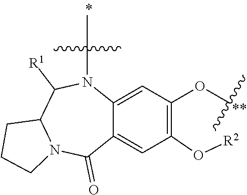
(IIgl); and/or
formula (IIc) is

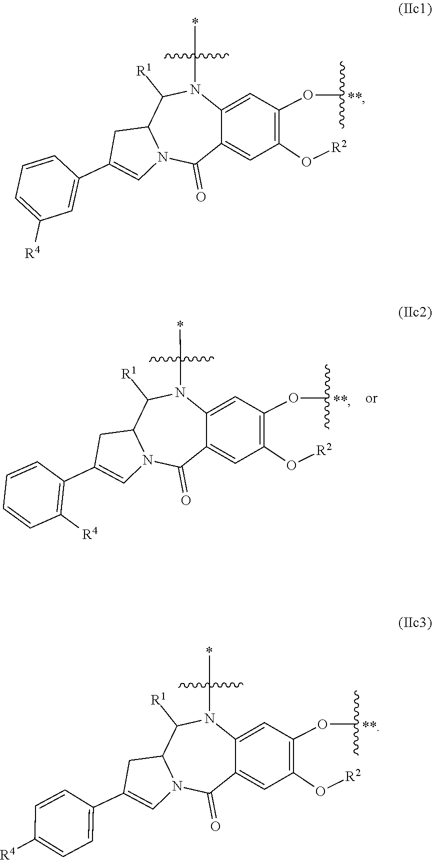

8. The compound of any one of claims 1-7, wherein ring A is

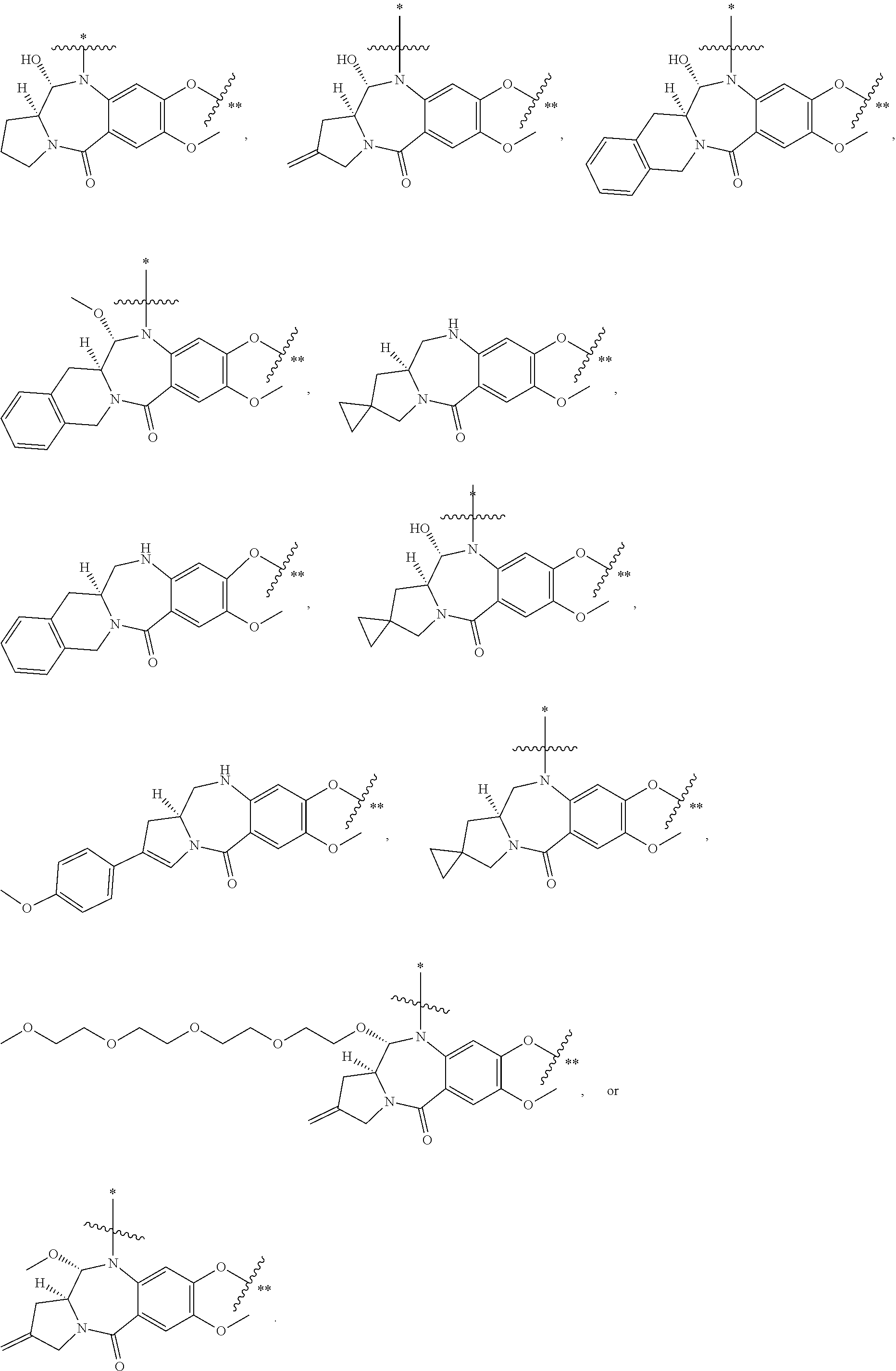

9. The compound of any one of claims 1-8, wherein ring B is

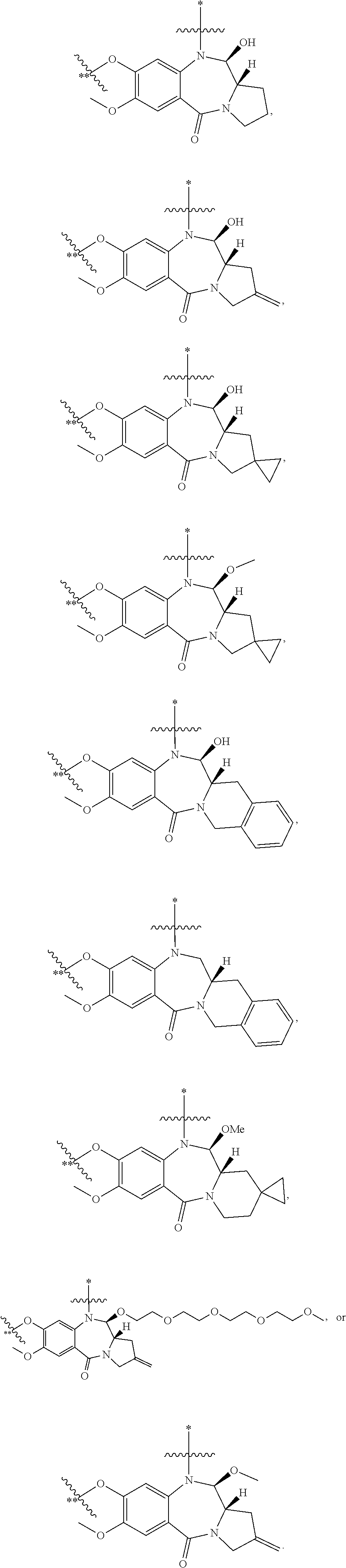

10. The compound of any one of claims 1-9, wherein
each Su is independently 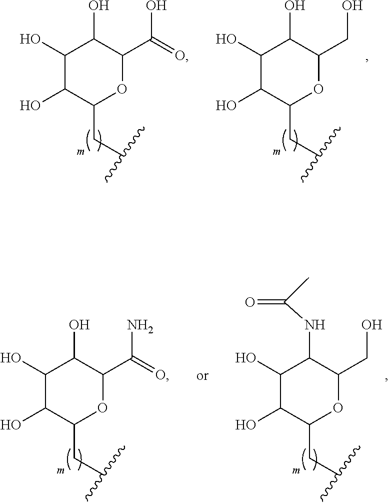
or a stereoisomer thereof;
each m is independently 0 or 1; and [symbol]
indicates a point of attachment to Cleavable1 or Cleavable2.

11. The compound of claim 10, wherein each Su is independently or 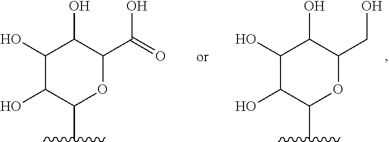 [SMILES: *C1OC(C(=O)O)C(O)C(O)C1O ; *C1OC(CO)C(O)C(O)C1O]
or a stereoisomer thereof.

12. The compound of claim 11, wherein each Su is

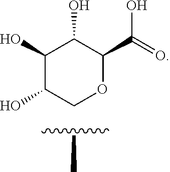

13. The compound of any one of claims 1-12, wherein s1 is 1.

14. The compound of claim 13, wherein Cleavable1 has formula (VIa).

15. The compound of claim 14, wherein Cleavable1 is

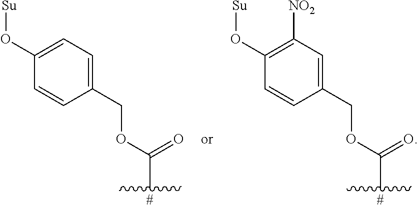

16. The compound of any one of claims 1-12, wherein s1 is 0.

17. The compound of any one of claims 1-16, wherein Cleavable2 has formula (VIIa1) or (VIId1):

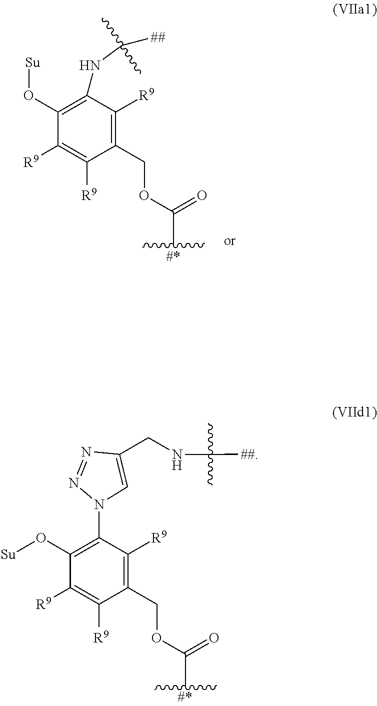

18. The compound of claim 17, wherein Cleavable2 is

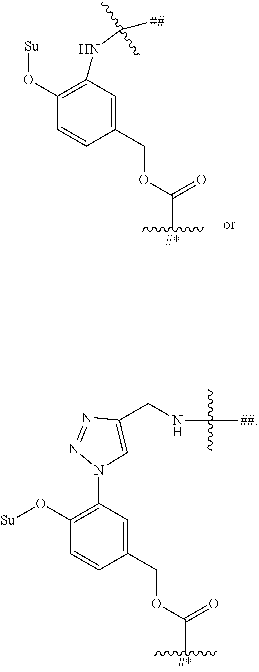

19. The compound of any one of claims 1-18, wherein Spacer is ^{###}—NH—(CH₂CH₂O)ₘ₂—CH₂CH₂—C(═O)— or ^{###}—NH—(CH₂CH₂O)ₘ₂—CH₂—C(═O)—.

20. The compound of any one of claims 1-19, wherein Spacer is ^{###}—NH—(CH₂CH₂O)₄—CH₂CH₂—C(═O)—.

21. The compound of any one of claims 1-20, wherein Conjugator has formula (III).

22. The compound of claim 21, wherein U2 is arylene.

23. The compound of claim 22, wherein U2 is

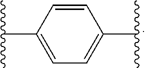 [SMILES: *c1ccc(*)cc1]

24. The compound of any one of claims 21-23, wherein V2 is a bond.

25. The compound of any one of claims 21-24, wherein W2 is —C(═O)—.

26. The compound of any one of claims 21-25, wherein Conjugator is

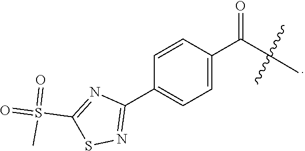 [SMILES: *C(=O)c1ccc(-c2nsc(S(C)(=O)=O)n2)cc1]

27. The compound of any one of claims 1-20, wherein Conjugator has formula (V).

28. The compound of claim 27, wherein RG4 is

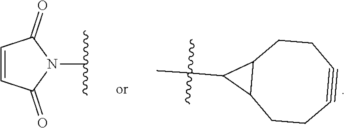 [SMILES: *N1C(=O)C=CC1=O ; *C1C2CCC#CCCC12]

29. The compound of claim 27 or 28, wherein t4 is 1, 2, 3, 4, or 5.

30. The compound of any one of claims 27-29, wherein s4 is 0.

31. The compound of any one of claims 27-30, wherein the sum of s4 and t4 is 1 or 5.

32. The compound of any one of claims 27-31, wherein RE4 is a bond or —O—.

33. The compound of any one of claims 27-32, wherein W4 is —C(═O)—.

34. The compound of any one of claims 27-33, wherein Conjugator is or

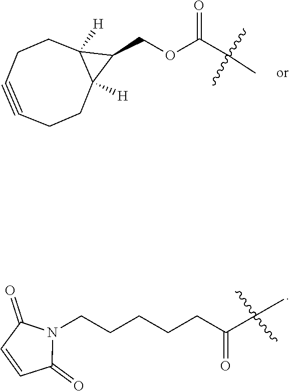 [SMILES: *C(=O)OC[C@@H]1[C@@H]2CCC#CCC[C@@H]21 ; *C(=O)CCCCCN1C(=O)C=CC1=O]

35. The compound of any one of claims 1-20, wherein Conjugator has formula (IV).

36. The compound of claim 35, wherein RG3 is

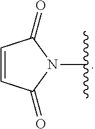 [SMILES: *N1C(=O)C=CC1=O]

37. The compound of claim 35 or 36, wherein RS3 is NH₂.

38. The compound of any one of claims 35-37, wherein t3 is 2.

39. The compound of any one of claims 35-38, wherein RE3 is —O—.

40. The compound of any one of claims 35-39, wherein s3 is 0.

41. The compound of any one of claims 35-40, wherein W3 is —C(═O)—.

42. The compound of any one of claims 35-41, wherein Conjugator is

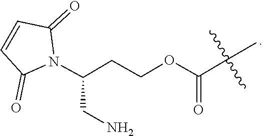 [SMILES: *C(=O)OCC[C@H](CN)N1C(=O)C=CC1=O]

43. The compound of claim 1, wherein the compound is 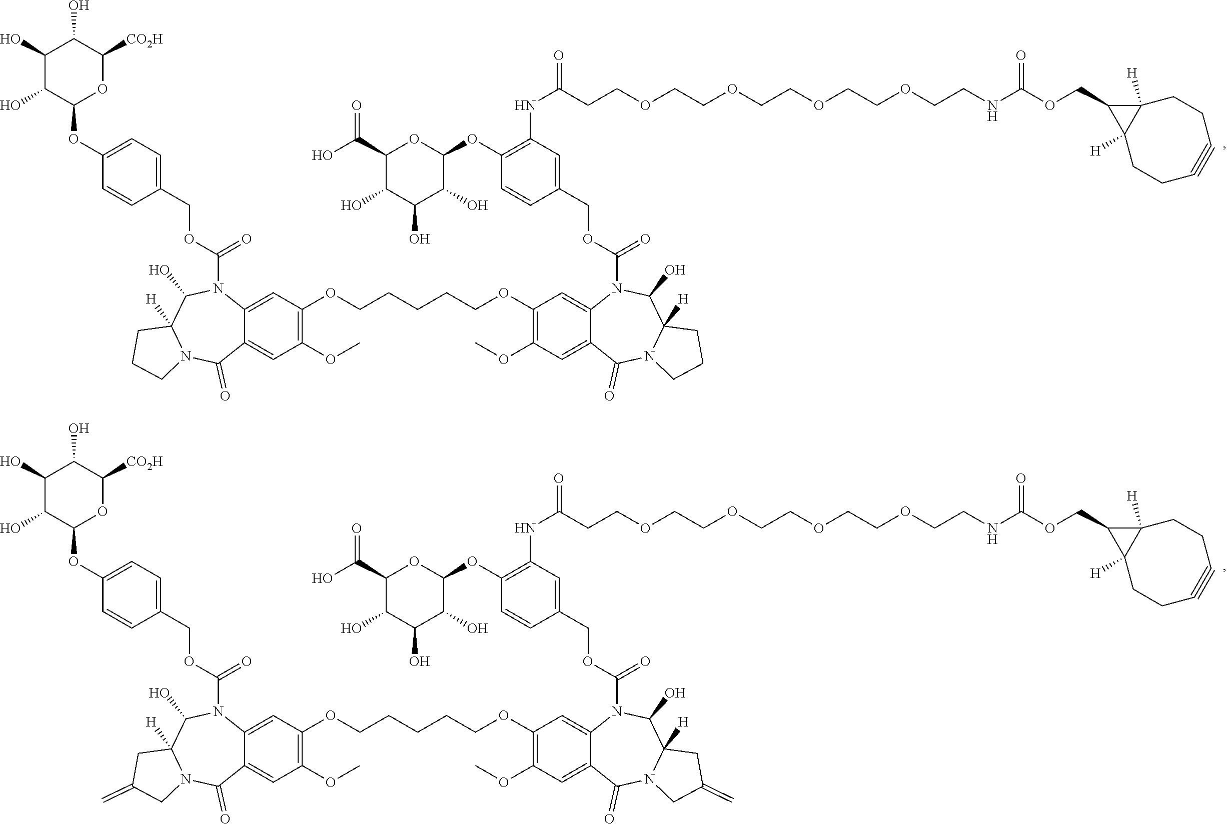 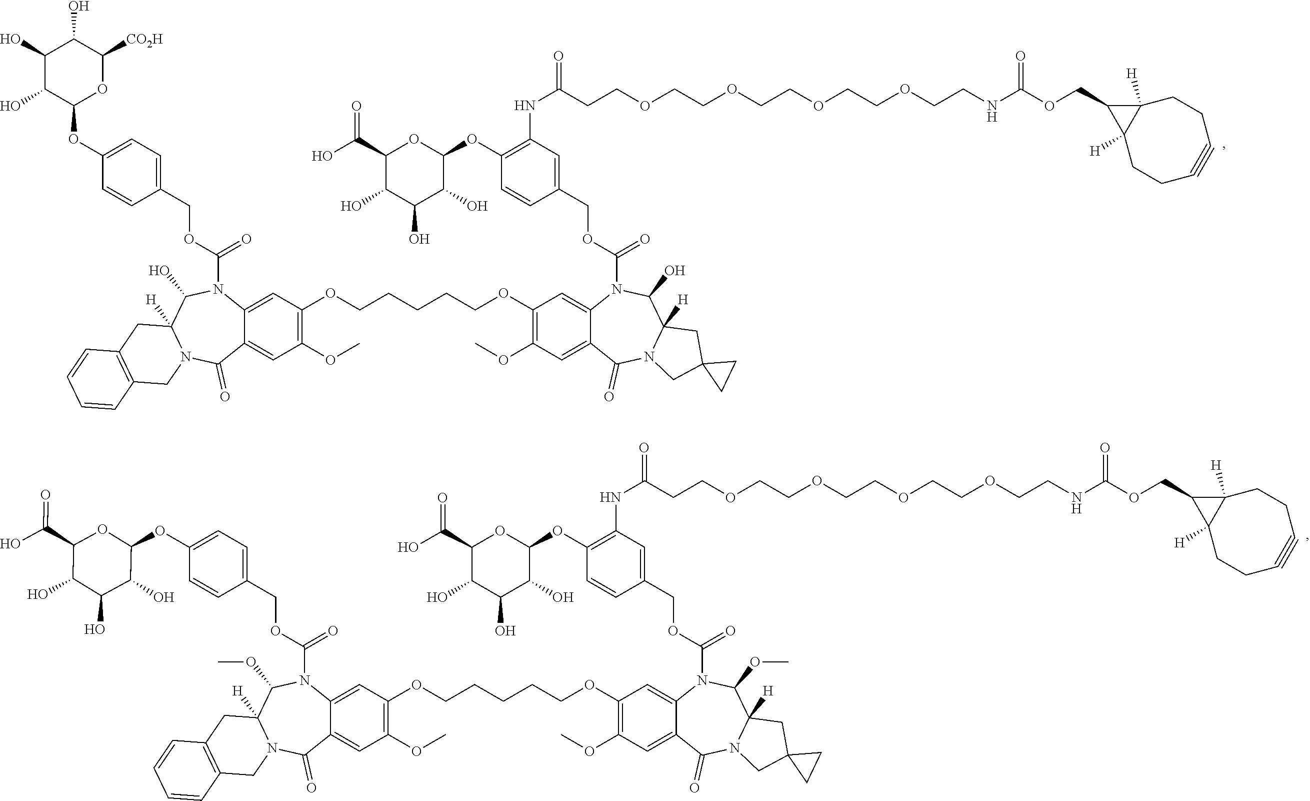 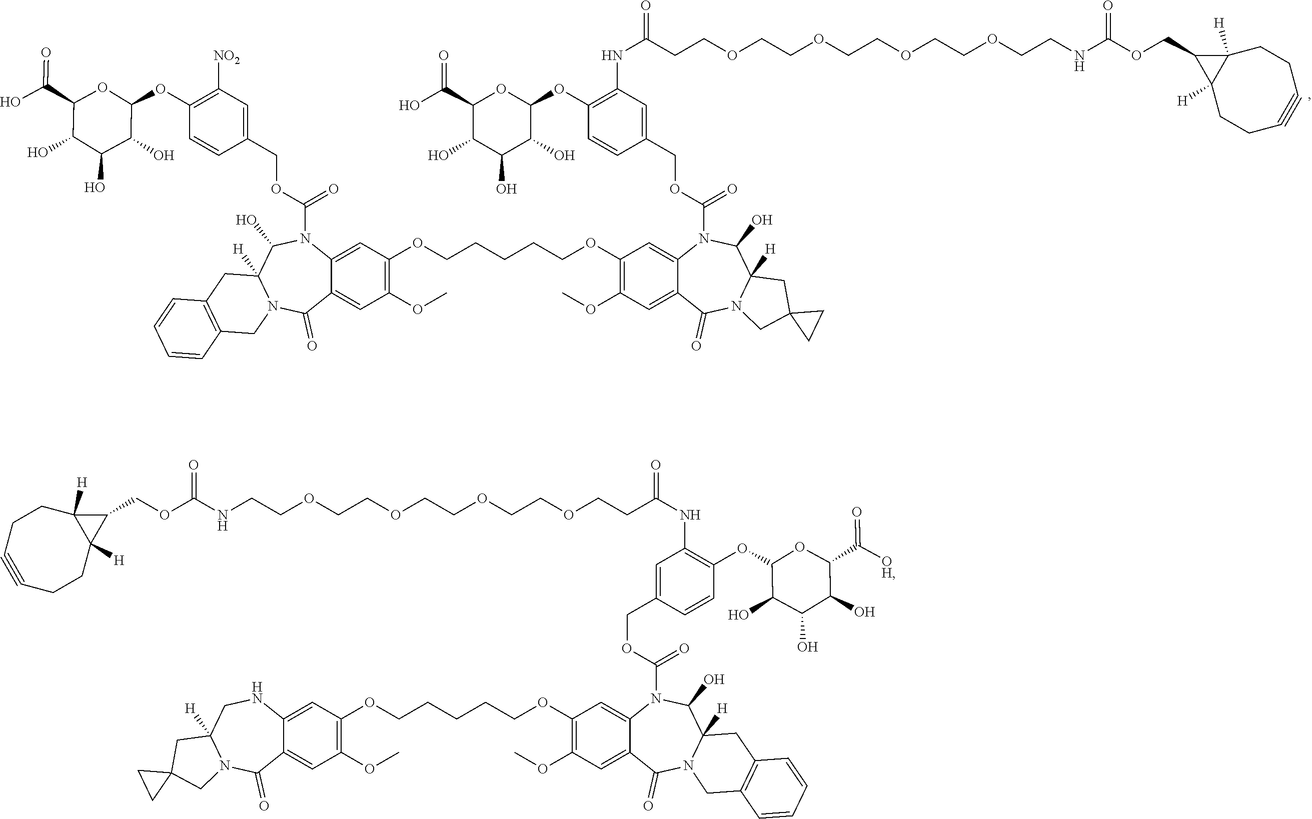 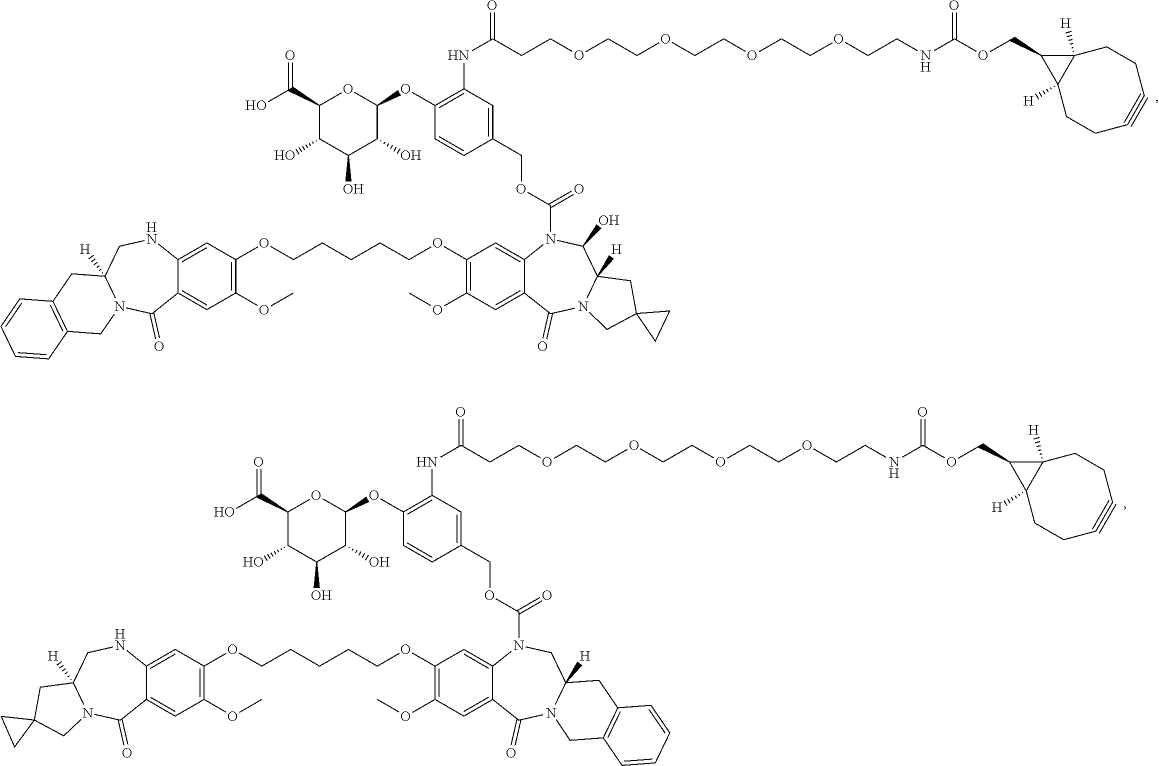 [SMILES: COc1cc2c(cc1OCCCCCOc1cc3c(cc1OC)C(=O)N1CC4(CC4)C[C@H]1[C@H](O)N3C(=O)OCc1ccc(O[C@@H]3O[C@H](C(=O)O)[C@@H](O)[C@H](O)[C@H]3O)c(NC(=O)CCOCCOCCOCCOCCNC(=O)OC[C@@H]3[C@@H]4CCC#CCC[C@@H]43)c1)NC[C@@H]1Cc3ccccc3CN1C2=O ; COc1cc2c(cc1OCCCCCOc1cc3c(cc1OC)C(=O)N1Cc4ccccc4C[C@H]1CN3C(=O)OCc1ccc(O[C@@H]3O[C@H](C(=O)O)[C@@H](O)[C@H](O)[C@H]3O)c(NC(=O)CCOCCOCCOCCOCCNC(=O)OC[C@@H]3[C@@H]4CCC#CCC[C@@H]43)c1)NC[C@@H]1CC3(CC3)CN1C2=O] 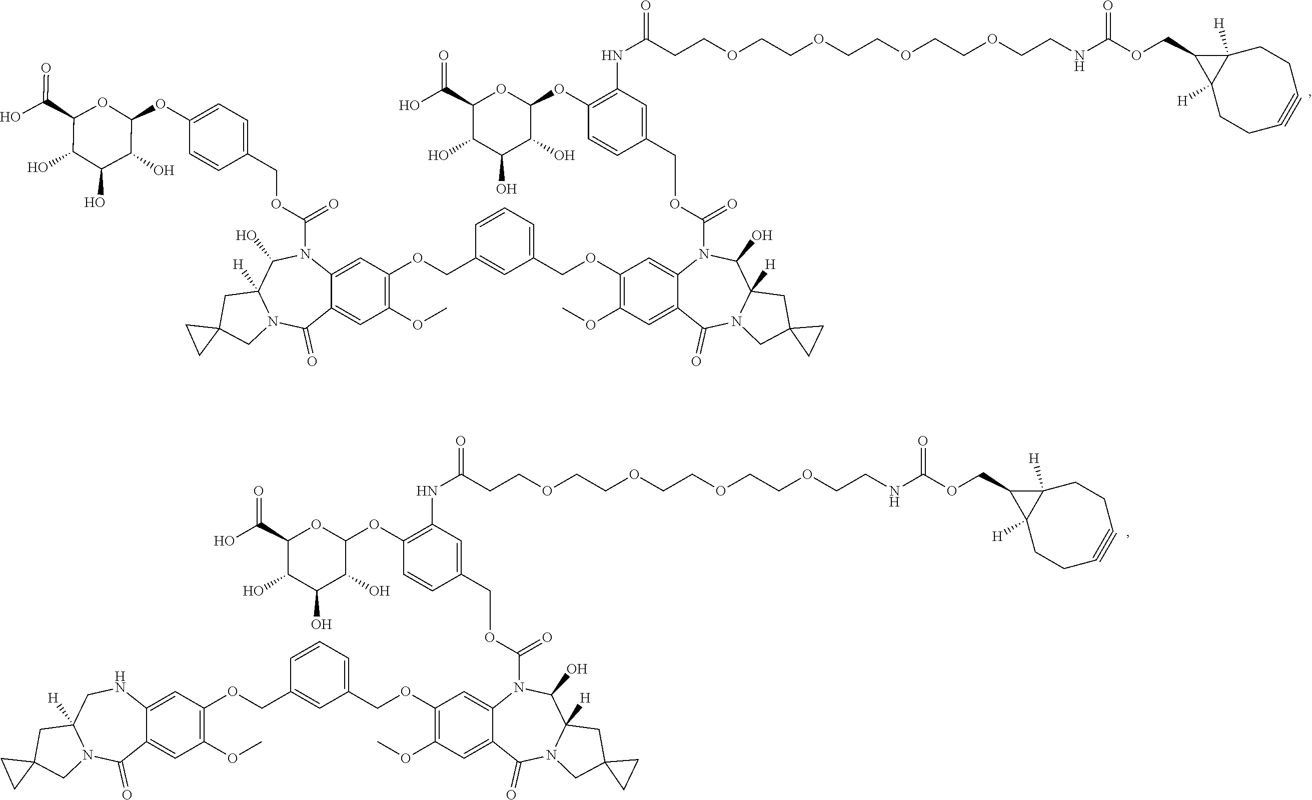 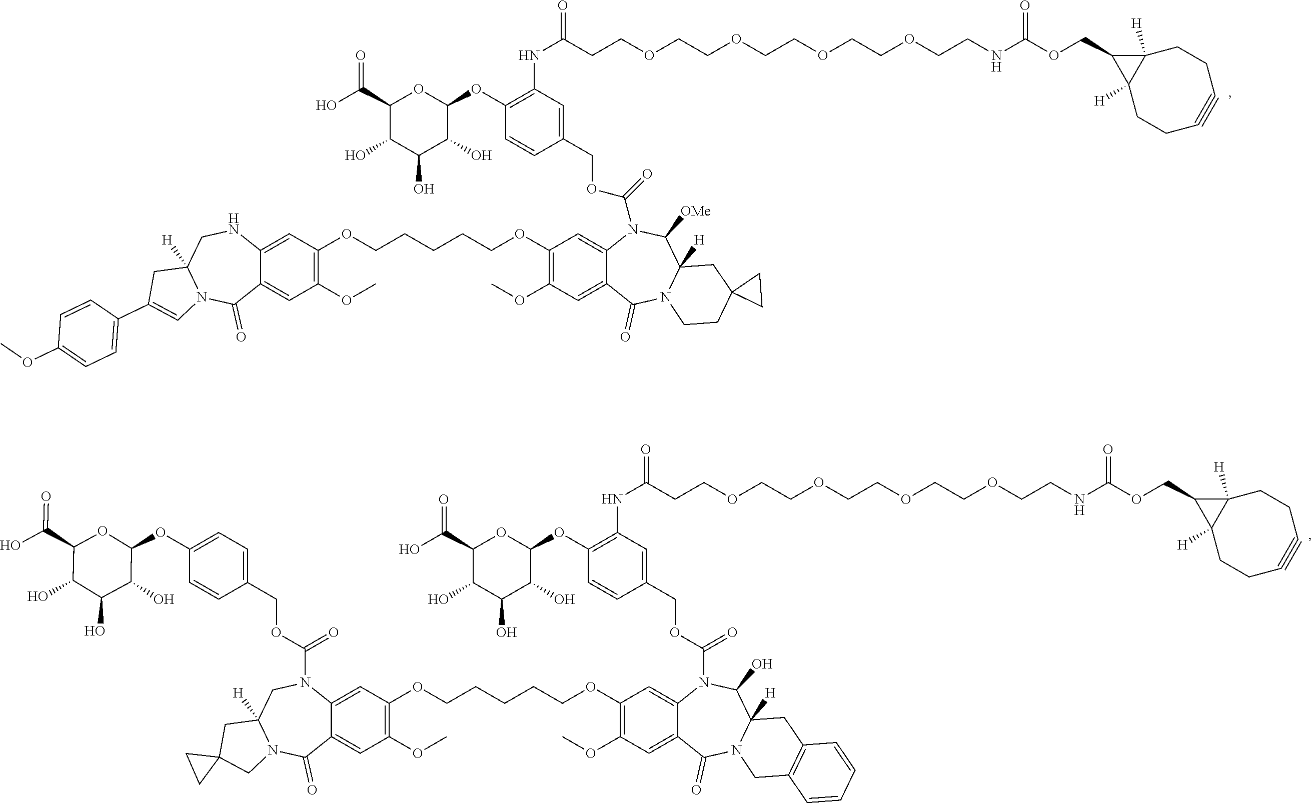 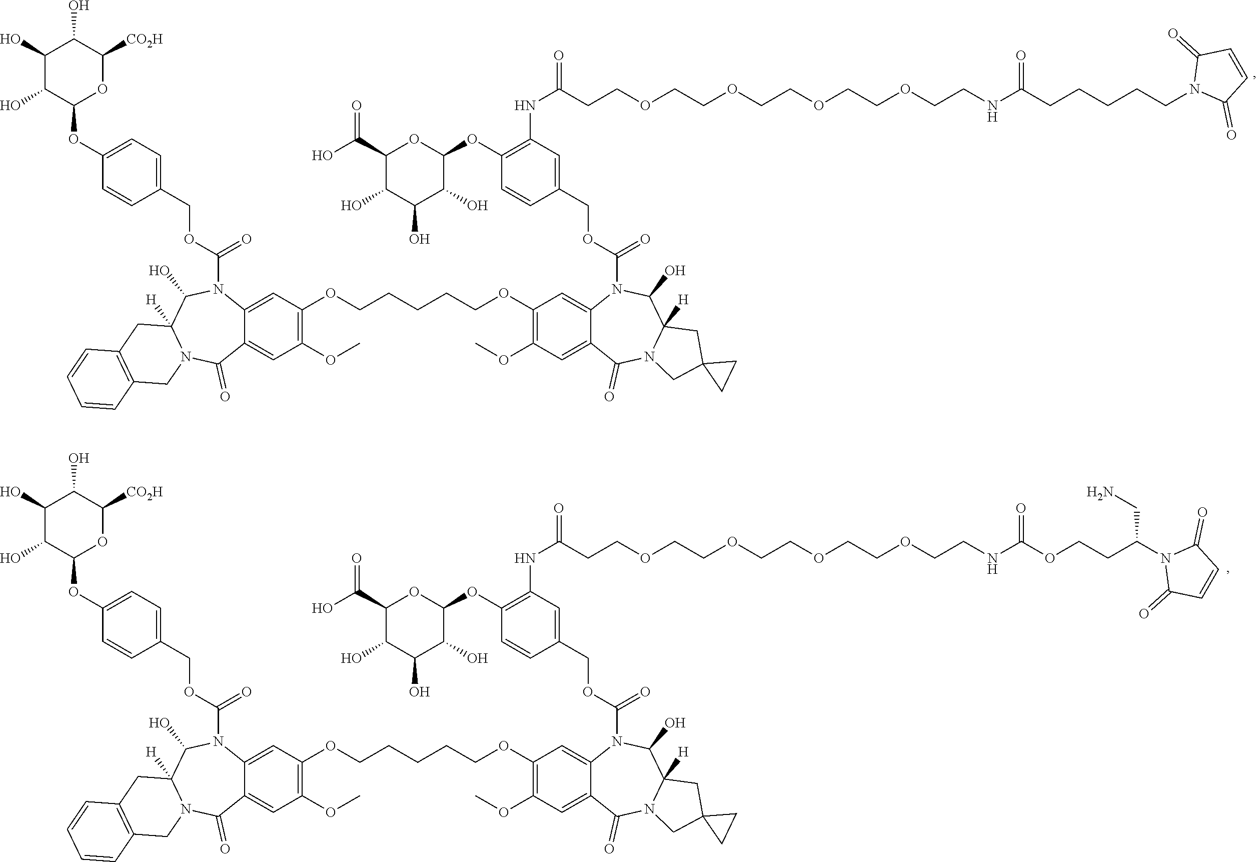 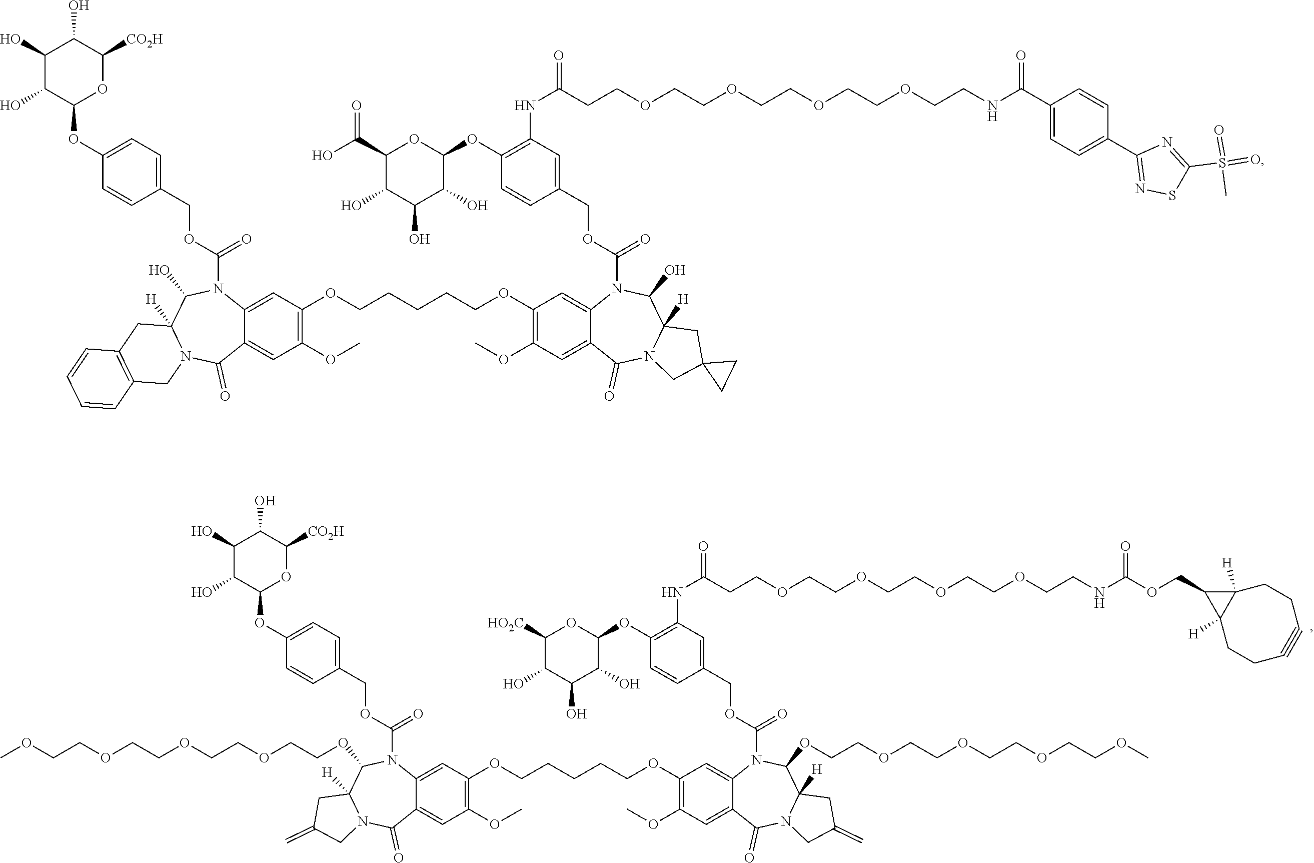 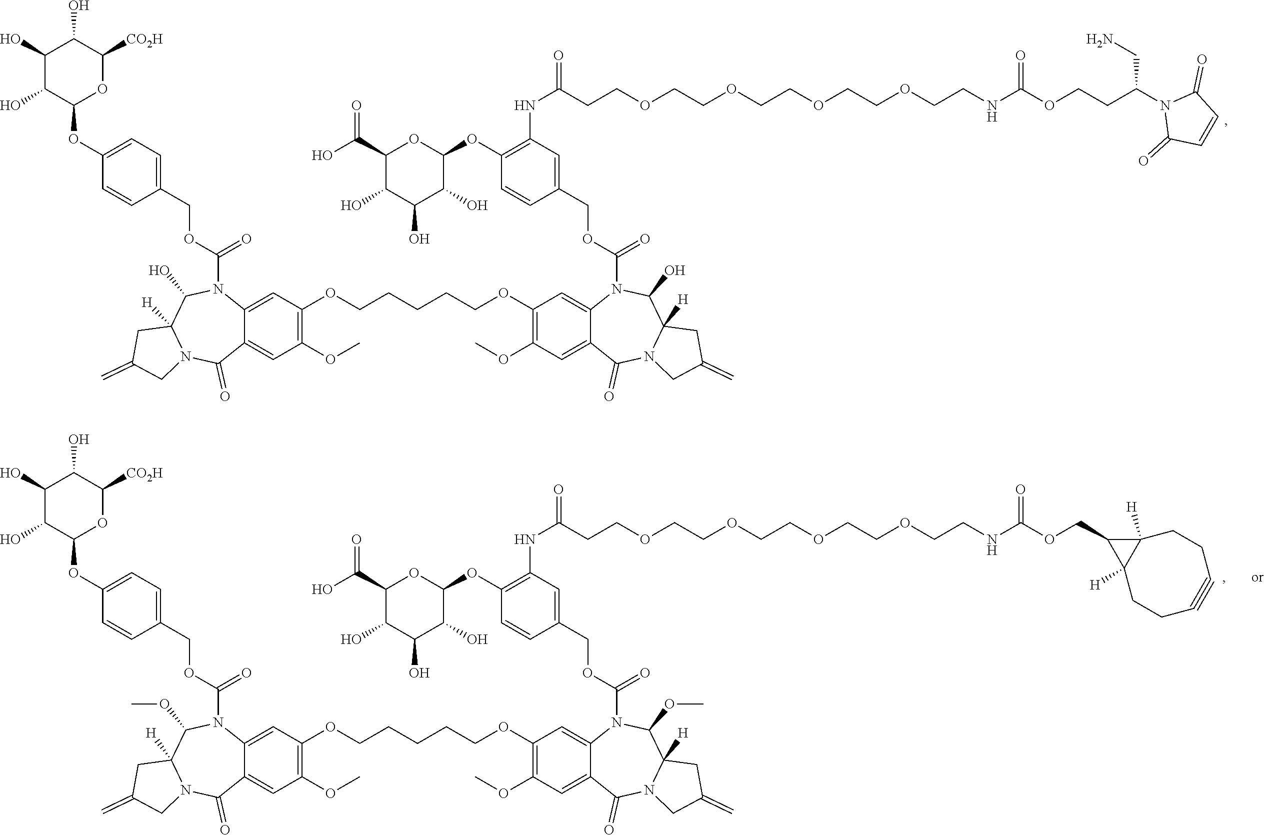 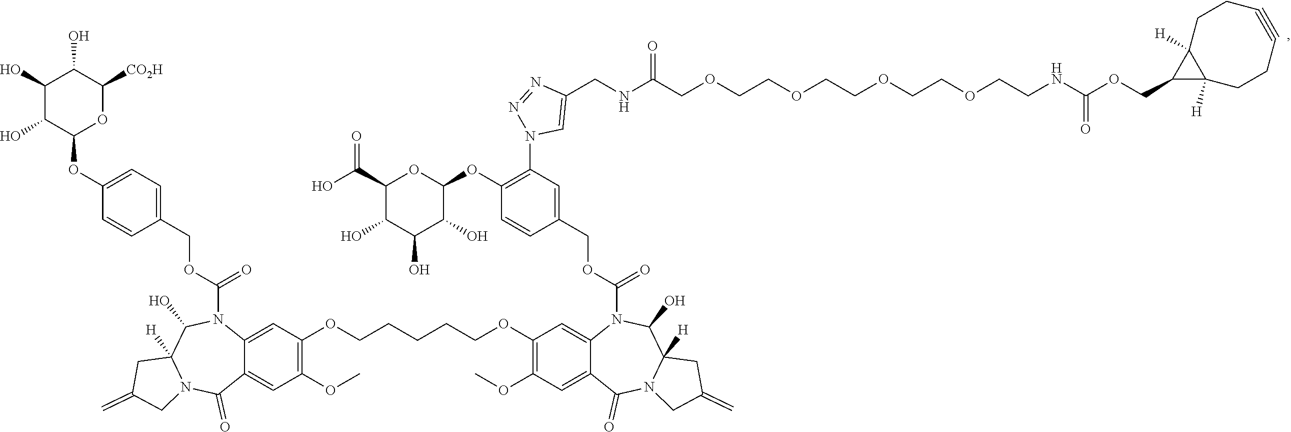 [SMILES: C=C1C[C@H]2[C@H](O)N(C(=O)OCc3ccc(O[C@@H]4O[C@H](C(=O)O)[C@@H](O)[C@H](O)[C@H]4O)cc3)c3cc(OCCCCCOc4cc5c(cc4OC)C(=O)N4CC(=C)C[C@H]4[C@H](O)N5C(=O)OCc4ccc(O[C@@H]5O[C@H](C(=O)O)[C@@H](O)[C@H](O)[C@H]5O)c(-n5cc(CNC(=O)COCCOCCOCCOCCNC(=O)OC[C@@H]6[C@@H]7CCC#CCC[C@@H]76)nn5)c4)c(OC)cc3C(=O)N2C1]
or a pharmaceutically acceptable salt, tautomer, solvate, stereoisomer, or isotopologue thereof.

44. A compound of Formula (IA): 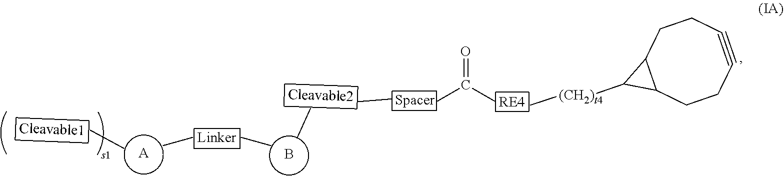
or a pharmaceutically acceptable salt, tautomer, solvate, stereoisomer, or isotopologue thereof, wherein:
each of ring A and ring B is, independently, one of formula (IIa), (IIb), (IIc), (IId), (IIe), (IIf), or (IIg): 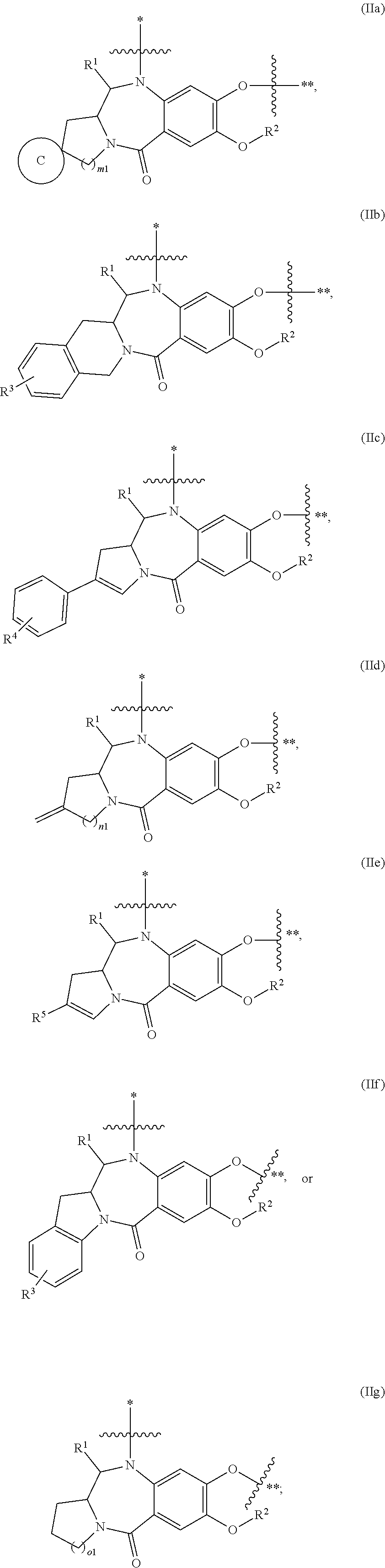
wherein:
each ring C is independently a cyclopropyl ring or a cyclobutyl ring;
each R¹ is independently H, OH, C₁₋₄ alkyl, C₁₋₄ alkoxyl, or —O—(CH₂CH₂O)ₜ₁—CH₃;
each R² is independently H or C₁₋₄ alkyl;
each R³ and R⁴ is, independently, H, NR^{a1}R^{b1}, OH, C₁₋₄ alkyl, C₁₋₄ alkoxyl, or aryl;
each of R^{a1} and R^{b1} is, independently, H or C₁₋₄ alkyl;
each R⁵ is independently H, C₁₋₄ alkyl, C₁₋₄ alkoxyl, or aryl;
each m1, n1, and o1 is, independently, 1 or 2;
t1 is 1, 2, 3, 4, 5, 6, 7, or 8;
* marks the bond where ring A connects to Cleavable1 when s1 is 1 or to —H when s1 is 0, or where ring B connects to Cleavable2; and
** marks the bond where ring A or ring B connects to Linker;
Linker is —(CH₂)ᵣ—, —(CH₂)ₚ—X—(CH₂)_{q}—, or —(CH₂)ₚ—CH═CH—(CH₂)_{q}—; wherein:
each r, p, and q, is, independently, 1, 2, 3, 4, 5, 6, 7, or 8;
the sum of p and q is 1, 2, 3, 4, 5, 6, 7, or 8;
X is NR⁶, NHC(═O), C(═O)NH, O, SO₂, a substituted or unsubstituted aryl ring, substituted or unsubstituted heteroaryl ring, substituted or unsubstituted heterocyclic ring, or substituted or unsubstituted cyclic ring; and
R⁶ is H or C₁₋₄ alkyl;
Cleavable1, if present, has the formula 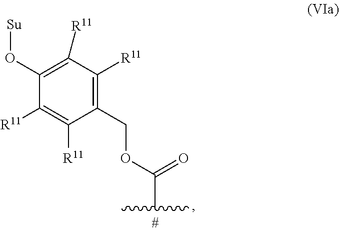
wherein:
Su is a sugar moiety;
each R¹¹ is, independently, hydrogen, halogen, substituted or unsubstituted C₁₋₄ alkyl, —CN, or —NO₂; and
# marks the bond where Cleavable1 connects to ring A;
s1 is 0 or 1;
Cleavable2 has formula (VIIa1) or (VIId1): 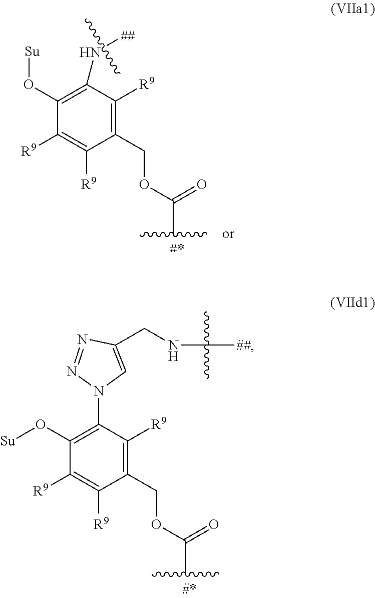
wherein:
Su is a sugar moiety;
each R⁹ is, independently, hydrogen or C₁₋₄ alkyl;
#* marks the bond where Cleavable2 connects to ring B; and
## marks the bond where Cleavable2 connects to Spacer;
Spacer is a bond, ^{###}—NH—(CH₂CH₂O)ₘ₂—CH₂CH₂—C(═O)— or ^{###}—NH—(CH₂CH₂O)ₘ₂—CH₂—C(═O)—; wherein:
each m2 is, independently, 1, 2, 3, 4, 6, 7, or 8; and
### marks the bond where Spacer connects to —C(═O)—;
RE4 is a bond or —O—; and
t4 is 1, 2, 3, 4, 5, 6, 7, or 8.

45. The compound of claim 44, wherein
Linker is —(CH₂)ᵣ or —(CH₂)ₚ—X—(CH₂)_{q}—;
ring A has formula (IIa), (IIb), (IIc), (IId), or (IIg); and
ring B has formula (IIa), (IIb), (IId), or (IIg).

46. The compound of claim 44 or claim 45, wherein
Linker is —(CH₂)ᵣ—, ring A has formula (IIg), and ring B has formula (IIg);
Linker is —(CH₂)ᵣ—, ring A has formula (IId), and ring B has formula (IId);
Linker is —(CH₂)ᵣ—, ring A has formula (IIb), and ring B has formula (IIa);
Linker is —(CH₂)ᵣ—, ring A has formula (IIa), and ring B has formula (IIb);
Linker is —(CH₂)ᵣ—, ring A has formula (IIa), and ring B has formula (IIa);
Linker is —(CH₂)ᵣ—, ring A has formula (IIc), and ring B has formula (IIa); or
Linker is —(CH₂)ₚ—X—(CH₂)_{q}—, ring A has formula (IIa), and ring B has formula (IIa).

47. The compound of any one of claims 44-46, wherein Linker is —(CH₂)ᵣ— and r is 5.

48. The compound of any one of claims 44-46, wherein Linker is —(CH₂)ₚ—X—(CH₂)_{q}—, X is an unsubstituted aryl ring, p is 1, and q is 1.

49. The compound of claim 48, wherein X is phenylene.

50. The compound of any one of claims 44-49, wherein
formula (IIa) is 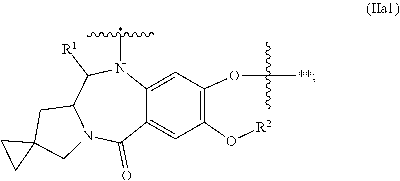
formula (IId) is 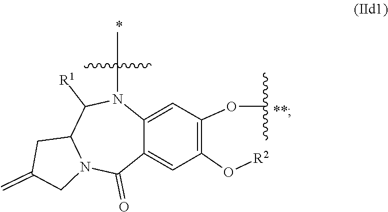
formula (IIg) is 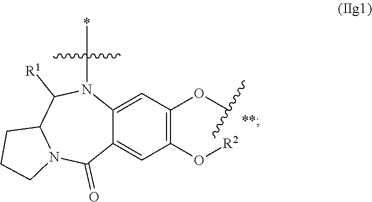
and/or
formula (IIc) is

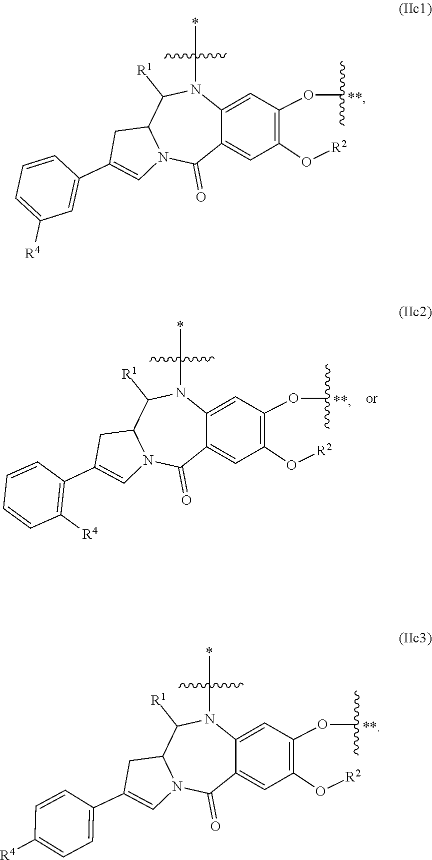

51. The compound of any one of claims 44-50, wherein ring A is

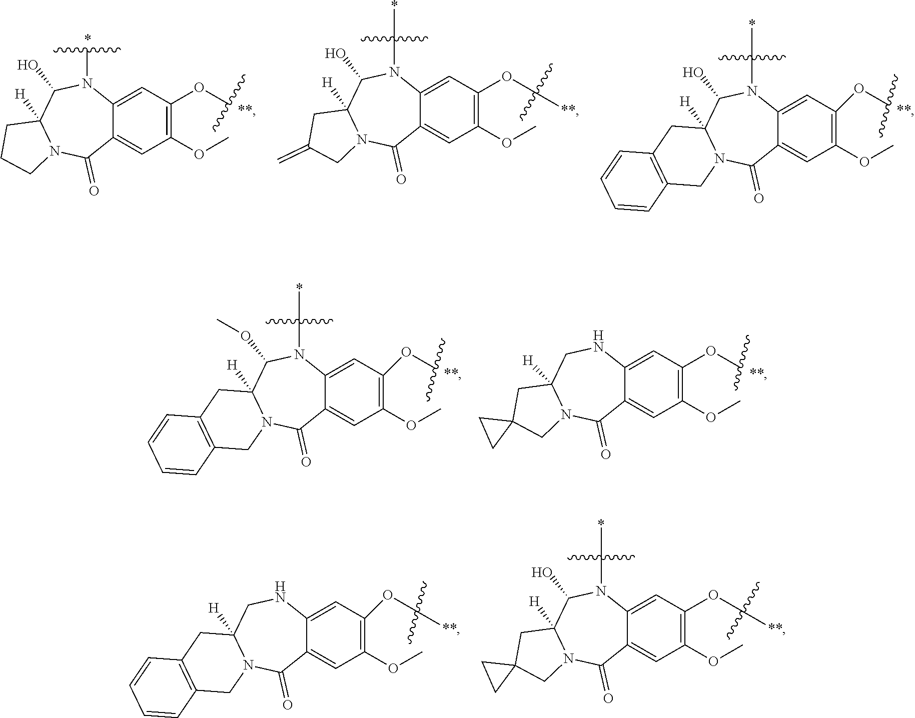

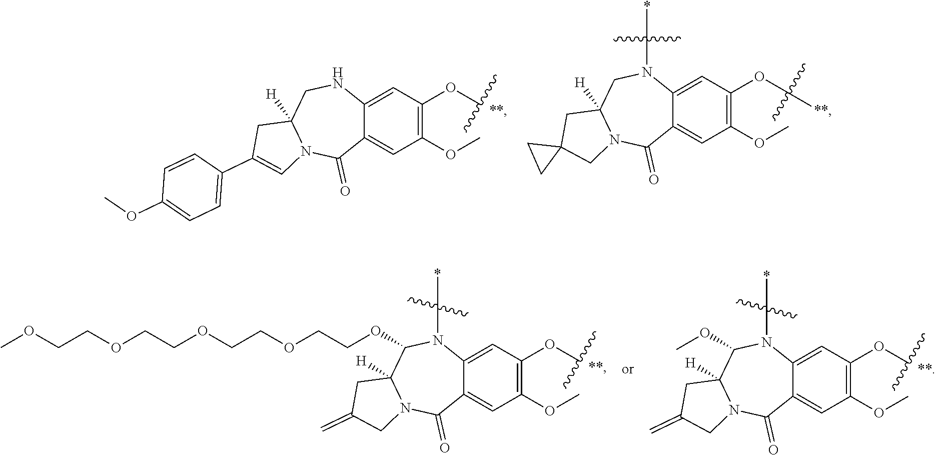

52. The compound of any one of claims 44-51, wherein ring B is

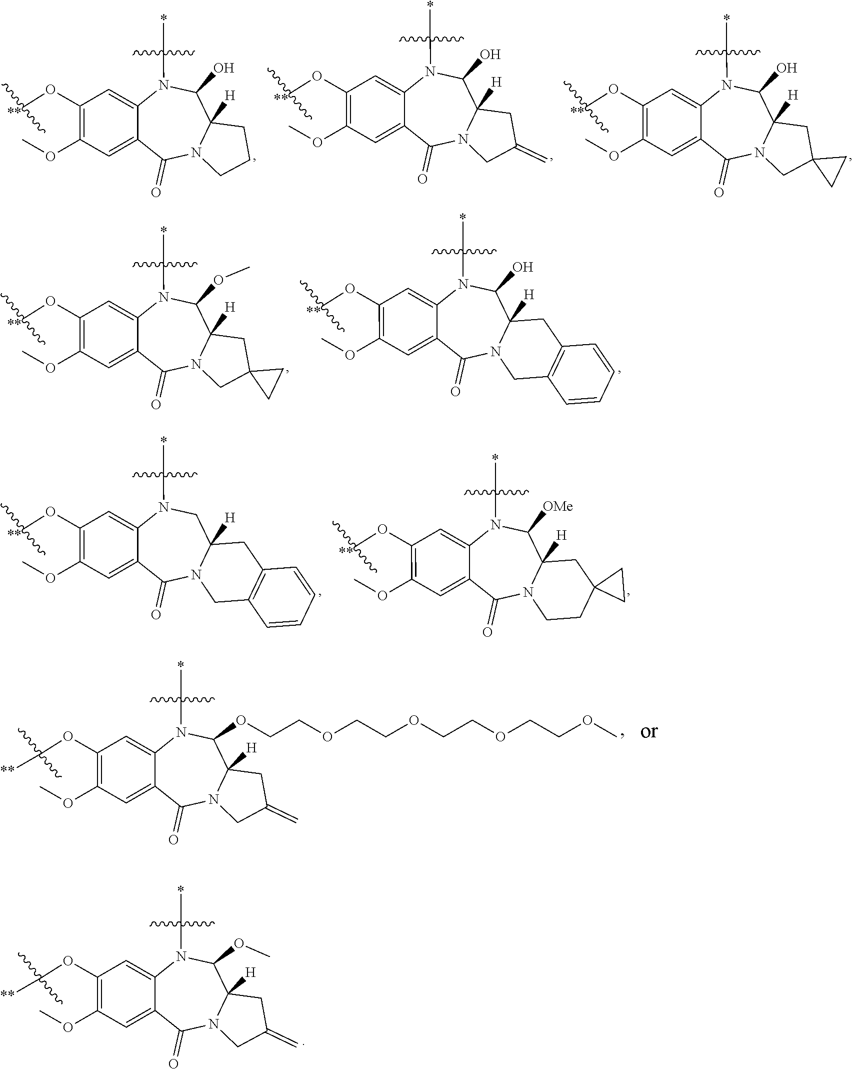

53. The compound of any one of claims 44-52, wherein
each Su is independently 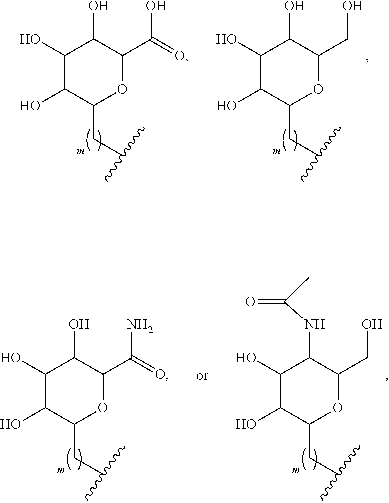
or a stereoisomer thereof;
each m is independently 0 or 1; and [symbol]
indicates a point of attachment to Cleavable1 or Cleavable2.

54. The compound of claim 53, wherein each Su is independently 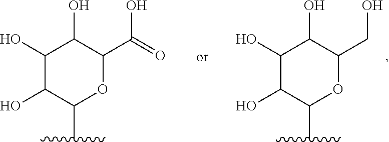 [SMILES: *C1OC(C(=O)O)C(O)C(O)C1O ; *C1OC(CO)C(O)C(O)C1O]
or a stereoisomer thereof.

55. The compound of claim 54, wherein each Su is

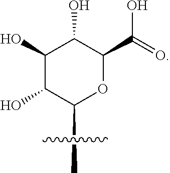

56. The compound of any one of claims 44-55, wherein s1 is 1.

57. The compound of claim 56, wherein each R¹¹ is independently hydrogen or —NO₂.

58. The compound of claim 57, wherein Cleavable1 is

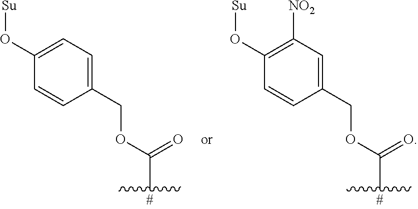

59. The compound of any one of claims 44-55, wherein s1 is 0.

60. The compound of any one of claims 44-59, wherein Cleavable2 is or

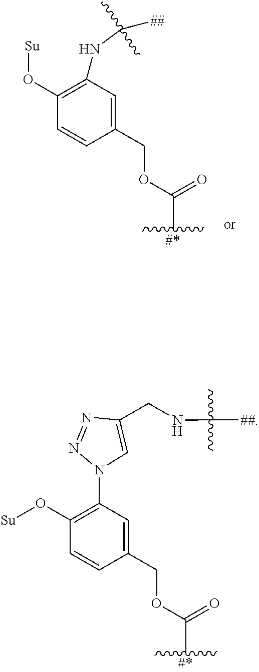

61. The compound of any one of claims 44-60, wherein Spacer is ^{###}—NH—(CH₂CH₂O)ₘ₂—CH₂CH₂—C(═O)— or ^{###}—NH—(CH₂CH₂O)ₘ₂—CH₂—C(═O)—.

62. The compound of any one of claims 44-61, wherein Spacer is ^{###}—NH—(CH₂CH₂O)₄—CH₂CH₂—C(═O)—.

63. The compound of any one of claims 44-62, wherein RE4 is

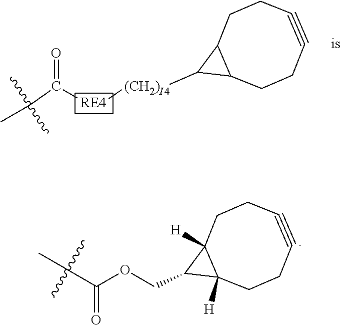

64. The compound of claim 44, wherein the compound is 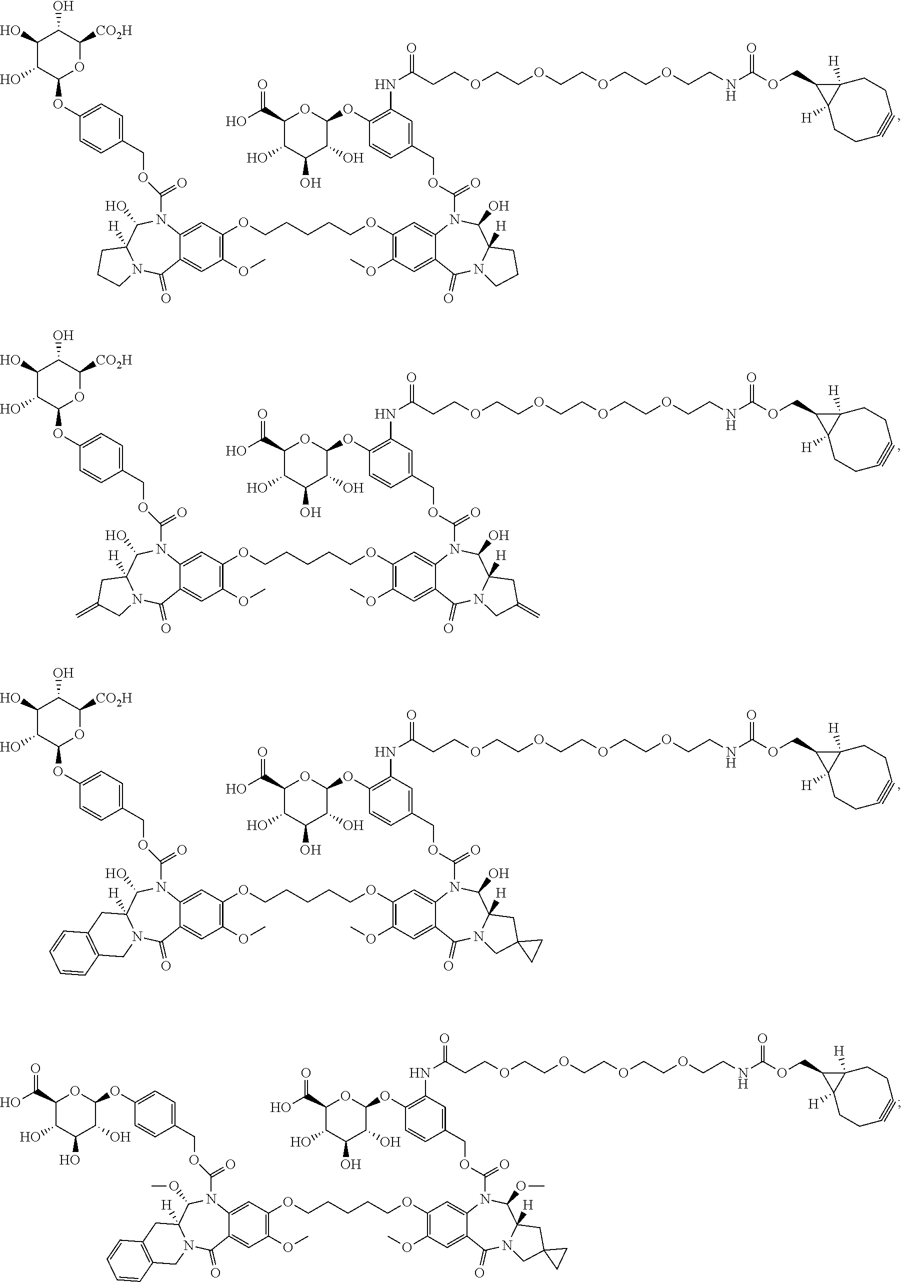 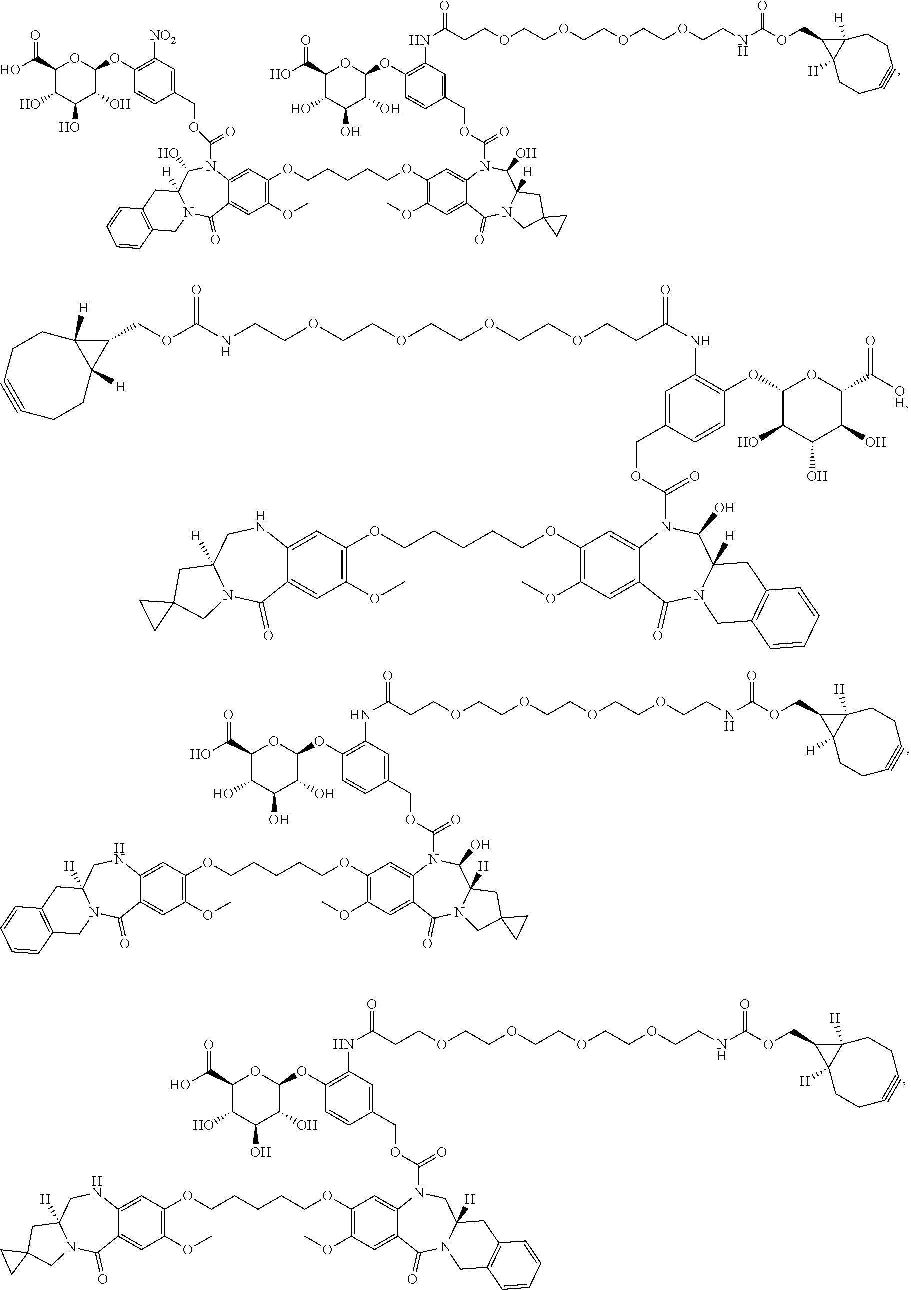 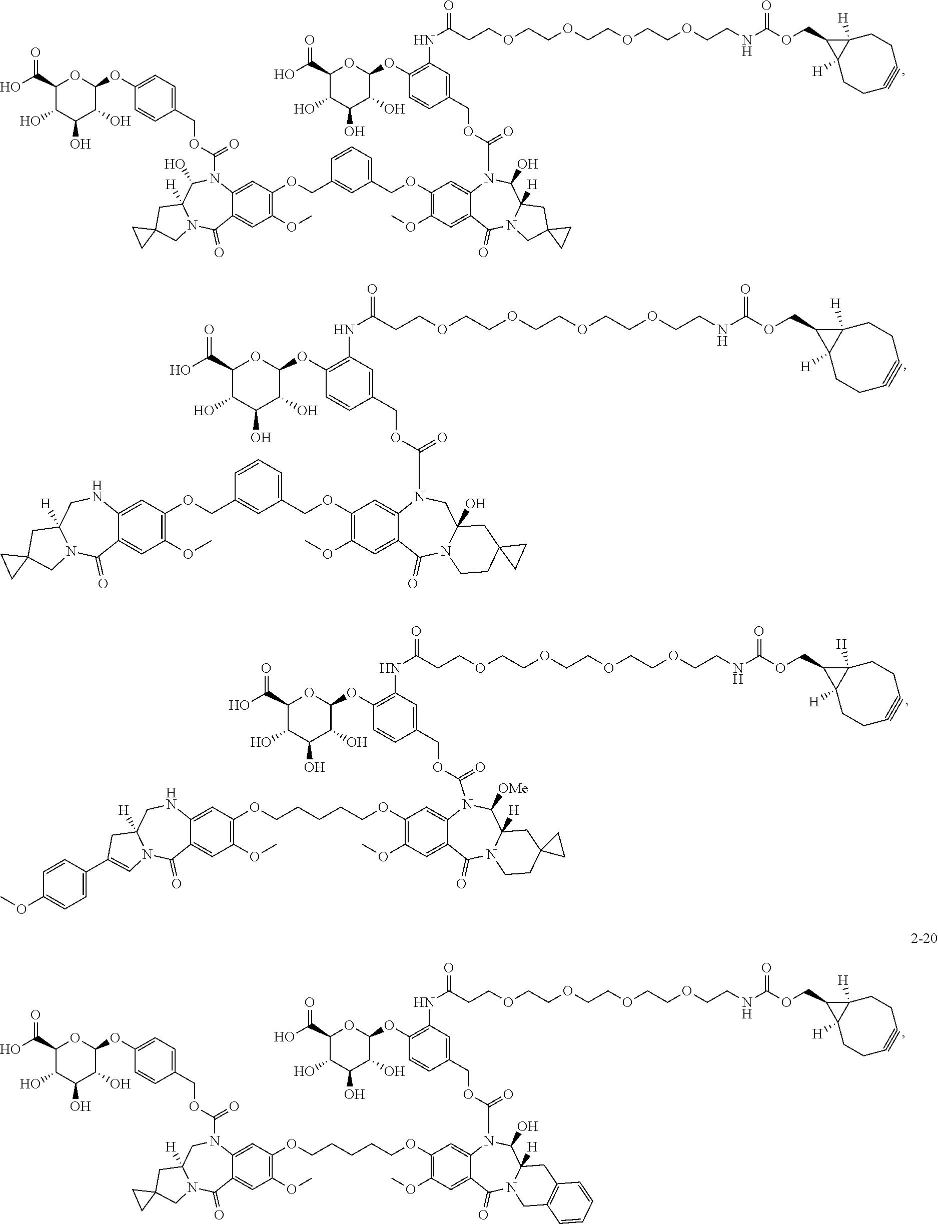 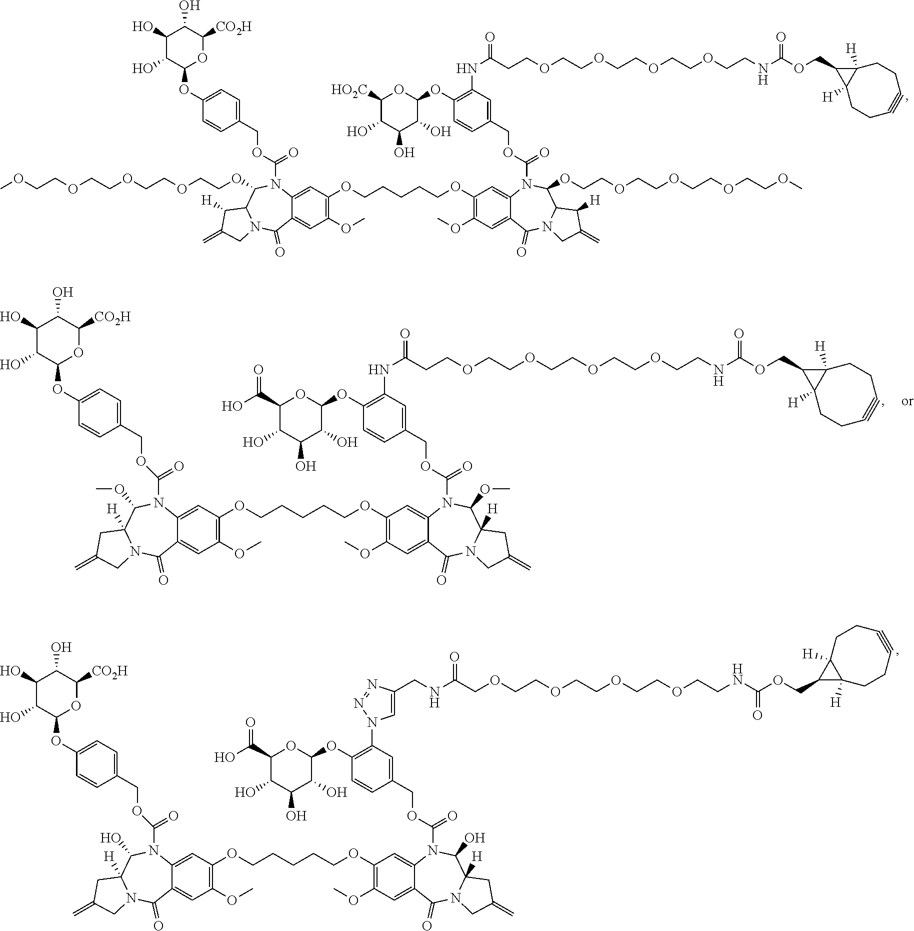
or a pharmaceutically acceptable salt, tautomer, solvate, stereoisomer, or isotopologue thereof.

65. A ligand-drug conjugate or a pharmaceutically acceptable salt or solvate thereof, wherein the conjugate comprises a ligand that is conjugated to the compound of any one of claims 1-64 via Conjugator or 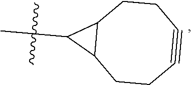 [SMILES: *C1C2CCC#CCCC12]
wherein the conjugate has the following formula:
Ligand-[Formula (I or Ia)]ₓ,
wherein x is from about 1 to about 10.

66. The conjugate of claim 65, wherein the ligand is a humanized, monoclonal, chimeric, or human antibody, or an antigen binding fragment thereof.

67. The conjugate of claim 65 or 66, wherein the conjugate has the following formula 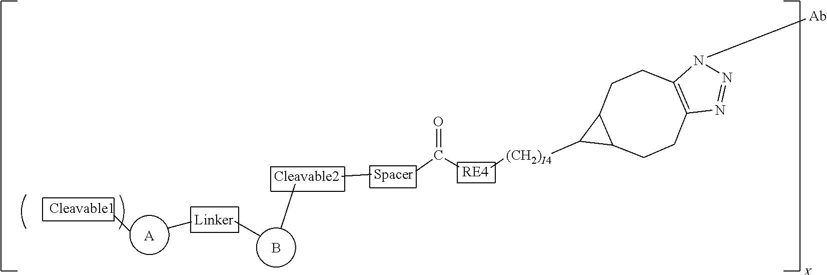
wherein Ab is a humanized, monoclonal, chimeric, or human antibody, or an antigen binding fragment thereof.

68. The conjugate of claim 66 or 67, wherein the ligand or Ab binds to one or more receptors chosen from B7-H3, cytokeratin 15, PTK7, HER3, HER2, CD7, CD19, CD20, CD22, CD25, CD27, CD30, CD33, CD37, CD38, CD46, CD70, CD71, CD74, CD79b, CD123, CD138, CD142, CD166, CD205, CD228, CCR2, CA6, p-Cadherin, CEA, CEACAM5, C4.4a, DLL3, EGFR, EGFRVIII, ENPP3, EphA2, EphrinA, FLOR1, FGFR2, GCC, cKIT, LIV1, LY6E, MSLN, MUC16, NaPi2b, Nectin4, gpNMB, PSMA, SLITRK6, STEAPI, TROP2, 5T4, SSEA4, GloboH, Gb5, STn, Tn, B7H3, BCMA, MUC1, cMet, ROR1 MSLN, FRa, CLDN18.2, CLDN6, PTK7, Axl, FGFR2b, CLL1, CCR7, GPC1, GPC3, ISAC, CDCP1, ITGB6, ADAM9, or CD45.

69. The conjugate of any one of claims 65-68, or a pharmaceutically acceptable salt or solvate thereof, selected from:

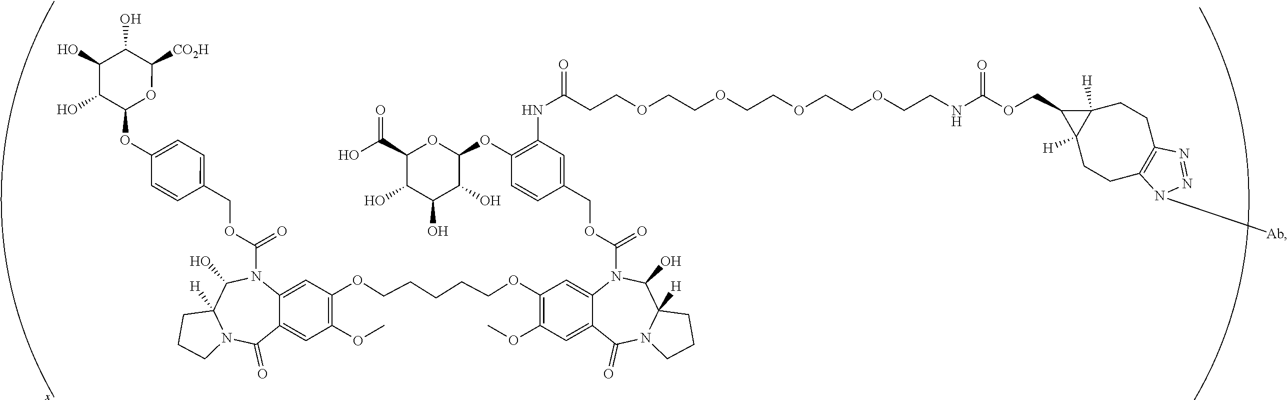

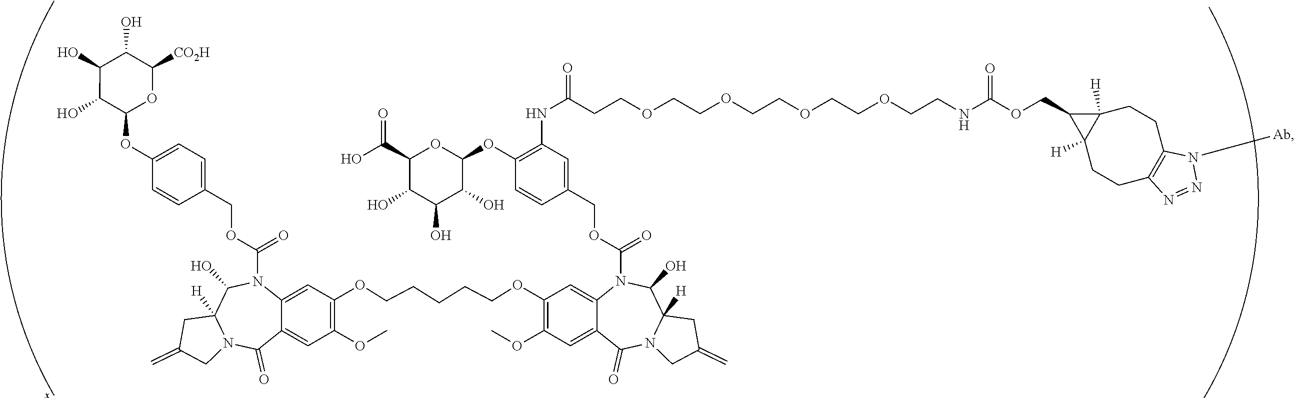

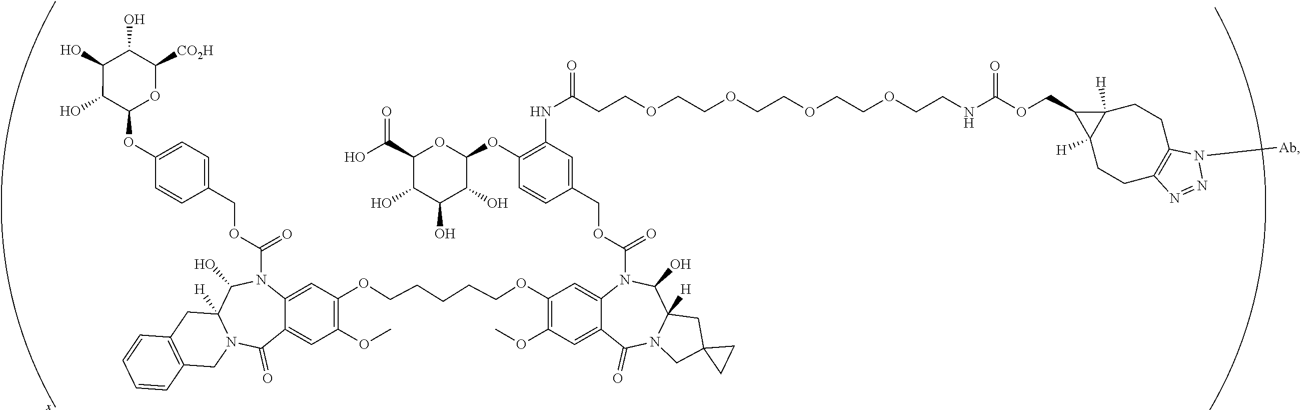

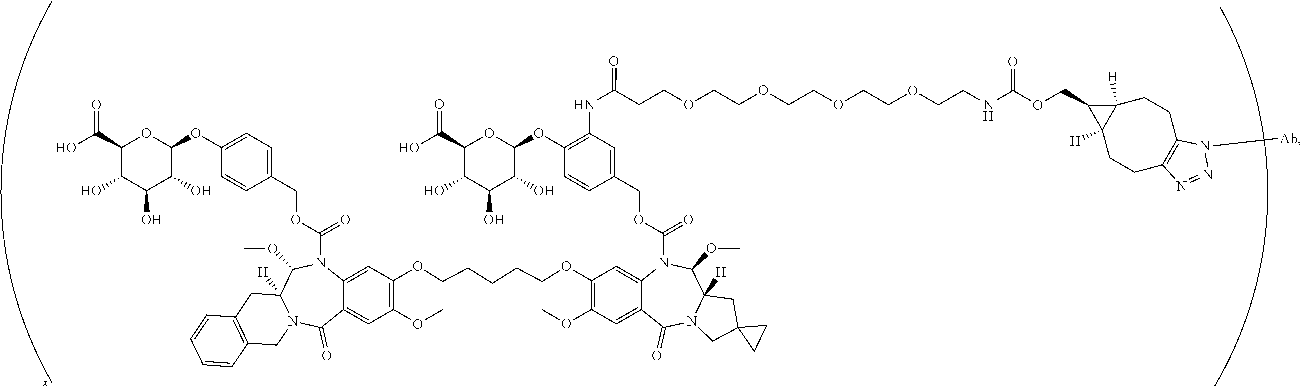

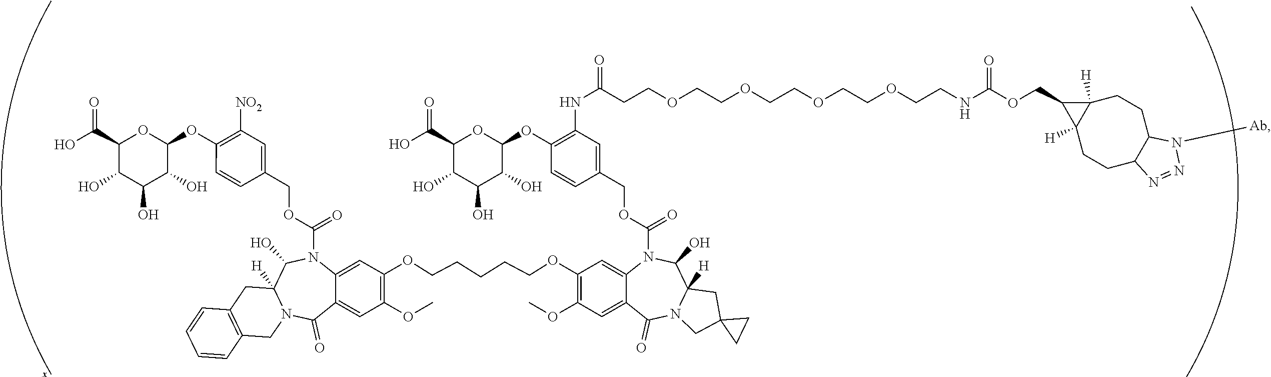

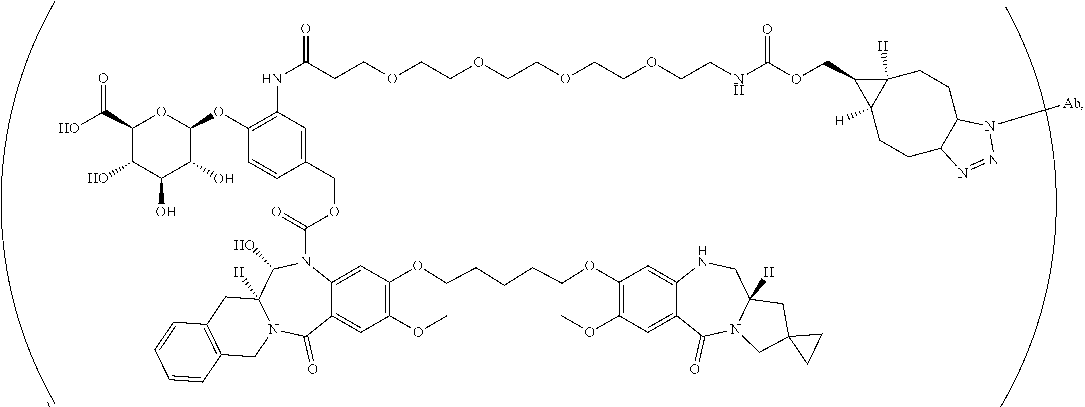

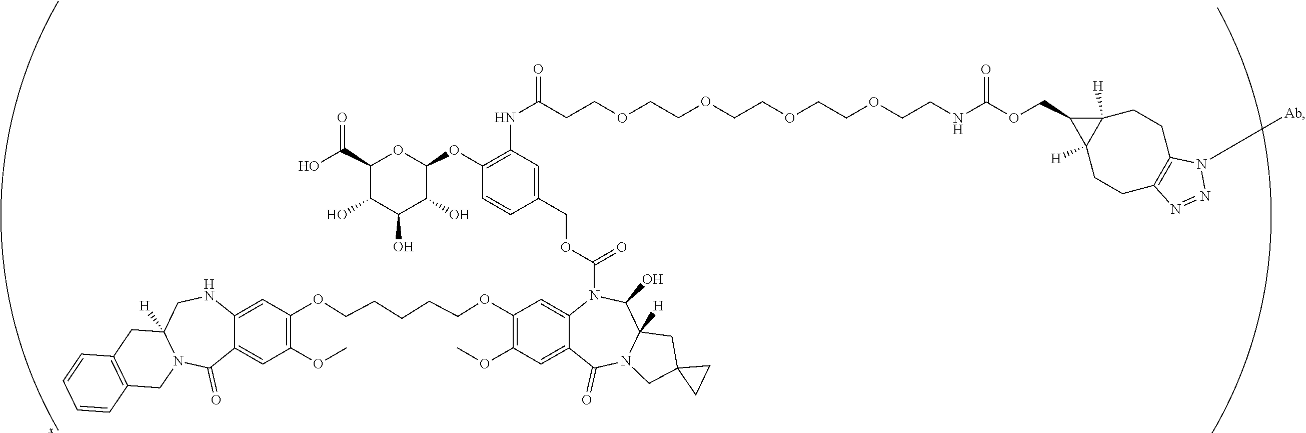

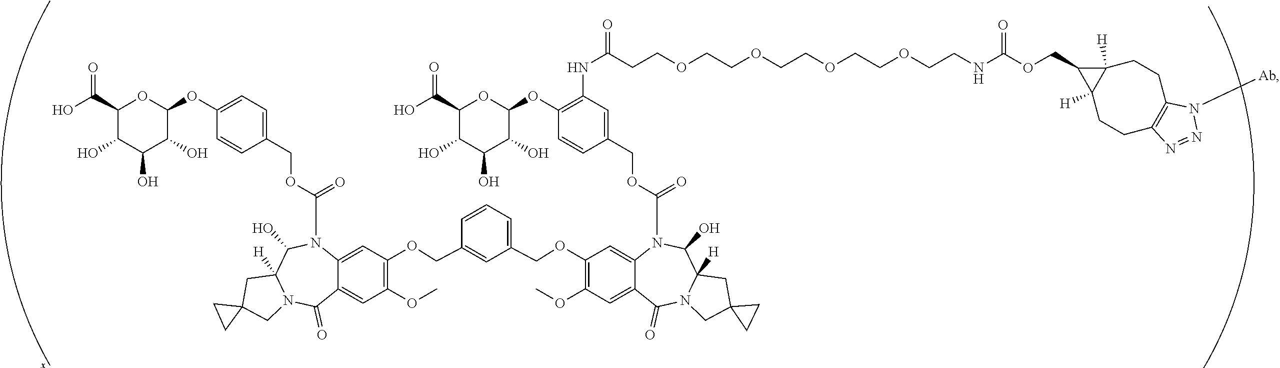

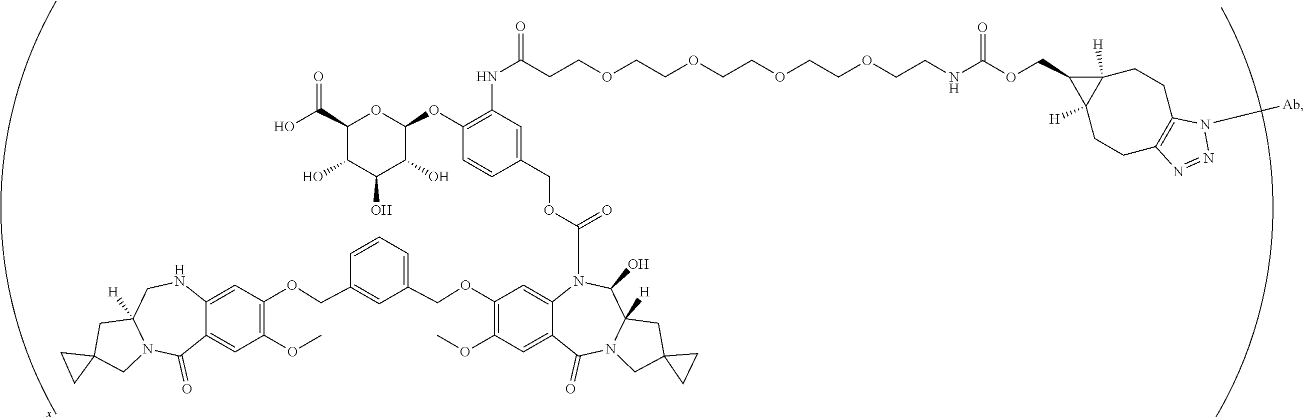

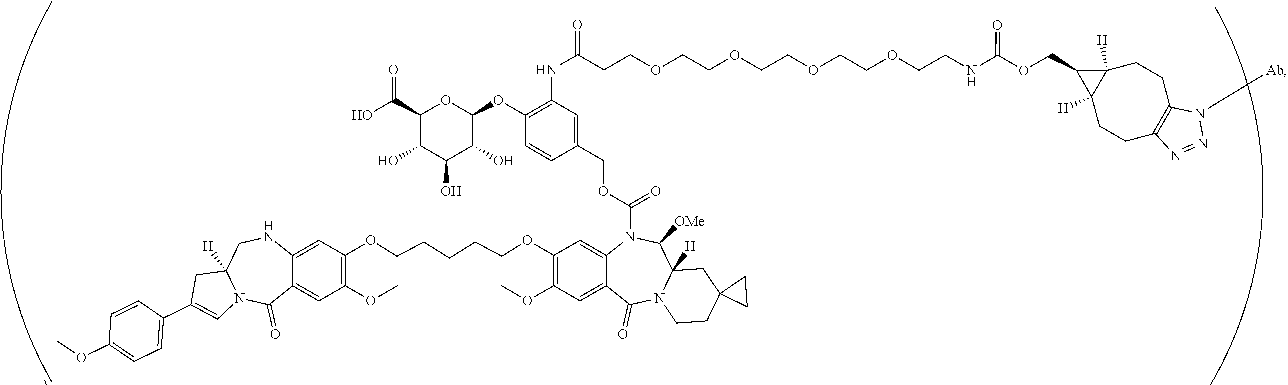

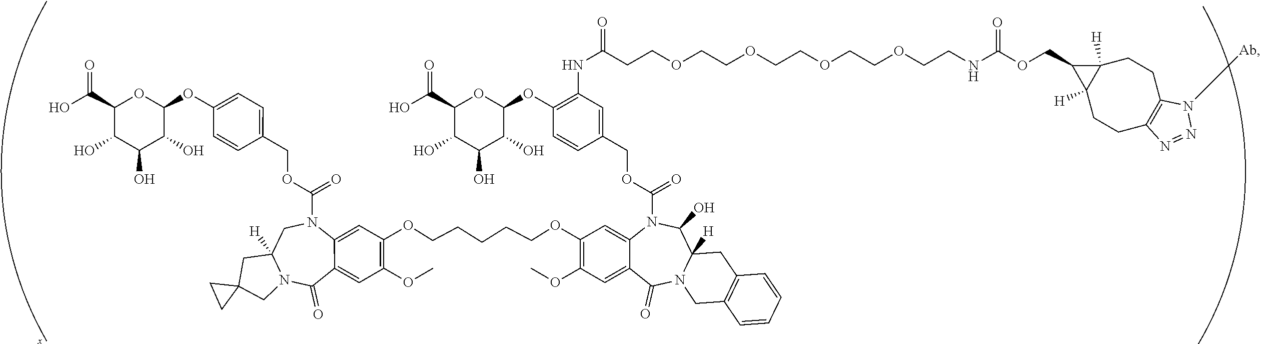

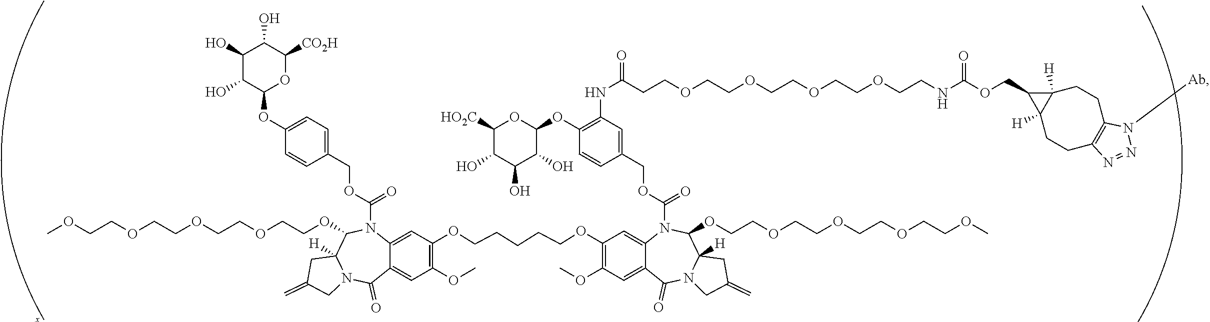

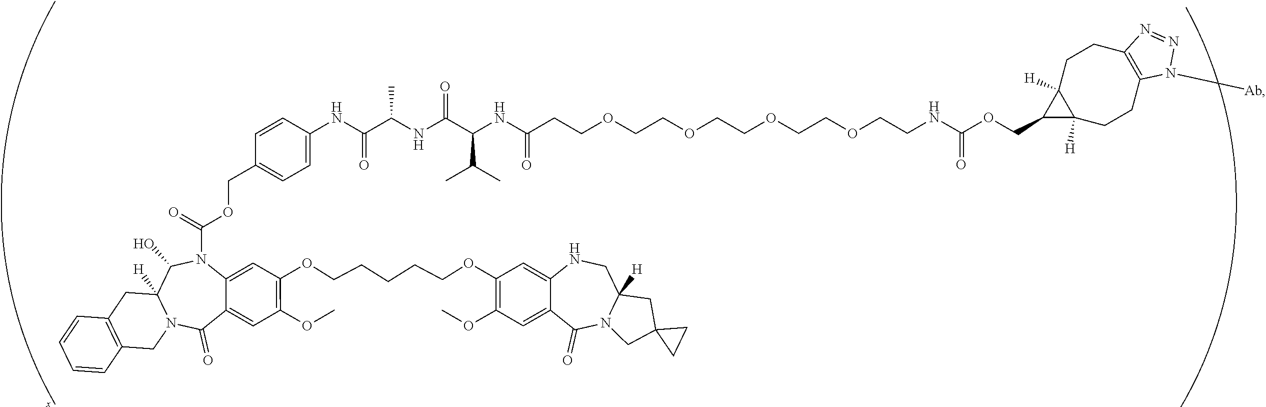

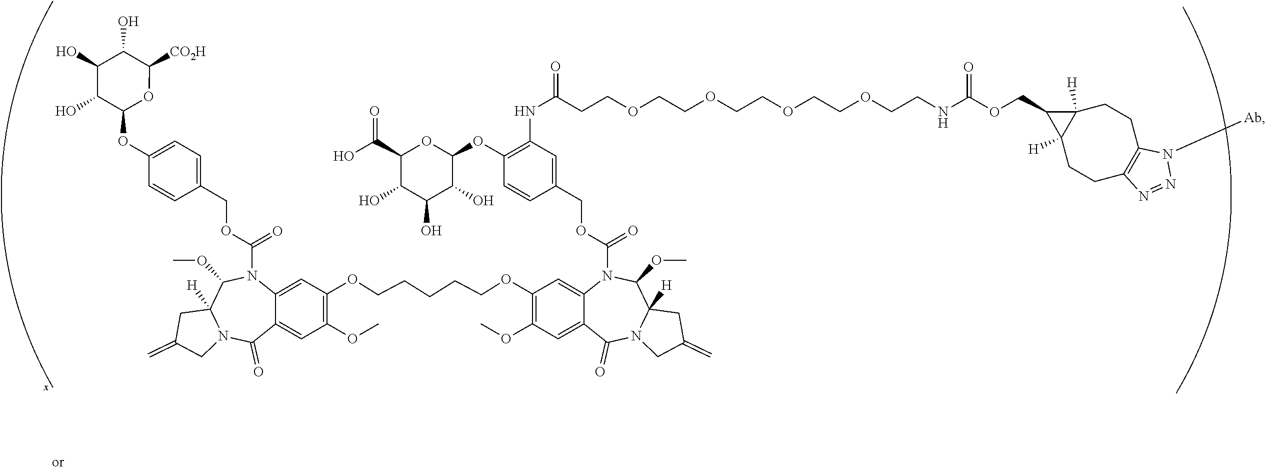

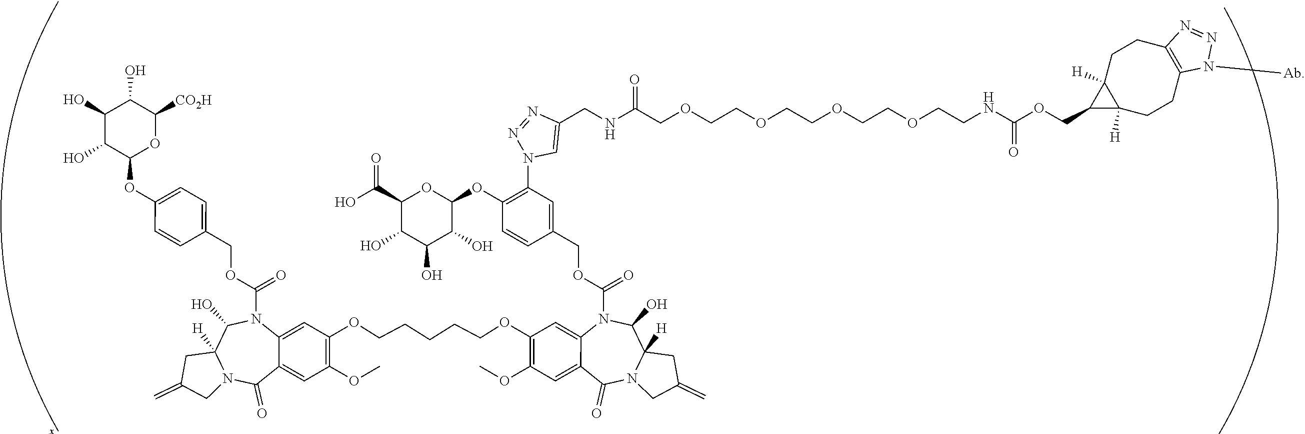

70. The conjugate of claim 69, wherein x is about 1.8 to about 2.5.

71. The conjugate of claim 69 or 70, wherein Ab is STRO-001.

72. A pharmaceutical composition comprising the conjugate of any one of claims 65-71, or a pharmaceutically acceptable salt, tautomer, solvate, stereoisomer, or isotopologue thereof, and a pharmaceutically acceptable excipient.

73. A method of treating a proliferative disease, a metabolic disease, inflammation, or a neurodegenerative disease in a subject in need thereof, comprising administering to the subject an effective amount of the conjugate of any one of claims 65-71, or a pharmaceutically acceptable salt, tautomer, solvate, stereoisomer, or isotopologue thereof, or a pharmaceutical composition of claim 72.
